(19) United States
(12) Patent Application Publication
(10) Pub. No.: US 2024/0299387 A1
(43) Pub. Date: Sep. 12, 2024

(54) HETEROCYCLIC EGFR INHIBITORS FOR USE IN THE TREATMENT OF CANCER

(71) Applicant: Blueprint Medicines Corporation, Cambridge, MA (US)

(72) Inventors: Natasja Brooijmans, Cambridge, MA (US); Jason D. Brubaker, Cambridge, MA (US); John Emmerson Campbell, Cambridge, MA (US); Christopher De Savi, Cambridge, MA (US); Thomas A. Dineen, Cambridge, MA (US); Meredith Suzanne Eno, Cambridge, MA (US); Joseph L. Kim, Cambridge, MA (US); Aysegul Ozen, Cambridge, MA (US); Emanuele Perola, Cambridge, MA (US); Brett D. Williams, Cambridge, MA (US); Douglas Wilson, Cambridge, MA (US); Kevin J. Wilson, Cambridge, MA (US)

(21) Appl. No.: 18/572,348

(22) PCT Filed: Jun. 21, 2022
(86) PCT No.: PCT/US2022/034411

§ 371 Date: Dec. 20, 2023

Related U.S. Application Data
(60) Provisional application No. 63/310,772, filed on Feb. 16, 2022.
(60) Provisional application No. 63/213,349, filed on Jun. 22, 2021.

(51) Int. Cl. A61K 31/506; A61K 31/517; A61K 31/5377; C07D 401/14; C07D 413/14; C07D 471/04; C07D 487/04; C07D 491/048

(52) U.S. Cl. CPC A61K 31/506; A61K 31/517; A61K 31/5377; C07D 401/14; C07D 413/14; C07D 471/04; C07D 487/04; C07D 491/048

(57) ABSTRACT

The present disclosure provides a compound represented by structural formula (I): or a pharmaceutically acceptable salt thereof useful for treating a cancer.

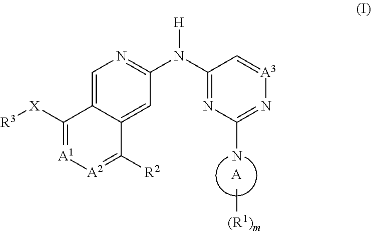

### CROSS-REFERENCE TO RELATED APPLICATIONS

This application claims priority to U.S. Provisional Application No. 63/213,349, filed on Jun. 22, 2021, and U.S. Provisional Application No. 63/310,772, filed on Feb. 16, 2022. The entire contents of the aforementioned applications are incorporated herein by reference.

### TECHNICAL FIELD

This disclosure relates to compounds and compositions useful for treating disorders related to certain mutant forms of EGFR.

### BACKGROUND

EGFR (Epidermal Growth Factor Receptor) is a member of the erbB receptor family, which includes transmembrane protein tyrosine kinase receptors. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors, EGFR gene amplification and alterations such as mutations, deletions and the like, can promote malignant transformation of cells and play an important role in tumor cell proliferation, invasion, metastasis and angiogenesis. For example, alterations such as mutations and deletions in the EGFR gene are found in non-small lung cancer (NSCLC) tumors. The two most frequent EGFR alternations found in NSCLC tumors are short in-frame deletions in exon 19 (del19) and L858R, a single missense mutation in exon 21 (Cancer Discovery 2016 6(6) 601). These two alterations cause ligand-independent EGFR activation and are referred to as primary or activating mutations in EGFR mutant NSCLC (EGFR M+). Clinical experience shows an objective response rate (ORR) of approximately 60-85% in EGFR M+ NSCLC patients treated first line (1 L) with EGFR tyrosine kinase inhibitors (TKIs) erlotinib, gefitinib, afatinib and osimertinib (Lancet Oncol. 2010 Vol. 11, 121; Lancet Oncol. 2016 Vol. 17, 577; N. Engl. J. Med. 2017 Nov. 18 Doi:10.1056/NEJMoa1713137; Lancet Oncol. 2011 Vol. 12, 735), thus demonstrating that EGFR mutant NSCLC tumors depend on oncogenic EGFR activity for survival and proliferation and establishing del19 and L858R mutated EGFR as oncogenic drivers of disease and thus, validating drug targets and biomarkers for the treatment of NSCLC.

However, after an average of 10-12 months of treatment with first generation (erlotinib and gefitinib) and second generation (afatinib) EGFR TKIs, resistance to these small molecule inhibitors has been observed in almost all NSCLC patients (Lancet Oncol. 2010 February; 11(2):121-8.; Lancet Oncol. 2016 May; 17(5):577-89; Lancet Oncol. 2011 August; 12(8):735-42). The most prominent resistance mechanism to first and second generation EGFR TKIs is due to the secondary mutation in EGFR of T790M, occurs in 50% to 70% of patients progressing on 1st and 2nd generation EGFR inhibitors. (Blakely, Cancer Discov; 2(10); 872-5, 2012; Kobayashi, Cancer Res., 65:(16), 2005). This secondary mutation reduces the affinity of the drug with the target, thereby producing drug resistance, and resulting in tumor recurrence or disease progression.

In view of the prevelance of this mutation in drug resistance produced in therapy targeting EGFR of lung cancer, a number of companies have attempted to develop new small molecule EGFR inhibitors for treating these patients with drug-resistant lung cancer by inhibiting the resistant mutant EGFR-T790M. For example, osimertinib (Tagrisso®), a third generation EGFR TKI, has been developed to treat NSCLC patients if the cancer cells are positive for the primary EGFR mutations del19 or L858R with or without the T790M mutation in the gene coding for EGFR.

Although the third generation EGFR TKI, osimertinib, has shown efficacy on NSCLC patients, unfortunately, resistance mediated by an exon 20 C797 mutation in EGFR usually develops within approximately 10 months (European Journal of Medicinal Chemistry 2017 Vol. 142: 32-47) and accounts for the majority of osimertinib resistance cases (Cancer Letters 2016 Vol. 385: 51-54). The EGFR del19/L858R T790M C797S cis mutant kinase variant typically emerges in second line (2 L) patients following treatment with osimertinib and is often referred to as “triple mutant” EGFR and it can no longer be inhibited by first, second, or third generation EGFR inhibitors.

No approved EGFR TKI can inhibit the triple mutant variant. Therefore, there is a need to develop new EGFR inhibitors, which can inhibit with high selectivity EGFR mutants with the triple mutant, del19/L858R T790M C797S, while at the same time have no or low activity to wild-type EGFR. In addition to treating a mutant form of EGFR for which there is no current therapy, such selective EGFR inhibitors are likely to be more suitable as therapeutic agents, particularly for the treatment of cancer, due to reduction of toxicologies (diarrhea, skin rash) associated with wild-type EGFR inhibition.

### SUMMARY

The applicant has discovered novel compounds which are effective inhibitors of certain mutant forms of EGFR (see Synthetic Examples 1-91). In particular, it has been demonstrated that the compounds of the present disclosure effectively inhibit certain mutant forms of EGFR. Compounds of the disclosure (also referred to herein as the “disclosed compounds”) or pharmaceutically acceptable salts thereof effectively inhibit EGFR with one or more alterations, including L858R and/or exon 19 deletion mutation, T790M mutation, and/or C797S mutation. Compounds of the disclosure or pharmaceutically acceptable salts thereof effectively inhibit EGFR with L858R and/or exon 19 deletion mutation, T790M mutation, and C797S mutation (hereinafter “EGFR with LRTMCS mutations” or “triple mutant EGFR”) (see Biological Example 1) and can be used treat various cancers, for example, lung cancer (see Biological Example 2). Importantly, the disclosed compounds are selective EGFR inhibitors, i.e., the disclosed compounds have no or low activity against wild-type EGFR and the kinome. Advantages associated with such selectivity may include facilitating efficacious dosing and reducing EGFR-mediated on-target toxicities. Some of the disclosed compounds exhibit good penetration of the brain and blood brain barrier (e.g., a PGP efflux ratio of less than 5). As such, the compounds of the disclosure or pharmaceutically acceptable salts thereof are expected to be effective for the treatment of metastatic cancer, including brain metastesis, including leptomeningeal disease and other systemic metastesis. Some of the disclosed compounds also have the advantage of having high microsomal stability. Compounds of the disclosure also may have favorable toxicity profiles related to other non-kinase targets.

In one aspect, the present disclosure provides a compound represented by the following structural Formula (I):

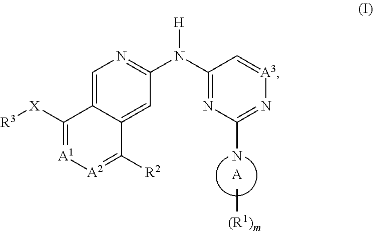

or a pharmaceutically acceptable salt thereof, wherein:
each A¹, A², and A³ is independently N or CR; wherein each R is independently H, halogen, or CH₃;
Ring A is 4-12 membered heterocyclyl;
each R¹ is independently halogen, CN, OH, NR^{a}R^{b}, C₁-C₄ alkyl, C₁-C₄ alkoxy, C₃-C₆ cycloalkyl, or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is H, halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H, or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4 to 12-membered heterocycyl, and 5 to 10 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl); or
R³ is C₃-C₆cycloalkyl optionally substituted with 1 to 3 groups selected from C₁-C₄alkyl, halogen, OR^{a}, CN, NR^{a}R^{b}, NHC(O)CH₃, and S(O)₂CH₃, or
R³ is 4 to 10 membered heterocyclyl or 5 to 6-membered heteroaryl, each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (I)) or a pharmaceutically acceptable salt thereof or a pharmaceutical composition of the disclosure. In one embodiment, the cancer is non-small cell lung cancer. In another embodiment, the subject cancer has metastasized to the brain. In another embodiment, the subject has brain metastasis from non-small cell lung cancer.

In one embodiment, the cancer to be treated has epidermal growth factor receptor (EGFR) L858R mutation and/or exon 19 deletion mutation and T790M mutation. In another embodiment, the cancer to be treated may further has epidermal growth factor receptor (EGFR) L858R mutation and/or exon 19 deletion mutation and the T790M mutation and the C797S mutation. In another embodiment, the cancer to be treated in either of the foregoing embodiments is lung cancer, e.g., non-small cell lung cancer. In a specific embodiment, the cancer is non-small cell lung cancer with brain metastasis.

The treatment method disclosed herein further comprises administering to the subject an effective amount of afatinib, osimertinib, erlotinib, or gefitinib.

The present disclosure also provides a method of inhibiting epidermal growth factor receptor (EGFR) in a subject in need thereof, comprising administering to the subject an effective amount of a compound of the disclosure (e.g., a compound of Formula (I)) or a pharmaceutically acceptable salt thereof or a pharmaceutical composition of the disclosure.

The present disclosure also provides the use of an effective amount of a compound of the disclosure (e.g., a compound of Formula (I)), or a pharmaceutically acceptable salt thereof, or a pharmaceutical composition of the disclosure, for the preparation of a medicament for the treatment of cancers.

In another aspect, provided herein a compound of Formula (I), or a pharmaceutically acceptable salt thereof, or a pharmaceutical composition of the disclosure for use in treating cancers.

### DETAILED DESCRIPTION

### Definitions

The term “halo” as used herein means halogen and includes chloro, fluoro, bromo and iodo.

The term “alkyl” used alone or as part of a larger moiety, such as “alkoxy” and the like, means saturated aliphatic straight-chain or branched monovalent hydrocarbon radical. Unless otherwise specified, an alkyl group typically has 1-4 carbon atoms, i.e. (C₁-C₄)alkyl. As used herein, a “(C₁-C₄)alkyl” group means a radical having from 1 to 4 carbon atoms in a linear or branched arrangement. Examples include methyl, ethyl, n-propyl, iso-propyl, n-butyl, iso-butyl, sec-butyl, and the like.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “aryl” refers to a monovalent radical of an aromatic hydrocarbon ring system. Representative aryl groups include fully aromatic ring systems, such as phenyl, naphthyl, and anthracenyl, and ring systems where an aromatic carbon ring is fused to one or more non-aromatic carbon rings, such as indanyl, phthalimidyl, naphthimidyl, or tetrahydronaphthyl, and the like.

The term “cycloalkyl” refers to a monocyclic saturated hydrocarbon ring system. Unless otherwise specified, cycloalkyl has from 3-6 carbon atoms. For example, a C₃-C₆ cycloalkyl includes cyclopropyl, cyclobutyl, cyclopentyl and cyclohexyl.

“Heteroaryl” refers to a monovalent radical of a 5- to 12-membered (or 5- to 10-membered) heteroaromatic ring system. A heteroaryl has ring carbon atoms and 1 to 4 ring heteroatoms, independently selected from O, N, and S. Representative heteroaryl groups include ring systems (e.g., monocyclic, bicyclic, or polycyclic) where: (i) each ring comprises a heteroatom and is aromatic, e.g., imidazolyl, oxazolyl, isoxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl; (ii) each ring is aromatic or carbocyclyl, at least one aromatic ring comprises a heteroatom and at least one other ring is a hydrocarbon ring or e.g., indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, carbazolyl, acridinyl, phenazinyl, phenothiazinyl, phenoxazinyl, pyrido[2,3-b]-1,4-oxazin-3-(4H)-one, 5,6,7,8-tetrahydroquinolinyl and 5,6,7,8-tetrahydroisoquinolinyl; and (iii) each ring is aromatic or carbocyclyl, and at least one aromatic ring shares a bridgehead heteroatom with another aromatic ring, e.g., 4H-quinolizinyl.

The term “heterocyclyl” or “heterocyclic” refers to a radical of a 4- to 12-(or 4 to 10)-membered saturated or partially saturated ring system (“4-12 membered heterocyclyl” or (“4-10 membered heterocyclyl”) having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone. In heterocyclyl groups that contain one or more nitrogen atoms, the point of attachment can be a carbon or nitrogen atom, as valency permits. Heterocyclic ring includes at least one saturated or partially saturated ring that contains a heteroatom. A heterocyclyl group can either be monocyclic (“monocyclic heterocyclyl”) or polycyclic (e.g., a bicyclic system (“bicyclic heterocyclyl”) or tricyclic system (“tricyclic heterocyclyl”); and bicyclic and polycyclic ring systems include fused, bridged, or spiro ring systems). Exemplary monocyclic heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system. Substituents (e.g., R¹) may be present on one or more rings in the polycyclic ring system.

Representative heterocyclyls include ring systems in which: (i) every ring is non-aromatic and at least one ring comprises a heteroatom, e.g., tetrahydrofuranyl, tetrahydropyranyl, oxetanyl, azetidinyl, tetrahydrothienyl, pyrrolidinyl, pyrrolidonyl, piperidinyl, pyrrolinyl, decahydroquinolinyl, oxazolidinyl, piperazinyl, dioxanyl, dioxolanyl, diazepinyl, oxazepinyl, thiazepinyl, morpholinyl, quinuclidinyl, and (3aR,6aS)-hexahydro-1□²-furo[3,4-b]pyrrole; (ii) at least one ring is non-aromatic and comprises a heteroatom and at least one other ring is an aromatic carbon ring, e.g., 1,2,3,4-tetrahydroquinolinyl, 1,2,3,4-tetrahydroisoquinolinyl; and (iii) at least one ring is non-aromatic and comprises a heteroatom and at least one other ring is aromatic and comprises a heteroatom e.g., 6,7-dihydro-5H-pyrrolo[1,2-c]imidazole.

In some embodiments, a heterocyclyl group is a 8-12 membered bicyclic heterocyclyl, e.g., wherein a saturated or partially saturated heterocyclyl is fused to an aromatic or heteroaromatic ring. The term “heterocyclyl” can also include 8-12 membered bicyclic heterocyclyls, wherein a saturated or partially saturated cycloalkyl is fused to an aromatic or heteroaromatic ring. The point of attachment of the heterocyclyl to the rest of the molecule can be through the saturated or partially saturated heterocyclyl or cycloalkyl, or through the aromatic or heteroaromatic ring.

In some embodiments, a bridged bicyclic system has at two non-aromatic rings containing from 7-12 ring atoms (heterocyclyl or cycloalkyl) and which share three or more atoms, with the two bridgehead atoms separated by a bridge containing at least one atom. “Bridged heterocyclyl” includes bicyclic or polycyclic hydrocarbon or aza-bridged hydrocarbon groups; examples include 2-azabicyclo[2.2.1]heptanyl, 3-azabicyclo[3.2.1]octanyl, 6-oxa-2-azabicyclo[3.2.1]octanyl, 6-oxa-3-azabicyclo[3.2.1]octanyl, and 8-oxa-3-azabicyclo[3.2.1]octanyl.

In some embodiments, a fused bicyclic system has two non-aromatic rings (heterocyclyl or cycloalkyl) containing from 7-12 ring atoms and which share two adjacent ring atoms. Examples of fused bicyclic systems include hexahydro-1H-furo[3,4-b]pyrrolyl, hexahydro-1H-furo[3,4-c]pyrrolyl, 6,7-dihydro-5H-pyrrolo[1,2-c]imidazole, (3aR,6aS)-hexahydro-1□²-furo[3,4-b]pyrrole.

In some embodiments, a spiro bicyclic system has two non-aromatic rings containing (heterocyclyl or cycloalkyl) from 7-12 ring atoms and which share one ring atom. Examples of spiro bicyclic systems include 1-oxa-7-azaspiro[3.5]nonan-7-yl, 1,4-dioxa-8-azaspiro[4.5]decan-8-yl, and 1,4-dioxa-9-azaspiro[5.5]undecan-9-yl.

### Compounds of the Present Disclosure

Disclosed herein are embodiments of compounds having a general structure of Formula (I). These compounds are selective inhibitors of LRTM and LRTMCS EGFR. In contrast to other EGFR inhibitors such as osimertinib which binds EGFR irreversibly, the compounds of the disclosure are non-covalent inhibitors.

In a first embodiment, the present disclosure provides a compound represented by the following structural formula (Ia):

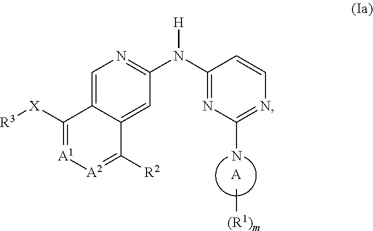

or a pharmaceutically acceptable salt thereof, wherein:
each A¹, and A² is independently N or CR, wherein each R is independently H, halogen, or CH₃;
Ring A is 6-membered heterocyclyl or a 8-membered bicyclic heterocyclyl;
each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy;
m is 0, 1, 2, 3, 4, 5, or 6;
R² is halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H, or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-5-membered heterocycyl, and 5 membered heteroaryl, wherein the heterocycyl and the heteroaryl are optionally substituted with 1 to 3 groups selected from C₁-C₄alkyl, halogen, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6 member heterocyclyl optionally substituted with 1 to 3 groups selected from halogen and C₁-C₄ alkyl, wherein the alkyl is optionally substituted with 1 to 3 halo
each R^{a} and R^{b} is independently H or C₁-C₄ alkyl.

In some embodiments, the present disclosure provides a compound represented by the structural Formula (I) or (Ia) above, wherein each A¹ and A² are each independently N or CR and A³ is CR; wherein each R is independently H, halogen, or CH₃. In some embodiments, the compound is a compound of Formula (I) or (Ia) above, wherein A³ is CR and A¹ and A² are both CR or one or one of A¹ and A² is N and one of A¹ and A² is CR; wherein each R is independently H, halogen, or CH₃.

In some embodiments, the compound is a compound of Formula (I) or (Ia) above, wherein A³ is CR and A¹ and A² are both CR, wherein each R is independently H, halogen, or CH₃. In some embodiments, the compound is a compound of Formula (I) or (Ia) above, wherein A³ is CR and A¹ is N and and A² is CR; wherein each R is independently H, halogen, or CH₃. In some embodiments, the compound is a compound of Formula (I) or (Ia) above, wherein A³ is CR and A² is N and and A¹ is CR; wherein each R is independently H, halogen, or CH₃.

In some embodiments, the present disclosure provides a compound represented by the structural Formula (I) or (Ia) above, wherein Ring A is 6-member heterocyclyl and each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and m is 0, 1, 2, 3, 4, 5, or 6.

In some embodiments, a compound is a compound of Formula (I) or (Ia) above, wherein Ring A is 6-member heterocyclyl and each R¹ is independently halogen, CN, OH, NR^{a}R^{b}, C₁-C₄ alkyl, C₁-C₄ alkoxy, and m is 0, 1, 2, 3, 4, 5, or 6. In some embodiments, a compound is a compound of Formula (I) or (Ia) above, wherein Ring A is 6-member heterocyclyl and each R¹ is independently halogen, OH, or methyl, and m is 0, 1, 2, 3, 4, 5, or 6.

In some embodiments, a compound is a compound of Formula (I) or (Ia) above, wherein R² is selected from the group consisting of: H, halogen, C₁-C₄ alkyl, C₃-C₆ cycloalkyl, wherein the alkyl or cycloalkyl is optionally substituted with 1 to 3 halogen. In some embodiments, R² is selected from the group consisting of: H, F, CH₃, and cyclopropyl, wherein the alkyl is optionally substituted with 1 to 3 F. In some embodiments, R² is selected from the group consisting of H, F, CH₃,

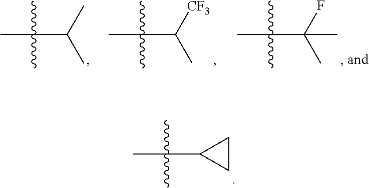

In some embodiments, a compound is a compound of Formula (I) or (Ia) above, wherein Ring A, R₁ and m are

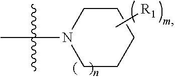

wherein each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy;
m is 0, 1, 2, 3, 4, 5, or 6;
n is 0 or 1; and
each R^{a} and R^{b} is independently H or C₁-C₄ alkyl.

In some embodiments, a compound is a compound of Formula (I) or (Ia) above, wherein Ring A and (R¹)ₘ are

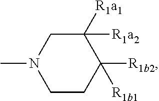

wherein each R₁ₐ₁, R₁ₐ₂, R_{1b2}, and R_{1b1} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R₁ₐ₁, R₁ₐ₂, R_{1b2}, and R_{1b1} is optionally substituted with 1 to 3 groups selected from deuterium, halogen, OH, NR^{a}R^{b}, C₁-C₂ alkyl, and C₁-C₂ alkoxy; and each R^{a} and R^{b} is independently H or C₁-C₄ alkyl.

In some embodiments, a compound is a compound of Formula (I) or (Ia) above, wherein Ring A and (R¹)ₘ are

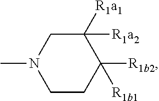

wherein each R₁ₐ₁, R₁ₐ₂, R_{1b2}, and R_{1b1} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and each R^{a} and R^{b} is independently H or C₁-C₄ alkyl.

In some embodiments, a compound is a compound of Formula (I) or (Ia) above, wherein Ring A and (R₁)ₘ are

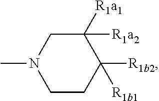

wherein R₁ₐ₁ is H, halogen, OH, or methyl; R₁ₐ₂ is H, or halogen; and

R_{1b2} is F, OH, methyl, or methoxy, wherein the methyl and methoxy is optionally substituted with OH, NR^{a}R^{b} or one or to 3 deuterium; and each R^{a} and R^{b} is independently H or C₁-C₄ alkyl; and R_{1b1} is H, or CH₃.

In some embodiments, a compound is a compound of Formula (I) or (Ia) above, wherein Ring A and (R₁)ₘ are

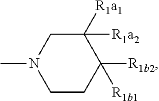

wherein R₁ₐ₁ is F and R₁ₐ₂ is H, F, or CH₃; or R₁ₐ₁ is OH and R₁ₐ₂ is H; or R₁ₐ₁ is H and R₁ₐ₂ is H;

R_{1b2} is OR^{a} where R^{a} is H or C₁-C₄ alkyl optionally substituted with OH, NR^{a}R^{b} or one or to 3 deuterium, and R_{1b1} is H or CH₃; and each R^{a} and R^{b} is independently H or methyl.

In some embodiments, a compound is a compound of Formula (I) or (Ia) above, wherein Ring A and (R₁)ₘ are

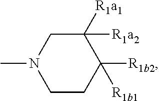

wherein R₁ₐ₁ is H, F or OH and R₁ₐ₂ is H, F or CH₃; R_{1b21} is OR^{a} where R^{a} is H or C₁-C₄ alkyl optionally substituted with OH, NR^{a}R^{b} or one to 3 deuterium, and R_{1b1} is H or CH₃; and each R^{a} and R^{b} is independently H or methyl. Alternatively, R₁ₐ₁ is H, F or OH and R₁ₐ₂ is H, F or CH₃; R_{1b2} is OH, OCH₃ or OCD₃, and R_{1b1} is H or CH₃.

In some embodiments, a compound is a compound of Formula (I) above, wherein Ring A and (R₁)ₘ are selected from the group consisting of:

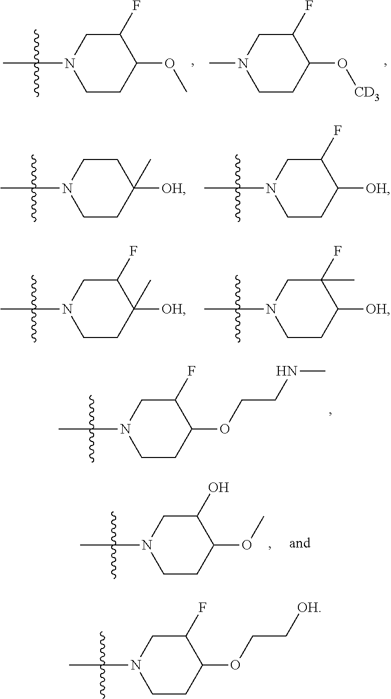

In some embodiments, a compound can be a compound of Formula (I) or (Ia) that is also a compound of Formula (II),

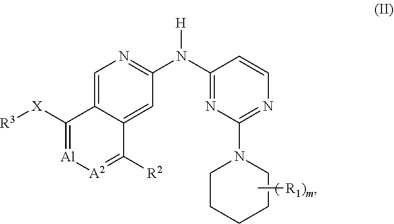

or a pharmaceutically acceptable salt thereof, wherein:
each A¹, and A² is independently N or CR; wherein each R is independently H, halogen, or CH₃;
each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H, or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-6-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6 member heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

In some embodiments, a compound can be a compound of Formula (II) that is also a compound of Formula (IIa)

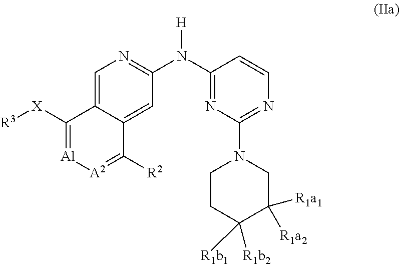

or a pharmaceutically acceptable salt thereof, wherein:
A¹ is N or CR and A² is CR; or A¹ is CR and A² is N, wherein each R is independently H, halogen, or CH₃;
each R₁ₐ₁, R₁ₐ₂, R_{1b1}, and R_{1b2} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R₁ₐ₁, R_{1a2, 1ba1}, and R_{1b2} is optionally substituted with 1 to 3 groups selected from deuterium, halogen, OH, NR^{a}R^{b}, C₁-C₂ alkyl, and C₁-C₂ alkoxy; and each R^{a} and R^{b} is independently H or C₁-C₄ alkyl;
R² is halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-5-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6 member heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

In some embodiments, a compound can be a compound of Formula (II) that is also a compound of Formula (IIb)

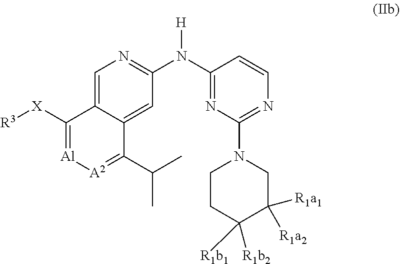

or a pharmaceutically acceptable salt thereof, wherein:
A¹ is N or CR and A² is CR or A¹ is CR and A² is N, wherein each R is independently H, halogen, or CH₃;
each R₁ₐ₁, R₁ₐ₂, R_{1b1}, and R_{1b2} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R₁ₐ₁, R₁ₐ₂, R_{1b1}, and R_{1b2} is optionally substituted with 1 to 3 groups selected from deuterium, halogen, OH, NR^{a}R^{b}, C₁-C₂ alkyl, and C₁-C₂ alkoxy; and each R^{a} and R^{b} is independently H or C₁-C₄ alkyl;
X is O, NH, or —C(O)—NH—, wherein the R³ is attached to the NH— of —C(O)NH—;
R³ is H or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-5-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6-member heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

In some embodiments, the compound is a compound of Formula (I), (Ia), (II), (IIa), or (IIb) wherein X is O and R³ is C₁-C₄alkyl optionally substituted with a 4 to 6-member heterocyclyl, 5-member heteroaryl, or 5-member heterocyclyl fused to a 5-member heteroaryl, wherein each heteroaryl or heterocyclyl is each optionally substituted with 1-3 groups selected from halogen, C₁-C₄alkyl, ═O, and —C(O)CH₃.

In some embodiments, the compound is a compound of Formula (I), (Ia), (II), (IIa), or (IIb) wherein X is O and R³ is C₁-C₄alkyl optionally substituted with NHC(O)CH₃.

In some embodiments, the compound is a compound of Formula (I), (Ia), (II), (IIa), or (IIb) wherein X is —O— and R³ is a C₁-C₄alkyl substituted with a 5-member heteroaryl comprising 1 to 3 nitrogens and 0 or 1 oxygen atoms wherein the heteroaryl is optionally substituted with 1 to 3 groups selected from C₁-C₄alkyl, C(O)CH₃, and S(O)₂CH₃. In some embodiments, the compound is a compound of Formula (I), (Ia), (II), (IIa), or (IIb) wherein X is —O— and R³ is a C₁-C₄alkyl substituted with a triazole, imidazole or isoxazole optionally substituted with one or more C₁-C₄alkyl. In some embodiments, the compound is a compound of Formula (I), (Ia), (II), (IIa), or (IIb) wherein X and R³ are together selected from the group consisting of:

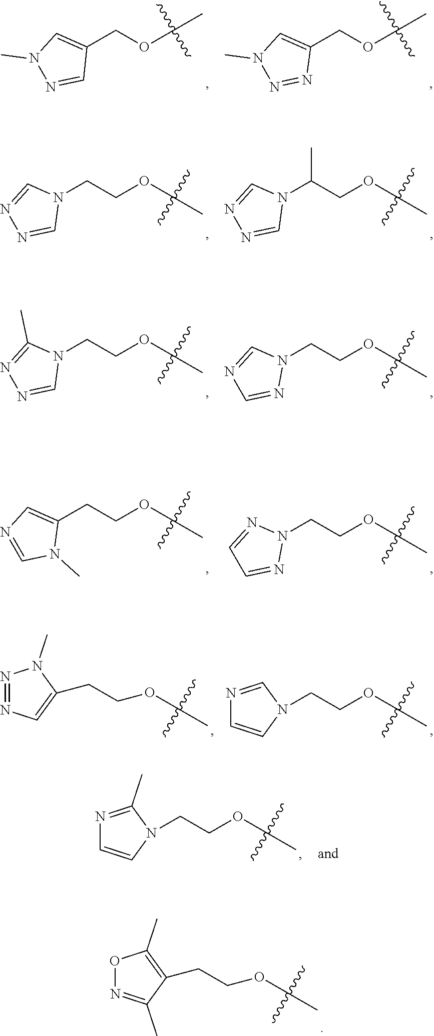

In some embodiments, the compound is a compound of Formula (I), (II), (IIa), or (IIb) wherein X is —O— and R³ is C₁-C₄alkyl substituted with a 4- or 5-member heterocyclyl comprising one or two N atoms and 0 or 1 oxygen atoms, wherein the heterocyclyl is each optionally substituted with C₁-C₄alkyl, acetyl, or S(O)₂CH₃, and. In some embodiments, the compound is a compound of Formula (I), (Ia), (II), (IIa), or (IIb) wherein X is —O— and R³ is a C₁-C₄alkyl substituted with pyrrolidone or oxazolidinone optionally substituted with one or more C₁-C₄alkyl or azetidine optionally substituted with acetyl or C₁-C₄alkyl optionally substituted with one or more halogen. In some embodiments, the compound is a compound of Formula (I), (Ia), (II), (IIa), or (IIb) wherein X and R³ are together selected from the group consisting of:

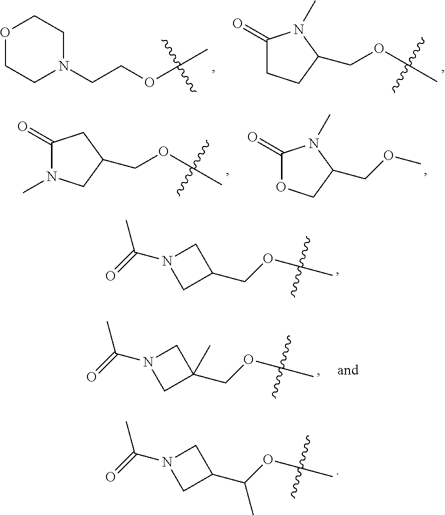

In some embodiments, the compound is a compound of Formula (I), (Ia), (II), (IIa), or (IIb) wherein X is NH and R³ is C₁-C₄alkyl optionally substituted with NR^{a}R^{b}, or S(O)₂CH₃, wherein each R^{a} and R^{b} is independently H or C₁-C₄ alkyl.

In some embodiments, the compound is a compound of Formula (I), (Ia), (II), (IIa), or (IIb) wherein X is NH and R³ is CH₃.

In some embodiments, the compound is a compound of Formula (I), (Ia), (II), (IIa), or (IIb) wherein X is NH and R³ is C₁-C₄alkyl substituted with 4 to 6-membered heterocyclyl, wherein the heterocyclyl is optionally substituted with S(O)₂CH₃.

In some embodiments, the compound is a compound of Formula (I), (Ia), (II), (IIa), or (IIb) wherein X is —C(O)—NH—, wherein the R³ is attached to the NH— of —C(O)NH—; and R³ is H.

In some embodiments, the compound is a compound of Formula (I), (Ia), (II), (IIa), or (IIb) wherein X is —C(O)—NH—, wherein the R³ is attached to the NH— of —C(O)NH—; and R³ is C₁-C₄alkyl optionally substituted with 1 to 3 groups selected from halogen, OR^{a}, and NR^{a}R^{b}, and wherein each R^{a} and R^{b} is independently H or C₁-C₄ alkyl.

In some embodiments, the compound is a compound of Formula (I), (Ia), (II), (IIa), or (IIb) wherein X is —C(O)—NH—, wherein the R³ is attached to the NH— of —C(O)NH—; and R³ is C₃-C₆cycloalkyl, optionally substituted with 1-3 groups selected from halogen, OR^{a}, CN, and C₁-C₄alkyl; and wherein each R^{a} and R^{b} is independently H or C₁-C₄ alkyl.

In some embodiments, the compound is a compound of Formula (I), (Ia), (II), (IIa), or (IIb) wherein X is —C(O)—NH—, wherein the R³ is 4 to 6-member heterocyclyl, optionally substituted with 1-3 groups selected from halogen, C₁-C₄alkyl, wherein the alkyl is optionally substituted with 1 to 3 halogen; and wherein each R^{a} and R^{b} is independently H or C₁-C₄ alkyl.

In some embodiments, the compound is a compound of Formula (I), (Ia), (II), (IIa), or (IIb) wherein X and R³ are together, are —C(O)NH₂, —C(O)NH(CH₃), —C(O)NH(CH₂CH₃), —C(O)NHCH(CH₃)₂, C(O)NHC(OH)(CH₃)₂,

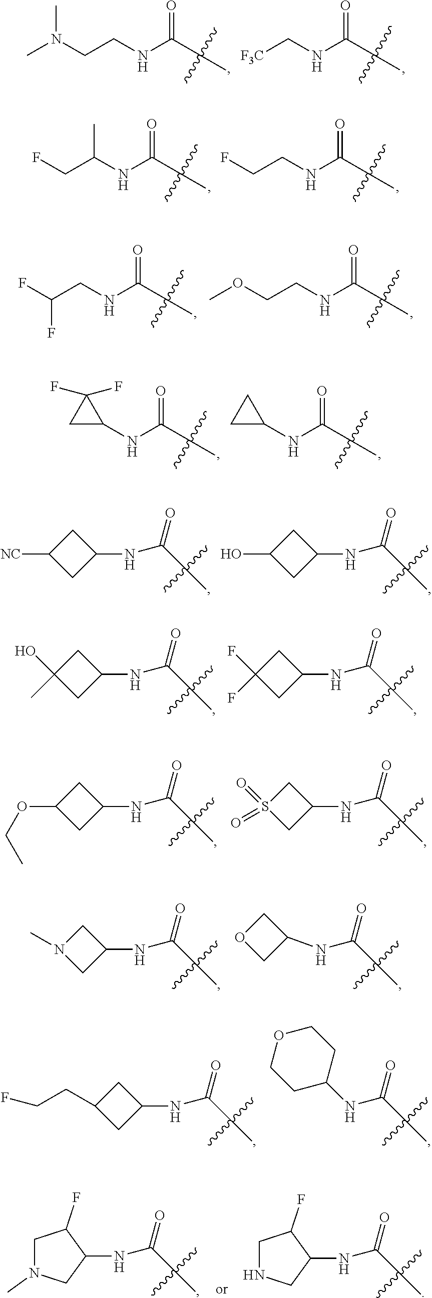

In one embodiment, a compound of the present disclosure is any one of the compounds disclosed in the examples and Table 1, or a pharmaceutically acceptable salt thereof.

In one embodiment, the compounds of Table 3 and pharmaceutically acceptable salts thereof are excluded from the disclosure.

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds disclosed herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” in the Experimental section refers to an intended reaction product compound obtained from a chromatography separation/purification that elutes earlier than a second intended reaction product compound from the same preceding reaction. The second intended product compound is referred to as “peak 2”.

When a disclosed compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are selective EGFR inhibitors. As used herein, the term “selective EGFR inhibitor” means a compound which selectively inhibits certain mutant EGFR kinases over wild-type EGFR and the kinome. Said another way, a selective EGFR inhibitor has no or low activity against wild-type EGFR and the kinome. A selective EGFR inhibitor's inhibitory activity against certain mutant EGFR kinases is more potent in terms of IC₅₀ value (i.e., the IC₅₀ value is subnanomolar) when compared with its inhibitory activity against wild-type EGFR and many other kinases. Potency can be measured using known biochemical assays.

Some compounds of the disclosure have the advantage of good penetration of the brain. The ability of a particular compound to cross the BBB and penetrate the brain can be assessed using a variety of known methods or combinations of such methods. One in vitro method that is frequently used to predict a compound's in vivo brain penetration is P-gp efflux ratio. P-glycoprotein (P-gp) is expressed at the blood-brain barrier (BBB) and restricts the penetration of its substrates into the central nervous system (CNS). Compounds that are found to be good P-gp substrates in vitro (i.e., have a high efflux ratio) are predicted to have poor in vivo brain penetration. In order to measure the P-gp efflux ratio, Madin-Darby canine kidney cells overexpressing P-gp (MDCK-MDR1 cells) the apparent apical to basolateral permeability (Papp[A-B]) and the apparent basolateral to apical permeability (Papp[B-A]) for compounds is determined. The P-gp efflux ratio is a measure of the ratio of Papp[B-A]/Papp[A-B]. In some embodiments, a compound of the disclosure has a P-gp efflux ratio of less than 2, less than 3, less than 4, less than 5.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes.

Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (I)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20^{th} edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The present disclosure provides a method of inhibiting certain mutant forms of epidermal growth factor receptor (EGFR) in a subject in need thereof, comprising administering to the subject an effective amount of a compound disclosed herein, a pharmaceutically acceptable salt thereof or a pharmaceutical composition disclosed herein. Mutant forms of EGFR include for example, EGFR with LRTMCS mutation (the exon 19 deletion (del19) or exon 21 (L858R) substitution mutation, T790M mutation, and C797S mutation). Subjects “in need of inhibiting EGFR” are those having a disease for which a beneficial therapeutic effect can be achieved by inhibiting at least one mutant EGFR, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by mutant EGFR, wherein the inhibition of the mutant EGFR is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. Cancers to be treated according to the disclosed methods include lung cancer, colon cancer, urothelial cancer, breast cancer, prostate cancer, brain cancers, ovarian cancer, gastric cancer, pancreatic cancer, head and neck cancer, bladder cancer, and mesothelioma, including metastasis (in particular brain metastasis) of all cancers listed. Typically, the cancer is characterized by at one or more EGFR mutations described herein. In a specific embodiment, the cancer has progressed on or after EGFR tyrosine kinase inhibitor (TKI) Therapy. In a specific embodiment, the disease has progressed on or after first line osimertinib.

In a specific embodiment, the cancer to be treated is lung cancer. In a more specific embodiment, the cancer is non-small cell lung cancer (NSCLC). In some embodiments, the lung cancer is locally advanced or metastatic NSCLC, NSCLC adenocarcinoma, NSCLC with squamous histology and NSCLC with non-squamous histology. In another embodiment, the lung cancer is NSCLC adenocarcinoma. In another specific embodiment, the lung cancer (or non-small cell lung cancer) has metastasized to the brain.

In another embodiment, the disease/condition/or cancer associated with or modulated by mutant EGFR that is characterized by an EGFR genotype selected from genotypes 1-17 according the Table below (del18=Exon 18 deletion, specifically, e.g., del E709_T710 insD; del19=Exon 19 deletion, specifically, e.g., delE746_A750 (most common), delE746_S752insV, de1747_A750insP, delL747_P753insS, and delS752_I759; ex20ins-Exon 20 insertion, specifically, e.g., D761-E762insX, A763-Y764insX, Y764-V765insX, V765-M766insX, A767-S768insX, S768-D769insX, V769-D770insX, N771-P772insX, P772-H773insX, H773-V774insX, and V774-C775insX):

EGFR Genotype

| | |
|---|---|
| 1 | EGFR del19 |
| 2 | EGFR del19 T790M |
| 3 | EGFR del19 C797S |
| 4 | EGFR del19 C797X (C797G or C797N) |
| 5 | EGFR del19 T790M C797S |
| 6 | EGFR del19 T790M C797S Q791P |
| 7 | EGFR del19 T790M (C797G or C797N) |
| 8 | EGFR del19 L792X (L792F, L792H or L792Y) |
| 9 | EGFR del19 T790M L792X (L792F, L792H, or L792Y) |
| 10 | EGFR del19 G796R (G796S) |
| 11 | EGFR del19 T790M G796R (G796S) C797S L792X (L792F, L792H or L792Y) |
| 12 | EGFR del19 L792R (L792V or L792P) |
| 13 | EGFR del19 L718Q (L718V) |
| 14 | EGFR del19 T790M L718Q (L718V) L792X (L792F, L792H or L792Y) |
| 15 | EGFR del19 T790M G796R (G796S) |
| 16 | EGFR del19 T790M L792R (L792V or L792P) |
| 17 | EGFR del19 T790M L718Q (L718V) |
| 18 | EGFR del19 T790M C797S L718Q (L718V) |
| 19 | EGFR del19 G724S |
| 20 | EGFR del19 T790M G724S |
| 21 | EGFR del19 S768I (SV768IL) |
| 22 | EGFR del19 T790M S768I (SV768IL) |
| 23 | EGFR del19 T790M C797S/G L792X (L792F, L792H, L792R, or L792Y) |
| 24 | EGFR del 19 V834L |
| 25 | EGFR del 19 T790M V834L |
| 27 | EGFR del19 T790M L792X (L792F, L792H, L792R, or L792Y) |
| 28 | EGFR del19 C797S L718Q (L718V) |
| 29 | EGFR del19 L718Q (L718V) A750P |
| 30 | EGFR del19 T790M L718Q (L718V) A750P L792V G796R |
| 31 | EGFR L858R |
| 32 | EGFR L858R T790M |
| 33 | EGFR L858R C797S |
| 34 | EGFR L858R C797X (797G or C797N) |
| 35 | EGFR L858R T790M C797S |
| 36 | EGFR L858R T790M C797S Q791P |
| 37 | EGFR L858R T790M C797X (C797G or C797N) |
| 38 | EGFR L858R L792X (L792F, L792H or L792Y) |
| 39 | EGFR L858R T790M L792X (L792F, L792H or L792Y) |
| 40 | EGFR L858R G796R (G796S) |
| 41 | EGFR L858R T790M G796R (G796S) C797S L792X (L792F, L792H or L792Y) |
| 42 | EGFR L858R L792R (L792V or L792P) |
| 43 | EGFR L858R L718Q (L718V) |
| 44 | EGFR L858R T790M G796R (G796S) |
| 45 | EGFR L858R T790M L792R (L792V or L792P) |
| 46 | EGFR L858R T790M L718Q (L718V) |
| 47 | EGFR L858R T790M C797S L718Q (L718V) |
| 48 | EGFR L858R T790M L718Q (L718V) L792X (L792F, L792H or L792Y) |
| 49 | EGFR L858R G724S |
| 50 | EGFR L858R T790M G724S |
| 51 | EGFR L858R S768I (SV768IL) |
| 52 | EGFR L858R T790M S768I (SV768IL) |
| 53 | EGFR L858R T790M C797S/G L792X (L792F, L792H, L792R, or L792Y) |
| 54 | EGFR L858R V834L |
| 55 | EGFR L858R T790M V834L |
| 57 | EGFR L858R T790M L792X (L792F, L792H, L792R, or L792Y) |
| 58 | EGFR L858R C797S L718Q (L718V) |
| 59 | EGFR L858R L718Q (L718V) A750P |
| 60 | EGFR L858R T790M L718Q (L718V) A750P L792V G796R |
| 61 | EGFR L861Q |
| 62 | EGFR L861Q T790M |
| 63 | EGFR L861Q T790M C797S/G/N |
| 64 | EGFR L861Q C797S/G/N |
| 65 | EGFR del18 |
| 66 | EGFR G719X (G719A, G719S, G719C, G719R, G719D, or G719V) |
| 67 | EGFR E709X (E709K, E709H, or E709A) |
| 68 | EGFR E709X (E709K, E709H, or E709A) (G719A, G719S, G719C, G719D, |
| | G719R, or G719V) |
| 69 | EGFR G719X (G719A, G719S, G719C, G719D, G719R, or G719V) S768I |
| 70 | EGFR ex20ins |
| 71 | EGFR ex20ins L718Q |
| 72 | EGFR ex20ins T790M |
| 73 | EGFR ex20ins C797S |
| 74 | EGFR S7681I |
| 75 | EGFR T790M |
| 76 | EGFR T790M C797S/G L792X (L792F, L792H, L792R, or L792Y) |

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797X (C797G or C797N).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 T790M (C797G or C797N).

In another embodiment, the disease/condition/or cancer (e.g., NSCLC) being treated with a disclosed compound, a pharmaceutically acceptable salt, or or a pharmaceutical composition disclosed herein is characterized by EGFR comprising EGFR del19 L792X (L792F, L792H or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 T790M L792X (L792F, L792H, or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 G796R (G796S).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L792R (L792V or L792P).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L718Q (L718V).

In another embodiment, the disease/condition/or cancer (e.g., NSCLC) being treated with a disclosed compound, a pharmaceutically acceptable salt thereof, or pharmaceutical composition described herein is characterized by EGFR comprising EGFR del19 T790M G796R (G796S).

In another embodiment, the disease/condition/or cancer (e.g., NSCLC) being treated with a disclosed compound, a pharmaceutically acceptable salt thereof, or pharmaceutical composition described herein is characterized by EGFR comprising EGFR del19 T790M L792R (L792V or L792P).

In another embodiment, the disease/condition/or cancer (e.g., NSCLC) being treated with a disclosed compound, a pharmaceutically acceptable salt thereof or pharmaceutical composition described herein is characterized by EGFR comprising EGFR del19 T790M L718Q (L718V).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797X (797G or C797N).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R T790M C797X (797G or C797N).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L792X (L792F, L792H or L792Y).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L790M L792X (L792F, L792H or L792Y).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L718Q (L718V).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R T790M L718Q (L718V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del18.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719R, G719D, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A) (G719A, G719S, G719C, G719D, G719R, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719D, G719R, or G719V) S7681.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins L718Q.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR S7681I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR T790M.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR T790M C797S/G L792X (L792F, L792H, L792R, or L792Y).

In another embodiment, the disease/condition/or cancer (e.g., NSCLC) being treated with a disclosed compound, a pharmaceutically acceptable salt or a pharmaceutical composition disclosed herein is characterized by an EGFR genotype selected from genotypes 1-76.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to gefitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to erlotinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and gefitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and erlotinib.

Another embodiment is the treatment a subject with metastatic NSCLC with tumors harboring activating Exon 19 Deletion or L858R EGFR mutations as well as a resistance mutation disclosed herein as detected by an approved molecular testing methodology. Another embodiment is a disclosed compound used in combination with a 1^{st} or 3^{rd} generation TKI indicated for the treatment of subject with metastatic NSCLC with tumors harboring T790M and C797S mutations as detected by an approved test, and whose disease has progressed on or after at least 2 prior EGFR TKI therapies.

Another embodiment is a disclosed compound for the treatment of subjects with metastatic NSCLC whose disease with on-target EGFR resistance has progressed on or after any EGFR TKI. In a specific embodiment, the disclosed compound is used in combination with a 1^{st} or 3^{rd} generation TKI indicated for the treatment of subject with metastatic NSCLC.

Another embodiment is a disclosed compound for the treatment of subjects with metastatic EGFR C797S mutation-positive NSCLC as detected by an approved molecular test, whose disease has progressed on or after first-line osimertinib. In a specific embodiment, the disclosed compound is used in combination with a 1^{st} or 3^{rd} generation TKI indicated for the treatment of subject with metastatic NSCLC.

In a particular embodiment, the deletions, mutations, and insertions disclosed herein are detected by an FDA-approved test.

A person of ordinary skill in the art can readily determine the certain EGFR alterations a subject possesses in a cell, cancer, gene, or gene product, e.g., whether a subject has one or more of the mutations or deletions described herein using a detection method selected from those known in the art such as hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect one or more EGFR deletions and/or mutations, a primary tumor sample, circulating tumor DNA (ctDNA), circulating tumor cells (CTC), and/or circulating exosomes may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples.

The compounds of the disclosure, pharmaceutically acceptable salts thereof or pharmaceutical compositions disclosed herein may be used for treating to a subject who has become refractory to treatment with one or more other EGFR inhibitors. “Refractory” means that the subject's cancer previously responded to drugs but later responds poorly or not at all. In some some embodiments, the subject has become refractory to one or more first generation EGFR inhibitors such as erlotinib, gefitinib, icotinib or lapatinib. In some embodiments, the subject has been become refractory to treatment with one or more second generation EGFR inhibitors such as afatinib, dacomitinib, poziotinib, or neratinib. In some embodiments the subject has become refractory to treatment with one or more first generation inhibitors and one or more second generation inhibitors.

In some embodiments, the subject has become refractory to treatment with one or more third generation inhibitors such as osimertinib, nazartinib, or avitinib. In one embodiment, the subject has become refractory to treatment with one or more first generation EGFR inhibitors and one or more third generation EGFR inhibitors. In some embodiments, the subject has become refractory to treatment with one or more second generation EGFR inhibitors and one or more third generation EGFR inhibitors. In some embodiments, the subject has become refractory to treatment with one or more first generation inhibitors, and one or more third generation EGFR inhibitors.

### Combinations

The compounds of the disclosure, pharmaceutically acceptable salts thereof, or pharmaceutical compositions disclosed herein can be used in combination with one or more additional pharmacologically active substances. For example, the disclosure includes methods of treating a condition/disease/ or cancer comprising administering to a subject in need thereof a compound of the disclosure or a pharmaceutically acceptable salt or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alflutinib (AST 2818), almonertinib (HS10296), BBT-176, BI-4020, CH7233163, gilitertinib, JND-3229, lazertinib, nazartinib (EGF 816), PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). For the treatment of cancer e.g., NSCLC using a compound of the disclosure or pharmaceutically acceptable salt thereof or pharmaceutical composition disclosed herein in combination with a first line therapy, for example a first, second, or third generation EGFR inhibitor (i.e., as an initial treatment before the cancer has become refractory) may forestall or delay the cancer from becoming refractory. Typically, the cancer is characterized by one of the EGFR genotypes described herein.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib, glumetinib, tepotinib) and MET antibodies (emibetuzumab, telisotuzumab vedotin (ABBV 339)); mitotic kinase inhibitors (CDK4/6 inhibitors such as palbociclib, ribociclib, abemacicilb, GIT38); anti-angiogenic agents e.g., bevacizumab, nintedanib; apoptosis inducers such as Bcl-2 inhibitors e.g, venetoclax, obatoclax, navitoclax, palcitoclax (APG-1252), and Mcl-1 inhibitors e.g., AZD-5991, AMG-176, S-64315; mTOR inhibitors e.g, rapamycin, temsirolimus, everolimus, ridoforolimus; RET inhibitors, like pralsetinib and selpercatinib, and PI3K inhibitors dactolisib (BEZ235), pictilisib (GDC-0941), LY294002, idelalisib (CAL-101); JAK inhibitors (e.g., AZD4205, itacitinib), Aurora A inhibitors (e.g., alisertib); BCR/ABL and/or Src family tyrosine kinase inhibitors (e.g., dasatinib); VEGF inhibitors (e.g., MP0250; ramucirumab); multi-kinase protein inhibitors (e.g., anlotinib, midostaurin); PARP inhibitors (e.g., niraparib); platinum therapies (e.g., cisplatin (CDDP), carboplatin (CBDCA), or nedaplatin (CDGP)); PD-L1 inhibitors (e.g., durvalumab (MEDI 4736)); HER2/neu receptor inhibitors (e.g., trastuzumab); anti-HER2 or anti-HER3 antibody-drug conjugates (e.g., patritumab deruxtecan (U3-1402), trastuzumab emtansine); or immunogene therapy (e.g., oncoprex).

A “subject” is a human in need of treatment.

Methods of Administration and Dosage Forms The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) being used by following, for example, dosages reported in the literature and recommended in the Physician's Desk Reference (57^{th} Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed EGFR inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The following examples are intended to be illustrative and are not intended to be limiting in any way to the scope of the disclosure.

### EXEMPLIFICATION

### Examples

### Preparation of Exemplary Compounds

### Definitions

Abbreviations and acronyms used herein include the following:
AcOH means acetic acid;
Aq. Means aqueous;
Bn means benzyl;
Boc means tert-butoxy carbonyl;
br means broad;
BrettPhos Pd G3 means [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate;
n-BuOH means butan-1-ol;
t-BuOH means tertiary butanol;
° C. means degrees Celsius;
CDCl₃ means deutero-chloroform;
Cphos means 2-Dicyclohexylphosphino-2′,6′-bis(N,N-dimethylamino)biphenyl;
Cs₂CO₃ means cesium carbonate;
δ means chemical shift;
d means doublet;
dd means double doublet;
DCM means dichloromethane;
DEA means diethylamine;
DEAD means diethyl azodicarboxylate;
DIAD means diisopropyl azodicarboxylate;
DIPEA means N-ethyldiisopropylamine or N,N-diisopropylethylamine;
DMF means N,N-dimethylformamide;
DMSO means Dimethylsulfoxide;
DMSO-d₆ means hexadeuterodimethyl sulfoxide;
EDC·HCl means N-Ethyl-N′-(3-dimethylaminopropyl)carbodiimide hydrochloride;
Et means ethyl;
EtOH means ethanol;
EtOAc means ethyl acetate;
Eq. means equivalent;
g means gram;
HATU means 1-[bis(dimethylamino)methylene]-1H-1,2,3-triazolo[4,5-b]pyridinium 3-oxid hexafluorophosphate;
HCl means hydrochloric acid;
HCOH means formaldehyde;
HCO₂H means formic acid
¹H NMR means proton nuclear magnetic resonance;
H₂O means water;
HOBt means 1-Hydroxybenzotriazole hydrate;
HPLC means high pressure liquid chromatography;
h means hour;
IPA means 2-propanol;
K₂CO₃ means potassium carbonate;
KOH means potassium hydroxide;
L means litre;
LCMS means liquid chromatography mass spectrometry;
LiOH means lithium hydroxide;
m means multiplet;
M means molar;
Me means methyl;
MeCN means acetonitrile;
Mel means iodomethane;
MeOH means methanol;
MeOH-d₄ means deutero-methanol;
mg means milligram;
MgSO₄ means magnesium sulfate;
MHz means mega Hertz;
mins means minutes;
mL means millilitres;
mmol means millimole;
MS m/z means mass spectrum peak;
MsCl means methanesulfonyl chloride;
MtBE means tert-butyl methyl ether;
N₂ means nitrogen;
NaBH₄ means sodium borohydride;
NaBH₃CN means sodium cyanoborohydride;
Na₂CO₃ means sodium carbonate;
NaH means sodium hydride;
NaHCO₃ means sodium bicarbonate;
NaOH means sodium hydroxide;
Na₂SO₄ means sodium sulfate;
NH₃ means ammonia;
NH₄Cl means ammonium chloride;
NH₄OH is ammonium hydroxide;
NIS means N-iodosuccinimide;
PE means petroleum ether;
Pd(amphos)Cl₂ means Bis(di-tert-butyl(4-dimethylaminophenyl)phosphine)dichloropalladium(II);
Pd(Oac)₂ means palladium acetate;
Pd₂(dba)₃ means tris(dibenzylideneacetone)dipalladium (0);
Pd₂(dba)₃·CH₃Cl means tris(dibenzylideneacetone)dipalladium (0)-chloroform adduct;
Pd(dppf)Cl₂ means [1,1′-bis(diphenylphosphino)ferrocene]dichloropalladium(II);
Pd(PPh₃)₄ means tetrakis(triphenylphosphine)palladium(0);
Pd/C means palladium on charcoal;
PPh₃ means triphenylphosphine;
PtO₂ means platinum (II) oxide;
q means quartet;
Rockphos Pd G3 means [(2-Di-tert-butylphosphino-3-methoxy-6-methyl-2′,4′,6′-triisopropyl-1,1′-biphenyl)-2-(2-aminobiphenyl)]palladium(II) methanesulfonate;
rt means room temperature;
RT means retention time;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat. means saturated;
SFC means supercritical fluid chromatography;
soln. means solution;
t means triplet;
TEA means triethylamine;
TFA means trifluoroacetic acid;
THF means tetrahydrofuran;
TLC means thin layer chromatography;
TsOH means para-toluenesulfonic acid
μL means micro litres;
μmol means micromole;
Xphos Pd G3 means (2-dicyclohexylphosphino-2′,4′,6′-triisopropyl-1,1′-biphenyl)[2-(2′-amino-1,1′-biphenyl)]palladium(II) methanesulfonate;

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5^{th} ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization:

LC-MS: The liquid chromatography-mass spectrometry (LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 4 minutes was utilized. The flow rate was constant at 1 mL/min. Alternatively, the liquid chromatography-mass spectrometry (LC-MS) data (sample analyzed for purity and identity) were obtained with a Shimadzu LCMS system using an Shimadzu LCMS mass spectrometer utilizing ESI ionization fitted with an Agilent (Poroshel HPH-C18 2.7 um particle size, 3.0×50 mm dimensions) reverse-phase column at 22.4 degrees Celsius. The mobile phase consisted of a mixture of solvent 5 mM NH₄HCO₃ (or 0.05% TFA) in water and acetonitrile. A constant gradient from 90% aqueous/10% organic to 5% aqueous/95% organic mobile phase over the course of 2 minutes was utilized. The flow rate was constant at 1.5 mL/min.

Prep LC-MS: Preparative HPLC was performed on a Shimadzu Discovery VP® Preparative system fitted with a Luna 5 u C18(2) 100A, AXIA packed, 250×21.2 mm reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 25 minutes was utilized. The flow rate was constant at 20 mL/min. Reactions carried out in a microwave were done so in a Biotage Initiator microwave unit. Alternatively, the preparative HPLC was performed on a Waters Preparative system fitted with Column: Xbridge Shield RP18 OBD Column, 30*150 mm, 5 um; The mobile phase consisted of a mixture of solvent Water (10 mmol/L NH₄HCO₃+0.05% NH3·H2O) and acetonitrile. A constant gradient from 95% aqueous/5% organic to 5% aqueous/95% organic mobile phase over the course of 11 minutes was utilized. The flow rate was constant at 60 mL/min. Reactions carried out in a microwave were done so in a Biotage Initiator microwave unit.

Silica gel chromatography: Silica gel chromatography was performed on a Teledyne Isco CombiFlash® Rf unit, a Biotage® Isolera Four unit, or a Biotage® Isolera Prime unit.

Proton NMR: ¹H NMR spectra were obtained with a Varian 400 MHz Unity Inova 400 MHz NMR instrument (acquisition time=3.5 seconds with a 1 second delay; 16 to 64 scans) or a Avance 400 MHz Unity Inova 400 MHz NMRinstrument (acquisition time=3.99 seconds with a 1 second delay; 4 to 64 scans) or a Avance 300 MHz Unity Inova 300 MHz NMR instrument (acquisition time=5.45 seconds with a 1 second delay; 4 to 64 scans). Unless otherwise indicated, all protons were reported in DMSO-d6 solvent as parts-per million (ppm) with respect to residual DMSO (2.50 ppm).

SFC: Waters Preparative system.

Chiral-HPLC: Agilent 1260 Preparative system.

One of ordinary skill in the art will recognize that modifications of the gradient, column length, and flow rate are possible and that some conditions may be more suitable for compound characterization than others, depending on the chemical species being analyzed.

### Preparative HPLC Purification:

The following codes refer to the preparative HPLC conditions used as indicated in the examples and preparation sections. Individual gradients were optimised for each example as appropriate.

| Prep-HPLC Code | Conditions |
|---|---|
| HPLC-1 | Chiralpak IA-3, 4.6 × 50 mm, 3 mm; MtBE (+0.1% DEA)/MeOH; 1:1 |
| HPLC-2 | Xbridge Shield OBD C18, 30 × 150 mm, 5 mm; MeCN/H₂O (10 mM |
| | NH₄HCO₃); gradient 0-100% optimised for each compound |
| HPLC-3 | Chiralpak IA-3, 4.6 × 50 mm, 3 mm; Hex (+0.1% DEA)/EtOH; 1:1; gradient |
| | 0-100% optimised for each example |
| HPLC-4 | Chiralpak ID-3, 4.6 × 50 mm, 3 mm; (3:1 Hex/DCM (+0.1% DEA))/EtOH; |
| | 4:1; gradient 0-100% optimised for each example |
| HPLC-5 | Chiralpak IA-3, 4.6 × 50 mm, 3 mm; (3:1 Hex/DCM (+0.1% DEA))/EtOH; |
| | 85:15; gradient 0-100% optimised for each example |
| HPLC-6 | Chiralpak IC, 20 × 250 mm, 5 mm; (3:1 Hex/DCM (+0.1% DEA))/MeOH |
| | (+10 mM NH₄OH); 30%; gradient 0-100% optimised for each example |
| HPLC-7 | Xbridge Shield OBD C18, 30 × 150 mm, 5 mm; MeCN/H₂O (+0.05% |
| | NH₄OH); gradient 0-100% optimised for each compound |
| HPLC-8 | Xbridge Shield OBD C18, 30 × 150 mm, 5 mm; MeCN/H₂O (10 mM |
| | NH₄HCO₃ + 0.1% NH₄OH); gradient 0-100% optimised for each example |
| HPLC-9 | Xbridge Shield OBD C18, 30 × 150 mm, 5 mm; MeCN/H2O (10 mM |
| | NH₄HCO₃ + 0.1% NH₄OH); gradient 0-100% optimised for each example |
| HPLC-10 | Chiralpak IF, 4.6 × 50 mm, 3 mm; (3:1 Hex/DCM (+0.1% DEA))/IPA; 50% |
| HPLC-11 | Kinetex EVO C18, 30 × 150 mm, 5 mm; MeCN/H₂O (10 mM NH₄HCO₃) |
| HPLC-12 | Xbridge Shield OBD C18, 19 × 250 mm, 10 mm; MeCN/H₂O (10 mM |
| | NH₄HCO₃ + 0.1% NH₄OH); gradient 0-100% optimised for each example |
| HPLC-13 | Xbridge Shield OBD C18, 30 × 150 mm, 5 mm; MeCN/H₂O (+0.1% |
| | HCO₂H); gradient 0-100% optimised for each example |
| HPLC-14 | YMC-Actus Triart C18, 30 × 250 mm, 5 mm; MeCN/H₂O (0.05% NH₄OH); |
| | gradient 0-100% optimised for each example |
| HPLC-15 | Chiralpak IA, 20 × 250 mm, 5 mm; 50% (1:1 Hex/DCM (10 mM |
| | NH₃/MeOH))/EtOH |

### General Synthesis Schemes

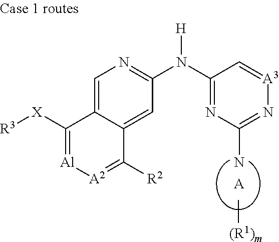

According to a first process, compounds of Formula (I) may be prepared from the compounds of Formulae (II) and (III), as illustrated by Scheme 1.

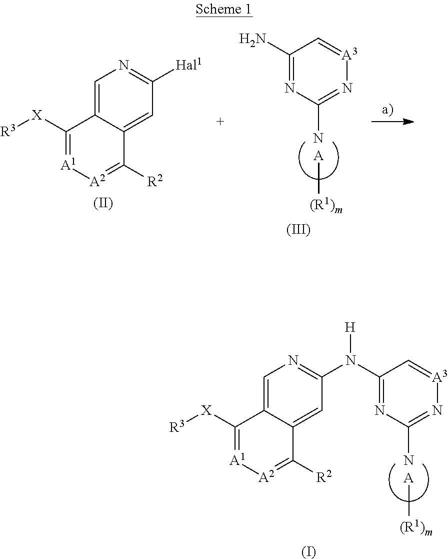

Hal¹ is a halogen, preferably Cl

The compound of Formula (I) may be prepared according to process step (a), a Buchwald-Hartwig cross coupling reaction. Typical conditions comprise, reaction of the amine of Formula (III) with the halide of Formula (II) in the presence of a suitable inorganic base, a suitable palladium catalyst in a suitable solvent at elevated temperature. Preferred conditions comprise, reaction of the compounds of Formulae (II) and (III) in the presence of, RuPhos Pd G3, BrettPhos Pd G3, BrettPhos Pd G4, Xphos Pd G4 or Xantphos Pd, or Cphos, Xantphos or BrettPhos in combination with Pd₂(dba)₃ or BrettPhos Pd G4, in the presence of a suitable base such as Cs₂CO₃ or Na₂CO₃, in a suitable solvent such as dioxane, at between 90° C. and 130° C.

Wherein X is NH, compounds of Formula (II)(A) may be prepared from compounds of Formulae (IV) and (V) as illustrated by Scheme 2.

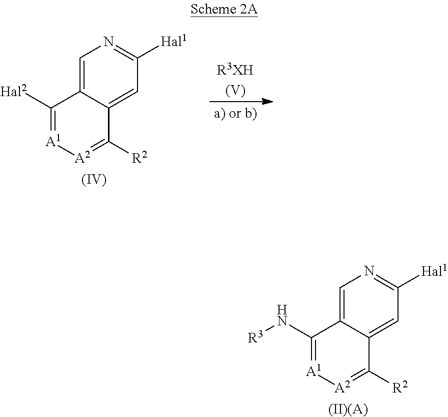

Hal¹ is a halogen, preferably Cl

Hal² is a halogen, preferably Br or Cl

Wherein X is NH, compounds of Formula (II)(A), may be prepared from the halide of Formula (IV) and the amine of Formula (V), wherein X is NH, according to process step a) a Buchwald-Hartwig cross coupling reaction, as previously described in Scheme 1.

Alternatively, compounds of Formula (II)(A), may be prepared according to process step b) an amination reaction. Typical conditions comprise, reaction of the amine of Formula (V) with the halide of Formula (IV) in the presence of a suitable organic base, such as TEA or DIPEA in a suitable solvent such as DMSO or IPA at elevated temperature, such as 100° C.

Wherein X is O, compounds of Formula (II)(B) may be prepared from compounds of Formulae (IV) and (V) as illustrated by Scheme 2B

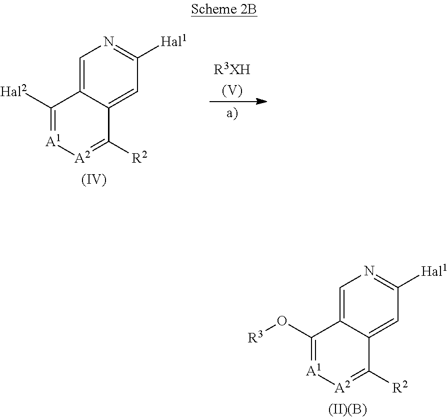

Compounds of Formula (II)(B), may be prepared according to process step a) a Buchwald cross coupling reaction. Typical conditions comprise, reaction of the alcohol of Formula (V) with the halide of Formula (IV) in the presence of a suitable inorganic base, a suitable palladium catalyst in a suitable solvent at elevated temperature. Preferred conditions comprise, reaction of the compounds of Formulae (IV) and (V) in the presence of, Rockphos Pd G3, in the presence of a suitable base such as Cs₂CO₃, in a suitable solvent such as toluene at elevated temperature, typically between 50 and 100° C.

According to a third process, wherein X is O or NH and R² is C₁-C₄ alkyl, C₁-C₄ alkoxy, or C₃-C₆ cycloalkyl, compounds of Formula (II)(A) and (B) may be prepared from compounds of Formulae (V), (VI), (VII), (VIII) and (IX) as illustrated by Scheme 3.

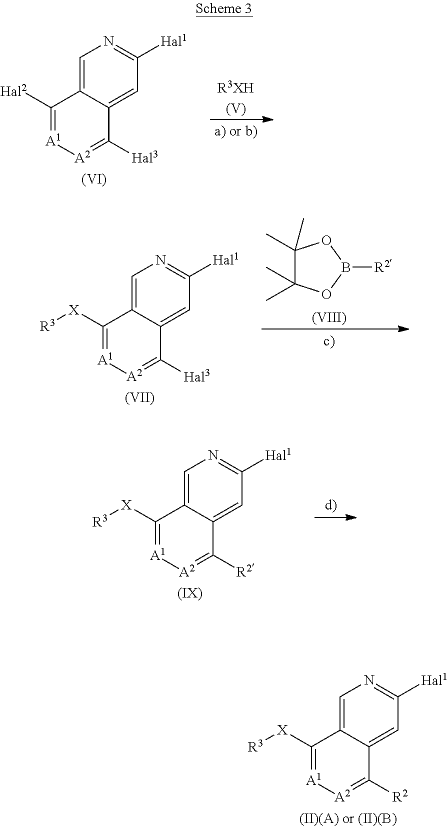

Hal³ is halogen, preferable Cl or Br

R^{2′} is the unsaturated analogue of R²,

The compound of Formula (VII) may be prepared from the halide of Formula (VI) and the compound of Formula (V), according to process steps a) or b), as previously described in Schemes 1 and 2.

The compound of Formula (IX) may be prepared from the aromatic halide of Formula (VII) with the boronate ester of Formula (VIII), according to process step c) an organometallic catalysed cross-coupling reaction. Typical cross-coupling conditions comprise a palladium catalyst containing suitable phosphine ligands, in the presence of an inorganic or organic base, in aqueous solvent at between rt and the reflux temperature of the reaction. Preferred conditions comprise reaction of the compounds of Formulae (VII) and (VIII) in the presence of Pd(dppf)Cl₂, and a suitable base such as Na₂CO₃ or K₂CO₃ in a suitable solvent such as aqueous dioxane at between 70° C. and 100° C.

The compound of Formula (II)(A) or (II)(B) may be prepared from the compound of Formula (IX) by process step d) a hydrogenation reaction in the presence of a suitable catalyst such as Pd/C or PtO₂ in a suitable solvent, such as EtOAc under an atmosphere of H₂ at about rt.

According to a fourth process, wherein X is O, compounds of Formula (II)(B) may be prepared from the compound of Formula (X), as illustrated by Scheme 4.

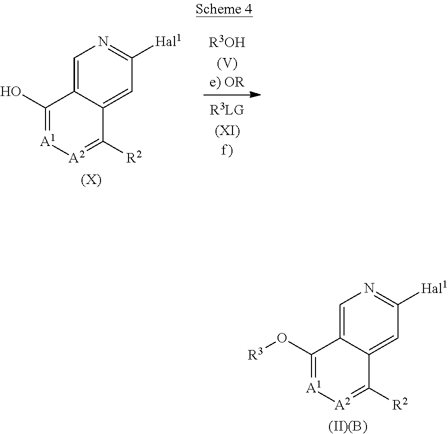

LG is a leaving group, typically a halogen or sulfonate group and preferably a bromide or mesylate.

The compound of Formula (II)(B) may be prepared from the alcohol or Formula (X) and the alcohol of Formula (V) according to process step e) a Mitsunobu reaction. Typical conditions comprise reaction of the alcohols of Formulae (X) and (V) in the presence of a suitable reagent such as DIAD or DEAD and PPh₃ in a suitable solvent such as THF at about rt.

Alternatively, the compound of Formula (II)(B) may be prepared from the alcohol of Formula (X) and the compound of Formula (XI) according to process step f) an alkylation reaction. Typical conditions comprise reaction of the alcohol of Formula (X) with the compound of Formula (XI) in the presence of a suitable inorganic base, such as K₂CO₃ in a suitable solvent such as DMF at elevated temperature, such as 80-100° C.

According to a fifth process, compounds of Formula (III) may be prepared from the compounds of Formulae (XII) and (XIII) as illustrated in Scheme 5.

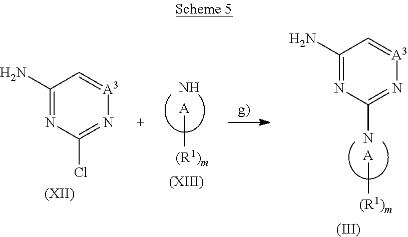

The compound of Formula (III) may be prepared from the chloride of Formula (XII) and the amine of Formula (XIII), according to process step g) an amination reaction. Preferred conditions comprise the reaction of the compounds of Formulae (XII) and (XIII) in the presence of a suitable organic base, such as TEA or DIPEA in a suitable solvent such as DMSO or IPA at elevated temperature, such as between 100 and 140° C.

According to a sixth process, wherein X is —C(O)—NH—, compounds of Formula (I)(C) may be prepared from compounds of Formulae (III), (XIV), (XV) and (XVI), as illustrated by Scheme 6.

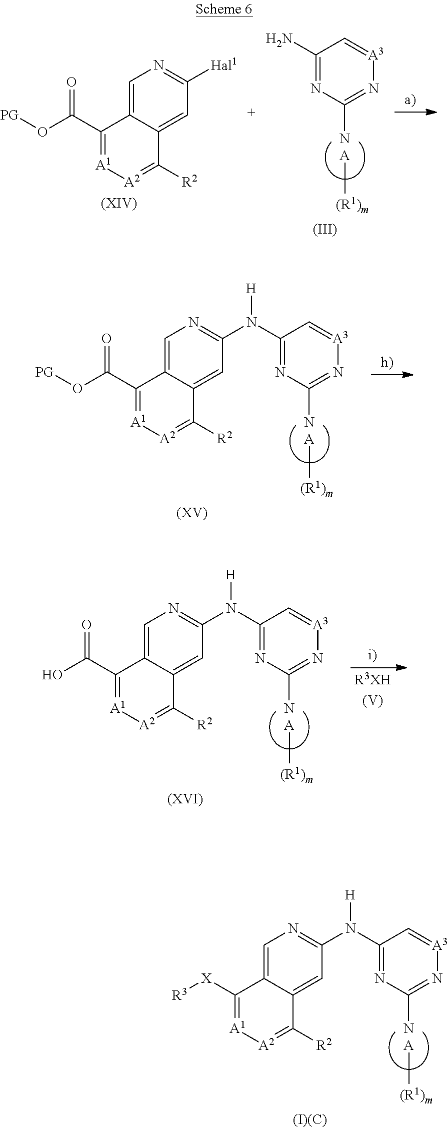

PG is a suitable ester protecting group, typically a C₁-C₄ alkyl and preferably Me.

The compound of Formula (XV) may be prepared from the amine of Formula (III) and the halide of Formula (XIV), according to process step a), as previously described in Scheme 1.

Compounds of Formula (XVI) may be prepared from the ester of Formula (XV), according to process step h) a hydrolysis reaction, under suitable acidic or basic conditions in a suitable aqueous solvent. Preferred conditions comprise the treatment of the ester of Formula (XV) with an alkali metal base such as LiOH, NaOH or K₂CO₃ in aqueous THF at between rt and the reflux temperature of the reaction.

The compound of Formula (I)(C) may be prepared from the acid of Formula (XVI) and the amine of Formula (V), by process step i) an amide bond formation, in the presence of a suitable coupling agent and organic base, optionally in a suitable polar aprotic solvent. Preferred conditions, comprise the reaction of the acid of Formula (XVI) with the amine of Formula (V) in the presence of coupling agent preferably, HATU or HOBt, in the presence of EDC, in the presence of a suitable organic base such as TEA or DIPEA, optionally in a suitable solvent, such as DMF, DCM, DMSO, EtOAc, dioxane or MeCN at about rt.

According to a seventh process, wherein X is —C(O)—NH—, compounds of Formula (II)(C) may be prepared from compounds of Formulae (V), (XIV) and (XVII) as illustrated by Scheme 7

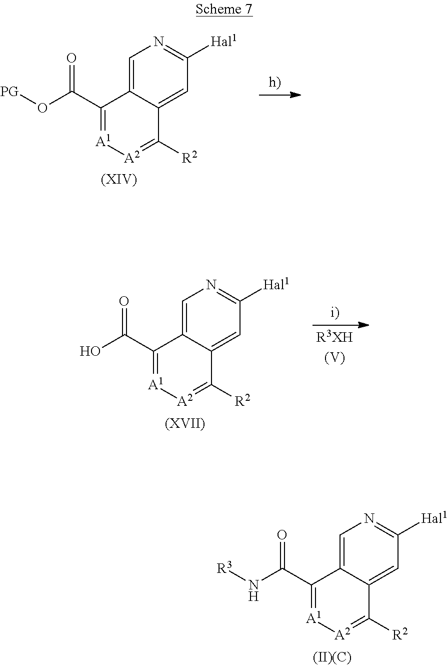

The compound of Formula (XVII) may be prepared from the ester of Formula (XIV) according to step h) as previously described in Scheme 6.

The compound of Formula (II)(C) may be prepared from the acid of Formula (XVII) and the amine of Formula (V), according to step i) as previously described in Scheme 6.

The compounds of Formulae (IV), (V), (VI), (VIII), (X), (XI), (XII), (XIII) and (XIV) are commercially available, or may be prepared by analogy to methods known in the literature, or the methods described in the Experimental section below.

It will be appreciated by those skilled in the art that it may be necessary to utilise a suitable protecting group strategy for the preparation of compounds of Formula (I). Typical protecting groups may comprise, carbamate and preferably Boc for the protection of amines, a TBS or benzyl group for the protection of a primary alcohol, a C1-C4 alkyl, phenyl or benzyl group for the protection of carboxylic acids.

Compounds of Formulae (I), may be converted to alternative compounds of Formulae (I), by standard chemical transformations such as for example, reductive amination, alkylation or acetylation of a heteroatom such as N, using methods well known to those skilled in the art.

### Synthesis of Intermediates

### Preparation 1

### Tert-butyl 5-methyl-4-(trimethylsilyloxy)-5,6-dihydropyridine-1(2H)-carboxylate

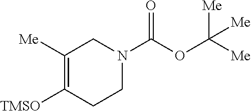

Trimethylsilyl trifluoromethanesulfonate (12.50 g, 56.25 mmol) was added drop wise to a pre-cooled solution of tert-butyl 3-methyl-4-oxopiperidine-1-carboxylate (10 g, 46.88 mmol) and TEA (11.38 g, 112.5 mmol) in toluene (100 mL) at 0° C. and the resulting mixture stirred for 4 h at 0° C. The solution was quenched with water (50 mL) and extracted with EtOAc (×2). The combined organics were washed (brine), dried (Na₂SO₄) and evaporated to dryness in vacuo to afford the title compound as a yellow oil (10.5 g, 78.5%). ¹H NMR (400 MHz, DMSO-d₆) δ: 3.68-3.66 (m, 2H), 3.43 (t, 2H), 2.05 (tq, 2H), 1.53-1.47 (m, 3H), 1.41 (s, 9H), 0.15 (s, 9H).

### Preparation 2

### tert-butyl 3-fluoro-3-methyl-4-oxopiperidine-1-carboxylate

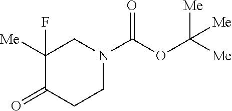

A mixture of tert-butyl 5-methyl-4-(trimethylsilyloxy)-5,6-dihydropyridine-1(2H)-carboxylate (Preparation 1, 10 g, 35.0 mmol) and SelectFluor (13.6 g, 38.5 mmol) in MeCN (100 mL) was stirred for 1 h at 0° C. The solution was diluted with water (100 mL) and extracted with EtOAc. The combined organics were washed (brine), dried (Na₂SO₄) and evaporated to dryness in vacuo to afford the title compound as a pale yellow oil (8 g, 98.8%).

### Preparation 3

### tert-butyl 3-fluoro-4-hydroxy-3-methylpiperidine-1-carboxylate

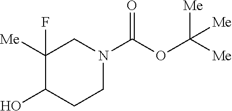

A mixture of tert-butyl 3-fluoro-3-methyl-4-oxopiperidine-1-carboxylate (Preparation 2, 7 g, 30.2 mmol) and NaBH₄ (1.37 g, 36.2 mmol) in MeOH (70 mL) was stirred for 3 h at rt. The reaction mixture was extracted with EtOAc and the combined organics were washed (brine), dried (Na₂SO₄) and evaporated to dryness in vacuo to give the title compound as a light-yellow oil (7 g, 99%).

### Preparation 4

### 3-fluoro-3-methylpiperidin-4-ol hydrochloride

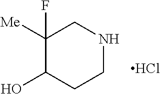

Hydrochloric acid (4 M in dioxane, 50 mL) was added to a solution of tert-butyl 3-fluoro-4-hydroxy-3-methylpiperidine-1-carboxylate (Preparation 3, 7 g, 30.0 mmol) in DCM (70 mL) and the resulting mixture stirred at rt for 3 h. The reaction precipitate was collected by filtration to afford the title compound as a white solid (4.5 g).

### Preparation 5

### 1-(4-aminopyrimidin-2-yl)-3-fluoro-3-methylpiperidin-4-ol

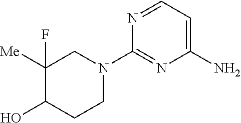 [SMILES: CC1(F)CN(c2nccc(N)n2)CCC1O]

A mixture of 2-chloropyrimidin-4-amine (2.7 g, 20.8 mmol), 3-fluoro-3-methylpiperidin-4-ol hydrochloride (Preparation 4, 3.86 g, 22.8 mmol) and TEA (6.30 g, 62.4 mmol) in IPA (45 mL) was stirred for 5 h at 130° C. in a sealed vial. The reaction mixture was cooled to rt and the solids removed by filtration. The filtrate was evaporated to dryness in vacuo to give the title compound as a yellow oil (6 g) which was used without further purification.

### Preparation 6 and 7

### Cis-rac-1-(4-aminopyrimidin-2-yl)-3-fluoro-3-methylpiperidin-4-ol and trans-rac-1-(4-aminopyrimidin-2-yl)-3-fluoro-3-methylpiperidin-4-ol

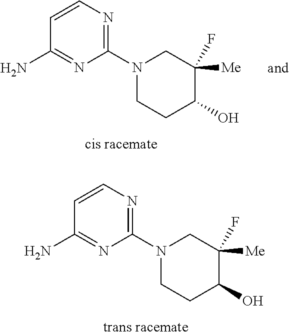 [SMILES: C[C@]1(F)CN(c2nccc(N)n2)CC[C@H]1O ; "and" ; C[C@]1(F)CN(c2nccc(N)n2)CC[C@@H]1O]

1-(4-aminopyrimidin-2-yl)-3-fluoro-3-methylpiperidin-4-ol (Preparation 5) was purified by HPLC-2 to afford the title compounds. Peak 1; Preparation 6, cis-rac-1-(4-aminopyrimidin-2-yl)-3-fluoro-3-methylpiperidin-4-ol as a white solid (1.3 g, 26.1%) and Peak 2; Preparation 7, trans-rac-1-(4-aminopyrimidin-2-yl)-3-fluoro-3-methylpiperidin-4-ol as a white solid (500 mg, 10%).

### Preparation 8 and 9

### (3S,4R)-1-(4-aminopyrimidin-2-yl)-3-fluoro-3-methylpiperidin-4-ol and (3R,4S)-1-(4-aminopyrimidin-2-yl)-3-fluoro-3-methylpiperidin-4-ol

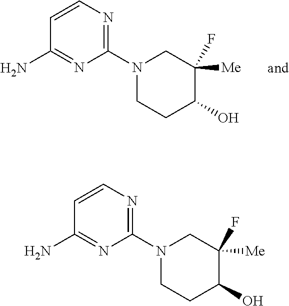 [SMILES: C[C@]1(F)CN(c2nccc(N)n2)CC[C@H]1O ; "and" ; C[C@@]1(F)CN(c2nccc(N)n2)CC[C@@H]1O]

Cis-rac-1-(4-aminopyrimidin-2-yl)-3-fluoro-3-methylpiperidin-4-ol (Preparation 6) was separated by prep-SFC- (Phenomenex Lux 5 u Cellulose-3, 5×25 cm, 5 mm; 50% MeOH (+0.1% DEA) in CO₂) to afford the title enantiomers.

Peak 1 (Preparation 8): (3S,4R)-1-(4-aminopyrimidin-2-yl)-3-fluoro-3-methylpiperidin-4-ol (Stereochemistry assigned by x-ray crystallography of a related compound (not shown)) as a white solid as a white solid (500 mg) and Peak 2 (Preparation 9): (3R,4S)-1-(4-aminopyrimidin-2-yl)-3-fluoro-3-methylpiperidin-4-ol (500 mg).

LCMS m/z=227 [M+H]⁺; ¹H NMR (400 MHz, DMSO-d₆) δ: 7.71 (d, 1H), 6.37 (s, 2H), 5.69 (d, 1H), 4.93 (d, 1H), 4.66 (ddd, 1H), 4.60-4.50 (m, 1H), 3.44 (ddt, 1H), 3.02-2.78 (m, 2H), 1.69-1.53 (m, 2H), 1.31 (d, 3H).

### Preparation 10

### Cis-rac-tert-butyl 3-fluoro-4-hydroxy-4-methylpiperidine-1-carboxylate

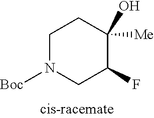 [SMILES: CC(C)(C)OC(=O)N1CC[C@@](C)(O)[C@@H](F)C1]

MeMgBr (9.2 mL, 27.6 mmol) was added to a solution of tert-butyl 3-fluoro-4-oxopiperidine-1-carboxylate (5 g, 2.3 mmol) in THF (50 mL) at −78° C. The mixture was stirred overnight at rt. The reaction mixture was carefully diluted with sat. NH₄Cl (aq), extracted with EtOAc and washed with brine. The combined organics were dried (Na₂SO₄) and evaporated to afford the title compound as a yellow solid (4.8 g, 90%). LCMS m/z=178 [M+H−56]⁺.

### Preparation 11

### Cis-rac-3-fluoro-4-methylpiperidin-4-ol hydrochloride

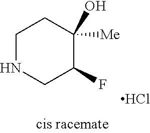

Rac-tert-butyl (3R,4S)-3-fluoro-4-hydroxy-4-methylpiperidine-1-carboxylate (Preparation 10, 4.8 g, 20 mmol) in HCl/dioxane (50 mL) was stirred at rt for 4 h. The reaction mixture was evaporated to afford the title compound as a yellow solid (3 g, crude) which was used directly for the next step. LCMS m/z=134 [M+H]⁺.

### Preparation 12

### cis-rac-1-(4-aminopyrimidin-2-yl)-3-fluoro-4-methylpiperidin-4-ol

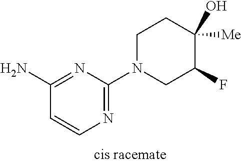 [SMILES: C[C@@]1(O)CCN(c2nccc(N)n2)C[C@@H]1F]

A mixture of 2-chloropyrimidin-4-amine (1.5 g, 11.5 mmol), cis-rac-3-fluoro-4-methylpiperidin-4-ol hydrochloride (Preparation 11, 3 g) and DIPEA (11.9 g, 92.3 mmol) in DMSO (40 mL) was stirred overnight at 120° C. The reaction mixture was diluted with water, extracted (EtOAc) and washed with brine. The combined organics were dried (Na₂SO₄) and evaporated to dryness in vacuo to afford the title compound as a light-yellow solid (1.3 g). LCMS m/z=227 [M+H]⁺.

### Preparation 13 and 14

### (3S,4R)-1-(4-aminopyrimidin-2-yl)-3-fluoro-4-methylpiperidin-4-ol and (3R,4S)-1-(4-aminopyrimidin-2-yl)-3-fluoro-4-methylpiperidin-4-ol

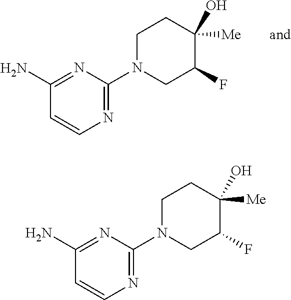 [SMILES: C[C@@]1(O)CCN(c2nccc(N)n2)C[C@@H]1F ; "and" ; C[C@]1(O)CCN(c2nccc(N)n2)C[C@H]1F]

Cis-rac-1-(4-aminopyrimidin-2-yl)-3-fluoro-4-methylpiperidin-4-ol (Preparation 12) was separated by preparative SFC using the following conditions: Column: CHIRAL Cellulose-SJ (4.6*150 mm, 5 um); Mobile Phase: CO₂/MeOH (0.1% DEA); Flow Rate: 4 g/min); to afford Peak 1, Preparation 13: (3S,4R)-1-(4-aminopyrimidin-2-yl)-3-fluoro-4-methylpiperidin-4-ol (450 mg, Stereochemistry assigned by x-ray crystallography of a related compound (not shown)) as a white solid and Peak 2, Preparation 14: (3R,4S)-1-(4-aminopyrimidin-2-yl)-3-fluoro-4-methylpiperidin-4-ol (470 mg) as a white solid. Peak 1, Preparation 13: (3S,4R)-1-(4-aminopyrimidin-2-yl)-3-fluoro-4-methylpiperidin-4-ol. ¹H NMR (300 MHz, DMSO-d₆) δ: 7.73 (d, 1H), 6.40 (s, 2H), 5.72 (d, 1H), 4.71 (s, 1H), 4.39-3.92 (m, 3H), 3.38 (dddd, 2H), 1.62 (q, 1H), 1.42 (td, 1H), 1.20 (s, 3H). Peak 2, Preparation 14: (3R,4S)-1-(4-aminopyrimidin-2-yl)-3-fluoro-4-methylpiperidin-4-ol. ¹H NMR (300 MHz, DMSO-d₆) δ: 7.73 (d, 1H), 6.40 (s, 2H), 5.72 (d, 1H), 4.71 (s, 1H), 4.36-4.07 (m, 2H), 4.07-3.95 (m, 1H), 3.44 (ddd, 1H), 3.31 (ddd, 1H), 1.61 (ddt, 1H), 1.41 (ddd, 1H), 1.20 (s, 3H).

### Preparation 15

### (3S,4R)-1-(4-aminopyrimidin-2-yl)-4-methoxypiperidin-3-ol and (3R,4S)-1-(4-aminopyrimidin-2-yl)-4-methoxypiperidin-3-ol

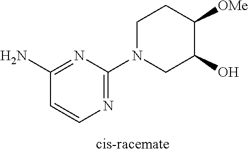 [SMILES: CO[C@@H]1CCN(c2nccc(N)n2)C[C@@H]1O]

A mixture of cis-rac-4-methoxypiperidin-3-ol (1.7 g, 13 mmol), 2-chloropyrimidin-4-amine (1.7 g, 13 mmol) and TEA (2.6 g, 26 mmol) in IPA (15 mL) was stirred overnight at 100° C. The mixture was concentrated and the residue purified by column chromatography (5% MeOH in DCM) to afford the title compound as a yellow solid (2.4 g, 82.7%). LCMS m/z=225 [M+H]⁺.

### Preparation 16 and 17

### (3S,4R)-1-(4-aminopyrimidin-2-yl)-4-methoxypiperidin-3-ol and (3R,4S)-1-(4-aminopyrimidin-2-yl)-4-methoxypiperidin-3-ol

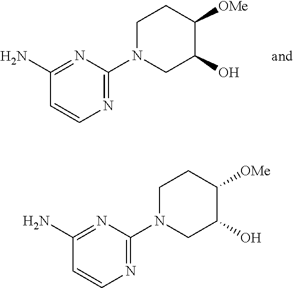 [SMILES: CO[C@@H]1CCN(c2nccc(N)n2)C[C@@H]1O ; "and" ; CO[C@H]1CCN(c2nccc(N)n2)C[C@H]1O]

Cis-rac-1-(4-aminopyrimidin-2-yl)-4-methoxypiperidin-3-ol (Preparation 15, 2.4 g) was separated by Chiral-SFC (Chiralpak IA, 4.6×150 mm, 5 mm; 10% MeOH (+0.1% DEA) in CO₂) to afford the title compounds. Peak 1: (3S,4R)-1-(4-aminopyrimidin-2-yl)-4-methoxypiperidin-3-ol or (3R,4S)-1-(4-aminopyrimidin-2-yl)-4-methoxypiperidin-3-ol (900 mg); Peak 2: (3R,4S)-1-(4-aminopyrimidin-2-yl)-4-methoxypiperidin-3-ol or (3S,4R)-1-(4-aminopyrimidin-2-yl)-4-methoxypiperidin-3-ol (890 mg).

### Preparation 18 and 19

### (3S,4S)-1-(4-aminopyrimidin-2-yl)-4-methoxypiperidin-3-ol and (3R,4R)-1-(4-aminopyrimidin-2-yl)-4-methoxypiperidin-3-ol

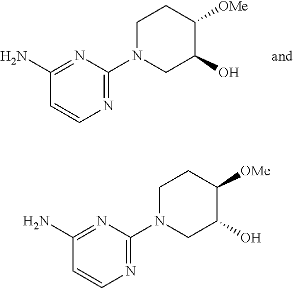 [SMILES: CO[C@H]1CCN(c2nccc(N)n2)C[C@@H]1O ; "and" ; CO[C@@H]1CCN(c2nccc(N)n2)C[C@H]1O]

Part 1: 2-Chloropyrimidin-4-amine (987 mg, 7.62 mmol) was added to trans-rac-4-methoxypiperidin-3-ol (1.0 g, 7.62 mmol) and TEA (2.30 g, 22.8 mmol) in IPA (20 mL) at rt and the mixture stirred at 100° C. for 16 h. The mixture was concentrated under vacuum and the residue was purified by a silica gel column (20:1 DCM/MeOH) to afford trans-rac-1-(4-aminopyrimidin-2-yl)-4-methoxypiperidin-3-ol as a colorless oil (1.2 g).

Part 2: Trans-rac-1-(4-aminopyrimidin-2-yl)-4-methoxypiperidin-3-ol (Part 1, 1.2 g, 5.35 mmol) was purified by chiral-SFC (CHIRALPAK IC, 20×250 mm, 5 mm; 25% EtOH (8 mM NH₃·MeOH) in CO₂) to afford the title compounds. Peak 1, Preparation 18; (3S,4S)-1-(4-aminopyrimidin-2-yl)-4-methoxypiperidin-3-ol or (3R,4R)-1-(4-aminopyrimidin-2-yl)-4-methoxypiperidin-3-ol as a white solid (450 mg); Peak 2, Preparation 19; (3R,4R)-1-(4-aminopyrimidin-2-yl)-4-methoxypiperidin-3-ol or (3S,4S)-1-(4-aminopyrimidin-2-yl)-4-methoxypiperidin-3-ol as a white solid (460 mg).

LCMS m/z=225 [M+H]⁺.

### Preparation 20

### (4R,5S)-1-(4-aminopyrimidin-2-yl)-5-fluoro-3,3-dimethylpiperidin-4-ol and (4S,5R)-1-(4-aminopyrimidin-2-yl)-5-fluoro-3,3-dimethylpiperidin-4-ol

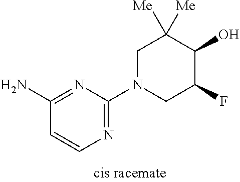 [SMILES: CC1(C)CN(c2nccc(N)n2)C[C@H](F)[C@@H]1O]

Part 1: Cis-tert-butyl 5-fluoro-4-hydroxy-3,3-dimethylpiperidine-1-carboxylate (4.7 g, 19.0 mmol) was added to a solution of HCl in 1,4-dioxane (30 mL) and the resulting mixture stirred at rt for 16 h. The solvent was removed under reduced pressure to afford cis-5-fluoro-3,3-dimethylpiperidin-4-ol hydrochloride as a white solid (3.6 g) which was used without further purification in Part 2.

Part 2: TEA (3.83 g, 38.0 mmol) was added to a mixture of cis-5-fluoro-3,3-dimethylpiperidin-4-ol hydrochloride (Part 1, 3.6 g, 19.0 mmol) and 2-chloropyrimidin-4-amine (2.46 g, 19.0 mmol) in IPA (10 mL) and the resulting mixture stirred at 100° C. for 3 h. The solids were removed by filtration and the filtrate was concentrated under reduced pressure. The residue was purified by HPLC-9 to afford the title compound as a white solid (1.8 g, 39.4%). LCMS m/z=241 [M+H]⁺.

### Preparation 21 and 22

### (4R,5S)-1-(4-aminopyrimidin-2-yl)-5-fluoro-3,3-dimethylpiperidin-4-ol and (4S,5R)-1-(4-aminopyrimidin-2-yl)-5-fluoro-3,3-dimethylpiperidin-4-ol

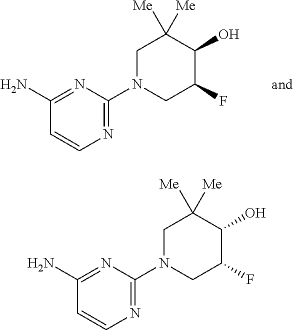 [SMILES: CC1(C)CN(c2nccc(N)n2)C[C@H](F)[C@@H]1O ; "and" ; CC1(C)CN(c2nccc(N)n2)C[C@@H](F)[C@H]1O]

(4R,5S)-1-(4-aminopyrimidin-2-yl)-5-fluoro-3,3-dimethylpiperidin-4-ol and (4S,5R)-1-(4-aminopyrimidin-2-yl)-5-fluoro-3,3-dimethylpiperidin-4-ol (Preparation 20, 1.8 g) were separated by preparative chiral-SFC (EnantioPak-A1-5(02), 50×250 mm, 5 mm; 60% EtOH (0.1% DEA) in CO₂) to afford the title compounds. Peak 1: (4R,5S)-1-(4-aminopyrimidin-2-yl)-5-fluoro-3,3-dimethylpiperidin-4-ol or (4S,5R)-1-(4-aminopyrimidin-2-yl)-5-fluoro-3,3-dimethylpiperidin-4-ol (776 mg, 43.3%) as a white solid and Peak 2: (4S,5R)-1-(4-aminopyrimidin-2-yl)-5-fluoro-3,3-dimethylpiperidin-4-ol or (4R,5S)-1-(4-aminopyrimidin-2-yl)-5-fluoro-3,3-dimethylpiperidin-4-ol (700 mg, 39.1%) as a white solid.

LCMS m/z=241 [M+H]⁺. ¹H NMR (300 MHz, DMSO-d₆) δ: 7.69 (d, 1H), 6.36 (s, 2H), 5.67 (d, 1H), 5.00 (d, 1H), 4.75-4.47 (m, 1H), 4.09-3.93 (m, 1H), 3.86-3.67 (m, 1H), 3.62 (d, 1H), 3.37 (ddd, 1H), 3.31-3.18 (m, 1H), 0.95-0.78 (m, 6H).

### Preparation 23

### 1-(4-aminopyrimidin-2-yl)-4-methylpiperidin-4-ol

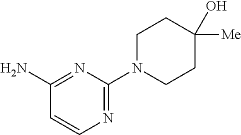 [SMILES: CC1(O)CCN(c2nccc(N)n2)CC1]

A mixture of 4-methylpiperidin-4-ol (230 mg, 2 mmol), 2-chloropyrimidin-4-amine (258 mg, 2 mmol) and TEA (300 mg, 3 mmol) in IPA (5 mL) was stirred overnight at rt. The solvent was evaporated to dryness in vacuo and the residue purified by prep-TLC (6% MeOH in DCM) to afford the title compound (210 mg, 50%). LCMS m/z=209 [M+H]⁺.

### Preparation 24

### tert-butyl (3S,4R)-3-fluoro-4-(2-hydroxyethoxy)piperidine-1-carboxylate

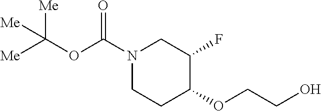 [SMILES: CC(C)(C)OC(=O)N1CC[C@@H](OCCO)[C@@H](F)C1]

NaH (1.35 g, 33.9 mmol) was added batchwise to tert-butyl (3S,4R)-3-fluoro-4-hydroxypiperidine-1-carboxylate (3.0 g, 13.6 mmol) in DMF (10 mL) at 0° C. and stirred at 0° C. for 20 min. (2-Bromoethoxy)(tert-butyl)dimethylsilane (9.76 g, 40.8 mmol) was added and the mixture stirred at rt for 16 h. The reaction mixture was diluted with EtOAc, washed with brine, dried (Na₂SO₄) and evaporated to dryness in vacuo. The residue was purified by column chromatography (10:1 PE/EtOAc) to afford the title compound as a colorless oil (3 g).

### Preparation 25

### 2-(((3S,4R)-1-(4-aminopyrimidin-2-yl)-3-fluoropiperidin-4-yl)oxy)ethan-1-ol

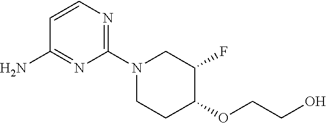 [SMILES: Nc1ccnc(N2CC[C@@H](OCCO)[C@@H](F)C2)n1]

TFA (15 mL) was added to tert-butyl (3S,4R)-3-fluoro-4-(2-hydroxyethoxy)piperidine-1-carboxylate (Preparation 24, 3.0 g, 7.94 mmol) in DCM (20 mL) at rt and the resulting mixture stirred at rt for 1 h. The mixture was concentrated under vacuum and the residue mixed with 2-chloropyrimidin-4-amine (873 mg, 6.74 mmol) and DIPEA (629 mg, 4.88 mmol) in DMSO (10 mL) and the mixture stirred overnight at 100° C. The reaction mixture was diluted (EtOAc, 50 mL) and washed (brine). The combined organics were dried (Na₂SO₄) and evaporated to dryness in vacuo and the residue purified by column chromatography (1:15 MeOH/EtOAc) to afford the title compound as a yellow solid (1.1 g). LCMS m/z=257 [M+H]⁺. ¹H NMR (400 MHz, DMSO-d₆) δ: 7.72 (d, 1H), 6.41 (s, 2H), 5.71 (d, 1H), 4.94-4.69 (m, 1H), 4.67-4.52 (m, 2H), 4.34 (d, 1H), 3.72-3.45 (m, 5H), 3.31-3.19 (m, 1H), 3.07 (t, 1H), 1.77-1.44 (m, 2H).

### Preparation 26

### tert-butyl (3R,4S)-3-fluoro-4-(methoxy-d3)piperidine-1-carboxylate

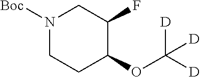 [SMILES: [2H]C([2H])([2H])O[C@H]1CCN(C(=O)OC(C)(C)C)C[C@H]1F]

NaH (218 mg, 9.08 mmol) was added to tert-butyl (3R,4S)-3-fluoro-4-hydroxypiperidine-1-carboxylate (1000 mg, 4.56 mmol) in DMF (20 mL) at 0° C. and stirred for 20 mins before CD₃I (3.30 g, 22.8 mmol) was added and the solution stirred at rt for 16 h. The reaction was quenched by the addition of H₂O (5 mL) and the solids removed by filtration. The filtrate was extracted into EtOAc and the combined organics washed with brine and evaporated to dryness in vacuo to afford the title compound as a light-yellow oil (1.14 g).

### Preparation 27

### tert-butyl (3S,4R)-3-fluoro-4-(methoxy-d3)piperidine-1-carboxylate

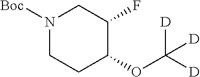 [SMILES: [2H]C([2H])([2H])O[C@@H]1CCN(C(=O)OC(C)(C)C)C[C@@H]1F]

The title compound was prepared from tert-butyl (3S,4R)-3-fluoro-4-hydroxypiperidine-1-carboxylate using an analogous method to that described for Preparation 26.

### Preparation 28

### 2-((3R,4S)-3-fluoro-4-(methoxy-d₃)piperidin-1-yl)pyrimidin-4-amine

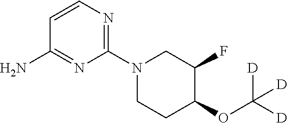 [SMILES: [2H]C([2H])([2H])O[C@H]1CCN(c2nccc(N)n2)C[C@H]1F]

TFA (2 mL) was added to tert-butyl (3R,4S)-3-fluoro-4-(methoxy-d₃)piperidine-1-carboxylate (Preparation 26, 1140 mg, 4.82 mmol) in DCM (6 mL) and the solution stirred for 2 h at rt. The mixture was evaporated to dryness in vacuo. The resulting residue was dissolved in IPA (20 mL), 2-chloropyrimidin-4-amine (496 mg, 3.83 mmol) and TEA (0.6 mL) added and the reaction mixture stirred overnight at 100° C. The mixture was evaporated to dryness and the residue purified by column chromatography (5% MeOH in EtOAc) to afford the title compound as a light-yellow solid (425 mg). LCMS m/z=230 [M+H]⁺.

### Preparation 29

### 2-((3S,4R)-3-fluoro-4-(methoxy-d3)piperidin-1-yl)pyrimidin-4-amine

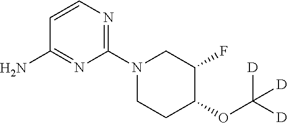 [SMILES: [2H]C([2H])([2H])O[C@@H]1CCN(c2nccc(N)n2)C[C@@H]1F]

The title compound was prepared from tert-butyl (3S,4R)-3-fluoro-4-(methoxy-d3)piperidine-1-carboxylate (Preparation 27) using an analogous method to that described for Preparation 28. LCMS m/z=230 [M+H]⁺.

### Preparation 30

### 2-((3R,4S)-3-fluoro-4-methoxypiperidin-1-yl)pyrimidin-4-amine

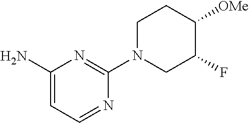 [SMILES: CO[C@H]1CCN(c2nccc(N)n2)C[C@H]1F]

Part 1: NaH (152 mg, 3.82 mmol) was added to a solution of tert-butyl (3R,4S)-3-fluoro-4-hydroxypiperidine-1-carboxylate (700 mg, 3.19 mmol) in THF (5 mL) at 0° C. Mel (497 mg, 3.5 mmol) was added and the mixture warmed to rt and stirred for 2 h. The reaction mixture was quenched with H₂O, extracted with EtOAc and washed with brine. The combined organics were dried (Na₂SO₄) and evaporated to dryness in vacuo to afford tert-butyl (3R,4S)-3-fluoro-4-methoxypiperidine-1-carboxylate as a yellow oil (750 mg, crude) which was used without further purification in Part 2. LCMS m/z=230 [M+H]⁺.

Part 2: TFA (2 mL) was added to a solution of tert-butyl (3R,4S)-3-fluoro-4-methoxypiperidine-1-carboxylate (Part 1, 750 mg, 3.21 mmol) in DCM (10 mL) and the mixture stirred at rt for 3 h. The reaction mixture was evaporated to afford (3R,4S)-3-fluoro-4-methoxypiperidine trifluoroacetate as a brown oil (700 mg, crude).

Part 3: A mixture of (3R,4S)-3-fluoro-4-methoxypiperidine trifluoroacetate (Part 2, 700 mg, 5.25 mmol), 2-chloropyrimidin-4-amine (488 mg, 3.76 mmol) and DIPEA (1.44 g, 11.2 mmol) in DMSO (5 mL) was stirred at 100° C. for 2 h. The reaction mixture was diluted with water, extracted with EtOAc and washed with brine. The combined organics were dried (Na₂SO₄) and evaporated to dryness. The residue was purified by column chromatography (50% EtOAc in PE) to afford the title compound as a yellow solid (550 mg).

### Preparation 31

### 2-((3S,4R)-3-fluoro-4-methoxypiperidin-1-yl)pyrimidin-4-amine

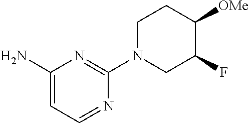 [SMILES: CO[C@@H]1CCN(c2nccc(N)n2)C[C@@H]1F]

The title compound was prepared from tert-butyl (3S,4R)-3-fluoro-4-hydroxypiperidine-1-carboxylate using an analogous method to that described for Preparation 30. LCMS m/z=227 [M+H]⁺. ¹H NMR (400 MHz, DMSO-d₆) δ: 7.72 (d, 1H), 6.39 (s, 2H), 5.71 (d, 1H), 4.83 (d, 1H), 4.60-4.49 (m, 1H), 4.29 (d, 1H), 3.55-3.42 (m, 1H), 3.28 (d, 1H), 3.20-3.04 (m, 1H), 1.76-1.48 (m, 2H).

### Preparation 32

### Cis-rac-2-((3aR,6aS)-hexahydro-1H-furo[3,4-b]pyrrol-1-yl)pyrimidin-4-amine

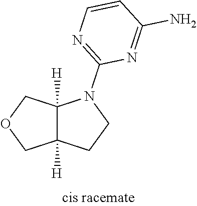 [SMILES: Nc1ccnc(N2CC[C@H]3COC[C@H]32)n1]

A mixture of 2-chloropyrimidin-4-amine (370 mg, 2.85 mmol), cis-rac-(3aR,6aS)-hexahydro-1H-furo[3,4-b]pyrrole (322 mg, 2.85 mmol) and DIPEA (1.10 g, 8.55 mmol) in DMSO (8 mL) was stirred for 12 h at 120° C. The reaction mixture was diluted with water, extracted with EtOAc and washed with brine. The organic layer was dried (Na₂SO₄) and evaporated to dryness in vacuo. The residue was purified by column chromatography (20:1 DCM/MeOH) to afford the title compound as a yellow solid (410 mg, 70%). LCMS m/z=207 [M+H]⁺.

### Preparation 33

### benzyl 3-(cyano(methylsulfonyl)methyl)azetidine-1-carboxylate

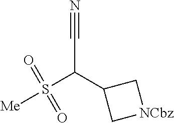 [SMILES: CS(=O)(=O)C(C#N)C1CN(C(=O)OCc2ccccc2)C1]

Cs₂CO₃ (544 mg, 1.67 mmol) was added to 2-(methylsulfonyl)acetonitrile (1 g, 8.39 mmol) and benzyl 3-iodoazetidine-1-carboxylate (3.96 g, 12.5 mmol) in DMF (5 mL) at rt and the resulting mixture was stirred at 80° C. for 8 h. The reaction mixture was diluted with EtOAc and washed with brine. The organic layer was dried (Na₂SO₄) and evaporated to dryness in vacuo to afford a residue which was purified by silica gel chromatography (5:1, PE/EtOAc) to afford the title compound as a colourless oil (1.2 g, 46%). LCMS m/z=308 [M+H]⁺

### Preparation 34

### benzyl 3-(2-amino-1-(methylsulfonyl)ethyl)azetidine-1-carboxylate

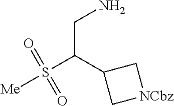 [SMILES: CS(=O)(=O)C(CN)C1CN(C(=O)OCc2ccccc2)C1]

Benzyl 3-(cyano(methylsulfonyl)methyl)azetidine-1-carboxylate (Preparation 33, 1.2 g, 3.89 mmol) and Raney-Ni (10 mg) in EtOH (4 mL) was stirred under H₂ at rt for 8 h. The solids were removed by filtration and the filtrate evaporated to dryness in vacuo to give the title compound as a colourless oil (600 mg, 49%). LCMS m/z=313 [M+H]⁺

### Preparation 35

### benzyl 3-(2-((tert-butoxycarbonyl)amino)-1-(methylsulfonyl)ethyl)azetidine-1-carboxylate

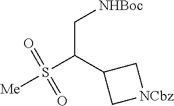 [SMILES: CC(C)(C)OC(=O)NCC(C1CN(C(=O)OCc2ccccc2)C1)S(C)(=O)=O]

(Boc)₂O (829 mg, 3.84 mmol) was added portion wise to a mixture of Na₂CO₃ (407 mg, 3.84 mmol) and benzyl 3-(2-amino-1-(methylsulfonyl)ethyl)azetidine-1-carboxylate (Preparation 34, 600 mg, 1.92 mmol) in dioxane/H₂O at 0° C. and the resulting mixture stirred at rt for 16 h. The reaction mixture was diluted with EtOAc and washed with brine. The combined organics were dried (Na₂SO₄) and evaporated to dryness in vacuo. The residue was purified by silica gel chromatography (5:1 PE/EtOAc) to afford the title compound as a colourless oil (650 mg, 82%). LCMS m/z=413 [M+H]⁺

### Preparation 36

### tert-butyl (2-(azetidin-3-yl)-2-(methylsulfonyl)ethyl)carbamate

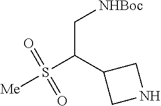 [SMILES: CC(C)(C)OC(=O)NCC(C1CNC1)S(C)(=O)=O]

Benzyl 3-(2-((tert-butoxycarbonyl)amino)-1-(methylsulfonyl)ethyl)azetidine-1-carboxylate (Preparation 35, 600 mg, 1.92 mmol) and Pearlman's catalyst (300 mg, 2.14 mmol) in MeOH (50 mL) was stirred under H₂ at rt for 16 h. The solids were removed by filtration and the filtrate evaporated to dryness in vacuo to give the title compound (300 mg). LCMS m/z=279 [M+H]⁺

### Preparation 37

### 8-bromo-3-chloro-5-methoxyisoquinoline

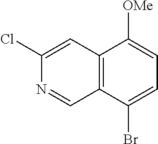 [SMILES: COc1ccc(Br)c2cnc(Cl)cc12]

Br₂ (0.4 mL, 7.75 mmol) was added slowly to a suspension of 3-chloro-5-methoxyisoquinoline (1000 mg, 5.16 mmol) suspended in AcOH (10 mL) at rt and the resulting mixture stirred overnight. Additional Br₂ (0.15 mL) was added and stirring continued for 3 h. The reaction mixture was diluted with EtOAc and slowly quenched with sat. aq. NaHCO₃ and extracted with EtOAc. The combined extracts were dried (Na₂SO₄) and evaporated to dryness in vacuo. The residue was purified by ISCO chromatography (0-70% EtOAc/Hex) to afford the title compound as an orange solid (1.08 g, 77%). ¹H NMR (400 MHz, DMSO-d₆) δ: 9.26 (d, 1H), 8.05 (s, 1H), 7.94 (d, 1H), 7.24 (d, 1H), 4.02 (s, 3H).

### Preparation 38

### 8-bromo-3-chloroisoquinolin-5-ol

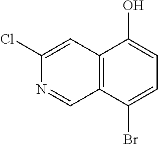 [SMILES: Oc1ccc(Br)c2cnc(Cl)cc12]

BBr₃ (30.8 mL, 30.8 mmol, 1M in DCM) was added to a solution of 8-bromo-3-chloro-5-methoxyisoquinoline (Preparation 37, 2.4 g, 8.81 mmol) in DCM (31.5 mL) at 0° C. and the reaction mixture stirred at rt overnight. The reaction mixture was slowly poured into cold H₂O with rapid stirring and the resulting solids collected via vacuum filtration and dried under high vacuum for 1 h to afford the title compound as a yellow solid (2.20 g, 97%). ¹H NMR (400 MHz, DMSO-d₆) δ: 9.19 (d, 1H), 8.01 (d, 1H), 7.79 (d, 1H), 7.06 (d, 1H).

### Preparation 39

### 8-bromo-3-chloroisoquinolin-5-yl trifluoromethanesulfonate

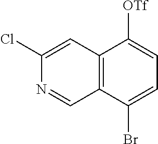 [SMILES: O=S(=O)(Oc1ccc(Br)c2cnc(Cl)cc12)C(F)(F)F]

Trifluoromethanesulfonic anhydride (2.59 mL, 15.32 mmol) was added dropwise to a solution of TEA (3.2 mL, 23 mmol) and 8-bromo-3-chloroisoquinolin-5-ol (1.98 g, 7.66 mmol) in dry DCM (70 mL) at −60° C. The reaction was warmed to rt and stirred for 1 h. The mixture was evaporated to dryness in vacuo and the residue purified by ISCO chromatography (100% DCM) to afford the title compound as a beige solid (2.513 g, 84%). ¹H NMR (400 MHz, DMSO-d₆) δ: 9.49 (d, 1H), 8.22 (d, 1H), 8.03 (d, 1H), 7.95 (s, 1H).

### Preparation 40

### 8-bromo-3-chloro-5-(prop-1-en-2-yl)isoquinoline

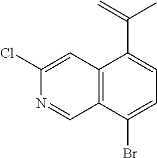

A solution of 4,4,5,5-tetramethyl-2-(prop-1-en-2-yl)-1,3,2-dioxaborolane (3.61 mL, 19.20 mmol), 8-bromo-3-chloroisoquinolin-5-yl trifluoromethanesulfonate (Preparation 39, 7.5 g, 19.2 mmol), K₂CO₃ (2.65 g, 19.20 mmol) and PdCl₂(dppf)·DCM (1.568 g, 1.92 mmol) in dioxane (87 mL) and H₂O (8.7 mL) was purged with N₂ for 5 min before stirring at 45° C. overnight. The mixture was diluted with EtOAc (100 mL) and washed with brine (2×40 mL). The combined organics were dried (Na₂SO₄) and evaporated to dryness in vacuo. The residue was purified using ISCO chromatography (0-10% EtOAc in Hexanes) to afford the title compound as a white solid (2.745 g, 50.6%).

### Preparation 41

### 8-bromo-3-chloro-5-isopropylisoquinoline

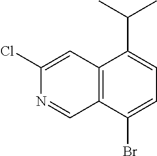

A mixture of 8-bromo-3-chloro-5-(prop-1-en-2-yl)isoquinoline (Preparation 40, 500 mg, 1.770 mmol) and PtO₂ (40.2 mg, 0.177 mmol) in EtOAc (12 mL) was placed under a balloon of H₂ and stirred at rt for 1.5 h. The solids were removed by filtration through a pad of Celite® and washed with EtOAc. The combined organics were evaporated to dryness in vacuo and the residue was purified by ISCO chromatography (0-15% EtOAc/Hex) to afford the title compound as a white solid (376 mg, 74.7%). LCMS m/z=286 [M+H]⁺

### Preparation 42

### 3-chloro-5-(prop-1-en-2-yl)isoquinolin-8-ol

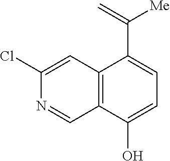

A mixture of 8-bromo-3-chloro-5-(prop-1-en-2-yl)isoquinoline (Preparation 40, 328 mg, 1.161 mmol), KOH (261 mg, 4.64 mmol), Pd₂(dba)₃ (21 mg, 0.023 mmol) and tetramethyl tBuXPhos (45 mg, 0.093 mmol) in dioxane (2 mL) and H₂O (2 mL) was heated under N₂ at 90° C. for 1 h. The reaction mixture was diluted with H₂O and the pH adjusted to ˜3-4 by the addition of 1M HCl (˜3-4 mL) and extracted with 5% MeOH/DCM (×2). The combined organics were dried (Na₂SO₄) and evaporated to dryness in vacuo. The residue was purified by ISCO chromatography (5-100% EtOAc/Hex) to give the title compound as a pale yellow solid (189 mg, 74.1%). LCMS m/z=220 [M+H]⁺

### Preparation 43

### 3-chloro-5-isopropylisoquinolin-8-ol

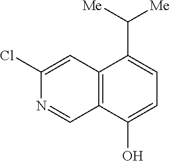 [SMILES: CC(C)c1ccc(O)c2cnc(Cl)cc12]

PtO₂ (28 mg) was added to a solution of 3-chloro-5-(prop-1-en-2-yl)isoquinolin-8-ol (Preparation 42, 270 mg, 1.229 mmol) in EtOAc (12 mL) and the mixture stirred under a balloon atmosphere of H₂ for 2 h at rt. The solids were removed by filtration and the filtrate was evaporated to dryness to afford the title compound as a yellow solid (255 mg, 94%). LCMS m/z=222 [M+H]⁺

### Preparation 44

### 1-(3-(((3-chloro-5-isopropylisoquinolin-8-yl)amino)methyl)azetidin-1-yl)ethan-1-one

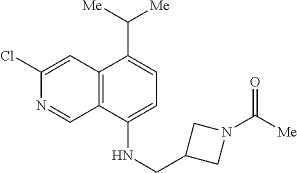 [SMILES: CC(=O)N1CC(CNc2ccc(C(C)C)c3cc(Cl)ncc23)C1]

Part 1. A mixture of 8-bromo-3-chloro-5-(prop-1-en-2-yl)isoquinoline (Preparation 40, 75 mg, 0.265 mmol), 1-(3-(aminomethyl)azetidin-1-yl)ethenone (37 mg, 0.292 mmol) and Cs₂CO₃ (173 mg, 0.531 mmol) in dioxane (1 mL) was heated at 90° C. under N₂ for 1 h. The mixture was diluted with H₂O and extracted with EtOAc. The combined organics were washed with brine, dried (Na₂SO₄) and evaporated to dryness in vacuo. The residue was purified by RP-ISCO (0 to 100% MeCN/H₂O (+0.1% TFA) and the resulting product diluted aq. NaHCO₃ and extracted with DCM (×2). The combined organics were dried (Na₂SO₄) and evaporated to dryness in vacuo to afford 1-(3-(((3-chloro-5-(prop-1-en-2-yl)isoquinolin-8-yl)amino)methyl)azetidin-1-yl)ethan-1-one as a pale yellow solid (23 mg, 26%) which was used without further purification.

Part 2. PtO₂ (1.58 mg) was added to a solution of 1-(3-(((3-chloro-5-(prop-1-en-2-yl)isoquinolin-8-yl)amino)methyl)azetidin-1-yl)ethan-1-one (Part 1, 23 mg, 0.070 mmol) in EtOAc (1.5 mL) and the mixture stirred under a balloon atmosphere of H₂ for 5 h at rt. The solids were removed by filtration and the filtrate was evaporated to dryness to afford the title compound as a yellow solid (21 mg, 91%). LCMS m/z=332 [M+H]⁺

### Preparation 45

### 3-chloro-5-isopropyl-8-((1-methyl-1H-pyrazol-4-yl)methoxy)isoquinoline

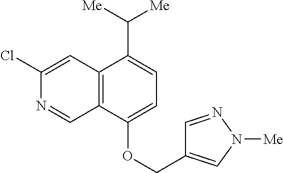

Part 1. A mixture of 8-bromo-3-chloro-5-(prop-1-en-2-yl)isoquinoline (Preparation 40, 66 mg, 0.234 mmol), (1-methyl-1H-pyrazol-4-yl)methanol (31 mg, 0.280 mmol), di-tert-butyl(2′,4′,6′-triisopropyl-3-methoxy-6-methyl-[1,1′-biphenyl]-2-yl)phosphine (3.28 mg, 0.007 mmol), allylpalladium(II) chloride dimer (0.86 mg, 0.0023 mmol) and Cs₂CO₃ (114 mg, 0.350 mmol) in toluene (0.75 mL) was heated under N₂ at 90° C. for 90 min. The reaction mixture was diluted with EtOAc and filtered through a pad of Celite®. The filtrate was evaporated to dryness in vacuo and the residue purified by ISCO chromatography (0-100% EtOAc/Hex) to give 3-chloro-8-((1-methyl-1H-pyrazol-4-yl)methoxy)-5-(prop-1-en-2-yl)isoquinoline as an off-white solid (31 mg, 42.3%).

Part 2. PtO₂ (2.24 mg, 0.0098 mmol) was added to a solution of 3-chloro-8-((1-methyl-1H-pyrazol-4-yl)methoxy)-5-(prop-1-en-2-yl)isoquinoline (Part 1, 31 mg) and the mixture hydrogenated under a balloon atmosphere of H₂ at rt for 90 min. The solids were removed by filtration and the filtrate was evaporated to dryness to afford the title compound as an off-white solid (30 mg, 96%). LCMS m/z=316 [M+H]⁺

### Preparation 46

### tert-butyl (2-(1-(3-chloro-5-isopropylisoquinolin-8-yl)azetidin-3-yl)-2-(methylsulfonyl)ethyl)carbamate

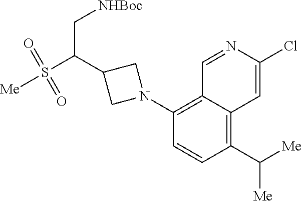 [SMILES: CC(C)c1ccc(N2CC(C(CNC(=O)OC(C)(C)C)S(C)(=O)=O)C2)c2cnc(Cl)cc12]

Cs₂CO₃ (34.2 mg, 0.105 mmol) was added to a mixture of 8-bromo-3-chloro-5-isopropylisoquinoline (Preparation 41, 200 mg, 0.702 mmol), tert-butyl (2-(azetidin-3-yl)-2-(methylsulfonyl)ethyl)carbamate (Preparation 36, 195 mg, 0.702 mmol) and XantPhos Pd G3 (11.3 mg, 0.007 mmol) in dioxane (2 mL) at rt and the resulting mixture stirred at 100° C. for 16 h. The mixture was diluted with EtOAc and washed with brine. The organic layer was dried (Na₂SO₄) and evaporated to dryness in vacuo and the residue purified by prep-TLC (25:1 DCM/MeOH) to afford the title compound as a yellow solid (180 mg). LCMS m/z=482 [M+H]⁺

### Preparation 47 and 48

### tert-butyl ((R)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate and tert-butyl ((S)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate

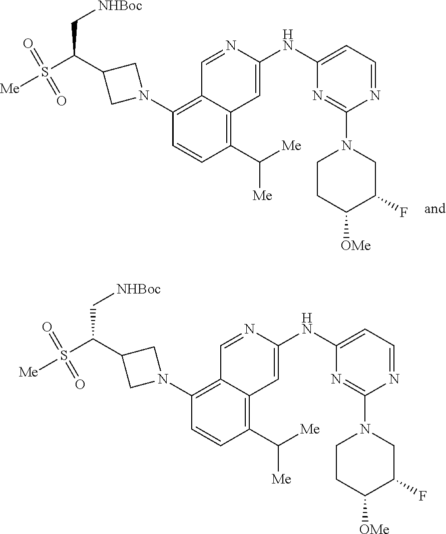 [SMILES: CO[C@@H]1CCN(c2nccc(Nc3cc4c(C(C)C)ccc(N5CC([C@H](CNC(=O)OC(C)(C)C)S(C)(=O)=O)C5)c4cn3)n2)C[C@@H]1F ; CO[C@@H]1CCN(c2nccc(Nc3cc4c(C(C)C)ccc(N5CC([C@@H](CNC(=O)OC(C)(C)C)S(C)(=O)=O)C5)c4cn3)n2)C[C@@H]1F]

Cs₂CO₃ (9.42 mg, 0.029 mmol) was added to BrettPhos Pd G3 (2.02 mg, 0.0022 mmol), tert-butyl (2-(1-(3-chloro-5-isopropylisoquinolin-8-yl)azetidin-3-yl)-2-(methylsulfonyl)ethyl)carbamate (Preparation 46, 10 mg, 0.022 mmol) and 2-((3S,4R)-3-fluoro-4-methoxypiperidin-1-yl)pyrimidin-4-amine (Preparation 31, 5.04 mg, 0.0223 mmol) in dioxane at rt and the resulting mixture stirred at 100° C. for 16 h. The mixture was diluted with EtOAc and washed with brine. The organic layer was dried (Na₂SO₄) and evaporated to dryness in vacuo and the residue purified by prep-TLC (20:1 DCM/MeOH) to afford tert-butyl (2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate as a yellow solid (130 mg). The residue was further purified by HPLC-15 to afford,

Peak 1: tert-butyl ((R)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate or tert-butyl ((S)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate as a yellow solid (25 mg). LCMS m/z=672 [M+H]⁺, and Peak 2: tert-butyl ((S)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate or tert-butyl ((R)-2-(1-(3-((2-((3S,4R)-3-fluoro-4-methoxypiperidin-1-yl)pyrimidin-4-yl)amino)-5-isopropylisoquinolin-8-yl)azetidin-3-yl)-2-(methylsulfonyl)ethyl)carbamate as a yellow solid (30 mg). LCMS m/z=672 [M+H]⁺

### Preparation 49

### 6-chloro-4-isopropyl-N-(2-(methylsulfonyl)ethyl)-2,7-naphthyridin-1-amine

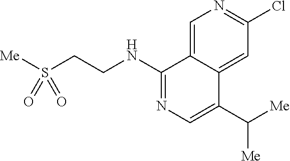 [SMILES: CC(C)c1cnc(NCCS(C)(=O)=O)c2cnc(Cl)cc12]

A mixture of 1,6-dichloro-4-isopropyl-2,7-naphthyridine (Preparation 113, 120 mg, 0.497 mmol), 2-(methylsulfonyl)ethan-1-amine (61.0 mg, 0.496 mmol) and DIPEA (192 mg, 1.49 mmol) in DMSO (8 mL) was stirred at 100° C. for 5 h. The cooled reaction was evaporated to dryness in vacuo and the residue purified using RP-HPLC (MeCN/H₂O) to afford the title compound as a yellow solid (100 mg). LCMS m/z=328 [M+H]⁺

### Preparation 50

### N1-(6-chloro-4-isopropyl-2,7-naphthyridin-1-yl)-N2,N2-dimethylethane-1,2-diamine

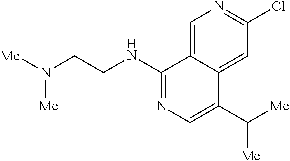 [SMILES: CC(C)c1cnc(NCCN(C)C)c2cnc(Cl)cc12]

A mixture of 1,6-dichloro-4-(propan-2-yl)-2,7-naphthyridine (Preparation 49, 200 mg, 1.65 mmol), (2-aminoethyl)dimethylamine (145 mg, 1.65 mmol) and TEA (167 mg, 1.65 mmol) in IPA was stirred at 100° C. for 3 h. The reaction mixture was evaporated to dryness in vacuo and the residue purified by prep-TLC (EtOAc) to afford the title compound as a light yellow solid (200 mg). LCMS m/z=293 [M+H]⁺

### Preparation 51

### N1-(4-bromo-6-chloro-2,7-naphthyridin-1-yl)-N2,N2-dimethylpropane-1,2-diamine

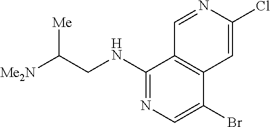 [SMILES: CC(CNc1ncc(Br)c2cc(Cl)ncc12)N(C)C]

N2,N2-dimethylpropane-1,2-diamine (218 mg, 2.14 mmol) was added to 4-bromo-1,6-dichloro-2,7-naphthyridine (500 mg, 1.79 mmol) and TEA (271 mg, 2.68 mmol) in IPA and the reaction mixture stirred at 100° C. for 4 h. The cooled reaction mixture was evaporated to dryness in vacuo and the residue washed with PE/EtOAc to afford the title compound as a yellow solid (500 mg). LCMS m/z=345 [M+H]⁺

### Preparation 52

### N1-(6-chloro-4-(prop-1-en-2-yl)-2,7-naphthyridin-1-yl)-N2,N2-dimethylpropane-1,2-diamine

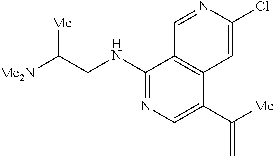 [SMILES: C=C(C)c1cnc(NCC(C)N(C)C)c2cnc(Cl)cc12]

A mixture of N1-(4-bromo-6-chloro-2,7-naphthyridin-1-yl)-N2,N2-dimethylpropane-1,2-diamine (Preparation 51, 600 mg, 1.74 mmol), 4,4,5,5-tetramethyl-2-(prop-1-en-2-yl)-1,3,2-dioxaborolane (349 mg, 2.08 mmol), K₂CO₃ (480 mg, 3.48 mmol) and Pd(dppf)Cl₂ (156 mg, 0.174 mmol) in 1,4-dioxane and H₂O was heated under N₂ at 80° C. for 2 h. The reaction mixture was extracted into EtOAc and the combined extracts washed with brine and evaporated to dryness in vacuo. The residue was purified by silica gel chromatography (DCM/MeOH=5/1) to afford the title compound as a yellow solid (400 mg). LCMS m/z=305 [M+H]⁺

### Preparation 53

### N1-(6-chloro-4-isopropyl-2,7-naphthyridin-1-yl)-N2,N2-dimethylpropane-1,2-diamine

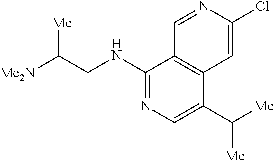 [SMILES: CC(C)c1cnc(NCC(C)N(C)C)c2cnc(Cl)cc12]

A mixture of N1-(6-chloro-4-(prop-1-en-2-yl)-2,7-naphthyridin-1-yl)-N2,N2-dimethylpropane-1,2-diamine (Preparation 52, 350 mg, 1.14 mmol) and PtO₂ (258 mg, 1.14 mmol) in EtOAc was stirred under a H₂ atmosphere at rt for 5 h. The catalyst was removed by filtration and the filtrate evaporated to dryness in vacuo to afford the title compound as a yellow solid (300 mg) which was used without further purification. LCMS m/z=307 [M+H]⁺

### Preparation 54

### 4-(((3-chloro-5-isopropylisoquinolin-8-yl)oxy)methyl)-1-methylpyrrolidin-2-one

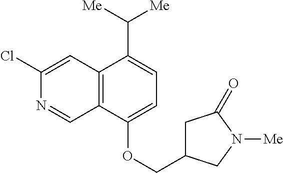 [SMILES: CC(C)c1ccc(OCC2CC(=O)N(C)C2)c2cnc(Cl)cc12]

The title compound was prepared from 8-bromo-3-chloro-5-(prop-1-en-2-yl)isoquinoline (Preparation 40) and 4-(hydroxymethyl)-1-methylpyrrolidin-2-one using an analogous method to that described for Preparation 45. Yield=27 mg; LCMS m/z=331 [M+H]⁺

### Preparation 55

### 6-chloro-4-isopropyl-N-(1-methyl-1H-pyrazol-4-yl)-2,7-naphthyridin-1-amine

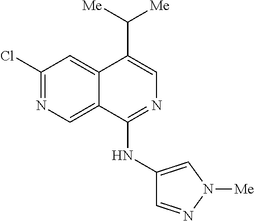 [SMILES: CC(C)c1cnc(Nc2cnn(C)c2)c2cnc(Cl)cc12]

A mixture of 1,6-dichloro-4-isopropyl-2,7-naphthyridine (Preparation 113, 120 mg, 0.497 mmol), 1-methyl-1H-pyrazol-4-amine (48.1 mg, 0.496 mmol), Pd₂(dba)₃·CHCl₃ (51.3 mg, 0.0496 mmol), Xantphos (28.7 mg, 0.0496 mmol) and Cs₂CO₃ (323 mg, 0.993 mmol) in dioxane (25 mL) was stirred at 100° C. for 5 h. The mixture was evaporated to dryness in vacuo and the residue purified by prep-TLC (30:1 DCM/MeOH) to afford the title compound as a yellow solid (90 mg, 60%). LCMS m/z=302 [M+H]⁺

### Preparation 56

### tert-butyl (3-chloro-5-isopropylisoquinolin-8-yl)carbamate

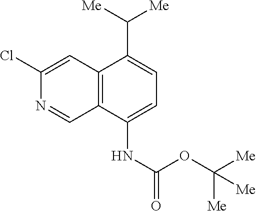 [SMILES: CC(C)c1ccc(NC(=O)OC(C)(C)C)c2cnc(Cl)cc12]

Tert-butyl carbamate (116 mg, 0.997 mmol) and Cs₂CO₃ (648 mg, 1.99 mmol) were added to a solution of 8-bromo-3-chloro-5-isopropylisoquinoline (Preparation 41, 284 mg, 0.997 mmol) in dry dioxane before XantPhos Pd G4 (95.8 mg, 0.100 mmol) was added under N₂. The resulting solution was stirred at 100° C. for 3 h. The cooled reaction mixture was evaporated to dryness and the residue purified by prep-TLC (5% MeOH/DCM) to afford the title compound (80 mg, 25%). LCMS m/z=321 [M+H]⁺

### Preparation 57

### tert-butyl (3-chloro-5-isopropylisoquinolin-8-yl)(methyl)carbamate

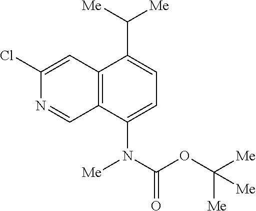 [SMILES: CC(C)c1ccc(N(C)C(=O)OC(C)(C)C)c2cnc(Cl)cc12]

To a solution of tert-butyl (3-chloro-5-isopropylisoquinolin-8-yl)carbamate (Preparation 56, 75 mg, 0.233 mmol) in DMF was added NaH (23.2 mg, 0.582 mmol) at 0° C. Mel (66.1 mg, 0.466 mmol) was added and the mixture warmed to 25° C. and stirred for 1.5 h. The reaction was quenched with H₂O and extracted with EtOAc. The combined organics were dried and evaporated to dryness in vacuo. The resulting residue was purified by prep-TLC (5% MeOH/DCM) to give the title compound (30 mg, 38%). LCMS m/z=335 [M+H]⁺

### Preparation 58

### tert-butyl (3-((2-((3S,4R)-3-fluoro-4-hydroxy-3-methylpiperidin-1-yl)pyrimidin-4-yl)amino)-5-isopropylisoquinolin-8-yl)(methyl)carbamate

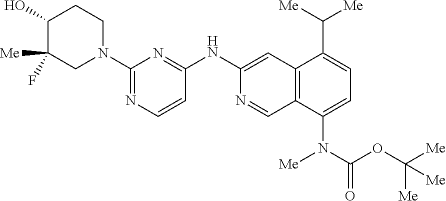 [SMILES: CC(C)c1ccc(N(C)C(=O)OC(C)(C)C)c2cnc(Nc3ccnc(N4CC[C@@H](O)[C@@](C)(F)C4)n3)cc12]

To a solution of tert-butyl (3-chloro-5-isopropylisoquinolin-8-yl)(methyl)carbamate (Preparation 57, 50 mg, 0.149 mmol) in dry dioxane were added (3S,4R)-1-(4-aminopyrimidin-2-yl)-3-fluoro-3-methylpiperidin-4-ol (Preparation 8, 33.7 mg, 0.149 mmol), Cs₂CO₃ (97.1 mg, 0.298 mmol), CPhos (6.49 mg, 0.015 mmol) and Pd₂(dba)₃·CHCl₃ (15.3 mg, 14.9 μmol) and the mixture stirred at 100° C. for 16 h under N₂. The mixture was evaporated to dryness and the residue purified by prep-TLC (5% MeOH/DCM) to give the title compound (50 mg, 64%). LCMS m/z=525 [M+H]⁺

### Preparation 59

### 8-(azetidin-3-ylmethoxy)-3-chloro-5-isopropylisoquinoline

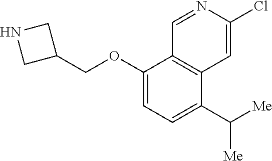 [SMILES: CC(C)c1ccc(OCC2CNC2)c2cnc(Cl)cc12]

Part 1: A mixture of 8-bromo-3-chloro-5-isopropylisoquinoline (Preparation 41, 45 mg, 0.158 mmol), tert-butyl 3-(hydroxymethyl)azetidine-1-carboxylate (36 mg, 0.190 mmol), Cs₂CO₃ (103 mg, 0.316 mmol) and RockPhos Pd G3 (6.63 mg, 7.9 mmol) was heated under N₂ at 90° C. for 2 h. The reaction was diluted with EtOAc, filtered through a pad of Celite® and the filtrate evaporated to dryness in vacuo. The residue was purified using ISCO chromatography (0-50% EtOAc/Hex) to give tert-butyl 3-(((3-chloro-5-isopropylisoquinolin-8-yl)oxy)methyl)azetidine-1-carboxylate as an off-white foam (20 mg 32%).

Part 2: TFA (excess) was added to a solution of tert-butyl 3-(((3-chloro-5-isopropylisoquinolin-8-yl)oxy)methyl)azetidine-1-carboxylate (Part 1, 20 mg, 0.051 mmol) in DCM (0.5 mL) and stirred at rt for 2 h. The reaction mixture was diluted with DCM and washed with sat. aq. NaHCO₃ solution. The combined organics were dried (Na₂SO₄) and evaporated to dryness in vacuo to afford the title compound as a yellow film (13 mg, 87%). LCMS m/z=291 [M+H]⁺

### Preparation 60

### 8-(1-(azetidin-3-yl)ethoxy)-3-chloro-5-isopropylisoquinoline

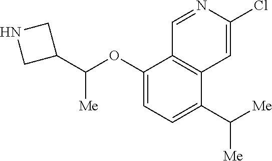 [SMILES: CC(C)c1ccc(OC(C)C2CNC2)c2cnc(Cl)cc12]

Part 1: DIAD (182 mg, 0.902 mmol) was added to an ice-cold solution of 3-chloro-5-isopropylisoquinolin-8-ol (Preparation 43, 100 mg, 0.451 mmol), tert-butyl 3-(1-hydroxyethyl)azetidine-1-carboxylate (182 mg, 0.902 mmol) and PPh₃ (237 mg, 0.902 mmol) in THF (2.5 mL) and the resulting solution allowed to warm to rt. The reaction was evaporated to dryness in vacuo and the residue combined with the residue obtained from an analogous procedure carried out on ¼ scale. Combined yield=180 mg, 80%). LCMS m/z=405 [M+H]⁺

Part 2: TFA (xs) was added to a solution of tert-butyl 3-(1-((3-chloro-5-isopropylisoquinolin-8-yl)oxy)ethyl)azetidine-1-carboxylate (Part 1, 180 mg, 0.445 mmol) in DCM (3 mL) and stirred at rt for 1 h. The reaction mixture was diluted with DCM and washed with sat. aq. NaHCO₃ solution. The combined organics were dried (Na₂SO₄) and evaporated to dryness to afford the title compound as a pale orange foam (135 mg, 100%). LCMS m/z=305 [M+H]⁺

### Preparation 61

### (R)-5-(((3-chloro-5-isopropylisoquinolin-8-yl)oxy)methyl)-3-methyloxazolidin-2-one

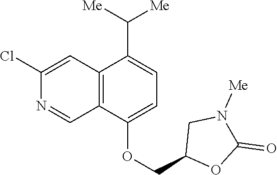 [SMILES: CC(C)c1ccc(OC[C@H]2CN(C)C(=O)O2)c2cnc(Cl)cc12]

A mixture of 8-bromo-3-chloro-5-isopropylisoquinoline (Preparation 41, 83 mg, 0.292 mmol), (R)-5-(hydroxymethyl)-3-methyloxazolidin-2-one (36 mg, 0.190 mmol), Cs₂CO₃ (190 mg, 0.583 mmol) and RockPhos Pd G3 (12 mg, 15 mmol) was heated under N₂ at 50° C. overnight. The reaction was diluted with EtOAc, filtered through a pad of Celite® and the filtrate evaporated to dryness in vacuo. The residue was purified using ISCO chromatography (0-10% MeOH/DCM) to give the title compound as a white foam (33 mg 33.8%). LCMS m/z=335 [M+H]⁺

### Preparation 62

### N-(3-((3-chloro-5-isopropylisoquinolin-8-yl)oxy)propyl)acetamide

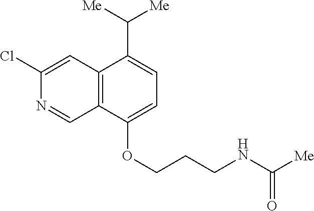 [SMILES: CC(=O)NCCCOc1ccc(C(C)C)c2cc(Cl)ncc12]

The title compound was prepared from 8-bromo-3-chloro-5-isopropylisoquinoline (Preparation 41) and N-(3-hydroxypropyl)acetamide using an analogous method to that described for Preparation 61. Yield=45 mg, 40%; LCMS m/z=321 [M+H]⁺

### Preparation 63

### 1-(3-(((3-chloro-5-isopropylisoquinolin-8-yl)oxy)methyl)azetidin-1-yl)ethan-1-one

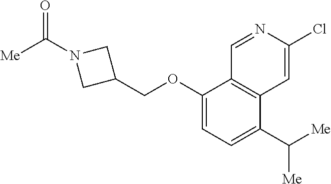 [SMILES: CC(=O)N1CC(COc2ccc(C(C)C)c3cc(Cl)ncc23)C1]

Acetyl chloride (5.26 mg, 0.067 mmol) was added to a mixture of 8-(azetidin-3-ylmethoxy)-3-chloro-5-isopropylisoquinoline (Preparation 59, 13 mg, 0.045 mmol) and DIPEA (8.67 mg, 0.067 mmol) in DCM (0.5 mL) and the resulting mixture stirred at rt for 30 min. The reaction was diluted with DCM and washed with H₂O. The combined organics were dried (Na₂SO₄) and evaporated to dryness in vacuo to afford the title compound as an off-white foam (16 mg) which was used without further purification. LCMS m/z=333 [M+H]⁺

### Preparation 64

### 1-(3-(1-((3-chloro-5-isopropylisoquinolin-8-yl)oxy)ethyl)azetidin-1-yl)ethan-1-one

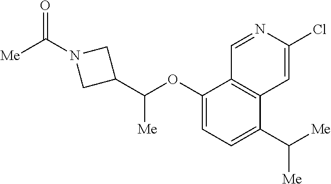 [SMILES: CC(=O)N1CC(C(C)Oc2ccc(C(C)C)c3cc(Cl)ncc23)C1]

The title compound was prepared from 8-(1-(azetidin-3-yl)ethoxy)-3-chloro-5-isopropylisoquinoline (Preparation 60) using an analogous method to that described for Preparation 63. Yield=55 mg, 31.6%; LCMS m/z=347 [M+H]⁺.

### Preparation 65

### (R)-5-(((3-chloro-5-isopropylisoquinolin-8-yl)oxy)methyl)-1-methylpyrrolidin-2-one

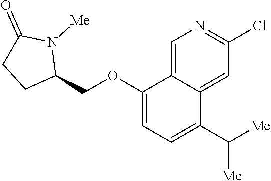 [SMILES: CC(C)c1ccc(OC[C@H]2CCC(=O)N2C)c2cnc(Cl)cc12]

DIAD (137 mg, 0.677 mmol) was added dropwise to an ice-cold solution of 3-chloro-5-isopropylisoquinolin-8-ol (Preparation 43, 75 mg, 0.338 mmol), (R)-5-(hydroxymethyl)-1-methylpyrrolidin-2-one (87 mg, 0.677 mmol) and PPh₃ (177 mg, 0.677 mmol) in THF (2 mL) under N₂. The reaction was allowed to warm slowly to rt and stirred at rt for 4 h. The reaction was evaporated to dryness in vacuo and the residue purified using ISCO chromatography (0-8% MeOH/DCM) to give the title compound as a pale yellow oil (61 mg, 54%). LCMS m/z=333 [M+H]⁺

### Preparation 66-75

The title compounds were prepared from 3-chloro-5-isopropylisoquinolin-8-ol (Preparation 43) and the appropriate alcohol (ROH) using an analogous method to that described for Preparation 65.

| Preparation | |
|---|---|
| No. | Name/Structure/Reactants/Data |
| 66 | 4-(2-((3-chloro-5-isopropylisoquinolin-8-yl)oxy)ethyl)morpholine |
| | 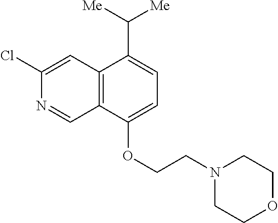 [SMILES: CC(C)c1ccc(OCCN2CCOCC2)c2cnc(Cl)cc12] |
| | ROH: 2-morpholinoethan-1-ol |
| | Yield: 64 mg, 85%; LCMS m/z = 335 [M + H]⁺ |
| 67 | 3-chloro-5-isopropyl-8-(2-(2-methyl-1H-imidazol-1-yl)ethoxy)isoquinoline |
| | 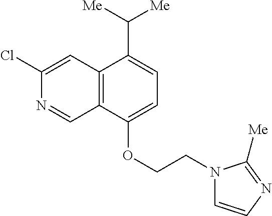 [SMILES: Cc1nccn1CCOc1ccc(C(C)C)c2cc(Cl)ncc12] |
| | ROH: 2-(2-methyl-1H-imidazol-1-yl)ethan-1-ol |
| | Yield: 73 mg, 98%; LCMS m/z = 330 [M + H]⁺ |
| 68 | (S)-5-(((3-chloro-5-isopropylisoquinolin-8-yl)oxy)methyl)-1-methylpyrrolidin-2- |
| | one |
| | 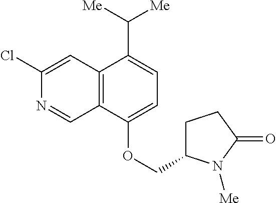 [SMILES: CC(C)c1ccc(OC[C@@H]2CCC(=O)N2C)c2cnc(Cl)cc12] |
| | ROH: (S)-5-(hydroxymethyl)-1-methylpyrrolidin-2-one |
| | Yield: 112 mg, 99%; LCMS m/z = 333 [M + H]⁺ |
| 69 | 3-chloro-5-isopropyl-8-(2-(1-methyl-1H-pyrazol-5-yl)ethoxy)isoquinoline |
| | 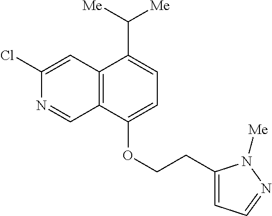 [SMILES: CC(C)c1ccc(OCCc2ccnn2C)c2cnc(Cl)cc12] |
| | ROH: 2-(1-methyl-1H-pyrazol-5-yl)ethan-1-ol |
| | Yield: 88 mg, 79%. LCMS m/z = 330 [M + H]⁺ |
| 70 | 3-chloro-5-isopropyl-8-(2-(1-methyl-1H-1,2,3-triazol-5-yl)ethoxy)isoquinoline |
| | 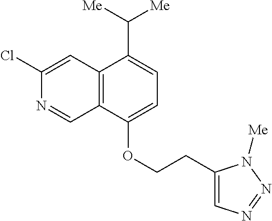 [SMILES: CC(C)c1ccc(OCCc2cnnn2C)c2cnc(Cl)cc12] |
| | ROH: 2-(1-methyl-1H-1,2,3-triazol-5-yl)ethan-1-ol |
| | Yield: 83 mg, 74%. LCMS m/z = 331 [M + H]⁺ |
| 71 | (S)-8-(2-(4H-1,2,4-triazol-4-yl)propoxy)-3-chloro-5-isopropylisoquinoline |
| | 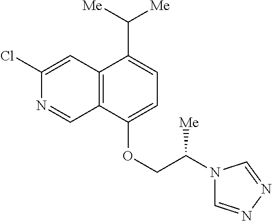 [SMILES: CC(C)c1ccc(OC[C@H](C)n2cnnc2)c2cnc(Cl)cc12] |
| | ROH: (S)-2-(4H-1,2,4-triazol-4-yl)propan-1-ol (Preparation 82) |
| | Yield: 92 mg, 103%. LCMS m/z = 331 [M + H]⁺ |
| 72 | (R)-8-(2-(4H-1,2,4-triazol-4-yl)propoxy)-3-chloro-5-isopropylisoquinoline |
| | 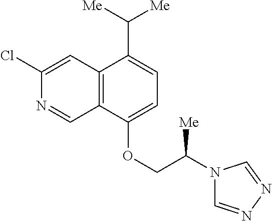 [SMILES: CC(C)c1ccc(OC[C@@H](C)n2cnnc2)c2cnc(Cl)cc12] |
| | ROH: (R)-2-(4H-1,2,4-triazol-4-yl)propan-1-ol (Preparation 83) |
| | Yield: 88 mg, 98%. LCMS m/z = 331 [M + H]⁺ |
| 73 | 4-(((3-chloro-5-isopropylisoquinolin-8-yl)oxy)methyl)-3-methyloxazolidin-2-one |
| | 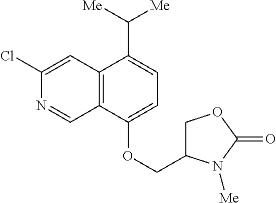 [SMILES: CC(C)c1ccc(OCC2COC(=O)N2C)c2cnc(Cl)cc12] |
| | ROH: 4-(hydroxymethyl)-3-methyloxazolidin-2-one |
| | Yield: 75 mg, 60%. LCMS m/z = 335 [M + H]⁺ |
| 74 | 3-chloro-5-isopropyl-8-(2-(1-methyl-1H-imidazol-5-yl)ethoxy)isoquinoline |
| | 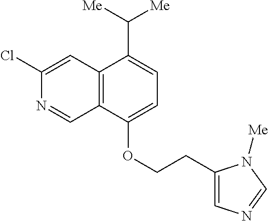 [SMILES: CC(C)c1ccc(OCCc2cncn2C)c2cnc(Cl)cc12] |
| | ROH: 2-(1-methyl-1H-imidazol-5-yl)ethan-1-ol |
| | Yield: 97 mg, 87%. LCMS m/z = 330 [M + H]⁺ |
| 75 | 4-(2-((3-chloro-5-isopropylisoquinolin-8-yl)oxy)ethyl)-3,5-dimethylisoxazole |
| | 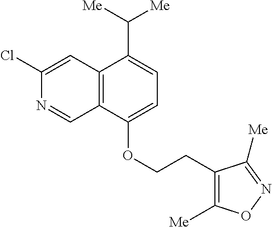 [SMILES: Cc1noc(C)c1CCOc1ccc(C(C)C)c2cc(Cl)ncc12] |
| | ROH: 2-(dimethyl-1,2-oxazol-4-yl)ethan-1-olYield: 54 mg, 48%. LCMS m/z = |
| | 345 [M + H]⁺ |

### Preparation 76

### 8-(2-(4H-1,2,4-triazol-4-yl)ethoxy)-3-chloro-5-isopropylisoquinoline

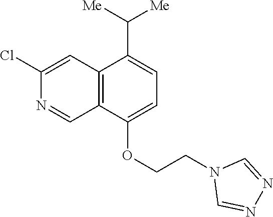 [SMILES: CC(C)c1ccc(OCCn2cnnc2)c2cnc(Cl)cc12]

Part 1: 4-(2-Bromoethyl)-4H-1,2,4-triazole hydrobromide (243 mg, 0.947 mmol) was dissolved in 10% MeOH/DCM and stirred with MP-carbonate resin for 15 min. The solids were removed by filtration and evaporated to dryness to afford 4-(2-bromoethyl)-4H-1,2,4-triazole as the free base which was used without further purification.

Part 2: A mixture of 4-(2-bromoethyl)-4H-1,2,4-triazole (Part 1, 167 mg, 0.947 mmol), 3-chloro-5-isopropylisoquinolin-8-ol (Preparation 43, 105 mg, 0.474 mmol) and K₂CO₃ (164 mg, 1.184 mmol) in DMF (2 mL) was heated at 90° C. overnight. The reaction was diluted with H₂O and extracted with EtOAc (×2). The combined organics were washed with H₂O (×3), brine, dried (Na₂SO₄) and evaporated to dryness in vacuo. The residue was purified by ISCO Chromatography (0-10% MeOH/DCM) to give the title compound as a pink solid (50 mg, 33%). LCMS m/z=317 [M+H]⁺

### Preparation 77

### 8-(2-(1H-1,2,4-triazol-1-yl)ethoxy)-3-chloro-5-isopropylisoquinoline

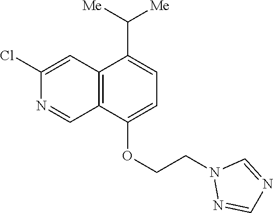 [SMILES: CC(C)c1ccc(OCCn2cncn2)c2cnc(Cl)cc12]

The title compound was prepared from 3-chloro-5-isopropylisoquinolin-8-ol (Preparation 43) and 1-(2-bromoethyl)-1H-1,2,4-triazole using an analogous method to that described for Preparation 76 (Part 2). Yield: 23 mg, 46%; LCMS m/z=317 [M+H]⁺

### Preparation 78

### 3-chloro-5-isopropyl-8-(2-(3-methyl-4H-1,2,4-triazol-4-yl)ethoxy)isoquinoline

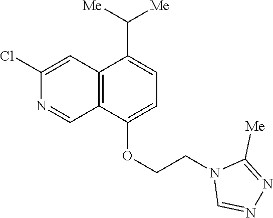 [SMILES: Cc1nncn1CCOc1ccc(C(C)C)c2cc(Cl)ncc12]

The title compound was prepared from 3-chloro-5-isopropylisoquinolin-8-ol (Preparation 43) and 4-(2-bromoethyl)-3-methyl-4H-1,2,4-triazole using an analogous method to that described for Preparation 76 (Part 2). Yield: 29 mg, 32%; LCMS m/z=331 [M+H]⁺

### Preparation 79

### 8-(2-(1H-1,2,3-triazol-1-yl)ethoxy)-3-chloro-5-isopropylisoquinoline

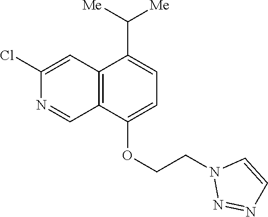 [SMILES: CC(C)c1ccc(OCCn2ccnn2)c2cnc(Cl)cc12]

The title compound was prepared using an analogous method to that described for Preparation 76 (Part 2) from 3-chloro-5-isopropylisoquinolin-8-ol (Preparation 43) and 1-(2-bromoethyl)-1H-1,2,3-triazole. Yield: 10 mg, 20%; LCMS m/z=317 [M+H]⁺

### Preparation 80

### 3-chloro-5-isopropyl-8-((1-methyl-1H-1,2,3-triazol-4-yl)methoxy)isoquinoline

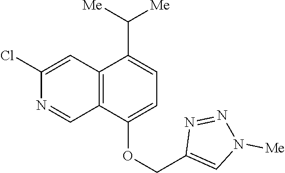 [SMILES: CC(C)c1ccc(OCc2cn(C)nn2)c2cnc(Cl)cc12]

The title compound was prepared using an analogous method to that described for Preparation 76 (Part 2) from 3-chloro-5-isopropylisoquinolin-8-ol (Preparation 43) and 4-(chloromethyl)-1-methyl-1H-1,2,3-triazole hydrochloride. Yield: 42 mg, 84%; LCMS m/z=317 [M+H]⁺

### Preparation 81

### 5-(((3-chloro-5-isopropylisoquinolin-8-yl)oxy)methyl)-1-methylpyrrolidin-2-one

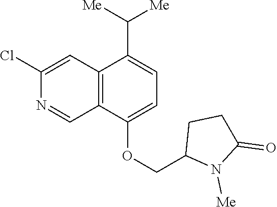 [SMILES: CC(C)c1ccc(OCC2CCC(=O)N2C)c2cnc(Cl)cc12]

The title compound was prepared from 3-chloro-5-isopropylisoquinolin-8-ol (Preparation 43) and 5-(bromomethyl)-1-methylpyrrolidin-2-one using an analogous method to that described for Preparation 76 (Step 2). Yield=47 mg, 41.7%; LCMS m/z=333 [M+H]⁺

### Preparation 82

### (S)-2-(4H-1,2,4-triazol-4-yl)propan-1-ol

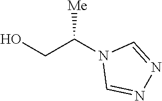

A mixture of triethyl orthoformate (800 mg, 5.39 mmol) and formic hydrazide (270 mg, 4.50 mmol) in MeOH (10 mL) was heated under reflux for 3 h. (S)-2-Aminopropan-1-ol (338 mg, 4.50 mmol) was added to the reaction and the mixture heated under reflux overnight. The reaction was evaporated to dryness in vacuo and the resulting gum triturated with hexane/IPA (1:1) to afford the title compound as a white solid (202 mg. 35%). LCMS m/z=128 [M+H]⁺

### Preparation 83

### (R)-2-(4H-1,2,4-triazol-4-yl)propan-1-ol

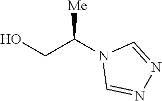

The title compound was prepared from (R)-2-aminopropan-1-ol, using an analogous method to that described for Preparation 82. Yield: 210 mg, 37%. LCMS m/z=128 [M+H]⁺

### Preparation 84

### (6,7-dihydro-5H-pyrrolo[1,2-c]imidazol-5-yl)methyl methanesulfonate

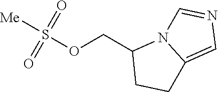 [SMILES: CS(=O)(=O)OCC1CCc2cncn21]

Mesyl chloride (48 mg, 0.418 mmol) was added to an ice-cold solution of TEA (40 mg, 0.398 mmol) and (6,7-dihydro-5H-pyrrolo[1,2-c]imidazol-5-yl)methanol (55 mg, 0.398 mmol) and the resulting mixture allowed to slowly warm to rt overnight. The reaction was diluted with DCM, washed (H₂O), dried (Na₂SO₄) and evaporated to dryness to afford the title compound as a pale yellow oil (56 mg, 65%). LCMS m/z=217 [M+H]⁺

### Preparation 85

### 2-(1H-imidazol-1-yl)ethyl methanesulfonate

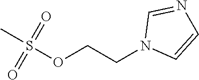

The title compound was prepared from 2-(1H-imidazol-1-yl)ethan-1-ol using an analogous method to that described for Preparation 84. Yield: 230 mg, 27%. LCMS m/z=191 [M+H]⁺

### Preparation 86

### 3-chloro-8-((6,7-dihydro-5H-pyrrolo[1,2-c]imidazol-5-yl)methoxy)-5-isopropylisoquinoline

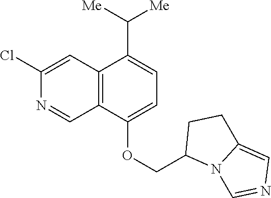 [SMILES: CC(C)c1ccc(OCC2CCc3cncn32)c2cnc(Cl)cc12]

A mixture of 3-chloro-5-isopropylisoquinolin-8-ol (Preparation 43, 28 mg, 0.126 mmol), (6,7-dihydro-5H-pyrrolo[1,2-c]imidazol-5-yl)methyl methanesulfonate (Preparation 84, 55 mg, 0.253 mmol) and K₂CO₃ (52 mg, 0.379 mmol) in DMF (0.75 mL) was heated at 80° C. for 2 h. The reaction was diluted with water and extracted with EtOAc. The combined organics were washed with water (×2), brine, dried (Na₂SO₄) and evaporated to dryness in vacuo. The residue was purified by ISCO chromatography (0-10% MeOH/DCM) to afford the title compound as a pale yellow solid (27 mg, 62%). LCMS m/z=342 [M+H]⁺

### Preparation 87

### 8-(2-(1H-imidazol-1-yl)ethoxy)-3-chloro-5-isopropylisoquinoline

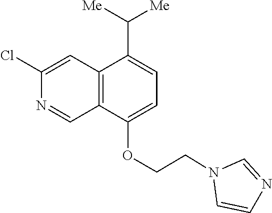 [SMILES: CC(C)c1ccc(OCCn2ccnc2)c2cnc(Cl)cc12]

The title compound was prepared from 3-chloro-5-isopropylisoquinolin-8-ol (Preparation 43) and 2-(1H-imidazol-1-yl)ethyl methanesulfonate using an analogous method to that described for Preparation 86. Yield: 29 mg, 51%. LCMS m/z=316 [M+H]⁺

### Preparation 88

### 1-(3-(((3-chloroisoquinolin-8-yl)oxy)methyl)azetidin-1-yl)ethan-1-one

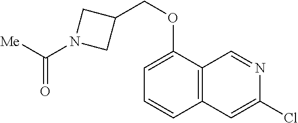 [SMILES: CC(=O)N1CC(COc2cccc3cc(Cl)ncc23)C1]

Part 1: A mixture of 8-bromo-3-chloroisoquinoline (121 mg, 0.499 mmol), KOH (112 mg, 2.00 mmol), Pd₂(dba)₃·CHCl₃ (9.14 mg, 0.01 mmol) and di-tert-butyl(2′,4′,6′-triisopropyl-3,4,5,6-tetramethyl-[1,1′-biphenyl]-2-yl)phosphine (19 mg, 0.04 mmol) in dioxane (1 mL) was heated at 90° C. under N₂ for 1 h. The reaction was diluted with EtOAc and neutralised with 1 M HCl. The organic layer was washed (brine), dried (Na₂SO₄) and evaporated to dryness in vacuo. The residue was purified by ISCO chromatography (5-75% EtOAc/Hex) to afford 3-chloroisoquinolin-8-ol as an off-white solid (67 mg). LCMS m/z=180 [M+H]⁺

Part 2: A mixture of 3-chloroisoquinolin-8-ol (Part 1, 67 mg, 0.373 mmol), tert-butyl 3-(iodomethyl)azetidine-1-carboxylate (166 mg, 0.560 mmol) and K₂CO₃ (77 mg, 0.560 mmol) in DMF (1 mL) was heated at 60° C. for 1.5 h. Additional tert-butyl 3-(iodomethyl)azetidine-1-carboxylate (100 mg) and K₂CO₃ (77 mg) was added and heating continued for a further 90 min. The reaction was diluted with water and extracted with EtOAc. The combined organics were washed with H₂O (×3), brine, dried (Na₂SO₄) and evaporated to dryness in vacuo. The residue was purified by ISCO chromatography (0-50% EtOAc/Hex) to afford tert-butyl 3-(((3-chloroisoquinolin-8-yl)oxy)methyl)azetidine-1-carboxylate as a white foam (111 mg, 85%).

Part 3: TFA (0.25 mL, 3.18 mmol) was added to a solution of tert-butyl 3-(((3-chloroisoquinolin-8-yl)oxy)methyl)azetidine-1-carboxylate (Part 2, 111 mg, 0.318 mmol) in DCM (2 mL) and the mixture stirred at rt for 2 h. The reaction mixture was evaporated to dryness in vacuo and the residue partitioned between DCM and sat. aq. NaHCO₃. The combined organics were dried (Na₂SO₄) and evaporated to dryness to afford 8-(azetidin-3-ylmethoxy)-3-chloroisoquinoline as a colourless oil (58 mg, 73%). LCMS m/z=249 [M+H]⁺

Part 4: Acetyl chloride (22 mg, 0.28 mmol) was added to a mixture of 8-(azetidin-3-ylmethoxy)-3-chloroisoquinoline (Part 3, 58 mg, 0.233 mmol) and TEA (35 mg, 0.35 mmol) in DCM (1 mL) and the resulting mixture stirred at rt for 1 h. The reaction was diluted with DCM and washed with water. The combined organics were dried (Na₂SO₄) and evaporated to dryness in vacuo to afford the title compound as a tan foam (59 mg, 87%). LCMS m/z=291 [M+H]⁺

### Preparation 89

### 2-(((3S,4R)-3-fluoro-1-(4-((5-isopropyl-8-(((R)-3-methyl-2-oxooxazolidin-4-yl)methoxy)isoquinolin-3-yl)amino)pyrimidin-2-yl)piperidin-4-yl)oxy)ethyl methanesulfonate

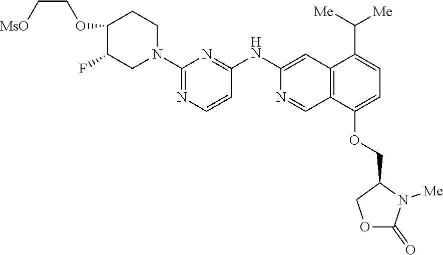 [SMILES: CC(C)c1ccc(OC[C@@H]2COC(=O)N2C)c2cnc(Nc3ccnc(N4CC[C@@H](OCCOS(C)(=O)=O)[C@@H](F)C4)n3)cc12]

To a solution of (R)-4-(((3-((2-((3S,4R)-3-fluoro-4-(2-hydroxyethoxy)-4-methylpiperidin-1-yl)pyrimidin-4-yl)amino)-5-isopropylisoquinolin-8-yl)oxy)methyl)-3-methyloxazolidin-2-one (Example 52, 60 mg, 0.108 mmol) in DCM (5 mL) was added TEA (10.9 mg, 0.108 mmol) and methanesulfonyl chloride (12.3 mg, 0.108 mmol) at 0° C. and the mixture stirred at 25° C. for 3 h. The reaction mixture was extracted with EtOAc and washed with brine. The combined organics were dried (Na₂SO₄) and evaporated to dryness in vacuo to afford the title compound as a yellow gum (60 mg) which was used without further purification. LCMS m/z=631 [M+H]⁺

### Preparation 90

### 7-chloro-1-isopropyl-2,6-naphthyridin-4-ol

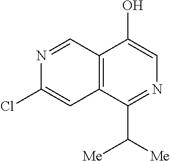 [SMILES: CC(C)c1ncc(O)c2cnc(Cl)cc12]

A solution of KOH (157 mg, 2.80 mmol), 4-bromo-7-chloro-1-isopropyl-2,6-naphthyridine (Preparation 121, 200 mg, 0.700 mmol), Pd₂(dba)₃ (19.2 mg, 0.021 mmol), and di-tert-butyl(2′,4′,6′-triisopropyl-3,4,5,6-tetramethyl-[1,1′-biphenyl]-2-yl)phosphane (30.3 mg, 0.063 mmol) in dioxane (1.2 mL) and H₂O (1.2 mL) was degassed with N₂ and stirred at 90° C. for 1 h. The reaction was diluted with H₂O and the pH adjusted to 3-4 with 1M HCl and extracted with DCM (2×5 mL). The combined organics were dried (Na₂SO₄) and evaporated to dryness in vacuo. The residue was purified by ISCO chromatography (0-70% EtOAc/hex) to afford the title compound as a pale yellow solid (41 mg, 26%). LCMS m/z=223 [M+H]⁺

### Preparation 91

### 4-(2-(4H-1,2,4-triazol-4-yl)ethoxy)-7-chloro-1-isopropyl-2,6-naphthyridine

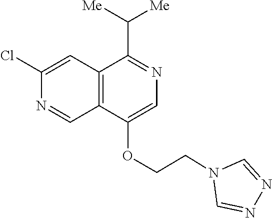 [SMILES: CC(C)c1ncc(OCCn2cnnc2)c2cnc(Cl)cc12]

A mixture of 4-(2-bromoethyl)-4H-1,2,4-triazole hydrobromide (92 mg, 0.539 mmol), 7-chloro-1-isopropyl-2,6-naphthyridin-4-ol (Preparation 91, 40 mg, 0.180 mmol) and K₂CO₃ (74 mg, 0.539 mmol) in DMF (1.5 mL) was heated at 80° C. overnight. The reaction was diluted with H₂O and extracted with EtOAc (×2). The combined organics were washed with H₂O (×2), brine, dried (Na₂SO₄) and evaporated to dryness in vacuo. The residue was purified by ISCO chromatography (0-10% MeOH/DCM) to give the title compound as a pink solid (27.3 mg, 48%). LCMS m/z=318 [M+H]⁺

### Preparation 92

### 1-(2-fluoroethyl)azetidin-3-amine hydrochloride

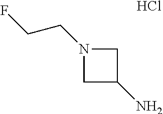

Part 1: To a solution of tert-butyl N-(azetidin-3-yl)carbamate (500 mg, 2.90 mmol) in DMF was added 1-bromo-2-fluoroethane (368 mg, 2.90 mmol), Cs₂CO₃ (2.83 g, 8.7 mmol) and NaI (434 mg, 2.9 mmol) and the reaction stirred at 60° C. for 1 h. The cooled reaction mixture was diluted with water (20 mL), the resulting solution extracted with EtOAc (2×20 mL) and the organic layers combined. The organic solution was washed with brine (20 mL) then dried over Na₂SO₄ and concentrated in vacuo. The crude product was purified by TLC to give tert-butyl (1-(2-fluoroethyl)azetidin-3-yl)carbamate, 300 mg, 47.4%, as colorless oil.

Part 2: A solution of tert-butyl (1-(2-fluoroethyl)azetidin-3-yl)carbamate (200 mg, 0.916 mmol) in HCl (5 mL) was stirred at rt for 3 h. The reaction mixture was evaporated under reduced pressure to afford the title compound. LCMS m/z=119 [M+H]⁺

### Preparation 93

### 6-chloro-4-iodo-2,7-naphthyridin-1(2H)-one

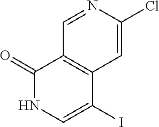 [SMILES: O=c1[nH]cc(I)c2cc(Cl)ncc12]

To a solution of 6-chloro-1,2-dihydro-2,7-naphthyridin-1-one (5 g, 27.6 mmol) in DMF (30 mL), was added NIS (7.44 g, 33.1 mmol) at 0° C. and the reaction stirred overnight at rt. The reaction mixture was filtered, the solid washed with water, and dried to afford the title compound (6 g, 70.8% yield) as a light yellow solid. LCMS m/z=307 [M+H]⁺.

### Preparation 94

### 6-chloro-4-(prop-1-en-2-yl)-2,7-naphthyridin-1(2H)-one

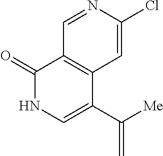

A mixture of 6-chloro-4-iodo-2,7-naphthyridin-1(2H)-one (Preparation 93, 6 g, 19.5 mmol), isopropenylboronic acid pinacol ester (4.9 g, 29.2 mmol), K₂CO₃ (5.46 g, 39 mmol) and Pd(amphos)Cl₂ (1.37 g, 1.95 mmol) in DMF/water (500 mL/100 mL) was heated to 80° C. for 8 h under N₂. The cooled reaction was filtered, the filtrate extracted with EtOAc (3×200 mL), the organic layers combined and dried over anhydrous Na₂SO₄ and concentrated in vacuo. The residue was purified by column chromatography on silica gel eluting with EtOAc/PE (1:10-1:1) to give the title compound, 1.8 g, 42% yield, as a light brown solid. LCMS m/z=221 [M+H]⁺

### Preparation 95

### 6-chloro-4-isopropyl-2,7-naphthyridin-1(2H)-one

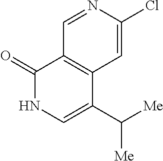 [SMILES: CC(C)c1c[nH]c(=O)c2cnc(Cl)cc12]

A mixture of 6-chloro-4-(prop-1-en-2-yl)-2,7-naphthyridin-1(2H)-one (Preparation 94, 1.8 g, 8.15 mmol) and PtO₂ (1.85 g, 8.15 mmol) in EtOAc (50 mL) was stirred at rt for 1.5 h under a H₂ atmosphere. The reaction was filtered and the filtrate concentrated in vacuo to give the title compound, 1.7 g, 94%, as a light brown solid. LCMS m/z=223 [M+H]⁺

### Preparation 96

### 6-Chloro-4-isopropyl-2,7-naphthyridin-1-yl trifluoromethanesulfonate

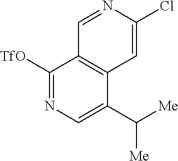 [SMILES: CC(C)c1cnc(OS(=O)(=O)C(F)(F)F)c2cnc(Cl)cc12]

To a solution of 6-chloro-4-isopropyl-2,7-naphthyridin-1(2H)-one (Preparation 95, 50 mg, 0.224 mmol) and TEA (120 mg, 1.2 mmol) in DCM (4 mL) was added dropwise trifluoromethanesulfonic anhydride (313 mg, 1.11 mmol) in DCM (1 mL) at −78° C. under N₂ and the reaction stirred for 30 min. The reaction was slowly warmed to rt and stirred for 1 h. The reaction mixture was quenched by adding water and extracted with DCM (3×20 mL), the combined organic layers dried over Na₂SO₄ and concentrated in vacuo to give the title compound as a brown oil. LCMS m/z=355 [M+H]⁺

### Preparation 97

### Methyl 6-chloro-4-isopropyl-2,7-naphthyridine-1-carboxylate

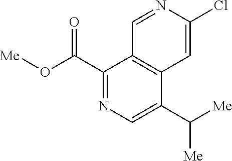 [SMILES: COC(=O)c1ncc(C(C)C)c2cc(Cl)ncc12]

A mixture of 6-chloro-4-isopropyl-2,7-naphthyridin-1-yl trifluoromethanesulfonate (Preparation 96, 220 mg, 0.62 mmol) was dissolved in MeOH (10 mL), TEA (250 mg, 2.48 mmol), Pd(dppf)Cl₂ (51 mg, 0.062 mmol) were added and the reaction heated to 50° C. under CO (5 atm) for 2 h. The reaction mixture was concentrated in vacuo and purified by TLC (1/1 PE/EtOAc) to give the title compound, 100 mg, 61.1%, as an off-white solid. LCMS m/z=265 [M+H]⁺

### Preparation 98

### 6-chloro-4-isopropyl-2,7-naphthyridine-1-carboxylic acid

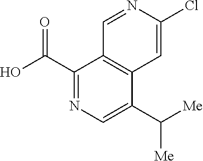 [SMILES: CC(C)c1cnc(C(=O)O)c2cnc(Cl)cc12]

LiOH (1.38 g, 57.56 mmol) was added to a solution of methyl 6-chloro-4-isopropyl-2,7-naphthyridine-1-carboxylate (Preparation 97, 3.8 g, 14.39 mmol) in THF/H₂O (80 mL/20 mL) and the reaction stirred for 3 h at rt. The reaction was acidified using 2N HCl, extracted with EtOAc and the combined organic extracts washed with water and dried over Na₂SO₄. The mixture was filtered and the filtrate evaporated under reduced pressure to give the title compound, 2.8 g, 77.5% as a light yellow solid. LCMS m/z=251 [M+H]⁺

### Preparation 99

### 6-chloro-4-isopropyl-N-methyl-2,7-naphthyridine-1-carboxamide

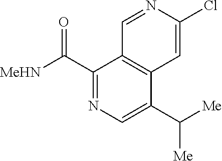 [SMILES: CNC(=O)c1ncc(C(C)C)c2cc(Cl)ncc12]

A mixture of 6-chloro-4-isopropyl-2,7-naphthyridine-1-carboxylic acid (Preparation 98, 2.1 g, 8.4 mmol), EDC·HCl (2.4 g, 12.5 mmol), HOBT (1.68 g, 12.5 mmol) and methanamine HCl (260 mg, 8.4 mmol) in DCM (80 mL) was stirred overnight at rt. The reaction was diluted with DCM and washed with water, brine, dried over Na₂SO₄ and concentrated in vacuo. The residue was purified by chromatography on silica gel (1/1 PE/EtOAc) to give the title compound, 1.6 g, 72.4%, as a light-yellow solid. LCMS m/z=264 [M+H]⁺

### Preparation 100

### 6-chloro-N-(2-(dimethylamino)ethyl)-4-isopropyl-2,7-naphthyridine-1-carboxamide

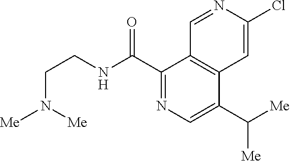 [SMILES: CC(C)c1cnc(C(=O)NCCN(C)C)c2cnc(Cl)cc12]

A mixture of 6-chloro-4-isopropyl-2,7-naphthyridine-1-carboxylic acid (Preparation 98, 120 mg, 0.478 mmol), EDC·HCl (137 mg, 0.717 mmol), HOBT (97 mg, 0.717 mmol) and (2-aminoethyl)dimethylamine (50.5 mg, 0.573 mmol) in DMF (4 mL) was stirred overnight at rt. The reaction was concentrated in vacuo and purified by preparative TLC (1/1 PE/EtOAc) to give the title compound, 80 mg, 52.3%, as an off-white solid. LCMS m/z=321 [M+H]⁺

### Preparation 101

### 6-chloro-4-isopropyl-N-(tetrahydro-2H-pyran-4-yl)-2,7-naphthyridine-1-carboxamide

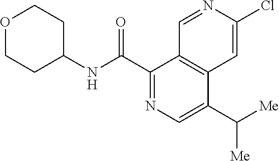 [SMILES: CC(C)c1cnc(C(=O)NC2CCOCC2)c2cnc(Cl)cc12]

The title compound was obtained as a white solid, 100 mg, 98.5% yield, from 6-chloro-4-isopropyl-2,7-naphthyridine-1-carboxylic acid (Preparation 98) and tetrahydro-2H-pyran-4-amine hydrochloride, following the method described in Preparation 100. LCMS m/z=333 [M+H]⁺

### Preparation 102

### 6-chloro-N-(1-(2-fluoroethyl)azetidin-3-yl)-4-isopropyl-2,7-naphthyridine-1-carboxamide

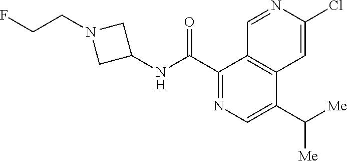 [SMILES: CC(C)c1cnc(C(=O)NC2CN(CCF)C2)c2cnc(Cl)cc12]

The title compound was obtained as a yellow solid, 50 mg, 35.9% yield, from 6-chloro-4-isopropyl-2,7-naphthyridine-1-carboxylic acid (Preparation 98) and 1-(2-fluoroethyl)azetidin-3-amine hydrochloride (Preparation 92), following the method described in Preparation 100. LCMS m/z=350 [M+H]⁺

### Preparation 103

### 6-chloro-N-((1r,3r)-3-cyanocyclobutyl)-4-isopropyl-2,7-naphthyridine-1-carboxamide

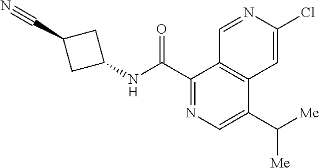 [SMILES: CC(C)c1cnc(C(=O)N[C@H]2C[C@H](C#N)C2)c2cnc(Cl)cc12]

The title compound was prepared as a light yellow solid, 80 mg, 61.0% yield, from 6-chloro-4-isopropyl-2,7-naphthyridine-1-carboxylic acid (Preparation 98) and trans-3-aminocyclobutane-1-carbonitrile following a similar procedure to that described in Preparation 100. LCMS m/z=329 [M+H]⁺

### Preparation 104

### Trans-6-chloro-N-(3-hydroxycyclobutyl)-4-isopropyl-2,7-naphthyridine-1-carboxamide

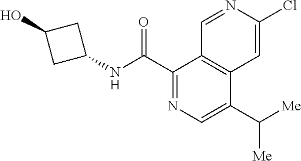 [SMILES: CC(C)c1cnc(C(=O)N[C@H]2C[C@H](O)C2)c2cnc(Cl)cc12]

A mixture of 6-chloro-4-isopropyl-2,7-naphthyridine-1-carboxylic acid (Preparation 98, 100 mg, 0.40 mmol), (1r,3r)-3-aminocyclobutane-1-carbonitrile (98.3 mg, 0.80 mmol), HATU (151 mg, 0.4 mmol) and DIPEA (102 mg, 0.8 mmol) in DMF (3 mL) was stirred at 40° C. for 3 h. The solvent was removed and the residue purified by preparative TLC (1/1 PE/EtOAc), to afford the title compound, 110 mg, 86.5% yield, as a pink solid. LCMS m/z=319 [M+H]⁺

### Preparation 105

### 6-chloro-N-((1r,3r)-3-ethoxycyclobutyl)-4-isopropyl-2,7-naphthyridine-1-carboxamide

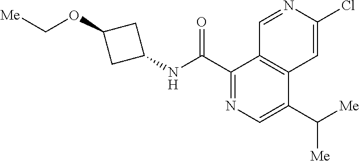 [SMILES: CCO[C@H]1C[C@H](NC(=O)c2ncc(C(C)C)c3cc(Cl)ncc23)C1]

The title compound was prepared, 120 mg, 72% yield, as a white solid, from 6-chloro-4-isopropyl-2,7-naphthyridine-1-carboxylic acid (Preparation 98) and trans-3-ethoxycyclobutan-1-amine hydrochloride, following a similar procedure to that described in Preparation 104. LCMS m/z=348 [M+H]⁺

### Preparation 106

### 6-chloro-N-((1s,3s)-3-hydroxy-3-methylcyclobutyl)-4-isopropyl-2,7-naphthyridine-1-carboxamide

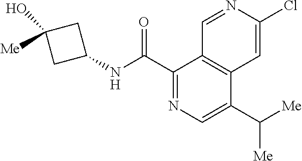 [SMILES: CC(C)c1cnc(C(=O)N[C@H]2C[C@@](C)(O)C2)c2cnc(Cl)cc12]

The title compound was prepared, 120 mg, 75.4% yield, as a white solid, from 6-chloro-4-isopropyl-2,7-naphthyridine-1-carboxylic acid (Preparation 98) and (1r,3r)-3-amino-1-methylcyclobutan-1-ol hydrochloride, following a similar procedure to that described in Preparation 104. LCMS m/z=334 [M+H]⁺

### Preparation 107

### methyl 6-(2-((3S,4R)-3-fluoro-4-hydroxy-3-methylpiperidin-1-yl)pyrimidin-4-yl)amino)-4-isopropyl-2,7-naphthyridine-1-carboxylate

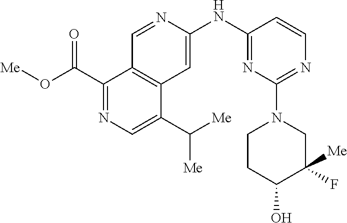 [SMILES: COC(=O)c1ncc(C(C)C)c2cc(Nc3ccnc(N4CC[C@@H](O)[C@@](C)(F)C4)n3)ncc12]

A solution of methyl 6-chloro-4-isopropyl-2,7-naphthyridine-1-carboxylate (Preparation 97, 3.9 g, 14.7 mmol), (3S,4R)-1-(4-aminopyrimidin-2-yl)-3-fluoro-3-methylpiperidin-4-ol (Preparation 8, 3.32 g, 14.7 mmol), Cs₂CO₃ (9.63 g, 29.4 mmol) and Brettphos Pd G3 (1.33 g, 1.47 mmol) in dioxane (50 mL) was heated to 120° C. for 2 h under N₂. The cooled mixture was diluted with water and the mixture extracted with EtOAc. The organic phase was washed with water and brine, dried over Na₂SO₄ and concentrated in vacuo. The residue was purified by silica gel column eluting with EtOAc/PE (1/1) to give the title compound, 3.8 g, 56.9% yield, as a light yellow solid. LCMS m/z=455 [M+H]⁺

### Preparation 108

### 6-((2-((3S,4R)-3-fluoro-4-hydroxy-3-methylpiperidin-1-yl)pyrimidin-4-yl)amino)-4-isopropyl-2,7-naphthyridine-1-carboxylic acid

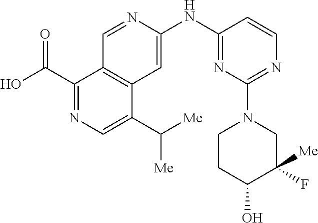 [SMILES: CC(C)c1cnc(C(=O)O)c2cnc(Nc3ccnc(N4CC[C@@H](O)[C@@](C)(F)C4)n3)cc12]

To a solution Preparation 107 (3.8 g, 8.36 mmol) and LiOH (799 mg, 33.4 mmol) in THF/water (80 mL/20 mL) was stirred at rt for 3 h. The reaction was acidified using 2 N HCl then concentrated in vacuo. The crude product was purified by reverse phase column chromatography to give the title compound, 3.4 g, 92.4% yield, as a yellow solid. LCMS m/z=441 [M+H]⁺

### Preparation 109

### tert-butyl (3S,4R)-3-fluoro-4-(6-((2-((3S,4R)-3-fluoro-4-hydroxy-3-methylpiperidin-1-yl)pyrimidin-4-yl)amino)-4-isopropyl-2,7-naphthyridine-1-carboxamido)pyrrolidine-1-carboxylate and tert-butyl (3R,4S)-3-fluoro-4-(6-((2-((3S,4R)-3-fluoro-4-hydroxy-3-methylpiperidin-1-yl)pyrimidin-4-yl)amino)-4-isopropyl-2,7-naphthyridine-1-carboxamido)pyrrolidine-1-carboxylate

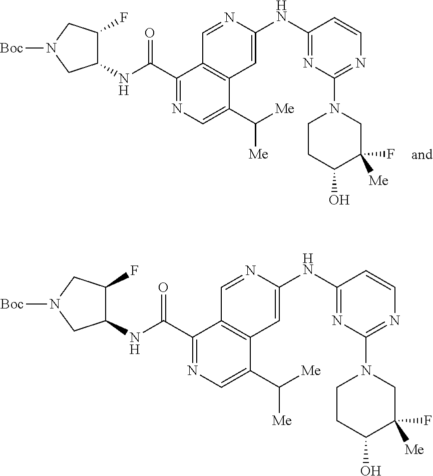 [SMILES: CC(C)c1cnc(C(=O)N[C@@H]2CN(C(=O)OC(C)(C)C)C[C@@H]2F)c2cnc(Nc3ccnc(N4CC[C@@H](O)[C@@](C)(F)C4)n3)cc12 ; "and" ; CC(C)c1cnc(C(=O)N[C@H]2CN(C(=O)OC(C)(C)C)C[C@H]2F)c2cnc(Nc3ccnc(N4CC[C@@H](O)[C@@](C)(F)C4)n3)cc12]

A mixture of 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 44 mg, 0.10 mmol), cis-rac-tert-butyl 3-amino-4-fluoropyrrolidine-1-carboxylate (40.8 mg, 0.20 mmol), HOBt (20.25 mg, 0.15 mmol) and EDCI (28.72 mg, 0.15 mmol) in DMF (5 mL) was stirred for 2 h at rt. The resulting solids were filtered off and the filtrate concentrated in vacuo. The residue was purified by silica gel column eluting with DCM/MeOH (10/1) to give the title compound, as a yellow solid, 20 mg, 32%. LCMS m/z=627 [M+H]⁺

### Preparation 110

### tert-butyl (3S,4S)-3-fluoro-4-(6-((2-((3S,4R)-3-fluoro-4-hydroxy-3-methylpiperidin-1-yl)pyrimidin-4-yl)amino)-4-isopropyl-2,7-naphthyridine-1-carboxamido)pyrrolidine-1-carboxylate and tert-butyl (3R,4R)-3-fluoro-4-(6-((2-((3S,4R)-3-fluoro-4-hydroxy-3-methylpiperidin-1-yl)pyrimidin-4-yl)amino)-4-isopropyl-2,7-naphthyridine-1-carboxamido)pyrrolidine-1-carboxylate

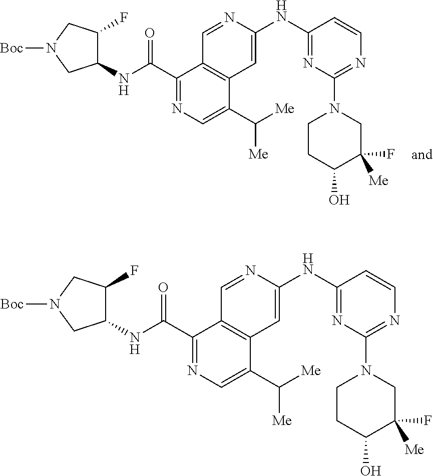 [SMILES: CC(C)c1cnc(C(=O)N[C@H]2CN(C(=O)OC(C)(C)C)C[C@@H]2F)c2cnc(Nc3ccnc(N4CC[C@@H](O)[C@@](C)(F)C4)n3)cc12 ; "and" ; CC(C)c1cnc(C(=O)N[C@@H]2CN(C(=O)OC(C)(C)C)C[C@H]2F)c2cnc(Nc3ccnc(N4CC[C@@H](O)[C@@](C)(F)C4)n3)cc12]

The title compound was obtained as a yellow solid, from 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108) and trans-rac-tert-butyl 3-amino-4-fluoropyrrolidine-1-carboxylate, following the method described in Preparation 109. LCMS m/z=627 [M+H]⁺

### Preparation 111

### 1,6-dichloro-4-iodo-1,2-dihydro-2,7-naphthyridine

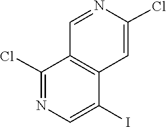 [SMILES: Clc1cc2c(I)cnc(Cl)c2cn1]

A mixture of 6-chloro-4-iodo-1,2-dihydro-2,7-naphthyridin-1-one (Preparation 93, 60 g, 0.196 mol) in POCl₃ (320 mL) was stirred at 100° C. for 1.5 h. The mixture was concentrated in vacuo and neutralized with cooled saturated aq. NaHCO₃. The mixture was extracted with EtOAc (3×300 mL), the combined organic layers dried over Na₂SO₄, filtered and evaporated under reduced pressure to give the title compound, 53 g (84%) as a yellow solid. LCMS m/z=325 [M+H]⁺.

### Preparation 112

### 1,6-dichloro-4-(prop-1-en-2-yl)-1,2-dihydro-2,7-naphthyridine

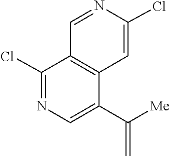

To a solution of 1,6-dichloro-4-iodo-2,7-naphthyridine (Preparation 111, 30 g, 92.5 mmol) in 1,4-dioxane/H₂O (300/70 mL) was added 4,4,5,5-tetramethyl-2-(prop-1-en-2-yl)-1,3,2-dioxaborolane (15 g, 93 mmol), K₂CO₃ (37.8 g, 276 mmol) and Pd(amphos)Cl₂ (3 g, 4.2 mmol) and the solution was stirred for 0.5 h at 50° C. The mixture was cooled to rt, diluted with water (200 mL) and extracted with EtOAc (2×300 mL). The combined organic layers were washed with brine (200 mL), dried over anhydrous Na₂SO₄ and concentrated in vacuo. The crude product was purified by silica gel column chromatography eluting with EtOAc: PE (1: 10) to give the title compound, 15 g (68.1%) as white solid. LCMS m/z=239 [M+H]⁺.

### Preparation 113

### 1,6-dichloro-4-isopropyl-2,7-naphthyridine

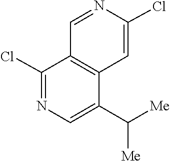 [SMILES: CC(C)c1cnc(Cl)c2cnc(Cl)cc12]

To a solution of 1,6-dichloro-4-(prop-1-en-2-yl)-2,7-naphthyridine (Preparation 112, 4 g, 16.8 mmol) in EtOAc (300 mL) was added PtO₂ (5 g, 22 mmol) and the resulting mixture was stirred at 25° C. for 24 h under H₂ atmosphere. The solid was filtered off and the filtrate was concentrated in vacuo. The residue was purified by silica gel column chromatography (EtOAc:PE, 1:8) to give the title compound, 3 g (75%) as a white solid. LCMS m/z=241 [M+H]⁺. ¹H NMR (300 MHz, DMSO-d₆) δ 9.47 (d, 1H), 8.47 (d, 1H), 8.26 (d, 1H), 3.64 (p, 1H), 1.33 (d, 6H).

### Preparation 114

### 5-bromo-N-tert-butyl-2-chloroisonicotinamide

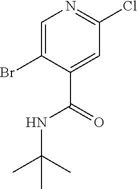

A solution of 5-bromo-2-chloropyridine-4-carboxylic acid (4 g, 16.9 mmol), 2-methylpropan-2-amine (1.47 g, 20.2 mmol), EDC HCl (4.85 g, 25.3 mmol) and HOBT (3.41 g, 25.3 mmol) in DMF (30 mL), under N₂ was stirred overnight at rt. The reaction was diluted with water, extracted with EtOAc, the organic layers were combined, dried over anhydrous Na₂SO₄ and concentrated in vacuo. The residue was purified by column chromatography on silica gel (PE/EtOAc, 2:1) to give the title compound, 3 g (60.9%) as a white solid. LCMS m/z=293 [M+H]⁺; ¹H NMR (300 MHz, DMSO-d₆) δ: 8.64 (s, 1H), 8.30 (s, 1H), 7.58 (s, 1H), 1.36 (s, 9H).

### Preparation 115

### (E)-N-(tert-butyl)-2-chloro-5-(2-ethoxyvinyl)isonicotinamide

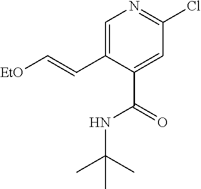 [SMILES: CCO/C=C/c1cnc(Cl)cc1C(=O)NC(C)(C)C]

A solution of 5-bromo-N-tert-butyl-2-chloropyridine-4-carboxamide (Preparation 114, 2 g, 6.85 mmol), 2-[(E)-2-ethoxyethenyl]-4,4,5,5-tetramethyl-1,3,2-dioxaborolane (1.49 g, 7.53 mmol), Cs₂CO₃ (4.46 g, 13.7 mmol) and Pd(dppf)Cl₂ (501 mg, 0.685 mmol) in dioxane (30 mL) and H₂O (6 mL) was stirred for 2 h at 80° C. The cooled solution was diluted with water and extracted with EtOAc, the combined organic layers dried over anhydrous Na₂SO₄ and concentrated in vacuo. The residue was purified by silica gel chromatography eluting with PE/EtOAc (2:1) to give the title compound, 1.2 g (62.1%) as a yellow solid. LCMS m/z=283 [M+H]⁺; ¹H NMR (300 MHz, DMSO-d₆) δ: 8.55 (s, 1H), 8.20 (s, 1H), 7.35 (d, 1H), 7.28 (s, 1H), 5.79 (d, 1H), 3.90 (q, 2H), 1.35 (s, 9H), 1.26 (t, 3H).

### Preparation 116

### 7-chloro-2,6-naphthyridin-1(2H)-one

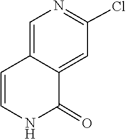 [SMILES: O=c1[nH]ccc2cnc(Cl)cc12]

A solution of (E)-N-(tert-butyl)-2-chloro-5-(2-ethoxyvinyl)isonicotinamide_(Preparation 115, 1.2 g, 4.24 mmol) in TFA (20 mL) was stirred overnight at 100° C. The resulting mixture was cooled and evaporated under reduced pressure to give the title compound, 600 mg, as a red solid. The crude product was used directly without any further purification. LCMS m/z=181 [M+H]⁺.

### Preparation 117

### 4-bromo-7-chloro-2,6-naphthyridin-1(2H)-one

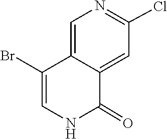 [SMILES: O=c1[nH]cc(Br)c2cnc(Cl)cc12]

A solution of 7-chloro-1,2-dihydro-2,6-naphthyridin-1-one (Preparation 116, 3 g, 16.6 mmol) and NBS (3.54 g, 19.9 mmol) in DCM (40 mL) was stirred for 1 h at rt. The resulting solid was collected by filtration to give the title compound, 3 g (69.7%) as a white solid. LCMS m/z=261 [M+H]⁺

### Preparation 118

### 4-bromo-7-chloro-2,6-naphthyridin-1-yl trifluoromethanesulfonate

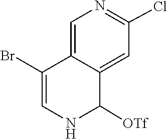 [SMILES: O=S(=O)(OC1NC=C(Br)c2cnc(Cl)cc21)C(F)(F)F]

A solution of 4-bromo-7-chloro-2,6-naphthyridin-1(2H)-one (Preparation 117, 1 g, 3.85 mmol) and TEA (777 mg, 7.70 mmol) in DCM (15 mL) was cooled to −78° C., and then Tf₂O (4.34 g, 15.4 mmol) was added drop wise over 10 min. The reaction was stirred for 0.5 h at −78° C., then warmed to rt and stirred for 0.5 h. The reaction was quenched with ice-water (2 mL), extracted with DCM, the organic layers combined, dried over anhydrous Na₂SO₄ and concentrated in vacuo. The residue was purified by silica gel column eluting with EtOAc:PE (0-10%) to give the title compound, 1 g (66.6%) as a white solid. LCMS m/z=393 [M+H]⁺; ¹H NMR (300 MHz, DMSO-d₆) δ: 9.47 (s, 1H), 8.78 (s, 1H), 8.14 (d, 1H).

### Preparation 119

### 4-bromo-7-chloro-1-iodo-2,6-naphthyridine

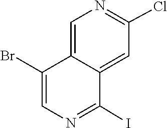 [SMILES: Clc1cc2c(I)ncc(Br)c2cn1]

A mixture of 4-bromo-7-chloro-2,6-naphthyridin-1-yl trifluoromethanesulfonate (Preparation 118, 500 mg, 1.27 mmol) and NaI (952 mg, 6.35 mmol) in MeCN (9 mL) was cooled to 0° C. and a solution of trifluoromethanesulfonate acid (381 mg, 2.54 mmol) in MeCN (1 mL) was added drop wise over 10 min. The reaction was then stirred at rt for 1.5 h. The reaction mixture was extracted with EtOAc, the organic layers combined, washed with brine, dried over anhydrous Na₂SO₄ and evaporated under reduced pressure to give the title compound, 500 mg as a dark solid. LCMS m/z=369 [M+H]⁺.

### Preparation 120

### 4-bromo-7-chloro-1-(prop-1-en-2-yl)-2,6-naphthyridine

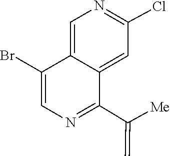

A solution of 4-bromo-7-chloro-1-iodo-2,6-naphthyridine (Preparation 119, 500 mg, 1.35 mmol), 4,4,5,5-tetramethyl-2-(prop-1-en-2-yl)-1,3,2-dioxaborolane (226 mg, 1.35 mmol), K₂CO₃ (372 mg, 2.7 mmol) and Pd(dppf)Cl₂ (0.99 mg, 0.135 mmol) in dioxane (5 mL) and H₂O (1 mL) was stirred for 2 h at 80° C. under N₂. The cooled reaction mixture was extracted with EtOAc, the organic layers combined, dried over anhydrous Na₂SO₄ and concentrated in vacuo. The residue was purified by Prep-TLC with PE/EtOAc (8:1) to give the title compound, 200 mg (52.3%) as a light-yellow oil. LCMS m/z=285 [M+H]⁺.

### Preparation 121

### 4-bromo-7-chloro-1-isopropyl-2,6-naphthyridine

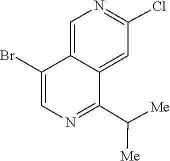 [SMILES: CC(C)c1ncc(Br)c2cnc(Cl)cc12]

A mixture of 4-bromo-7-chloro-1-(prop-1-en-2-yl)-2,6-naphthyridine (Preparation 120, 160 mg, 0.564 mmol) in EtOAc (6 mL) and PtO₂ (166 mg, 0.733 mmol) was stirred under H₂ for 3 h at rt. The resulting solids were filtered off and the filtrate evaporated under reduced pressure to give the title compound, 100 mg (62.1%) as a yellow solid. LCMS m/z=287 [M+H].

### SYNTHESIS OF EXEMPLARY COMPOUNDS

### Example 1

### N3-(2-((3S,4R)-3-fluoro-4-methoxypiperidin-1-yl)pyrimidin-4-yl)-5-isopropyl-N8-(((4R)-4-(methylsulfonyl)pyrrolidin-3-yl)methyl)isoquinoline-3,8-diamine or N3-(2-((3S,4R)-3-fluoro-4-methoxypiperidin-1-yl)pyrimidin-4-yl)-5-isopropyl-N8-(((4S)-4-(methylsulfonyl)pyrrolidin-3-yl)methyl)isoquinoline-3,8-diamine

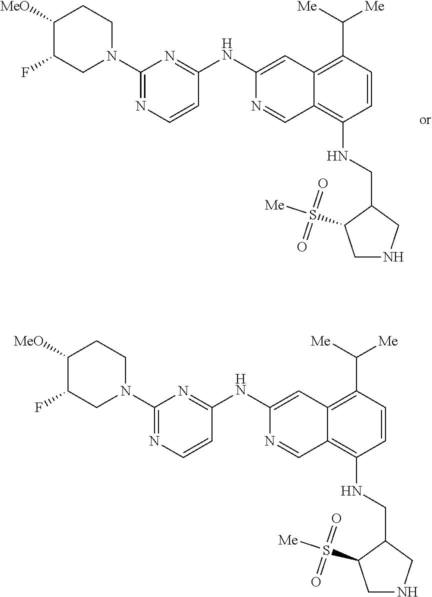 [SMILES: CO[C@@H]1CCN(c2nccc(Nc3cc4c(C(C)C)ccc(NCC5CNC[C@@H]5S(C)(=O)=O)c4cn3)n2)C[C@@H]1F ; CO[C@@H]1CCN(c2nccc(Nc3cc4c(C(C)C)ccc(NCC5CNC[C@H]5S(C)(=O)=O)c4cn3)n2)C[C@@H]1F]

TFA (xs) was added to the compound of Peak 1 of Preparation 47 and 48 (15 mg, 0.0223 mmol) in DCM at rt and the resulting mixture stirred at rt for 1 h. The reaction mixture was evaporated to dryness in vacuo and the residue purified by HPLC-1 to afford the title compound as a yellow solid (4 mg, 31%). LCMS m/z=572 [M+H]⁺; ¹H NMR (300 MHz, DMSO-d₆) δ: 9.91 (s, 1H), 9.07 (s, 1H), 8.63 (s, 1H), 8.01 (d, 1H), 7.43 (d, 1H), 6.46 (dd, 2H), 4.95 (d, 1H), 4.83-4.66 (m, 1H), 4.02 (dt, 2H), 3.69-3.38 (m, 5H), 3.37 (s, 3H), 3.32-3.16 (m, 3H), 3.10-2.88 (m, 5H), 1.91-1.68 (m, 2H), 1.31 (dd, 6H).

### Example 2

### (3S,4R)-3-fluoro-1-(4-((5-isopropyl-8-((2-(methylsulfonyl)ethyl)amino)-2,7-naphthyridin-3-yl)amino)pyrimidin-2-yl)-3-methylpiperidin-4-ol

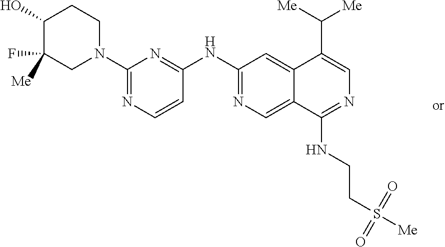 [SMILES: CC(C)c1cnc(NCCS(C)(=O)=O)c2cnc(Nc3ccnc(N4CC[C@@H](O)[C@@](C)(F)C4)n3)cc12]

A mixture of 6-chloro-4-isopropyl-N-(2-(methylsulfonyl)ethyl)-2,7-naphthyridin-1-amine (Preparation 49, 70 mg, 0.213 mmol), (3S,4R)-1-(4-aminopyrimidin-2-yl)-3-fluoro-3-methylpiperidin-4-ol (Preparation 8, 48.1 mg, 0.213 mmol), Cs₂CO₃ (138 mg, 0.426 mmol) and RuPhos Pd G3 (53.4 mg, 0.064 mmol) in dioxane (15 mL) was stirred at 130° C. for 2 h. The mixture was evaporated to dryness in vacuo and the residue purified by prep-TLC (DCM:MeOH=20:1) followed by HPLC-2 (Gradient (% organic): 10-50%) to afford the title compound as a light yellow sold (20 mg). LCMS m/z=518 [M+H]⁺; ¹H NMR (300 MHz, DMSO-d₆) δ: 10.09 (s, 1H), 9.31 (s, 1H), 8.48 (s, 1H), 8.02 (d, 1H), 7.94 (s, 1H), 7.90 (d, 1H), 6.46 (d, 1H), 5.03 (d, 1H), 4.81-4.51 (m, 2H), 3.88 (q, 2H), 3.68-3.41 (m, 3H), 3.30-3.07 (m, 3H), 3.05 (s, 3H), 1.73 (s, 2H), 1.46-1.19 (m, 9H).

### Example 3

### N¹-(2-(dimethylamino)ethyl)-N⁶-(2-((3S,4R)-3-fluoro-4-methoxypiperidin-1-yl)pyrimidin-4-yl)-4-isopropyl-2,7-naphthyridine-1,6-diamine

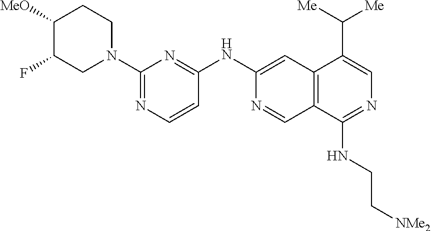 [SMILES: CO[C@@H]1CCN(c2nccc(Nc3cc4c(C(C)C)cnc(NCCN(C)C)c4cn3)n2)C[C@@H]1F]

The title compound was prepared from 2-((3S,4R)-3-fluoro-4-methoxypiperidin-1-yl)pyrimidin-4-amine (Preparation 31) and N¹-(6-chloro-4-isopropyl-2,7-naphthyridin-1-yl)-N2,N²-dimethylethane-1,2-diamine (Preparation 50) using an analogous method to that described for Example 2. Yield: 38.9 mg, 36%; LCMS m/z=483 [M+H]⁺; ¹H NMR (300 MHz, DMSO-d₆) δ: 10.11 (s, 1H), 9.32 (s, 1H), 8.48 (s, 1H), 8.04 (d, 1H), 7.90 (s, 1H), 7.55 (t, 1H), 6.47 (d, 1H), 5.11-4.78 (m, 1H), 4.72 (t, 1H), 4.47 (d, 1H), 3.75-3.43 (m, 4H), 3.37 (s, 3H), 3.34-3.21 (m, 2H), 2.57-2.51 (m, 2H), 2.22 (s, 6H), 1.87-1.65 (m, 2H), 1.31 (dd, 6H).

### Example 4

### (3S,4R)-1-(4-((8-(((S)-2-(dimethylamino)propyl)amino)-5-isopropyl-2,7-naphthyridin-3-yl)amino)pyrimidin-2-yl)-3-fluoro-3-methylpiperidin-4-ol or (3S,4R)-1-(4-((8-(((R)-2-(dimethylamino)propyl)amino)-5-isopropyl-2,7-naphthyridin-3-yl)amino)pyrimidin-2-yl)-3-fluoro-3-methylpiperidin-4-ol

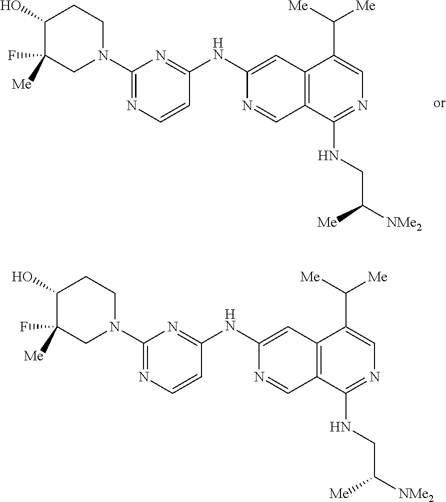 [SMILES: CC(C)c1cnc(NC[C@H](C)N(C)C)c2cnc(Nc3ccnc(N4CC[C@@H](O)[C@@](C)(F)C4)n3)cc12 ; CC(C)c1cnc(NC[C@@H](C)N(C)C)c2cnc(Nc3ccnc(N4CC[C@@H](O)[C@@](C)(F)C4)n3)cc12]

A mixture of N1-(6-chloro-4-isopropyl-2,7-naphthyridin-1-yl)-N2,N2-dimethylpropane-1,2-diamine (Preparation 53, 300 mg, 0.97 mmol), (3S,4R)-1-(4-aminopyrimidin-2-yl)-3-fluoro-3-methylpiperidin-4-ol (Preparation 8, 221 mg, 0.97 mmol), BrettPhos Pd G3 and Cs₂CO₃ in dioxane (5 mL) was stirred overnight at 100° C. under N₂. The mixture was evaporated and the residue purified by prep TLC (5% MeOH/DCM). The resulting residue was further purified by HPLC-3 to afford the title compound as a light yellow solid (56 mg, peak 2). LCMS m/z=497 [M+H]⁺; ¹H NMR (300 MHz, DMSO-d₆) δ: 10.06 (s, 1H), 9.34 (s, 1H), 8.46 (s, 1H), 8.02 (d, 1H), 7.90 (s, 1H), 7.46 (s, 1H), 6.47 (d, 1H), 5.04 (d, 1H), 4.85-4.59 (m, 2H), 3.71-3.39 (m, 3H), 3.28-3.03 (m, 3H), 2.97 (q, 1H), 2.24 (s, 6H), 1.83-1.67 (m, 2H), 1.49-1.22 (m, 9H), 0.96 (d, 3H).

### Example 5

### 1-(3-(((3-((2-((3S,4R)-3-fluoro-4-hydroxy-3-methylpiperidin-1-yl)pyrimidin-4-yl)amino)-5-isopropylisoquinolin-8-yl)amino)methyl)azetidin-1-yl)ethan-1-one

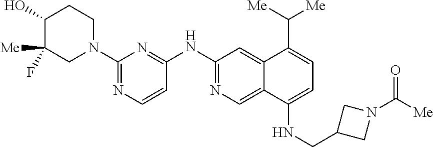 [SMILES: CC(=O)N1CC(CNc2ccc(C(C)C)c3cc(Nc4ccnc(N5CC[C@@H](O)[C@@](C)(F)C5)n4)ncc23)C1]

A mixture of 1-(3-(((3-chloro-5-isopropylisoquinolin-8-yl)amino)methyl)azetidin-1-yl)ethan-1-one (Preparation 44, 21 mg, 0.063 mmol), (3S,4R)-1-(4-aminopyrimidin-2-yl)-3-fluoro-3-methylpiperidin-4-ol (Preparation 8, 14 mg, 0.063 mmol), BrettPhos Pd G4 and Cs₂CO₃ in dioxane (0.75 mL) was stirred at 90° C. under N₂ for 1 h. The reaction mixture was diluted with DCM and washed with H₂O. The combined organics were dried, evaporated to dryness in vacuo and the residue purified using RP-ISCO (0-60% MeCN/H₂O+0.1% TFA). The residue was diluted with sat. aq. NaHCO₃ solution and extracted with 5% MeOH/DCM (×2). The combined organics were dried (Na₂SO₄) and evaporated to dryness in vacuo to give the title compound as a pale yellow solid (18.3 mg, 55%). LCMS m/z=522 [M+H]⁺; ¹H NMR (400 MHz, DMSO-d₆) δ: 9.82 (s, 1H), 9.30 (s, 1H), 8.55 (s, 1H), 7.95 (d, 1H), 7.33 (d, 1H), 6.59 (t, 1H), 6.43 (dd, 2H), 4.99 (d, 1H), 4.79-4.60 (m, 2H), 4.20 (t, 1H), 3.91 (t, 1H), 3.85 (dd, 1H), 3.64-3.36 (m, 5H), 3.19-3.04 (m, 2H), 3.00-2.90 (m, 1H), 1.77-1.66 (m, 5H), 1.40-1.22 (m, 9H).

### Example 6

### (3S,4R)-3-fluoro-1-(4-((5-isopropyl-8-((1-methyl-1H-pyrazol-4-yl)amino)-2,7-naphthyridin-3-yl)amino)pyrimidin-2-yl)-3-methylpiperidin-4-ol

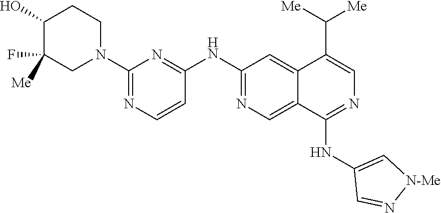

A mixture of 6-chloro-4-isopropyl-N-(1-methyl-1H-pyrazol-4-yl)-2,7-naphthyridin-1-amine (Preparation 55, 60 mg, 0.199 mmol), (3S,4R)-1-(4-aminopyrimidin-2-yl)-3-fluoro-3-methylpiperidin-4-ol (Preparation 8, 44.9 mg, 0.199 mmol), BrettPhos Pd G3 (36.0 mg, 0.040 mmol) and Cs₂CO₃ (129 mg, 0.3976 mmol) in dioxane (1 mL) was heated in a sealed tube at 130° C. for 8 h. The resulting solution was evaporated to dryness in vacuo and the residue purified by prep-TLC (40:1 DCM/MeOH) followed by HPLC-2 (Gradient (% organic): 10-50%) to afford the title compound as an off-white solid (19.8 mg, 20%). LCMS m/z=492 [M+H]⁺; ¹H NMR (300 MHz, MeOH-d₄) δ: 9.37 (s, 1H), 8.54 (s, 1H), 8.08 (s, 1H), 8.03-7.92 (m, 2H), 7.65 (s, 1H), 6.42 (d, 1H), 4.955 (s, 1H), 4.76-4.65 (m, 2H), 3.90 (s, 3H), 3.68 (ddd, 1H), 3.45 (q, 1H), 3.21 (d, 1H), 1.89 (d, 2H), 1.51-1.36 (m, 9H).

### Example 7

### 1-(3-(((3-((2-((3S,4R)-3-fluoro-4-hydroxy-3-methylpiperidin-1-yl)pyrimidin-4-yl)amino)-5-isopropylisoquinolin-8-yl)oxy)methyl)-3-methylazetidin-1-yl)ethan-1-one

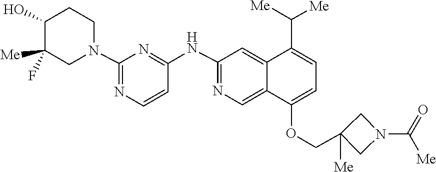 [SMILES: CC(=O)N1CC(C)(COc2ccc(C(C)C)c3cc(Nc4ccnc(N5CC[C@@H](O)[C@@](C)(F)C5)n4)ncc23)C1]

Part 1: A mixture of 3-chloro-5-isopropylisoquinolin-8-ol (Preparation 43, 84 mg, 0.379 mmol), tert-butyl 3-(bromomethyl)-3-methylazetidine-1-carboxylate (105 mg, 0.398 mmol) and K₂CO₃ (131 mg, 0.947 mmol) in DMF (1.5 mL) was heated at 80° C. overnight. The reaction mixture was diluted with water and extracted into EtOAc. The combined extracts were washed with H₂O, brine, dried (Na₂SO₄) and evaporated to dryness in vacuo. The residue (128 mg) was dissolved in DCM (1.5 mL) and TFA (0.25 mL) added and the resulting mixture was stirred at rt for 90 min. The reaction mixture was evaporated to dryness in vacuo and the residue dissolved in DCM and washed with sat. aq. NaHCO₃. The combined organics were dried (Na₂SO₄) and evaporated to dryness to afford 3-chloro-5-isopropyl-8-((3-methylazetidin-3-yl)methoxy)isoquinoline as a dark yellow oil (93 mg, 80.5%). LCMS m/z=331 [M+H]⁺

Part 2: Acetyl chloride (29 mg, 0.366 mmol) was added to a mixture of 3-chloro-5-isopropyl-8-((3-methylazetidin-3-yl)methoxy)isoquinoline (Part 1, 93 mg, 0.305 mmol) and TEA (46 mg, 0.458 mmol) in DCM (2 mL) and the resulting mixture stirred at rt for 15 min. The reaction was diluted with DCM and washed with water. The combined organics were dried (Na₂SO₄) and evaporated to dryness in vacuo to afford 1-(3-(((3-chloro-5-isopropylisoquinolin-8-yl)oxy)methyl)-3-methylazetidin-1-yl)ethan-1-one (62 mg, 18%). LCMS m/z=347 [M+H]⁺

Part 3: A mixture of 1-(3-(((3-chloro-5-isopropylisoquinolin-8-yl)oxy)methyl)-3-methylazetidin-1-yl)ethan-1-one (Part 2, 59 mg, 0.170 mmol), (3S,4R)-1-(4-aminopyrimidin-2-yl)-3-fluoro-3-methylpiperidin-4-ol (Preparation 8, 38 mg, 0.170 mmol), XPhos Pd G4 (7.32 mg, 8.51 mmol) and Cs₂CO₃ (111 mg, 0.34 mmol) in dioxane (1 mL) was heated under N₂ at 90° C. for 1 h. The reaction was diluted with DCM and washed with H₂O. The combined organics were dried (Na₂SO₄) and evaporated to dryness in vacuo. The residue was purified using ISCO chromatography (0-60% MeCN/H₂O+0.1% TFA) followed by treatment of the residue with aq. NaHCO₃ and extraction into 10% MeOH/DCM, drying (Na₂SO₄) and evaporation to dryness in vacuo to afford the title compound as a pale yellow solid (64 mg, 37.8%). LCMS m/z=537 [M+H]⁺; ¹H NMR (400 MHz, DMSO-d₆) δ: 9.96 (s, 1H), 9.21 (d, 1H), 8.66 (s, 1H), 7.98 (d, 1H), 7.48 (d, 1H), 6.87 (d, 1H), 6.44 (d, 1H), 5.00 (d, 1H), 4.79-4.61 (m, 2H), 4.19-4.10 (m, 3H), 3.89 (dd, 2H), 3.64-3.45 (m, 3H), 3.23-3.05 (m, 2H), 1.78 (s, 3H), 1.77-1.66 (m, 2H), 1.41 (s, 3H), 1.39-1.28 (m, 9H).

### Example 8

### 1-(3-(((3-((2-((3S,4R)-3-fluoro-4-hydroxy-3-methylpiperidin-1-yl)pyrimidin-4-yl)amino)-5-isopropylisoquinolin-8-yl)oxy)methyl)azetidin-1-yl)ethan-1-one

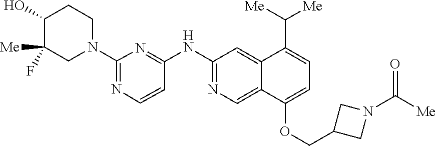

A mixture of 1-(3-(((3-chloro-5-isopropylisoquinolin-8-yl)oxy)methyl)azetidin-1-yl)ethan-1-one (Preparation 63, 16 mg, 0.048 mmol), (3S,4R)-1-(4-aminopyrimidin-2-yl)-3-fluoro-3-methylpiperidin-4-ol (Preparation 8, 10.9 mg, 0.048 mmol), XPhos Pd G4 (2.1 mg, 2.4 mmol) and Cs₂CO₃ (31 mg, 0.096 mmol) in dioxane (0.5 mL) was heated under N₂ at 90° C. for 1 h. The reaction was diluted with DCM and washed with H₂O. The combined organics were dried (Na₂SO₄) and evaporated to dryness in vacuo. The residue was purified using ISCO chromatography (0-8% MeOH/DCM) to afford the title compound as a white solid (11 mg, 44%). LCMS m/z=492 [M+H]⁺; ¹H NMR (400 MHz, DMSO-d₆) δ: 9.96 (s, 1H), 9.22 (s, 1H), 8.66 (s, 1H), 7.97 (d, 1H), 7.48 (d, 1H), 6.87 (d, 1H), 6.43 (d, 1H), 5.00 (d, 1H), 4.80-4.60 (m, 2H), 4.29 (t, 3H), 4.09-3.95 (m, 2H), 3.77 (dd, 1H), 3.61-3.45 (m, 2H), 3.22-3.03 (m, 3H), 1.79-1.67 (m, 5H), 1.40-1.26 (m, 9H).

### Example 9-18

The title compounds were prepared from the appropriate amine and the appropriate halide (RCl) using an analogous method to that described for Example 7, except using the following isolation procedure. RP-ISCO (0-70% MeCN/H₂O+0.1% TFA) followed by treatment of residue with aq. NaHCO₃ and extraction into 10% MeOH/DCM, drying (Na₂SO₄) and evaporation to dryness in vacuo.

Amine-1, (3S,4R)-1-(4-aminopyrimidin-2-yl)-3-fluoro-3-methylpiperidin-4-ol (Preparation 8);

Amine-2, (3S,4R)-1-(4-aminopyrimidin-2-yl)-3-fluoro-4-methylpiperidin-4-ol (Preparation 13);

Amine-3, 1-(4-aminopyrimidin-2-yl)-4-methylpiperidin-4-ol (Preparation 23)

| Example | |
|---|---|
| No. | Name/Structure/Reactants/Data |
| 9 | (3S,4R)-3-fluoro-1-(4-((5-isopropyl-8-((1-methyl-1H-pyrazol-4- |
| | yl)methoxy)isoquinolin-3-yl)amino)pyrimidin-2-yl)-3-methylpiperidin-4-ol |
| | 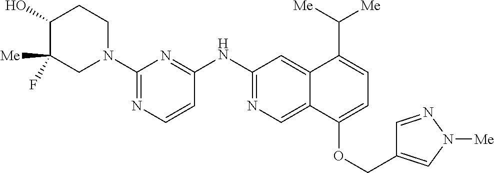 [SMILES: CC(C)c1ccc(OCc2cnn(C)c2)c2cnc(Nc3ccnc(N4CC[C@@H](O)[C@@](C)(F)C4)n3)cc12] |
| | Amine-1; RCl: 3-chloro-5-isopropyl-8-((1-methyl-1H-pyrazol-4- |
| | yl)methoxy)isoquinoline (Preparation 45). |
| | Yield: 24.7 mg, 51%; LCMS m/z = 506 [M + H]⁺; ¹H NMR (400 MHz, DMSO-d₆) δ: |
| | 9.96 (s, 1H), 9.21 (s, 1H), 8.65 (s, 1H), 7.97 (d, 1H), 7.88 (s, 1H), 7.58 (s, |
| | 1H), 7.47 (d, 1H), 6.97 (d, 1H), 6.42 (d, 1H), 5.14 (s, 2H), 5.00 (d, 1H), 4.78- |
| | 4.60 (m, 2H), 3.83 (s, 3H), 3.62-3.44 (m, 2H), 3.21-3.05 (m, 2H), 1.79-1.66 |
| | (m, 2H), 1.40-1.28 (m, 9H). |
| 10 | (3S,4R)-3-fluoro-1-(4-((5-isopropyl-8-((1-methyl-1H-1,2,3-triazol-4- |
| | yl)methoxy)isoquinolin-3-yl)amino)pyrimidin-2-yl)-3-methylpiperidin-4-ol |
| | 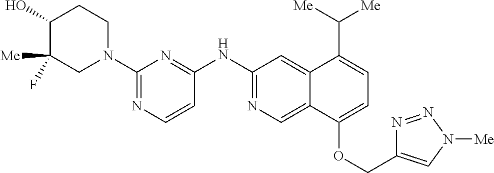 [SMILES: CC(C)c1ccc(OCc2cn(C)nn2)c2cnc(Nc3ccnc(N4CC[C@@H](O)[C@@](C)(F)C4)n3)cc12] |
| | Amine-1; RCl: 3-chloro-5-isopropyl-8-((1-methyl-1H-1,2,3-triazol-4- |
| | yl)methoxy)isoquinoline (Preparation 80). |
| | Yield: 14.2 mg, 44%; LCMS m/z = 507 [M + H]⁺; ¹H NMR (400 MHz, DMSO-d₆) δ: |
| | 9.97 (s, 1H), 9.22 (s, 1H), 8.66 (s, 1H), 8.28 (s, 1H), 7.97 (d, 1H), 7.49 (d, |
| | 1H), 7.05 (d, 1H), 6.42 (d, 1H), 5.34 (s, 2H), 5.00 (d, 1H), 4.78-4.60 (m, 2H), |
| | 4.06 (s, 3H), 3.63-3.44 (m, 2H), 3.20-3.05 (m, 2H), 1.80-1.66 (m, 2H), 1.40- |
| | 1.26 (m, 9H). |
| 11 | 1-(3-(((3-((2-((3S,4R)-3-fluoro-4-hydroxy-4-methylpiperidin-1-yl)pyrimidin-4- |
| | yl)amino)-5-isopropylisoquinolin-8-yl)oxy)methyl)azetidin-1-yl)ethanone |
| | 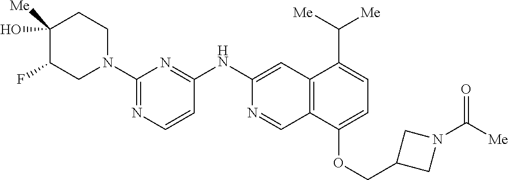 [SMILES: CC(=O)N1CC(COc2ccc(C(C)C)c3cc(Nc4ccnc(N5CC[C@@](C)(O)[C@@H](F)C5)n4)ncc23)C1] |
| | Amine-2; RCl: 1-(3-(((3-chloro-5-isopropylisoquinolin-8-yl)oxy)methyl)azetidin-1- |
| | yl)ethan-1-one (Preparation 63). |
| | Yield: 32 mg, 65.7%; LCMS m/z = 523 [M + H]⁺; ¹H NMR (400 MHz, DMSO-d₆) δ: |
| | 10.01 (s, 1H), 9.22 (s, 1H), 8.70 (s, 1H), 7.99 (d, 1H), 7.47 (d, 1H), 6.87 (d, |
| | 1H), 6.45 (d, 1H), 4.82 (s, 1H), 4.51-4.24 (m, 5H), 4.17 (d, 1H), 4.08-3.95 (m, |
| | 2H), 3.77 (dd, 1H), 3.60 (ddd, 1H), 3.56-3.42 (m, 2H), 3.16-3.03 (m, 1H), |
| | 1.77 (s, 3H), 1.71 (d, 1H), 1.62-1.47 (m, 1H), 1.30 (dd, 6H), 1.24 (s, 3H). |
| 12 | (3S,4R)-1-(4-((8-(2-(1H-imidazol-1-yl)ethoxy)-5-isopropylisoquinolin-3- |
| | yl)amino)pyrimidin-2-yl)-3-fluoro-4-methylpiperidin-4-ol |
| | 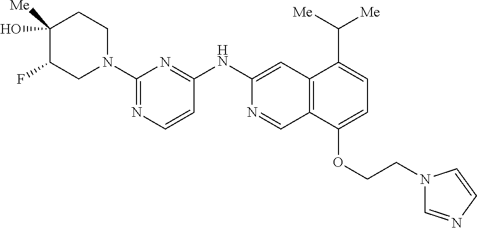 [SMILES: CC(C)c1ccc(OCCn2ccnc2)c2cnc(Nc3ccnc(N4CC[C@@](C)(O)[C@@H](F)C4)n3)cc12] |
| | Cat: BrettPhos Pd G4 |
| | Amine-2; RCl: 8-(2-(1H-imidazol-1-yl)ethoxy)-3-chloro-5-isopropylisoquinoline |
| | (Preparation 87). |
| | Yield: 26.8 mg, 46.5%; LCMS m/z = 506 [M + H]⁺; ¹H NMR (400 MHz, DMSO-d₆) |
| | δ: 9.96 (s, 1H), 9.20 (s, 1H), 8.67 (s, 1H), 8.00 (d, 1H), 7.80 (s, 1H), 7.44 (d, |
| | 1H), 7.32 (d, 1H), 6.89 (s, 1H), 6.83 (d, 1H), 6.46 (d, 1H), 4.81 (s, 1H), 4.51 |
| | (t, 2H), 4.47-4.23 (m, 4H), 4.17 (d, 1H), 3.60 (ddd, 1H), 3.53-3.42 (m, 2H), |
| | 1.70 (dp, 1H), 1.54 (ddd, 1H), 1.32-1.22 (m, 9H). |
| 13 | (3S,4R)-3-fluoro-1-(4-((5-isopropyl-8-(2-(2-methyl-1H-imidazol-1- |
| | yl)ethoxy)isoquinolin-3-yl)amino)pyrimidin-2-yl)-4-methylpiperidin-4-ol |
| | 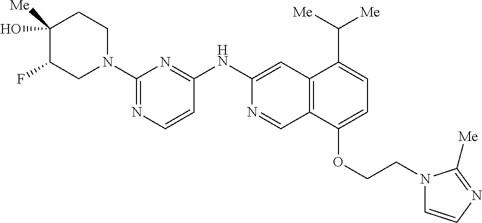 [SMILES: Cc1nccn1CCOc1ccc(C(C)C)c2cc(Nc3ccnc(N4CC[C@@](C)(O)[C@@H](F)C4)n3)ncc12] |
| | Cat: BrettPhos Pd G4 |
| | Amine-2; RCl: 3-chloro-5-isopropyl-8-(2-(2-methyl-1H-imidazol-1- |
| | yl)ethoxy)isoquinoline (Preparation 67). |
| | Yield: 30 mg, 58%; LCMS m/z = 520 [M + H]⁺; ¹H NMR (400 MHz, DMSO-d₆) δ: |
| | 9.98 (s, 1H), 9.17 (s, 1H), 8.68 (s, 1H), 8.00 (d, 1H), 7.45 (d, 1H), 7.22 (d, |
| | 1H), 6.84 (d, 1H), 6.73 (d, 1H), 6.44 (d, 1H), 4.81 (s, 1H), 4.49-4.24 (m, 6H), |
| | 4.17 (d, 1H), 3.66-3.54 (m, 1H), 3.54-3.42 (m, 2H), 2.37 (s, 3H), 1.75-1.65 |
| | (m, 1H), 1.60-1.49 (m, 1H), 1.28 (dd, 6H), 1.24 (s, 3H). |
| 14 | 1-(3-(((3-((2-(4-hydroxy-4-methylpiperidin-1-yl)pyrimidin-4-yl)amino)-5- |
| | isopropylisoquinolin-8-yl)oxy)methyl)azetidin-1-yl)ethanone |
| | 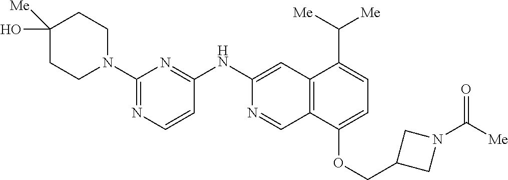 [SMILES: CC(=O)N1CC(COc2ccc(C(C)C)c3cc(Nc4ccnc(N5CCC(C)(O)CC5)n4)ncc23)C1] |
| | Amine-3; RCl: 1-(3-(((3-chloro-5-isopropylisoquinolin-8-yl)oxy)methyl)azetidin-1- |
| | yl)ethan-1-one (Preparation 63). |
| | Yield: 15 mg, 49.5%; LCMS m/z = 505 [M + H]⁺; ¹H NMR (400 MHz, DMSO-d₆) δ: |
| | 9.93 (s, 1H), 9.10 (s, 1H), 8.83 (s, 1H), 7.98 (d, 1H), 7.53 (d, 1H), 7.45 (d, |
| | 1H), 6.59 (s, 1H), 6.40 (d, 1H), 4.73 (d, 1H), 4.49 (dd, 2H), 4.38 (s, 1H), 4.29- |
| | 4.20(m, 2H), 4.16 (d, 1H), 3.58 (p, 1H), 3.52-3.42 (m, 2H), 1.80 (s, 3H), 1.59- |
| | 1.44 (m, 4H), 1.32 (d, 6H), 1.17 (s, 3H). |
| 15 | 1-(4-((8-(2-(1H-1,2,4-triazol-1-yl)ethoxy)-5-isopropylisoquinolin-3- |
| | yl)amino)pyrimidin-2-yl)-4-methylpiperidin-4-ol |
| | 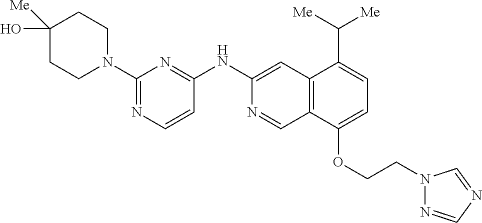 [SMILES: CC(C)c1ccc(OCCn2cncn2)c2cnc(Nc3ccnc(N4CCC(C)(O)CC4)n3)cc12] |
| | Amine-3; RCl: 8-(2-(1H-1,2,4-triazol-1-yl)ethoxy)-3-chloro-5- |
| | isopropylisoquinoline (Preparation 77). |
| | Yield: 11 mg, 31%; LCMS m/z = 490 [M + H]⁺; ¹H NMR (400 MHz, DMSO-d₆) δ: |
| | 9.89 (s, 1H), 9.21 (s, 1H), 8.72 (s, 1H), 8.67 (s, 2H), 7.97 (d, 1H), 7.44 (d, |
| | 1H), 6.82 (d, 1H), 6.39 (d, 1H), 4.59 (t, 2H), 4.43 (t, 2H), 4.37 (s, 1H), 4.23 |
| | (d, 2H), 3.52-3.40 (m, 3H), 1.59-1.41 (m, 4H), 1.28 (d, 6H), 1.16 (s, 3H). |
| 16 | (R)-5-(((3-((2-(4-hydroxy-4-methylpiperidin-1-yl)pyrimidin-4-yl)amino)-5- |
| | isopropylisoquinolin-8-yl)oxy)methyl)-3-methyloxazolidin-2-one |
| | 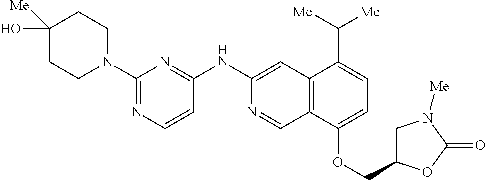 [SMILES: CC(C)c1ccc(OC[C@H]2CN(C)C(=O)O2)c2cnc(Nc3ccnc(N4CCC(C)(O)CC4)n3)cc12] |
| | Amine-3, RCl: (R)-5-(((3-chloro-5-isopropylisoquinolin-8-yl)oxy)methyl)-3- |
| | methyloxazolidin-2-one (Preparation 61). |
| | Yield: 20.7 mg, 41.5%; LCMS m/z = 507 [M + H]⁺; ¹H NMR (400 MHz, DMSO-d₆) |
| | δ: 9.96 (s, 1H), 9.18 (s, 1H), 8.76 (s, 1H), 7.97 (d, 1H), 7.47 (d, 1H), 6.86 (d, |
| | 1H), 6.36 (d, 1H), 5.03-4.91 (m, 1H), 4.36 (d, 2H), 4.34-4.19 (m, 3H), 3.77 (t, |
| | 1H), 3.56-3.41 (m, 4H), 2.82 (s, 3H), 1.59-1.43 (m, 4H), 1.17 (s, 3H). |
| 17 | 1-(4-((8-(2-(1H-imidazol-1-yl)ethoxy)-5-isopropylisoquinolin-3- |
| | yl)amino)pyrimidin-2-yl)-4-methylpiperidin-4-ol |
| | 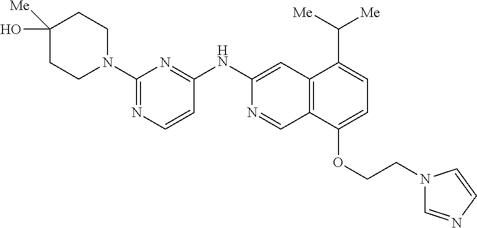 [SMILES: CC(C)c1ccc(OCCn2ccnc2)c2cnc(Nc3ccnc(N4CCC(C)(O)CC4)n3)cc12] |
| | Amine-3; RCl: 8-(2-(1H-imidazol-1-yl)ethoxy)-3-chloro-5-isopropylisoquinoline |
| | (Preparation 87). |
| | Yield: 12.4 mg, 27.7%; LCMS m/z = 489 [M + H]⁺; ¹H NMR (400 MHz, DMSO-d₆) |
| | δ: 9.89 (s, 1H), 9.19 (d, 1H), 8.72 (s, 1H), 7.97 (d, 1H), 7.79 (s, 1H), 7.44 (d, |
| | 1H), 7.31 (d, 1H), 6.89 (t, 1H), 6.83 (d, 1H), 6.38 (d, 1H), 4.50 (t, 2H), 4.43- |
| | 4.35 (m, 3H), 4.29-4.18 (m, 2H), 3.54-3.41 (m, 3H), 1.59-1.44 (m, 4H), 1.28 |
| | (d, 6H), 1.16 (s, 3H). |
| 18 | 1-(4-((8-((6,7-dihydro-5Hpyrrolo[1,2-c]imidazol-5-yl)methoxy)-5- |
| | isopropylisoquinolin-3-yl)amino)pyrimidin-2-yl)-4-methylpiperidin-4-ol |
| | 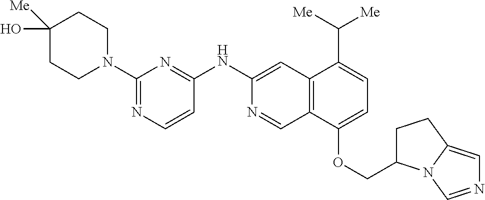 [SMILES: CC(C)c1ccc(OCC2CCc3cncn32)c2cnc(Nc3ccnc(N4CCC(C)(O)CC4)n3)cc12] |
| | Amine-3; RCl: 3-chloro-8-((6,7-dihydro-5H-pyrrolo[1,2-c]imidazol-5-yl)methoxy)- |
| | 5-isopropylisoquinoline (Preparation 86). |
| | Yield: 11.7 mg, 28.8%; LCMS m/z = 514 [M + H]⁺; ¹H NMR (400 MHz, DMSO-d₆) |
| | δ: 9.90 (s, 1H), 9.15 (s, 1H), 8.73 (s, 1H), 7.97 (d, 1H), 7.46 (d, 1H), 7.16 (d, |
| | 1H), 6.90-6.82 (m, 2H), 6.38 (d, 1H), 4.83-4.71 (m, 1H), 4.41 (dd, 1H), 4.37 |
| | (s, 1H), 4.31 (dd, 1H), 4.25 (dd, 2H), 3.53-3.40 (m, 3H), 2.93-2.74 (m, 3H), |
| | 1.60-1.42 (m, 4H), 1.28 (d, 6H), 1.16 (s, 3H). |

### Example 19

### (R)-5-(((3-((2-(4-hydroxy-4-methylpiperidin-1-yl)pyrimidin-4-yl)amino)-5-isopropylisoquinolin-8-yl)oxy)methyl)-1-methylpyrrolidin-2-one or (S)-5-(((3-((2-(4-hydroxy-4-methylpiperidin-1-yl)pyrimidin-4-yl)amino)-5-isopropylisoquinolin-8-yl)oxy)methyl)-1-methylpyrrolidin-2-one

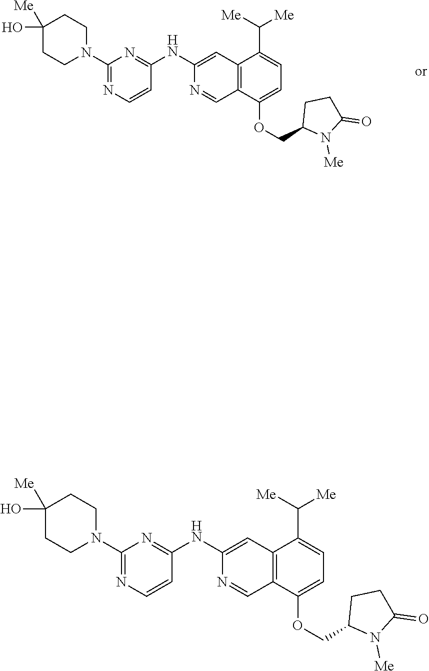 [SMILES: CC(C)c1ccc(OC[C@H]2CCC(=O)N2C)c2cnc(Nc3ccnc(N4CCC(C)(O)CC4)n3)cc12 ; CC(C)c1ccc(OC[C@@H]2CCC(=O)N2C)c2cnc(Nc3ccnc(N4CCC(C)(O)CC4)n3)cc12]

Part 1: 5-(((3-((2-(4-Hydroxy-4-methylpiperidin-1-yl)pyrimidin-4-yl)amino)-5-isopropylisoquinolin-8-yl)oxy)methyl)-1-methylpyrrolidin-2-one was prepared from 5-(((3-chloro-5-isopropylisoquinolin-8-yl)oxy)methyl)-1-methylpyrrolidin-2-one (Preparation 81) and 1-(4-aminopyrimidin-2-yl)-4-methylpiperidin-4-ol (Preparation 23) using an analogous method to that described for Example 7. Yield=58 mg, 81%. LCMS m/z=505 [M+H]⁺

Part 2: The title compound was obtained from 5-(((3-((2-(4-hydroxy-4-methylpiperidin-1-yl)pyrimidin-4-yl)amino)-5-isopropylisoquinolin-8-yl)oxy)methyl)-1-methylpyrrolidin-2-one (Part 1) by HPLC-4. Peak 1; Yield: 6.7 mg; LCMS m/z=505 [M+H]⁺; ¹H NMR (300 MHz, DMSO-d₆) δ: 9.94 (s, 1H), 9.19 (s, 1H), 8.77 (s, 1H), 8.00 (d, 1H), 7.50 (d, 1H), 6.91 (d, 1H), 6.41 (d, 1H), 4.42 (s, 1H), 4.30 (ddd, 4H), 4.12-3.96 (m, 1H), 3.57-3.42 (m, 3H), 2.83 (s, 3H), 2.48-2.37 (m, 3H), 2.34-2.14 (m, 3H), 2.08-1.83 (m, 1H), 1.66-1.40 (m, 4H), 1.32 (d, 6H), 1.19 (s, 3H).

### Example 20 and 21

### (R)-4-(((3-((2-((3S,4R)-3-fluoro-4-hydroxy-3-methylpiperidin-1-yl)pyrimidin-4-yl)amino)-5-isopropylisoquinolin-8-yl)oxy)methyl)-1-methylpyrrolidin-2-one and (S)-4-(((3-((2-((3S,4R)-3-fluoro-4-hydroxy-3-methylpiperidin-1-yl)pyrimidin-4-yl)amino)-5-isopropylisoquinolin-8-yl)oxy)methyl)-1-methylpyrrolidin-2-one

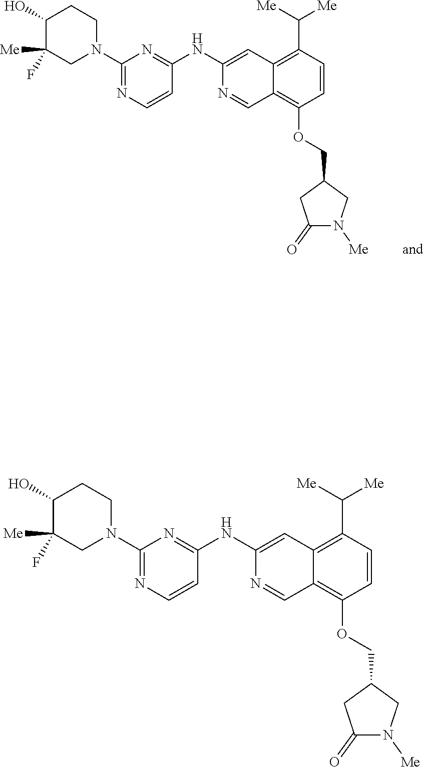 [SMILES: CC(C)c1ccc(OC[C@@H]2CC(=O)N(C)C2)c2cnc(Nc3ccnc(N4CC[C@@H](O)[C@@](C)(F)C4)n3)cc12 ; CC(C)c1ccc(OC[C@H]2CC(=O)N(C)C2)c2cnc(Nc3ccnc(N4CC[C@@H](O)[C@@](C)(F)C4)n3)cc12]

Part 1: A mixture of 4-(((3-chloro-5-isopropylisoquinolin-8-yl)oxy)methyl)-1-methylpyrrolidin-2-one (Preparation 54, 27 mg, 0.081 mmol), (3S,4R)-1-(4-aminopyrimidin-2-yl)-3-fluoro-3-methylpiperidin-4-ol (Preparation 8, 18 mg, 0.081 mmol), BrettPhos Pd G4 (3.49 mg, 0.004 mmol) and Cs₂CO₃ (53 mg, 0.162 mmol) in dioxane (0.8 mL) was heated at 90° C. under N₂ for 1 h. The reaction mixture was diluted with DCM and washed with H₂O. The combined organics were dried, evaporated to dryness in vacuo and the residue purified using reverse phase ISCO (0-60% MeCN/H₂O+0.1% TFA). The residue was diluted with sat. aq. NaHCO₃ solution and extracted with 5% MeOH/DCM (×2). The combined organics were dried (Na₂SO₄) and evaporated to dryness in vacuo to give 4-(((3-((2-((3S,4R)-3-fluoro-4-hydroxy-3-methylpiperidin-1-yl)pyrimidin-4-yl)amino)-5-isopropylisoquinolin-8-yl)oxy)methyl)-1-methylpyrrolidin-2-one as a pale yellow solid (16.5 mg, 39%). LCMS m/z=523 [M+H]⁺

Part 2: The title compounds were obtained by HPLC-5 from the compound of Part 1.

Peak 1; LCMS m/z=523 [M+H]⁺; ¹H NMR (300 MHz, DMSO-d₆) δ: 9.97 (s, 1H), 9.22 (s, 1H), 8.66 (s, 1H), 7.98 (d, 1H), 7.47 (d, 1H), 6.84 (d, 1H), 6.44 (d, 1H), 5.02 (d, 1H), 4.86-4.61 (m, 2H), 4.26-4.03 (m, 2H), 3.72-3.34 (m, 4H), 3.22-2.99 (m, 2H), 2.94-2.82 (m, 1H), 2.75 (s, 3H), 2.61-2.47 (m, 1H), 2.21 (dd, 1H), 1.83-1.67 (m, 2H), 1.44-1.25 (m, 9H)

Peak 2; LCMS m/z=523 [M+H]⁺; ¹H NMR (300 MHz, DMSO-d₆) δ: 9.96 (s, 1H), 9.22 (s, 1H), 8.66 (s, 1H), 7.98 (d, 1H), 7.47 (d, 1H), 6.84 (d, 1H), 6.44 (d, 1H), 5.02 (d, 1H), 4.78-4.53 (m, 2H), 4.30-3.98 (m, 2H), 3.74-3.32 (m, 4H), 3.22-3.02 (m, 2H), 2.97-2.80 (m, 1H), 2.75 (s, 3H), 2.59-2.50 (m, 1H), 2.21 (dd, 1H), 1.77-1.67 (m, 2H), 1.47-1.24 (m, 9H)

### Example 22 and 23

### (S)-4-(((3-((2-((3S,4R)-3-fluoro-4-hydroxy-4-methylpiperidin-1-yl)pyrimidin-4-yl)amino)-5-isopropylisoquinolin-8-yl)oxy)methyl)-3-methyloxazolidin-2-one and (R)-4-(((3-((2-((3S,4R)-3-fluoro-4-hydroxy-4-methylpiperidin-1-yl)pyrimidin-4-yl)amino)-5-isopropylisoquinolin-8-yl)oxy)methyl)-3-methyloxazolidin-2-one

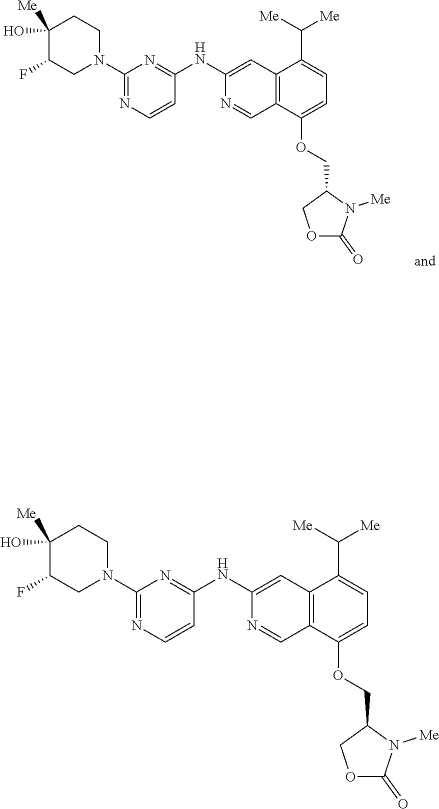 [SMILES: CC(C)c1ccc(OC[C@H]2COC(=O)N2C)c2cnc(Nc3ccnc(N4CC[C@@](C)(O)[C@@H](F)C4)n3)cc12 ; CC(C)c1ccc(OC[C@@H]2COC(=O)N2C)c2cnc(Nc3ccnc(N4CC[C@@](C)(O)[C@@H](F)C4)n3)cc12]

Part 1: 4-(((3-((2-((3S,4R)-3-fluoro-4-hydroxy-4-methylpiperidin-1-yl)pyrimidin-4-yl)amino)-5-isopropylisoquinolin-8-yl)oxy)methyl)-3-methyloxazolidin-2-one was prepared from (3S,4R)-1-(4-aminopyrimidin-2-yl)-3-fluoro-4-methylpiperidin-4-ol (Preparation 13) and 4-(((3-chloro-5-isopropylisoquinolin-8-yl)oxy)methyl)-3-methyloxazolidin-2-one (Preparation 73) using an analogous method to that described for Example 40. Yield=61 mg, 52%; LCMS m/z=525 [M+H]⁺

Part 2: The title compounds were obtained from 4-(((3-((2-((3S,4R)-3-fluoro-4-hydroxy-4-methylpiperidin-1-yl)pyrimidin-4-yl)amino)-5-isopropylisoquinolin-8-yl)oxy)methyl)-3-methyloxazolidin-2-one (Part 1, 55 mg, 0.105 mmol) by HPLC-6.

Peak 1; Yield: 19.4 mg, 35%; LCMS m/z=525 [M+H]⁺; ¹H NMR (300 MHz, DMSO-d₆) δ: 10.01 (s, 1H), 9.20 (s, 1H), 8.72 (s, 1H), 8.03 (d, 1H), 7.50 (d, 1H), 6.91 (d, 1H), 6.49 (d), 4.87 (s, 1H), 4.58-4.36 (m, 4H), 4.35-4.06 (m, 4H), 3.74-3.40 (m, 3H), 2.85 (s, 3H), 1.81-1.67 (m, 1H), 1.65-1.48 (m, 1H), 1.32 (dd, 6H), 1.26 (s, 3H).

Peak 2; Yield: 18.7 mg, 34%; LCMS m/z=525 [M+H]⁺; ¹H NMR (300 MHz, DMSO-d₆) δ: 10.01 (s, 1H), 9.20 (s, 1H), 8.72 (s, 1H), 8.03 (d, 1H), 7.51 (d, 1H), 6.91 (d, 1H), 6.49 (d, 1H), 4.87 (s, 1H), 4.57-4.35 (m, 4H), 4.33-4.10 (m, 4H), 3.70-3.42 (m, 3H), 2.85 (s, 3H), 1.81-1.67 (m, 1H), 1.65-1.48 (m, 1H), 1.32 (dd, 6H), 1.26 (s, 3H).

### Example 24

### 1-(3-((R)-1-((3-((2-((3S,4R)-3-fluoro-4-hydroxy-3-methylpiperidin-1-yl)pyrimidin-4-yl)amino)-5-isopropylisoquinolin-8-yl)oxy)ethyl)azetidin-1-yl)ethan-1-one or 1-(3-((S)-1-((3-((2-((3S,4R)-3-fluoro-4-hydroxy-3-methylpiperidin-1-yl)pyrimidin-4-yl)amino)-5-isopropylisoquinolin-8-yl)oxy)ethyl)azetidin-1-yl)ethan-1-one

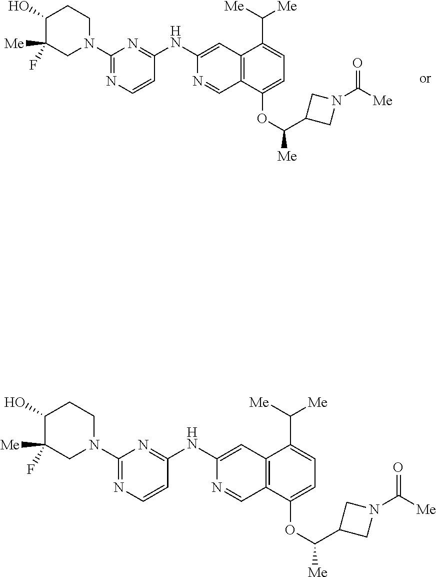 [SMILES: CC(=O)N1CC([C@@H](C)Oc2ccc(C(C)C)c3cc(Nc4ccnc(N5CC[C@@H](O)[C@@](C)(F)C5)n4)ncc23)C1 ; CC(=O)N1CC([C@H](C)Oc2ccc(C(C)C)c3cc(Nc4ccnc(N5CC[C@@H](O)[C@@](C)(F)C5)n4)ncc23)C1]

Part 1: A mixture of 1-(3-(1-((3-chloro-5-isopropylisoquinolin-8-yl)oxy)ethyl)azetidin-1-yl)ethan-1-one (Preparation 64, 55 mg, 0.159 mmol), (3S,4R)-1-(4-aminopyrimidin-2-yl)-3-fluoro-3-methylpiperidin-4-ol (Preparation 8, 36 mg, 0.159 mmol), XPhos Pd G4 (6.82 mg, 0.008 mmol) and Cs₂CO₃ (103 mg, 0.317 mmol) in dioxane (1 mL) was heated under N₂ at 90° C. for 1 h. The reaction was diluted with DCM and washed with H₂O. The combined organics were dried (Na₂SO₄) and evaporated to dryness in vacuo. The residue was purified using RP-ISCO (0-60% MeCN/H₂O+0.1% TFA) followed by treatment of the residue with aq. NaHCO₃ and extraction into 10% MeOH/DCM, drying (Na₂SO₄) and evaporation to dryness in vacuo to afford 1-(3-(1-((3-((2-((3S,4R)-3-fluoro-4-hydroxy-3-methylpiperidin-1-yl)pyrimidin-4-yl)amino)-5-isopropylisoquinolin-8-yl)oxy)ethyl)azetidin-1-yl)ethan-1-one as a pale yellow solid (46 mg, 54%). LCMS m/z=537 [M+H]⁺

Part 2: The title compound was obtained from the compound of Part 1 by HPLC-7. Peak 1: Example 24; LCMS m/z=537 [M+H]⁺; ¹H NMR (300 MHz, DMSO-d₆) δ: 10.01 (d, 1H), 9.23 (d, 1H), 8.69 (s, 1H), 8.00 (d, 1H), 7.50 (d, 1H), 6.95 (dd, 1H), 6.44 (d, 1H), 5.04 (d, 1H), 4.90-4.55 (m, 3H), 4.33-4.06 (m, 2H), 4.05-3.84 (m, 2H), 3.77-3.41 (m, 3H), 3.26-3.05 (m, 2H), 3.03-2.87 (m, 1H), 1.77 (d, 5H), 1.45-1.24 (m, 12H).

### Example 25

### (R)-5-(((3-((2-((3R,4S)-3-fluoro-4-hydroxy-4-methylpiperidin-1-yl)pyrimidin-4-yl)amino)-5-isopropylisoquinolin-8-yl)oxy)methyl)-1-methylpyrrolidin-2-one

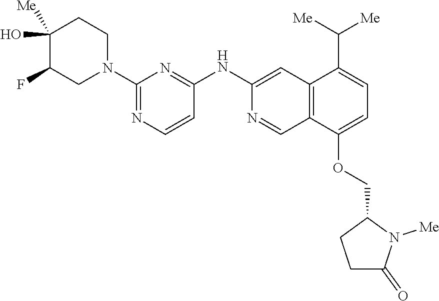 [SMILES: CC(C)c1ccc(OC[C@H]2CCC(=O)N2C)c2cnc(Nc3ccnc(N4CC[C@](C)(O)[C@H](F)C4)n3)cc12]

A mixture of (R)-5-(((3-chloro-5-isopropylisoquinolin-8-yl)oxy)methyl)-1-methylpyrrolidin-2-one (Preparation 65, 17 mg, 0.051 mmol), (3R,4S)-1-(4-aminopyrimidin-2-yl)-3-fluoro-4-methylpiperidin-4-ol (Preparation 14, 12 mg, 0.051 mmol), XPhos Pd G4 (2.35 mg, 2.6 mmol) and Cs₂CO₃ (33 mg, 0.102 mmol) in dioxane (0.5 mL) was heated under N₂ at 90° C. for 2 h. The reaction was diluted with DCM and washed with H₂O. The combined organics were dried (Na₂SO₄) and evaporated to dryness in vacuo. The residue was purified using RP-ISCO (0-70% MeCN/H₂O+0.1% TFA) followed by treatment of the residue with aq. NaHCO₃ and extraction into 10% MeOH/DCM, drying (Na₂SO₄) and evaporation to dryness in vacuo to afford the title compound as a pale yellow solid (12 mg, 44%). LCMS m/z=523 [M+H]⁺; ¹H NMR (400 MHz, DMSO-d₆) δ: 10.01 (s, 1H), 9.22 (s, 1H), 8.70 (s, 1H), 7.99 (d, 1H), 7.47 (d, 1H), 6.87 (d, 1H), 6.44 (d, 1H), 4.82 (s, 1H), 4.50-4.24 (m, 5H), 4.17 (d, 1H), 4.09-3.94 (m, 2H), 3.77 (dd, 1H), 3.60 (ddd, 1H), 3.54-3.42 (m, 2H), 3.17-3.04 (m, 1H), 1.77 (s, 3H), 1.70 (d, 1H), 1.61-1.50 (m, 1H), 1.29 (dd, 6H), 1.24 (s, 3H).

### Example 26-39

The title compounds were prepared from the appropriate amine (Amine-1 or Amine-2) and the appropriate halide (R-Hal) using an analogous procedure to that described for Example 25. Amine-1: (3R,4S)-1-(4-aminopyrimidin-2-yl)-3-fluoro-4-methylpiperidin-4-ol (Preparation 14); Amine-2: (3S,4R)-1-(4-aminopyrimidin-2-yl)-3-fluoro-4-methylpiperidin-4-ol (Preparation 13).

| Example | |
|---|---|
| No. | Name/Structure/Reactants/Data |
| 26 | 1-(3-(((3-((2-((3R,4S)-3-fluoro-4-hydroxy-4-methylpiperidin-1-yl)pyrimidin-4- |
| | yl)amino)-5-isopropylisoquinolin-8-yl)oxy)methyl)azetidin-1-yl)ethan-1-one |
| | 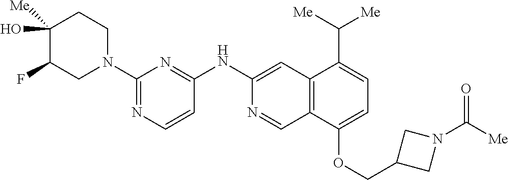 [SMILES: CC(=O)N1CC(COc2ccc(C(C)C)c3cc(Nc4ccnc(N5CC[C@](C)(O)[C@H](F)C5)n4)ncc23)C1] |
| | Amine-1; R-Hal: 1-(3-(((3-chloro-5-isopropylisoquinolin-8-yl)oxy)methyl)azetidin-1- |
| | yl)ethan-1-one (Preparation 63). |
| | Yield: 17.8 mg, 38%; LCMS m/z = 523 [M + H]⁺ |
| 27 | (3R,4S)-1-(4-((8-(2-(4H-1,2,4-triazol-4-yl)ethoxy)-5-isopropylisoquinolin-3- |
| | yl)amino)pyrimidin-2-yl)-3-fluoro-4-methylpiperidin-4-ol |
| | 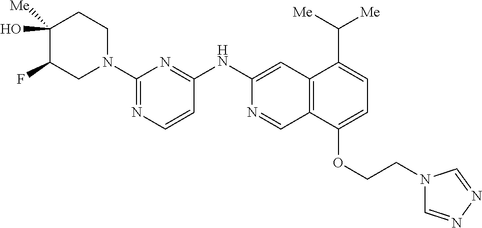 [SMILES: CC(C)c1ccc(OCCn2cnnc2)c2cnc(Nc3ccnc(N4CC[C@](C)(O)[C@H](F)C4)n3)cc12] |
| | Amine-1; R-Hal: 8-(2-(4H-1,2,4-triazol-4-yl)ethoxy)-3-chloro-5-isopropylisoquinoline |
| | (Preparation 76). |
| | Yield: 21 mg, 26%; LCMS m/z = 507 [M + H]⁺; ¹H NMR (400 MHz, DMSO-d₆) δ: |
| | 9.92 (s, 1H), 9.21 (s, 1H), 8.66 (s, 3H), 8.00 (d, 1H), 7.45 (d, 1H), 6.83 (d, 1H), 6.48 |
| | (d, 1H), 4.77 (s, 1H), 4.59 (t, 2H), 4.48-4.23 (m, 4H), 4.17 (dd, 1H), 3.60 (ddd, 1H), |
| | 3.48 (dd, 2H), 1.71 (ddd, 1H), 1.54 (ddd, 1H), 1.28 (dd, 6H), 1.24 (s, 3H). |
| 28 | (3S,4R)-1-(4-((8-(2-(4H-1,2,4-triazol-4-yl)ethoxy)-5-isopropylisoquinolin-3- |
| | yl)amino)pyrimidin-2-yl)-3-fluoro-4-methylpiperidin-4-ol |
| | 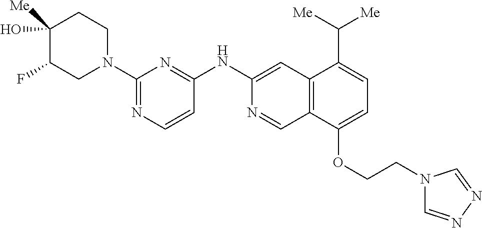 [SMILES: CC(C)c1ccc(OCCn2cnnc2)c2cnc(Nc3ccnc(N4CC[C@@](C)(O)[C@@H](F)C4)n3)cc12] |
| | Amine-2; R-Hal: 8-(2-(4H-1,2,4-triazol-4-yl)ethoxy)-3-chloro-5-isopropylisoquinoline |
| | (Preparation 76). |
| | Yield: 11.9 mg, 29.7%; LCMS m/z = 507 [M + H]⁺; ¹H NMR (400 MHz, DMSO-d₆) δ: |
| | 9.93 (s, 1H), 9.21 (s, 1H), 8.66 (s, 3H), 8.00 (d, 1H), 7.45 (d, 1H), 6.83 (d, 1H), 6.48 |
| | (d, 1H), 4.78 (s, 1H), 4.59 (t, 2H), 4.48-4.22 (m, 4H), 4.21-4.12 (m, 1H), 3.60 (ddd, |
| | 1H), 3.48 (dd, 2H), 1.75-1.65 (m, 1H), 1.60-1.49 (m, 1H), 1.28 (dd, 6H), 1.24 (s, 3H). |
| 29 | (3R,4S)-1-(4-((8-(2-(1H-imidazol-1-yl)ethoxy)-5-isopropylisoquinolin-3- |
| | yl)amino)pyrimidin-2-yl)-3-fluoro-4-methylpiperidin-4-ol |
| | 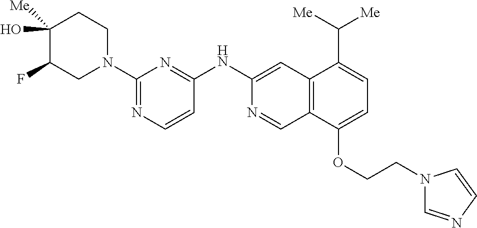 [SMILES: CC(C)c1ccc(OCCn2ccnc2)c2cnc(Nc3ccnc(N4CC[C@](C)(O)[C@H](F)C4)n3)cc12] |
| | Amine-1; R-Hal: 8-(2-(1H-imidazol-1-yl)ethoxy)-3-chloro-5-isopropylisoquinoline |
| | (Preparation 87). |
| | Yield: 6.6 mg, 11%; LCMS m/z = 506 [M + H]⁺; ¹H NMR (400 MHz, DMSO-d₆) δ: |
| | 9.96 (s, 1H), 9.20 (s, 1H), 8.67 (s, 1H), 8.00 (d, 1H), 7.80 (s, 1H), 7.45 (d, 1H), 7.32 (s, |
| | 1H), 6.89 (s, 1H), 6.83 (d, 1H), 6.46 (d, 1H), 4.81 (s, 1H), 4.51 (t, 2H), 4.45-4.23 (m, |
| | 4H), 4.17 (d, 1H), 3.60 (ddd, 1H), 3.54-3.41 (m, 2H), 1.75-1.66 (m, 1H), 1.61-1.47 (m, |
| | 1H), 1.28 (dd, 6H), 1.24 (s, 3H). |
| 30 | (3R,4S)-3-fluoro-1-(4-((5-isopropyl-8-(2-(3-methyl-4H-1,2,4-triazol-4- |
| | yl)ethoxy)isoquinolin-3-yl)amino)pyrimidin-2-yl)-4-methylpiperidin-4-ol |
| | 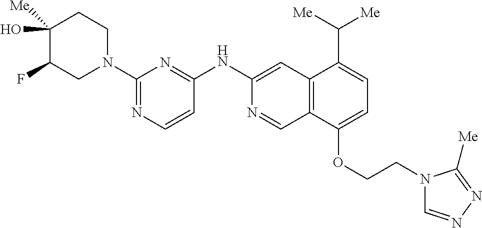 [SMILES: Cc1nncn1CCOc1ccc(C(C)C)c2cc(Nc3ccnc(N4CC[C@](C)(O)[C@H](F)C4)n3)ncc12] |
| | Amine-1; R-Hal: 3-chloro-5-isopropyl-8-(2-(3-methyl-4H-1,2,4-triazol-4- |
| | yl)ethoxy)isoquinoline (Preparation 78). |
| | Yield: 20.2 mg, 44%; LCMS m/z = 521.2 [M + H]⁺; ¹H NMR (400 MHz, DMSO-d₆) δ: |
| | 9.97 (s, 1H), 9.19 (s, 1H), 8.68 (s, 1H), 8.58 (s, 1H), 8.00 (d, 1H), 7.45 (d, 1H), 6.84 |
| | (d, 1H), 6.46 (d, 1H), 4.81 (s, 1H), 4.52-4.24 (m, 6H), 4.17 (d, 1H), 3.66-3.54 (m, 1H), |
| | 3.54-3.41 (m, 2H), 2.43 (s, 3H), 1.75-1.66 (m, 1H), 1.61-1.48 (m, 1H), 1.28 (dd, 6H), |
| | 1.24 (s, 3H). |
| 31 | (3R,4S)-3-fluoro-1-(4-((5-isopropyl-8-(2-morpholinoethoxy)isoquinolin-3- |
| | yl)amino)pyrimidin-2-yl)-4-methylpiperidin-4-ol |
| | 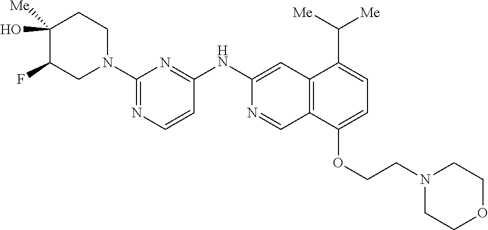 [SMILES: CC(C)c1ccc(OCCN2CCOCC2)c2cnc(Nc3ccnc(N4CC[C@](C)(O)[C@H](F)C4)n3)cc12] |
| | Amine-1; R-Hal: 4-(2-((3-chloro-5-isopropylisoquinolin-8-yl)oxy)ethyl)morpholine |
| | (Preparation 66). |
| | Yield: 38 mg, 76%; LCMS m/z = 525 [M + H]⁺; ¹H NMR (400 MHz, DMSO-d₆) δ: |
| | 9.97 (s, 1H), 9.26 (s, 1H), 8.69 (s, 1H), 7.99 (d, 1H), 7.46 (d, 1H), 6.86 (d, 1H), 6.45 |
| | (d, 1H), 4.51-4.22 (m, 4H), 4.17 (d, 1H), 3.60-3.44 (m, 8H), 2.84 (t, 2H), 2.53 (t, 4H), |
| | 2.42-2.31 (m, 2H), 1.70 (dd, 1H), 1.60-1.50 (m, 1H). |
| 32 | (3R,4S)-1-(4-((8-(2-(1H-1,2,3-triazol-1-yl)ethoxy)-5-isopropylisoquinolin-3- |
| | yl)amino)pyrimidin-2-yl)-3-fluoro-4-methylpiperidin-4-ol |
| | 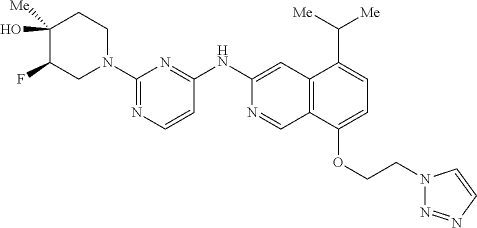 [SMILES: CC(C)c1ccc(OCCn2ccnn2)c2cnc(Nc3ccnc(N4CC[C@](C)(O)[C@H](F)C4)n3)cc12] |
| | Amine-1; R-Hal: 8-(2-(1H-1,2,3-triazol-1-yl)ethoxy)-3-chloro-5-isopropylisoquinoline |
| | (Preparation 79). |
| | Yield: 11 mg, 69%; LCMS m/z = 507 [M + H]⁺; ¹H NMR (400 MHz, DMSO-d₆) δ: |
| | 9.95 (s, 1H), 9.10 (s, 1H), 8.66 (s, 1H), 8.32 (d, 1H), 8.00 (d, 1H), 7.74 (d, 1H), 7.45 |
| | (d, 1H), 6.86 (d, 1H), 6.45 (d, 1H), 4.93 (t, 2H), 4.80 (s, 1H), 4.56 (t, 2H), 4.49-4.23 |
| | (m, 2H), 4.16 (d, 1H), 3.65-3.54 (m, 1H), 3.53-3.42 (m, 2H), 1.75-1.64 (m, 1H), 1.60- |
| | 1.50 (m, 1H), 1.28 (dd, 6H), 1.24 (s, 3H). |
| 33 | (S)-5-(((3-((2-((3R,4S)-3-fluoro-4-hydroxy-4-methylpiperidin-1-yl)pyrimidin-4- |
| | yl)amino)-5-isopropylisoquinolin-8-yl)oxy)methyl)-1-methylpyrrolidin-2-one |
| | 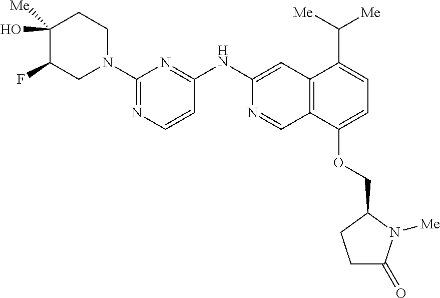 [SMILES: CC(C)c1ccc(OC[C@@H]2CCC(=O)N2C)c2cnc(Nc3ccnc(N4CC[C@](C)(O)[C@H](F)C4)n3)cc12] |
| | Amine-1; R-Hal: (S)-5-(((3-chloro-5-isopropylisoquinolin-8-yl)oxy)methyl)-1- |
| | methylpyrrolidin-2-one (Preparation 68). |
| | Yield: 12.2 mg, 22.6%; LCMS m/z = 523 [M + H]⁺; ¹H NMR (400 MHz, DMSO-d₆) δ: |
| | 9.95 (s, 1H), 9.18 (s, 1H), 8.68 (s, 1H), 8.00 (d, 1H), 7.47 (d, 1H), 6.89 (d, 1H), 6.47 |
| | (d, 1H), 4.81 (s, 1H), 4.50-4.12 (m, 5H), 4.02 (dd, 1H), 3.66-3.54 (m, 1H), 3.55-3.41 |
| | (m, 2H), 2.81 (s, 3H), 2.45-2.35 (m, 1H), 2.33-2.18 (m, 2H), 2.01-1.88 (m, 1H), 1.69 |
| | (s, 1H), 1.62-1.50 (m, 1H), 1.30 (dd, 6H), 1.24 (s, 3H). |
| 34 | (3R,4S)-3-fluoro-1-(4-((5-isopropyl-8-(2-(1-methyl-1H-pyrazol-5- |
| | yl)ethoxy)isoquinolin-3-yl)amino)pyrimidin-2-yl)-4-methylpiperidin-4-ol |
| | 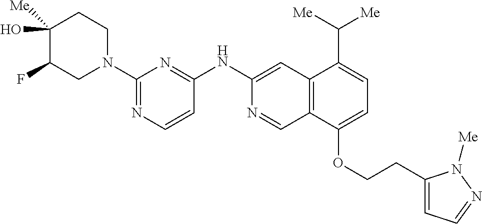 [SMILES: CC(C)c1ccc(OCCc2ccnn2C)c2cnc(Nc3ccnc(N4CC[C@](C)(O)[C@H](F)C4)n3)cc12] |
| | Amine-1; R-Hal: 3-chloro-5-isopropyl-8-(2-(1-methyl-1H-pyrazol-5- |
| | yl)ethoxy)isoquinoline (Preparation 69). |
| | Yield: 40.8 mg, 64.7%; LCMS m/z = 520 [M + H]⁺; ¹H NMR (400 MHz, DMSO-d₆) δ: |
| | 9.99 (s, 1H), 9.20 (s, 1H), 8.69 (s, 1H), 7.99 (d, 1H), 7.47 (d, 1H), 7.30 (d, 1H), 6.90 |
| | (d, 1H), 6.44 (d, 1H), 6.20 (d, 1H), 4.81 (s, 1H), 4.49-4.23 (m, 4H), 4.17 (d, 1H), 3.82 |
| | (s, 3H), 3.60 (ddd, 1H), 3.50 (dd, 2H), 3.25 (t, 2H), 1.70 (d, 1H), 1.60-1.49 (m, 1H), |
| | 1.29 (dd, 6H), 1.24 (s, 3H). |
| 35 | (3R,4S)-3-fluoro-1-(4-((5-isopropyl-8-(2-(1-methyl-1H-1,2,3-triazol-5- |
| | yl)ethoxy)isoquinolin-3-yl)amino)pyrimidin-2-yl)-4-methylpiperidin-4-ol |
| | 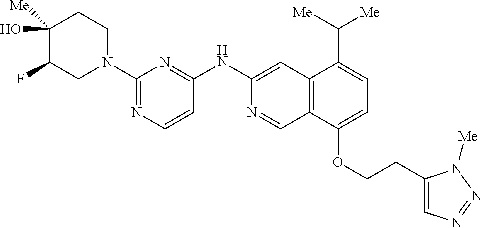 [SMILES: CC(C)c1ccc(OCCc2cnnn2C)c2cnc(Nc3ccnc(N4CC[C@](C)(O)[C@H](F)C4)n3)cc12] |
| | Amine-1; R-Hal: 3-chloro-5-isopropyl-8-(2-(1-methyl-1H-1,2,3-triazol-5- |
| | yl)ethoxy)isoquinoline (Preparation 70). |
| | Yield: 38 mg, 60.4%; LCMS m/z = 521 [M + H]⁺; ¹H NMR (400 MHz, DMSO-d₆) δ: |
| | 9.98 (s, 1H), 9.19 (s, 1H), 8.69 (s, 1H), 8.00 (d, 1H), 7.66 (s, 1H), 7.47 (d, 1H), 6.90 |
| | (d, 1H), 6.45 (d, 1H), 4.81 (s, 1H), 4.50-4.23 (m, 4H), 4.17 (d, 1H), 4.02 (s, 3H), 3.66- |
| | 3.56 (m, 1H), 3.56-3.42 (m, 2H), 3.31 (t, 2H), 1.76-1.65 (m, 1H), 1.61-1.49 (m, 1H), |
| | 1.29 (dd, 6H), 1.24 (s, 3H). |
| 36 | (3R,4S)-1-(4-((8-((S)-2-(4H-1,2,4-triazol-4-yl)propoxy)-5-isopropylisoquinolin-3- |
| | yl)amino)pyrimidin-2-yl)-3-fluoro-4-methylpiperidin-4-ol |
| | 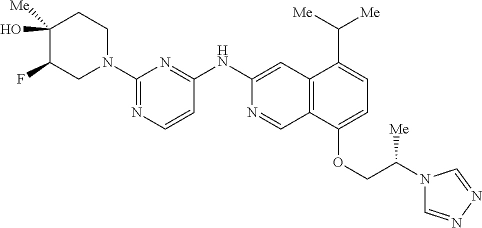 [SMILES: CC(C)c1ccc(OC[C@H](C)n2cnnc2)c2cnc(Nc3ccnc(N4CC[C@](C)(O)[C@H](F)C4)n3)cc12] |
| | Amine-1; R-Hal: (S)-8-(2-(4H-1,2,4-triazol-4-yl)propoxy)-3-chloro-5- |
| | isopropylisoquinoline (Preparation 71). |
| | Yield: 27.3 mg, 43.4%; LCMS m/z = 521 [M + H]⁺; ¹H NMR (400 MHz, DMSO-d₆) δ: |
| | 9.93 (s, 1H), 9.12 (s, 1H), 8.75 (s, 2H), 8.66 (s, 1H), 8.00 (d, 1H), 7.44 (d, 1H), 6.83 |
| | (d, 1H), 6.47 (d, 1H), 5.01-4.90 (m, 1H), 4.80 (s, 1H), 4.47-4.23 (m, 4H), 4.16 (d, 1H), |
| | 3.60 (ddd, 1H), 3.54-3.42 (m, 2H), 1.74-1.61 (m, 4H), 1.60-1.48 (m, 1H), 1.28 (d, 6H), |
| | 1.24 (s, 3H). |
| 37 | (3R,4S)-1-(4-((8-((R)-2-(4H-1,2,4-triazol-4-yl)propoxy)-5-isopropylisoquinolin-3- |
| | yl)amino)pyrimidin-2-yl)-3-fluoro-4-methylpiperidin-4-ol |
| | 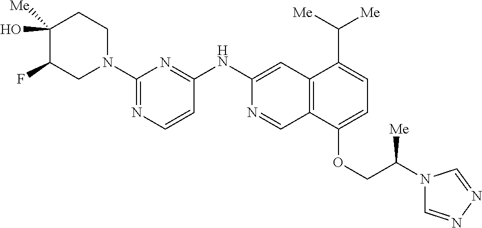 [SMILES: CC(C)c1ccc(OC[C@@H](C)n2cnnc2)c2cnc(Nc3ccnc(N4CC[C@](C)(O)[C@H](F)C4)n3)cc12] |
| | Amine-1; R-Hal: (R)-8-(2-(4H-1,2,4-triazol-4-yl)propoxy)-3-chloro-5- |
| | isopropylisoquinoline (Preparation 72). |
| | Yield: 22.6 mg, 35.9%; LCMS m/z = 521 [M + H]⁺; ¹H NMR (400 MHz, DMSO-d₆) δ: |
| | 9.93 (s, 1H), 9.12 (s, 1H), 8.75 (s, 2H), 8.66 (s, 1H), 8.00 (d, 1H), 7.44 (d, 1H), 6.83 |
| | (d, 1H), 6.47 (d, 1H), 4.96 (td, 1H), 4.80 (s, 1H), 4.49-4.23 (m, 4H), 4.16 (d, 1H), 3.59 |
| | (ddd, 1H), 3.48 (dt, 2H), 1.74-1.60 (m, 4H), 1.54 (td, 1H), 1.28 (dd, 6H), 1.23 (s, 3H). |
| 38 | (3R,4S)-3-fluoro-1-(4-((5-isopropyl-8-(2-(1-methyl-1H-imidazol-5- |
| | yl)ethoxy)isoquinolin-3-yl)amino)pyrimidin-2-yl)-4-methylpiperidin-4-ol |
| | 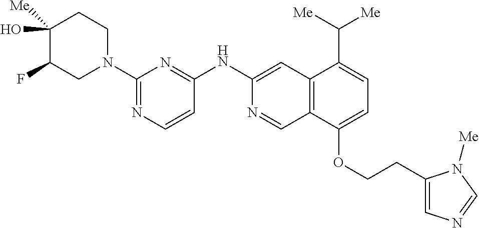 [SMILES: CC(C)c1ccc(OCCc2cncn2C)c2cnc(Nc3ccnc(N4CC[C@](C)(O)[C@H](F)C4)n3)cc12] |
| | Amine-1; R-Hal: 3-chloro-5-isopropyl-8-(2-(1-methyl-1H-imidazol-5- |
| | yl)ethoxy)isoquinoline (Preparation 74). |
| | Yield: 25.6 mg, 42.8%; LCMS m/z = 520 [M + H]⁺; ¹H NMR (400 MHz, DMSO-d₆) δ: |
| | 9.99 (s, 1H), 9.23 (d, 1H), 8.69 (s, 1H), 7.99 (d, 1H), 7.51 (d, 1H), 7.46 (d, 1H), 6.90 |
| | (d, 1H), 6.81 (d, 1H), 6.44 (d, 1H), 4.81 (s, 1H), 4.49-4.23 (m, 4H), 4.17 (d, 1H), 3.62 |
| | (s, 4H), 3.55-3.42 (m, 2H), 3.16 (t, 2H), 1.77-1.65 (m, 1H), 1.60-1.50 (m, 1H), 1.29 |
| | (dd, 6H), 1.24 (s, 3H). |
| 39 | (3R,4S)-1-(4-((8-(2-(1H-1,2,4-triazol-1-yl)ethoxy)-5-isopropylisoquinolin-3- |
| | yl)amino)pyrimidin-2-yl)-3-fluoro-4-methylpiperidin-4-ol |
| | 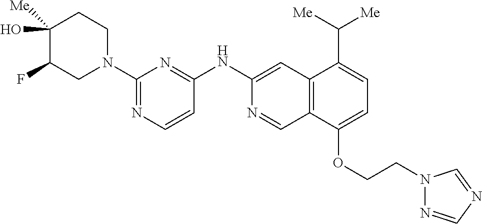 [SMILES: CC(C)c1ccc(OCCn2cncn2)c2cnc(Nc3ccnc(N4CC[C@](C)(O)[C@H](F)C4)n3)cc12] |
| | Amine-1; R-Hal: 8-(2-(1H-1,2,4-triazol-1-yl)ethoxy)-3-chloro-5-isopropylisoquinoline |
| | (Preparation 77). |
| | Yield: 21.8 mg, 34%; LCMS m/z = 507 [M + H]⁺; ¹H NMR (400 MHz, DMSO-d₆) δ: |
| | 9.95 (s, 1H), 9.12 (s, 1H), 8.73 (s, 1H), 8.66 (s, 1H), 8.03-7.95 (m, 2H), 7.44 (d, 1H), |
| | 6.85 (d, 1H), 6.45 (d, 1H), 4.80 (s, 1H), 4.72 (t, 2H), 4.49 (t, 2H), 4.47-4.23 (m, 2H), |
| | 4.16 (d, 1H), 3.59 (ddd, 1H), 3.53-3.42 (m, 2H), 1.75-1.63 (m, 1H), 1.60-1.48 (m, 1H), |
| | 1.28 (dd, 6H), 1.23 (s, 3H). |

### Example 40

### 8-(2-(4H-1,2,4-triazol-4-yl)ethoxy)-N-(2-((3S,4R)-3-fluoro-4-methoxypiperidin-1-yl)pyrimidin-4-yl)-5-isopropylisoquinolin-3-amine

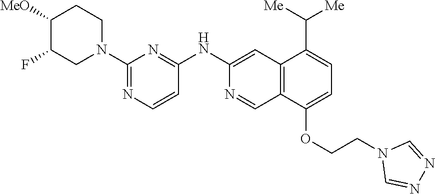 [SMILES: CO[C@@H]1CCN(c2nccc(Nc3cc4c(C(C)C)ccc(OCCn5cnnc5)c4cn3)n2)C[C@@H]1F]

A mixture of 8-(2-(4H-1,2,4-triazol-4-yl)ethoxy)-3-chloro-5-isopropylisoquinoline (Preparation 76, 25 mg, 0.079 mmol), 2-((3S,4R)-3-fluoro-4-methoxypiperidin-1-yl)pyrimidin-4-amine (Preparation 31, 18 mg, 0.079 mmol), BrettPhos Pd G4 (3.4 mg, 3.95 mmol) and Cs₂CO₃ (51 mg, 0.158 mmol) in dioxane (0.75 mL) was stirred at 90° C. under N₂ for 1 h. The reaction mixture was diluted with DCM and washed with H₂O. The combined organics were dried (Na₂SO₄) and evaporated to dryness in vacuo. The residue was purified using reverse phase ISCO (0-70% MeCN/H₂O+0.1% TFA). The residue was diluted with sat. aq. NaHCO₃ solution and extracted with 5% MeOH/DCM (×2). The combined organics were dried (Na₂SO₄) and evaporated to dryness in vacuo to give the title compound as an off-white solid (13.3 mg, 33%). LCMS m/z=507 [M+H]⁺; ¹H NMR (400 MHz, DMSO-d₆) δ: 9.96 (s, 1H), 9.22 (s, 1H), 8.68 (s, 2H), 8.65 (s, 1H), 8.00 (d, 1H), 7.45 (d, 1H), 6.83 (d, 1H), 6.47 (d, 1H), 5.03-4.83 (m, 1H), 4.76-4.64 (m, 1H), 4.59 (t, 2H), 4.50-4.37 (m, 3H), 3.66-3.41 (m, 3H), 3.35 (s, 3H), 1.85-1.65 (m, 2H), 1.29 (dd, 6H).

### Example 41

### 8-(2-(4H-1,2,4-triazol-4-yl)ethoxy)-N-(2-((3R,4S)-3-fluoro-4-methoxypiperidin-1-yl)pyrimidin-4-yl)-5-isopropylisoquinolin-3-amine

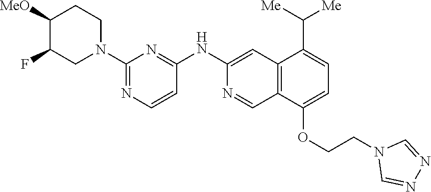 [SMILES: CO[C@H]1CCN(c2nccc(Nc3cc4c(C(C)C)ccc(OCCn5cnnc5)c4cn3)n2)C[C@H]1F]

The title compound was prepared from 8-(2-(4H-1,2,4-triazol-4-yl)ethoxy)-3-chloro-5-isopropylisoquinoline (Preparation 76), 2-((3R,4S)-3-fluoro-4-methoxypiperidin-1-yl)pyrimidin-4-amine (Preparation 30) using an analogous method to that described for Example 40. Yield: 31 mg, 40%. LCMS m/z=507 [M+H]⁺; ¹H NMR (400 MHz, DMSO-d₆) δ: 9.95 (s, 1H), 9.22 (s, 1H), 8.68 (s, 2H), 8.65 (s, 1H), 8.00 (d, 1H), 7.45 (d, 1H), 6.83 (d, 1H), 6.47 (d, 1H), 5.04-4.82 (m, 1H), 4.75-4.65 (m, 1H), 4.59 (t, 2H), 4.49-4.40 (m, 3H), 3.65-3.39 (m, 3H), 3.35 (s, 3H), 1.86-1.66 (m, 2H), 1.29 (dd, 6H).

### Example 42

### 1-(3-(((3-((2-((4S,5R)-5-fluoro-4-hydroxy-3,3-dimethylpiperidin-1-yl)pyrimidin-4-yl)amino)isoquinolin-8-yl)oxy)methyl)azetidin-1-yl)ethenone or 1-(3-(((3-((2-((4R,5S)-5-fluoro-4-hydroxy-3,3-dimethylpiperidin-1-yl)pyrimidin-4-yl)amino)isoquinolin-8-yl)oxy)methyl)azetidin-1-yl)ethenone

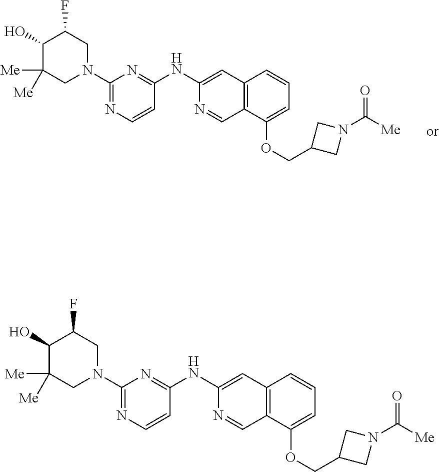 [SMILES: CC(=O)N1CC(COc2cccc3cc(Nc4ccnc(N5C[C@@H](F)[C@@H](O)C(C)(C)C5)n4)ncc23)C1 ; CC(=O)N1CC(COc2cccc3cc(Nc4ccnc(N5C[C@H](F)[C@H](O)C(C)(C)C5)n4)ncc23)C1]

The title compound was prepared from 1-(3-(((3-chloroisoquinolin-8-yl)oxy)methyl)azetidin-1-yl)ethanone (Preparation 88), the compound of Peak 2 from Preparation 22 and 23 using an analogous method to that described for Example 40. Yield: 28.4 mg, 49%. LCMS m/z=495 [M+H]⁺; ¹H NMR (400 MHz, DMSO-d₆) δ: 9.98 (s, 1H), 9.19 (d, 1H), 8.48 (s, 1H), 7.97 (d, 1H), 7.59 (t, 1H), 7.24 (d, 1H), 6.91 (d, 1H), 6.39 (d, 1H), 5.10 (d, 1H), 4.85-4.64 (m, 1H), 4.39-4.19 (m, 4H), 4.10-3.97 (m, 2H), 3.91-3.74 (m, 3H), 3.46 (ddd, 1H), 3.37 (d, 1H), 3.11 (ddd, 1H), 1.77 (s, 3H), 0.96 (s, 5H), 0.94 (s, 3H).

### Example 43

### 2-(((3S,4R)-1-(4-((8-(2-(3,5-dimethylisoxazol-4-yl)ethoxy)-5-isopropylisoquinolin-3-yl)amino)pyrimidin-2-yl)-3-fluoropiperidin-4-yl)oxy)ethan-1-ol

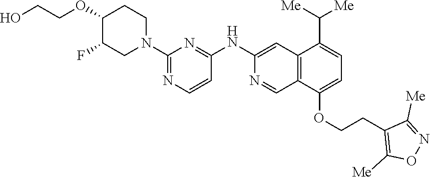 [SMILES: Cc1noc(C)c1CCOc1ccc(C(C)C)c2cc(Nc3ccnc(N4CC[C@@H](OCCO)[C@@H](F)C4)n3)ncc12]

The title compound was prepared from 4-(2-((3-chloro-5-isopropylisoquinolin-8-yl)oxy)ethyl)-3,5-dimethylisoxazole (Preparation 75) and 2-(((3S,4R)-1-(4-aminopyrimidin-2-yl)-3-fluoropiperidin-4-yl)oxy)ethan-1-ol (Preparation 25) using an analogous method to that described for Example 40. Yield: 14.4 mg, 25%. LCMS m/z=565 [M+H]⁺; ¹H NMR (400 MHz, DMSO-d₆) δ: 9.95 (s, 1H), 9.18 (s, 1H), 8.68 (s, 1H), 8.00 (d, 1H), 7.47 (d, 1H), 6.89 (d, 1H), 6.47 (d, 1H), 4.81 (s, 1H), 4.48-4.21 (m, 4H), 4.17 (d, 1H), 4.02 (dd, 1H), 3.60 (ddd, 1H), 3.56-3.42 (m, 2H), 2.81 (s, 3H), 2.45-2.36 (m, 1H), 2.25 (td, 2H), 2.00-1.90 (m, 1H), 1.69 (s, 1H), 1.60-1.50 (m, 1H), 1.30 (dd, 6H), 1.24 (s, 3H).

### Example 44

### 8-(2-(4H-1,2,4-triazol-4-yl)ethoxy)-N-(2-((3R,4S)-3-fluoro-4-(methoxy-d₃)piperidin-1-yl)pyrimidin-4-yl)-5-isopropylisoquinolin-3-amine

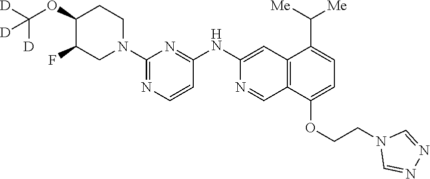 [SMILES: [2H]C([2H])([2H])O[C@H]1CCN(c2nccc(Nc3cc4c(C(C)C)ccc(OCCn5cnnc5)c4cn3)n2)C[C@H]1F]

Into a 8-mL pressure tank reactor purged and maintained with an inert atmosphere of N₂ was placed 8-(2-(4H-1,2,4-triazol-4-yl)ethoxy)-3-chloro-5-isopropylisoquinoline (Preparation 76, 50 mg, 0.158 mmol), Cs₂CO₃ (103 mg, 0.316 mmol), 2-((3R,4S)-3-fluoro-4-(methoxy-d₃)piperidin-1-yl)pyrimidin-4-amine (Preparation 28, 36.2 mg, 0.158 mmol), Brettphos Pd G3 (14.3 mg, 0.016 mmol) and dioxane (3 mL) and the resulting solution stirred for 3 h at 100° C. The reaction was quenched with H₂O (30 mL) and the solids removed by filtration. The filtrate was extracted with EtOAc (3×50 mL) and the combined organics evaporated to dryness in vacuo. The residue was purified by HPLC-8 (Gradient (% organic): 36-42%) to afford the title compound as a pale yellow solid (12 mg, 15%). LCMS m/z=510 [M+H]⁺; ¹H NMR (400 MHz, DMSO-d₆) δ: 9.98 (s, 1H), 9.24 (s, 1H), 8.70 (s, 2H), 8.67 (s, 1H), 8.01 (d, 1H), 7.47 (d, 1H), 6.85 (d, 1H), 6.49 (d, 1H), 4.94 (d, 1H), 4.79-4.63 (m, 1H), 4.61 (t, 2H), 4.51-4.32 (m, 3H), 3.70-3.39 (m, 3H), 1.91-1.59 (m, 2H), 1.31 (t, 6H).

### Example 45

### 8-(2-(4H-1,2,4-triazol-4-yl)ethoxy)-N-(2-((3S,4R)-3-fluoro-4-(methoxy-d₃)piperidin-1-yl)pyrimidin-4-yl)-5-isopropylisoquinolin-3-amine

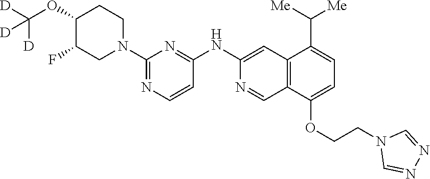 [SMILES: [2H]C([2H])([2H])O[C@@H]1CCN(c2nccc(Nc3cc4c(C(C)C)ccc(OCCn5cnnc5)c4cn3)n2)C[C@@H]1F]

Into a 8-mL pressure tank reactor purged and maintained with an inert atmosphere of N₂ was placed 8-(2-(4H-1,2,4-triazol-4-yl)ethoxy)-3-chloro-5-isopropylisoquinoline (Preparation 76, 50 mg, 0.158 mmol), Cs₂CO₃ (103 mg, 0.316 mmol), 2-((3S,4R)-3-fluoro-4-(methoxy-d₃)piperidin-1-yl)pyrimidin-4-amine (Preparation 29, 36.2 mg, 0.158 mmol), Brettphos Pd G3 (14.3 mg, 0.016 mmol) and dioxane (3 mL) and the resulting solution stirred for 3 h at 100° C. The reaction was quenched with H₂O (30 mL) and the solids removed by filtration. The filtrate was extracted with EtOAc (3×50 mL) and the combined organics evaporated to dryness in vacuo. The residue was purified by HPLC-9 (Gradient (% organic): 32-41%) to afford the title compound as a pale yellow solid (10 mg, 12.4%). LCMS m/z=510 [M+H]⁺; ¹H NMR (400 MHz, DMSO-d₆) δ: 9.98 (s, 1H), 9.24 (s, 1H), 8.70 (s, 2H), 8.67 (s, 1H), 8.01 (d, 1H), 7.47 (d, 1H), 6.85 (d, 1H), 6.49 (d, 1H), 5.06-4.85 (m, 1H), 4.78-4.68 (m, 1H), 4.61 (t, 2H), 4.49-4.35 (m, 3H), 3.69-3.36 (m, 3H), 1.94-1.63 (m, 2H), 1.31 (t, 6H).

### Example 46-49

The title compounds were prepared from the appropriate amine (Amine-1 or Amine-2) and appropriate halide (R-Hal) using an analogous procedure to that described for Example 44. Amine-1, 2-((3R,4S)-3-fluoro-4-(methoxy-d₃)piperidin-1-yl)pyrimidin-4-amine (Preparation 28); Amine-2, 2-((3S,4R)-3-fluoro-4-(methoxy-d₃)piperidin-1-yl)pyrimidin-4-amine (Preparation 29).

| Example | |
|---|---|
| No. | Name/Structure/Reactants/Data |
| 46 | (R)-5-(((3-((2-((3R,4S)-3-fluoro-4-(methoxy-d3)piperidin-1-yl)pyrimidin-4-yl)amino)- |
| | 5-isopropylisoquinolin-8-yl)oxy)methyl)-1-methylpyrrolidin-2-one |
| | 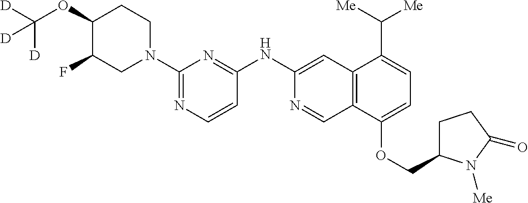 [SMILES: [2H]C([2H])([2H])O[C@H]1CCN(c2nccc(Nc3cc4c(C(C)C)ccc(OC[C@H]5CCC(=O)N5C)c4cn3)n2)C[C@H]1F] |
| | Amine-1; R-Hal: (R)-5-(((3-chloro-5-isopropylisoquinolin-8-yl)oxy)methyl)-1- |
| | methylpyrrolidin-2-one (Preparation 65). |
| | Yield: 10 mg, 10.6%; LCMS m/z = 526 [M + H]⁺; ¹H NMR (400 MHz, DMSO-d₆) δ: |
| | 9.96 (s, 1H), 9.20 (s, 1H), 8.68 (s, 1H), 8.02 (d, 1H), 7.50 (d, 1H), 6.91 (d, 1H), |
| | 6.49 (d, 1H), 4.94 (d, 1H), 4.80-4.66 (m, 1H), 4.54-4.41 (m, 1H), 4.37-4.21 (m, |
| | 2H), 4.08-4.01 (m, 1H), 3.66-3.44 (m, 4H), 2.46-2.37 (m, 1H), 2.35-2.17 (m, |
| | 2H), 2.02-1.94 (m, 1H), 1.86-1.70 (m, 2H), 1.32 (t, 6H). |
| 47 | (R)-5-(((3-((2-((3S,4R)-3-fluoro-4-(methoxy-d3)piperidin-1-yl)pyrimidin-4-yl)amino)- |
| | 5-isopropylisoquinolin-8-yl)oxy)methyl)-1-methylpyrrolidin-2-one |
| | 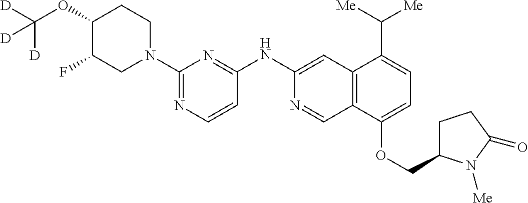 [SMILES: [2H]C([2H])([2H])O[C@@H]1CCN(c2nccc(Nc3cc4c(C(C)C)ccc(OC[C@H]5CCC(=O)N5C)c4cn3)n2)C[C@@H]1F] |
| | Amine-2; R-Hal: (R)-5-(((3-chloro-5-isopropylisoquinolin-8-yl)oxy)methyl)-1- |
| | methylpyrrolidin-2-one (Preparation 65). |
| | Yield: 10 mg, 10.6%; LCMS m/z = 526 [M + H]⁺; ¹H NMR (400 MHz, DMSO-d₆) δ: |
| | 9.96 (s, 1H), 9.20 (s, 1H), 8.68 (s, 1H), 8.02 (d, 1H), 7.50 (d, 1H), 6.91 (d, 1H), |
| | 6.49 (d, 1H), 4.94 (d, 1H), 4.78-4.62 (m, 1H), 4.54-4.42 (m, 1H), 4.37-4.19 (m, |
| | 2H), 4.13-3.98 (m, 1H), 3.71-3.40 (m, 4H), 2.47-2.38 (m, 1H), 2.36-2.18 (m, |
| | 2H), 2.03-1.90 (m, 1H), 1.90-1.67 (m, 2H), 1.32 (t, 6H). |
| 48 | 8-(2-(1H-imidazol-1-yl)ethoxy)-N-(2-((3R,4S)-3-fluoro-4-(methoxy-d3)piperidin-1- |
| | yl)pyrimidin-4-yl)-5-isopropylisoquinolin-3-amine |
| | 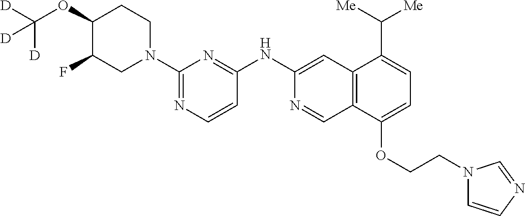 [SMILES: [2H]C([2H])([2H])O[C@H]1CCN(c2nccc(Nc3cc4c(C(C)C)ccc(OCCn5ccnc5)c4cn3)n2)C[C@H]1F] |
| | Amine-1; R-Hal: 8-(2-(1H-imidazol-1-yl)ethoxy)-3-chloro-5-isopropylisoquinoline |
| | (Preparation 87). |
| | Yield: 6 mg, 6.2%; LCMS m/z = 509 [M + H]⁺; ¹H NMR (400 MHz, DMSO-d₆) δ: 9.98 |
| | (s, 1H), 9.22 (s, 1H), 8.67 (s, 1H), 8.01 (d, 1H), 7.81 (s, 1H), 7.47 (d, 1H), 7.34 |
| | (s, 1H), 6.91 (s, 1H), 6.85 (d, 1H), 6.52-6.45 (m, 1H), 4.94 (d, 1H), 4.78-4.66 |
| | (m, 1H), 4.58-4.35 (m, 5H), 3.65-3.38 (m, 4H), 1.92-1.72 (m, 2H), 1.31 (t, 6H). |
| 49 | 8-(2-(1H-imidazol-1-yl)ethoxy)-N-(2-((3S,4R)-3-fluoro-4-(methoxy-d₃)piperidin-1- |
| | yl)pyrimidin-4-yl)-5-isopropylisoquinolin-3-amine |
| | 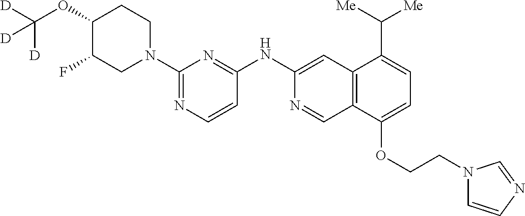 [SMILES: [2H]C([2H])([2H])O[C@@H]1CCN(c2nccc(Nc3cc4c(C(C)C)ccc(OCCn5ccnc5)c4cn3)n2)C[C@@H]1F] |
| | Amine-2; R-Hal: 8-(2-(1H-imidazol-1-yl)ethoxy)-3-chloro-5-isopropylisoquinoline |
| | (Preparation 87). |
| | Yield: 10 mg, 10.4%; LCMS m/z = 509 [M + H]⁺; ¹H NMR (400 MHz, DMSO-d₆) δ: |
| | 9.98 (s, 1H), 9.22 (s, 1H), 8.67 (s, 1H), 8.01 (d, 1H), 7.81 (s, 1H), 7.47 (d, 1H), |
| | 7.34 (s, 1H), 6.91 (s, 1H), 6.85 (d, 1H), 6.48 (d, 1H), 4.94 (d, 1H), 4.56-4.35 (m, |
| | 5H), 3.65-3.37 (m, 4H), 1.83-1.66 (m, 2H), 1.31 (t, 6H). |

### Example 50 and 51

### 8-(2-(4H-1,2,4-triazol-4-yl)ethoxy)-N-(2-((3aR,6aS)-hexahydro-1H-furo[3,4-b]pyrrol-1-yl)pyrimidin-4-yl)-5-isopropylisoquinolin-3-amine and 8-(2-(4H-1,2,4-triazol-4-yl)ethoxy)-N-(2-((3aS,6aR)-hexahydro-1H-furo[3,4-b]pyrrol-1-yl)pyrimidin-4-yl)-5-isopropylisoquinolin-3-amine

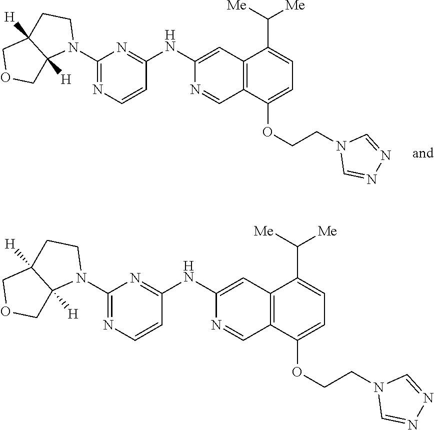 [SMILES: CC(C)c1ccc(OCCn2cnnc2)c2cnc(Nc3ccnc(N4CC[C@H]5COC[C@H]54)n3)cc12 ; "and" ; CC(C)c1ccc(OCCn2cnnc2)c2cnc(Nc3ccnc(N4CC[C@@H]5COC[C@@H]54)n3)cc12]

Into a 8-mL pressure tank reactor purged and maintained with an inert atmosphere of N₂ was placed 8-(2-(4H-1,2,4-triazol-4-yl)ethoxy)-3-chloro-5-isopropylisoquinoline (Preparation 76, 100 mg, 0.316 mmol), Cs₂CO₃ (206 mg, 0.631 mmol), cis-rac-2-((3aR,6aS)-hexahydro-1H-furo[3,4-b]pyrrol-1-yl)pyrimidin-4-amine (Preparation 32, 71.6 mg, 0.347 mmol), Brettphos Pd G3 and dioxane (6 mL) and the resulting solution stirred for 3 h at 100° C. The reaction was quenched with H₂O (30 mL) and the solids removed by filtration. The filtrate was extracted with EtOAc (3×50 mL) and the combined organics evaporated to dryness in vacuo. The residue was purified by HPLC-10 to afford 8-(2-(4H-1,2,4-triazol-4-yl)ethoxy)-N-(2-(hexahydro-1H-furo[3,4-b]pyrrol-1-yl)pyrimidin-4-yl)-5-isopropylisoquinolin-3-amine as a pale yellow solid (50 mg, 32.6%). The residue was purified further by chiral-HPLC (Chiralpak IF-3, 0.46×5 cm, 3 mm; (50% (3:1 Hex/DCM+0.1% DEA)/IPA) to afford the title compounds.

Peak 1; Yield: 20 mg; LCMS m/z=490 [M+H]⁺; ¹H NMR (400 MHz, DMSO-d₆) δ: 9.93 (s, 1H), 9.23 (s, 1H), 8.92-8.54 (m, 3H), 7.98 (d, 1H), 7.46 (d, 1H), 6.84 (d, 1H), 6.54 (s, 1H), 4.61 (t, 2H), 4.45 (t, 2H), 4.34-4.02 (m, 3H), 3.79 (dt, 2H), 3.63-3.36 (m, 3H), 2.78-2.57 (m, 1H), 2.15-2.01 (m, 1H), 1.97-1.82 (m, 1H), 1.29 (d, 6H).

Peak 2; Yield: 20 mg; LCMS m/z=490 [M+H]⁺; ¹H NMR (400 MHz, DMSO-d₆) δ: 9.93 (s, 1H), 9.23 (s, 1H), 8.78 (s, 1H), 8.70 (s, 2H), 7.98 (d, 1H), 7.46 (d, 1H), 6.84 (d, 1H), 6.54 (s, 1H), 4.61 (t, 2H), 4.45 (t, 2H), 4.26-3.99 (m, 3H), 3.84 (t, 1H), 3.75 (td, 1H), 3.51 (dt, 2H), 3.44 (d, 1H), 2.68-2.60 (m, 1H), 2.08 (dd, 1H), 1.91 (p, 1H), 1.29 (d, 6H).

### Example 52

### (R)-4-(((3-((2-((3S,4R)-3-fluoro-4-(2-hydroxyethoxy)-4-methylpiperidin-1-yl)pyrimidin-4-yl)amino)-5-isopropylisoquinolin-8-yl)oxy)methyl)-3-methyloxazolidin-2-one

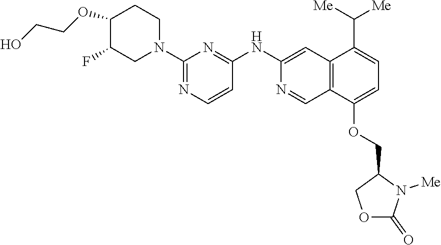 [SMILES: CC(C)c1ccc(OC[C@@H]2COC(=O)N2C)c2cnc(Nc3ccnc(N4CC[C@@H](OCCO)[C@@H](F)C4)n3)cc12]

Under an atmosphere of N₂ into a 40-mL sealed tube, was placed (R)-5-(((3-chloro-5-isopropylisoquinolin-8-yl)oxy)methyl)-3-methyloxazolidin-2-one (Preparation 61, 30 mg, 0.090 mmol) in dioxane (6 mL). 2-(((3S,4R)-1-(4-Aminopyrimidin-2-yl)-3-fluoropiperidin-4-yl)oxy)ethan-1-ol (Preparation 25, 27.6 mg, 0.108 mmol), Cs₂CO₃ (58.6 mg, 0.180 mmol), C-Phos (15.6 mg, 0.036 mmol) and Pd(dba)3 (18.6 mg, 0.018 mmol) were added and the resulting solution stirred at 100° C. for 3 h. The mixture was diluted with EtOAc (20 mL) and washed with brine. The combined organics were dried (Na₂SO₄) and evaporated to dryness in vacuo. The residue was purified by prep-TLC (10:1 DCM/MeOH) to afford the title compound as a yellow solid (35 mg, 70%). LCMS m/z=553 [M+H]⁺;

### Example 53

### (R)-4-(((3-((2-((3S,4R)-4-(2-(dimethylamino)ethoxy)-3-fluoro-piperidin-1-yl)pyrimidin-4-yl)amino)-5-isopropylisoquinolin-8-yl)oxy)methyl)-3-methyloxazolidin-2-one

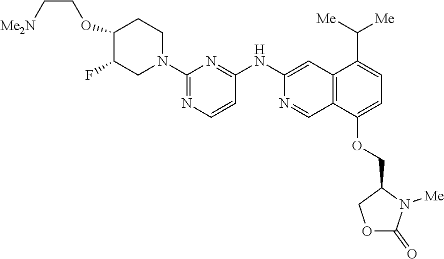 [SMILES: CC(C)c1ccc(OC[C@@H]2COC(=O)N2C)c2cnc(Nc3ccnc(N4CC[C@@H](OCCN(C)C)[C@@H](F)C4)n3)cc12]

2-(((3S,4R)-3-Fluoro-1-(4-((5-isopropyl-8-(((R)-3-methyl-2-oxooxazolidin-4-yl)methoxy)isoquinolin-3-yl)amino)pyrimidin-2-yl)-4-methylpiperidin-4-yl)oxy)ethyl methanesulfonate (Preparation 89, 40 mg, 0.063 mmol) was stirred in methanolic dimethylamine (4 mL, 30% in MeOH) at 70° C. for 3 h. The reaction mixture was diluted with EtOAc and washed with brine. The organic solution was dried (Na₂SO₄) and evaporated to dryness in vacuo. The residue was purified by HPLC-9 (Gradient (% organic): 33-43%) to give the title compound as a white solid (15 mg, 41%). LCMS m/z=553 [M+H]⁺; ¹H NMR (300 MHz, DMSO-d₆) δ: 9.25 (s, 1H), 8.73 (s, 1H), 7.98 (d, 1H), 7.52 (d, 1H), 6.89 (d, 1H), 6.38 (d, 1H), 4.90-4.73 (m, 1H), 4.81 (s, 1H), 4.66-4.52 (m, 1H), 4.47-4.09 (m, 4H), 3.89-3.69 (m, 3H), 3.67-3.44 (m, 2H), 2.64 (t, 3H), 2.53-2.37 (m, 2H), 2.34 (s, 6H), 2.21-2.07 (m, 1H), 2.01-1.79 (m, 2H), 1.40-1.31 (m, 6H).

### Example 54

### (R)-4-(((3-((2-((3S,4R)-3-fluoro-4-(2-(methylamino)ethoxy)piperidin-1-yl)pyrimidin-4-yl)amino)-5-isopropylisoquinolin-8-yl)oxy)methyl)-3-methyloxazolidin-2-one

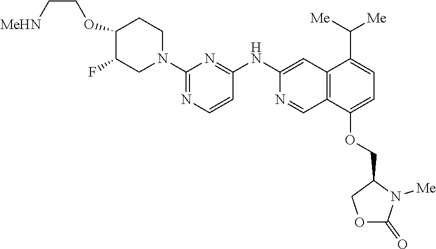 [SMILES: CNCCO[C@@H]1CCN(c2nccc(Nc3cc4c(C(C)C)ccc(OC[C@@H]5COC(=O)N5C)c4cn3)n2)C[C@@H]1F]

The title compound was prepared from 2-(((3S,4R)-3-fluoro-1-(4-((5-isopropyl-8-(((R)-3-methyl-2-oxooxazolidin-4-yl)methoxy)isoquinolin-3-yl)amino)pyrimidin-2-yl)-4-methylpiperidin-4-yl)oxy)ethyl methanesulfonate (Preparation 89) and methylamine in EtOH using an analogous method to that described for Example 53. Yield: 15 mg, 42%; LCMS m/z=566 [M+H]⁺; ¹H NMR (300 MHz, DMSO-d₆) δ: 9.26 (s, 1H), 8.72 (s, 1H), 7.98 (d, 1H), 7.52 (d, 1H), 6.90 (d, 1H), 6.39 (d, 1H), 5.04-4.88 (m, 2H), 4.70-4.52 (m, 1H), 4.50-4.10 (m, 4H), 3.96-3.45 (m, 6H), 3.36 (s, 2H), 2.97 (s, 3H), 2.88-2.78 (m, 2H), 2.72-2.56 (m, 1H), 2.55-2.30 (m, 5H), 2.21-2.08 (m, 1H), 1.92 (s, 2H), 1.39 (dd, 6H).

### Example 55

### 8-(2-(4H-1,2,4-triazol-4-yl)ethoxy)-N-(2-((3S,4R)-3-fluoro-4-methoxypiperidin-1-yl)pyrimidin-4-yl)-5-isopropyl-2,6-naphthyridin-3-amine

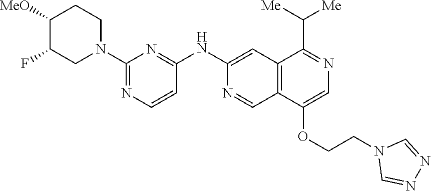 [SMILES: CO[C@@H]1CCN(c2nccc(Nc3cc4c(C(C)C)ncc(OCCn5cnnc5)c4cn3)n2)C[C@@H]1F]

A solution of 2-((3S,4R)-3-fluoro-4-methoxypiperidin-1-yl)pyrimidin-4-amine (Preparation 31, 19.4 mg, 0.086 mmol), 4-(2-(4H-1,2,4-triazol-4-yl)ethoxy)-7-chloro-1-isopropyl-2,6-naphthyridine (Preparation 91, 27.3 mg, 0.086 mmol), BrettPhos (2.3 mg, 0.004 mmol)/BrettPhos Pd G4 (3.95 mg, 0.004 mmol) and Cs₂CO₃ (56.0 mg, 0.172 mmol) in dioxane (0.86 mL) was purged with N₂ for 5 min before stirring at 90° C. for 2 hours. The reaction mixture was filtered through Celite® (5% MeOH/DCM) and the filtrate evaporated to dryness in vacuo. The residue was purified by ISCO chromatography (0-20% MeOH/DCM) to afford the title compound as a beige solid (19.7 mg, 45%). LCMS m/z=508 [M+H]⁺; ¹H NMR (400 MHz, DMSO-d₆) δ: 10.25 (s, 1H), 9.34 (d, 1H), 8.82 (s, 1H), 8.74 (s, 2H), 8.11 (s, 1H), 8.09 (d, 1H), 6.52 (d, 1H), 4.98 (d, 1H), 4.82-4.69 (m, 1H), 4.62 (dt, 4H), 4.51 (d, 1H), 3.76 (p, 1H), 3.59 (ddd, 2H), 3.40 (s, 3H), 3.20 (d, 1H), 1.98-1.72 (m, 2H), 1.35 (dd, 6H).

### Example 56

### 1-(3-(((3-((2-((3S,4R)-3-hydroxy-4-methoxypiperidin-1-yl)pyrimidin-4-yl)amino)-5-isopropylisoquinolin-8-yl)oxy)methyl)azetidin-1-yl)ethan-1-one or 1-(3-(((3-((2-((3R,4S)-3-hydroxy-4-methoxypiperidin-1-yl)pyrimidin-4-yl)amino)-5-isopropylisoquinolin-8-yl)oxy)methyl)azetidin-1-yl)ethan-1-one

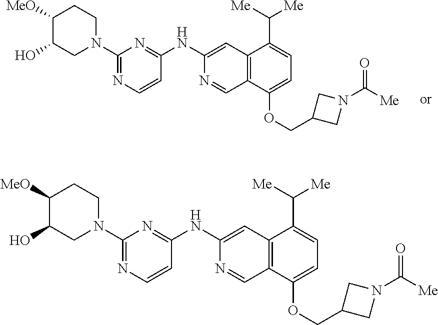 [SMILES: CO[C@@H]1CCN(c2nccc(Nc3cc4c(C(C)C)ccc(OCC5CN(C(C)=O)C5)c4cn3)n2)C[C@@H]1O ; "or" ; CO[C@H]1CCN(c2nccc(Nc3cc4c(C(C)C)ccc(OCC5CN(C(C)=O)C5)c4cn3)n2)C[C@H]1O]

A mixture of 1-(3-(((3-chloro-5-isopropylisoquinolin-8-yl)oxy)methyl)azetidin-1-yl)ethan-1-one (Preparation 63, 50 mg, 0.150 mmol), the compound of Peak 1 of Preparation 16 and 17 (33.6 mg, 0.150 mmol), Cs₂CO₃ (97.8 mg, 0.30 mmol), Brettphos Pd G4 (13.6 mg, 0.015 mmol) in dioxane (2 mL) under N₂ and heated to 100° C. for 3h. The reaction mixture was diluted with H₂O and extracted with EtOAc. The combined organics were dried (Na₂SO₄) and evaporated to dryness in vacuo. The residue was purified by column chromatography (10:1 DCM/MeOH) and prep-HPLC to afford the title compound as a white solid (28 mg, 36%). LCMS m/z=521 [M+H]⁺; ¹H NMR (300 MHz, DMSO-d₆) δ: 9.98 (s, 1H), 9.23 (s, 1H), 8.77 (s, 1H), 7.99 (d, 1H), 7.49 (d, 1H), 6.88 (d, 1H), 6.42 (s, 1H), 4.64 (d, 1H), 4.32 (d, 3H), 4.14-3.88 (m, 4H), 3.84-3.41 (m, 6H), 3.37 (s, 3H), 3.13 (s, 1H), 1.98-1.74 (m, 4H), 1.64 (s, 1H), 1.32 (d, 6H).

### Example 57

### 1-(3-(((3-((2-((3S,4S)-3-hydroxy-4-methoxypiperidin-1-yl)pyrimidin-4-yl)amino)-5-isopropylisoquinolin-8-yl)oxy)methyl)azetidin-1-yl)ethan-1-one or 1-(3-(((3-((2-((3R,4R)-3-hydroxy-4-methoxypiperidin-1-yl)pyrimidin-4-yl)amino)-5-isopropylisoquinolin-8-yl)oxy)methyl)azetidin-1-yl)ethan-1-one

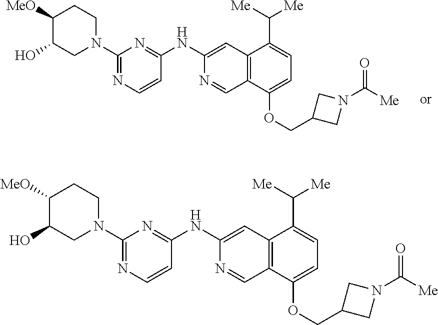 [SMILES: CO[C@H]1CCN(c2nccc(Nc3cc4c(C(C)C)ccc(OCC5CN(C(C)=O)C5)c4cn3)n2)C[C@@H]1O ; "or" ; CO[C@@H]1CCN(c2nccc(Nc3cc4c(C(C)C)ccc(OCC5CN(C(C)=O)C5)c4cn3)n2)C[C@H]1O]

The title compound was prepared from 1-(3-(((3-chloro-5-isopropylisoquinolin-8-yl)oxy)methyl)azetidin-1-yl)ethan-1-one (Preparation 63) and the compound of Peak 1 of Preparation 18 and 19 using an analogous method to that described for Example 56. Yield=30 mg, 32%); LCMS m/z=521 [M+H]⁺; ¹H NMR (300 MHz, DMSO-d₆) δ: 10.01 (s, 1H), 9.24 (s, 1H), 8.74 (s, 1H), 8.00 (d, 1H), 7.49 (d, 1H), 6.88 (d, 1H), 6.44 (d, 1H), 5.11 (d, 1H), 4.47-4.19 (m, 5H), 4.13-3.97 (m, 2H), 3.80 (dd, 1H), 3.64-3.47 (m, 1H), 3.45-3.34 (m, 4H), 3.30-2.98 (m, 4H), 2.15-2.00 (m, 1H), 1.48-1.19 (m, 7H).

### Example 58

### N-(3-((3-((2-((3S,4S)-3-hydroxy-4-methoxypiperidin-1-yl)pyrimidin-4-yl)amino)-5-isopropylisoquinolin-8-yl)oxy)propyl)acetamide or N-(3-((3-((2-((3R,4R)-3-hydroxy-4-methoxypiperidin-1-yl)pyrimidin-4-yl)amino)-5-isopropylisoquinolin-8-yl)oxy)propyl)acetamide

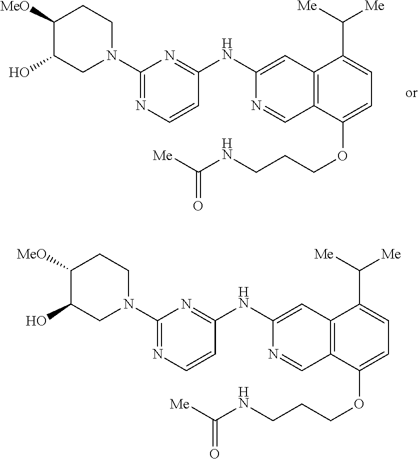 [SMILES: CO[C@H]1CCN(c2nccc(Nc3cc4c(C(C)C)ccc(OCCCNC(C)=O)c4cn3)n2)C[C@@H]1O ; CO[C@@H]1CCN(c2nccc(Nc3cc4c(C(C)C)ccc(OCCCNC(C)=O)c4cn3)n2)C[C@H]1O]

A mixture of N-(3-((3-chloro-5-isopropylisoquinolin-8-yl)oxy)propyl)acetamide (Preparation 62, 40 mg, 0.125 mmol), the compound of Peak 1 of Preparation 18 and 19 (28 mg, 0.125 mmol), Pd₂(dba)₃·CHCl₃ (12.9 mg, 0.0125 mmol), CPhos (5.46 mg, 0.0125 mmol), Cs₂CO₃ (81 mg, 0.250 mmol) in dioxane (10 mL) was stirred at 100° C. for 2 h. The mixture was evaporated to dryness and the residue was purified by prep-TLC (25:1 DCM/MeOH) followed by HPLC-9 (Gradient (% organic): 25-55%) to afford the title compound as an off-white solid (15.7 mg, 24.7%). LCMS m/z=509 [M+H]⁺; ¹H NMR (300 MHz, DMSO-d₆) δ: 9.99 (s, 1H), 9.29 (s, 1H), 8.72 (s, 1H), 8.08-7.91 (m, 2H), 7.47 (d, 1H), 6.83 (d, 1H), 6.44 (d, 1H), 5.10 (d, 1H), 4.52-4.28 (m, 2H), 4.25-4.09 (m, 2H), 3.64-3.43 (m, 1H), 3.41-3.35 (m, 5H), 3.30-3.07 (m, 3H), 2.14-1.91 (m, 3H), 1.80 (s, 3H), 1.49-1.13 (m, 8H).

### Example 59

### 6-((2-((3S,4R)-3-fluoro-4-hydroxy-3-methylpiperidin-1-yl)pyrimidin-4-yl)amino)-4-isopropyl-N-(1-methylazetidin-3-yl)-2,7-naphthyridine-1-carboxamide

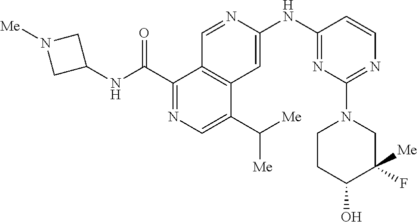 [SMILES: CC(C)c1cnc(C(=O)NC2CN(C)C2)c2cnc(Nc3ccnc(N4CC[C@@H](O)[C@@](C)(F)C4)n3)cc12]

6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 40 mg, 0.090 mmol), TEA (27.5 mg, 0.272 mmol) and HATU (51.7 mg, 0.136 mmol) were dissolved in DMF (2 mL) and the mixture stirred at 25° C. for 1 h. 1-Methylazetidin-3-amine (23.4 mg, 0.272 mmol) was added and the reaction stirred at rt for a further 8 h. The crude mixture was diluted with H₂O, extracted with EtOAc and the combined organic extracts concentrated in vacuo. The crude product was purified by silica gel column eluting with MeOH-DCM (1:50) and the product was further purified by HPLC-11 (Gradient (% organic): 26-40%) to provide the title compound, 21 mg, 45.9%, as a yellow solid. LCMS m/z=509 [M+H]^{+ 1}HNMR (400 MHz, DMSO-d₆) δ: 10.41 (s, 1H), 10.08 (s, 1H), 9.24 (d, 1H), 8.75 (s, 1H), 8.54 (s, 1H), 8.07 (d, 1H), 6.48 (d, 1H), 5.05 (d, 1H), 4.82-4.58 (m, 2H), 4.53 (q, 1H), 3.58 (dt, 4H), 3.25-3.11 (m, 2H), 3.05 (t, 2H), 2.26 (s, 3H), 1.86-1.59 (m, 2H), 1.51-1.26 (m, 9H)

### Example 60

### 6-((2-((3S,4R)-3-fluoro-4-hydroxy-3-methylpiperidin-1-yl)pyrimidin-4-yl)amino)-4-isopropyl-N-(oxetan-3-yl)-2,7-naphthyridine-1-carboxamide

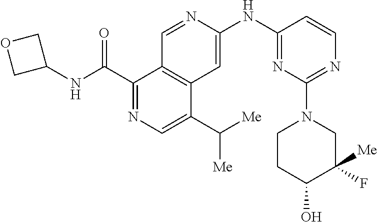 [SMILES: CC(C)c1cnc(C(=O)NC2COC2)c2cnc(Nc3ccnc(N4CC[C@@H](O)[C@@](C)(F)C4)n3)cc12]

The title compound was obtained as a yellow solid, 28 mg, 50%, from 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108) and oxetan-3-amine, following the procedure described in Example 59.

LCMS m/z=496 [M+H]^{+ 1}HNMR (400 MHz, DMSO-d₆) δ: 10.41 (s, 1H), 10.09 (s, 1H), 9.63 (d, 1H), 8.75 (s, 1H), 8.56 (s, 1H), 8.07 (d, 1H), 6.48 (d, 1H), 5.24-4.97 (m, 2H), 4.84-4.47 (m, 6H), 3.72-3.49 (m, 2H), 3.26-3.05 (m, 2H), 1.88-1.70 (m, 2H), 1.53-1.26 (m, 9H)

### Example 61

### 6-((2-((3S,4R)-3-fluoro-4-hydroxy-3-methylpiperidin-1-yl)pyrimidin-4-yl)amino)-4-isopropyl-2,7-naphthyridine-1-carboxamide or 6-((2-((3R,4S)-3-fluoro-4-hydroxy-3-methylpiperidin-1-yl)pyrimidin-4-yl)amino)-4-isopropyl-2,7-naphthyridine-1-carboxamide

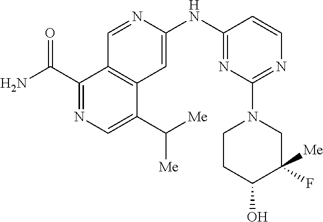 [SMILES: CC(C)c1cnc(C(N)=O)c2cnc(Nc3ccnc(N4CC[C@@H](O)[C@@](C)(F)C4)n3)cc12]

A mixture of 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 31 mg, 0.070 mmol), HATU (36 mg, 0.095 mmol), NH₄Cl (16 mg, 0.30 mmol) and DIPEA (0.048 mL, 0.275 mmol) were stirred in DMF (0.6 mL) at rt overnight. The reaction mixture was filtered and purified directly by mass-triggered prep-HPLC (basic modifier) to give the title compound, 15 mg, 48.5% yield as a pale yellow solid. LCMS m/z=441 [M+H]^{+ 1}HNMR (400 MHz, DMSO-d₆) δ: 10.36 (s, 1H), 10.16 (d, 1H), 8.72 (s, 1H), 8.50 (s, 1H), 8.24 (d, 1H), 8.04 (d, 1H), 7.80 (dd, 1H), 6.46 (d, 1H), 5.04-5.02 (m, 1H), 4.72-4.63 (m, 2H), 3.59-3.51 (m, 2H), 3.20-3.13 (m, 2H), 1.72-1.76 (m, 2H), 1.41-1.33 (m, 9H)

### Example 62

### 6-((2-((3S,4R)-3-fluoro-4-hydroxy-3-methylpiperidin-1-yl)pyrimidin-4-yl)amino)-N-(2-fluoroethyl)-4-isopropyl-2,7-naphthyridine-1-carboxamide

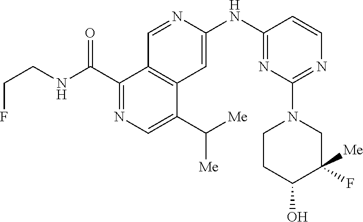 [SMILES: CC(C)c1cnc(C(=O)NCCF)c2cnc(Nc3ccnc(N4CC[C@@H](O)[C@@](C)(F)C4)n3)cc12]

A mixture of 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 50 mg, 0.114 mmol), 2-fluoroethan-1-amine (7.88 mg, 0.125 mmol), HATU (86.32 mg, 0.227 mmol) and DIPEA (44.01 mg, 0.341 mmol) in DMF (5 mL) was stirred for 3 h at rt. The reaction was quenched by the addition of water (5 mL), the mixture extracted with EtOAc (3×5 mL) and the combined organic layers dried over Na₂SO₄ and concentrated in vacuo. The residue was purified by silica gel column (EtOAc/PE, 1:3) and the product further purified by HPLC-12 (Gradient (% organic): 30-57%) to afford the title compound, 7.3 mg, 13.3%, as a white solid. LCMS m/z=486 [M+H]⁺; ¹HNMR (300 MHz, DMSO-d₆) δ: 10.39 (s, 1H), 10.19 (d, 1H), 9.09 (t, 1H), 8.73 (s, 1H), 8.53 (s, 1H), 8.05 (d, 1H), 6.47 (s, 1H), 5.05 (d, 1H), 4.80-4.57 (m, 3H), 4.52 (t, 1H), 3.78-3.47 (m, 4H), 3.27-3.02 (m, 2H), 1.84-1.68 (m, 2H), 1.53-1.23 (m, 9H)

### Example 63

### N—((S)-2,2-difluorocyclopropyl)-6-((2-((3S,4R)-3-fluoro-4-hydroxy-3-methylpiperidin-1-yl)pyrimidin-4-yl)amino)-4-isopropyl-2,7-naphthyridine-1-carboxamide or N—((R)-2,2-difluorocyclopropyl)-6-((2-((3S,4R)-3-fluoro-4-hydroxy-3-methylpiperidin-1-yl)pyrimidin-4-yl)amino)-4-isopropyl-2,7-naphthyridine-1-carboxamide

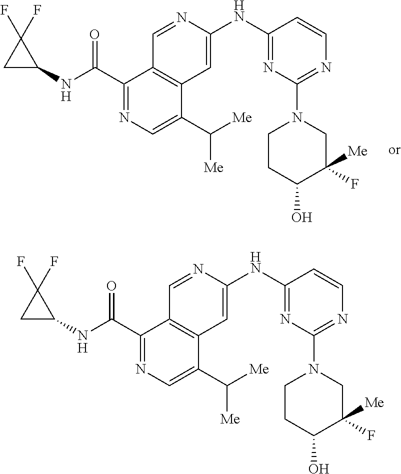 [SMILES: CC(C)c1cnc(C(=O)N[C@H]2CC2(F)F)c2cnc(Nc3ccnc(N4CC[C@@H](O)[C@@](C)(F)C4)n3)cc12 ; "or" ; CC(C)c1cnc(C(=O)N[C@@H]2CC2(F)F)c2cnc(Nc3ccnc(N4CC[C@@H](O)[C@@](C)(F)C4)n3)cc12]

A mixture of HATU (172.64 mg, 0.454 mmol), DIPEA (88.02 mg, 0.681 mmol), 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 100 mg, 0.227 mmol) and 2,2-difluorocyclopropan-1-amine (21.13 mg, 0.227 mmol) in DMF (5 mL), was stirred for 2 h at rt. The reaction was quenched with water (5 mL), the mixture extracted with EtOAc (3×5 mL) and the organic layers combined and concentrated in vacuo. The crude product was purified by HPLC-12 (Gradient (% organic): 30-57%). The product was further purified by prep-chiral SFC (Cellulose-SB 4.6×100 mm, 3 mm; Hexane (+0.1% DEA):EtOH=50:50)) to afford: Peak 1, 7 mg, 6.0% yield, as a light yellow solid. LCMS m/z=516 [M+H]⁺; 1H-NMR (400 MHz, DMSO-d₆) δ: 10.51 (s, 1H), 10.11 (s, 1H), 9.32 (t, 1H), 8.74 (s, 1H), 8.54 (s, 1H), 8.06 (d, 1H), 6.48 (d, 1H), 5.22-4.95 (m, 1H), 4.82-4.46 (m, 2H), 3.71-3.48 (m, 3H), 3.26-3.02 (m, 2H), 2.13-1.64 (m, 4H), 1.51-1.28 (m, 9H)

### Example 64

### 6-((2-((3S,4R)-3-fluoro-4-hydroxy-3-methylpiperidin-1-yl)pyrimidin-4-yl)amino)-4-isopropyl-N-methyl-2,7-naphthyridine-1-carboxamide

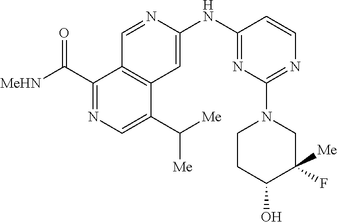 [SMILES: CNC(=O)c1ncc(C(C)C)c2cc(Nc3ccnc(N4CC[C@@H](O)[C@@](C)(F)C4)n3)ncc12]

A mixture of 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108, 2 g, 4.54 mmol), EDC·HCl (1.0 g, 6.81 mmol), HOBT (919 mg, 6.81 mmol) and methanamine HCl (183 mg, 5.9 mmol) in DMF was stirred overnight at rt. Water was added, the mixture extracted with EtOAc, the organic layers combined and dried over Na₂SO₄ and concentrated in vacuo. The crude product was purified by HPLC-12 (Gradient (% organic): 27-40%) to afford the title compound, 572 mg, 27.8% as an off-white solid. LCMS m/z=454 [M+H]^{+ 1}HNMR (400 MHz, DMSO-d₆) δ: 10.41 (s, 1H), 10.22 (s, 1H), 8.90 (q, 1H), 8.75 (s, 1H), 8.52 (s, 1H), 8.07 (d, 1H), 6.48 (d, 1H), 5.07 (d, 1H), 4.83-4.60 (m, 2H), 3.58 (td, 2H), 3.24-3.05 (m, 2H), 2.89 (d, 3H), 1.83-1.66 (m, 2H), 1.50-1.27 (m, 9H)

### Examples 65 to 76

The compounds in the following table were prepared from 6-((2-((3S,4R)-3-fluoro-4-hydroxy-3-methylpiperidin-1-yl)pyrimidin-4-yl)amino)-4-isopropyl-2,7-naphthyridine-1-carboxylic acid (Preparation 108) and the appropriate amine (R³NH₂), following a similar procedure to that described in Example 64.

| Example No. | Name/Structure/R³NH₂/HPLC/Data |
|---|---|
| 65 | N-ethyl-6-((2-((3S,4R)-3-fluoro-4-hydroxy-3-methylpiperidin-1-yl)pyrimidin-4- |
| | yl)amino)-4-isopropyl-2,7-naphthyridine-1-carboxamide |
| | 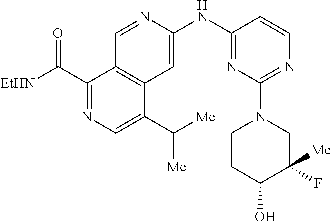 |
| | R³NH₂: ethylamine |
| | HPLC-9 (Gradient (% organic): 35-39%) |
| | 160 mg, 43%, off-white solid. LCMS m/z = 468 [M + H]⁺ ¹HNMR (400 MHz, |
| | DMSO-d₆) δ: 10.39 (s, 1H), 10.17 (d, 1H), 8.91 (t, 1H), 8.73 (s, 1H), 8.50 |
| | (s, 1H), 8.05 (d, 1H), 6.47 (s, 1H), 5.04 (d, 1H), 4.84-4.51 (m, 2H), 3.57 |
| | (dd, 2H), 3.37 (dd, 2H), 3.26-3.04 (m, 2H), 1.85-1.64 (m, 2H), 1.44-1.27 |
| | (m, 9H), 1.16 (t, 3H) |
| 66 | 6-((2-((3S,4R)-3-fluoro-4-hydroxy-3-methylpiperidin-1-yl)pyrimidin-4- |
| A | yl)amino)-4-isopropyl-N-(2-methoxyethyl)-2,7-naphthyridine-1- |
| | carboxamide |
| | 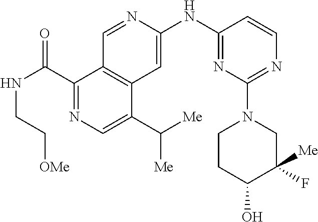 [SMILES: COCCNC(=O)c1ncc(C(C)C)c2cc(Nc3ccnc(N4CC[C@@H](O)[C@@](C)(F)C4)n3)ncc12] |
| | R³NH₂: 2-methoxyethan-1-amine |
| | HPLC-13 |
| | 13 mg, 23.1%, as light-yellow solid |
| | LCMS m/z = 498 [M + H]⁺ ¹HNMR (300 MHz, DMSO-d₆) δ: 10.39 (s, 1H), 10.22 |
| | (s, 1H), 8.96-8.81 (m, 1H), 8.73 (s, 1H), 8.52 (s, 1H), 8.05 (d, 1H), 6.46 (d, |
| | 1H), 5.04 (d, 1H), 4.88-4.42 (m, 2H), 3.71-3.43 (m, 6H), 3.25-2.99 (m, |
| | 4H), 1.85-1.62 (m, 2H), 1.50-1.20 (m, 9H) |
| 67 | N-(2,2-difluoroethyl)-6-((2-((3S,4R)-3-fluoro-4-hydroxy-3-methylpiperidin-1- |
| A | yl)pyrimidin-4-yl)amino)-4-isopropyl-2,7-naphthyridine-1-carboxamide |
| | 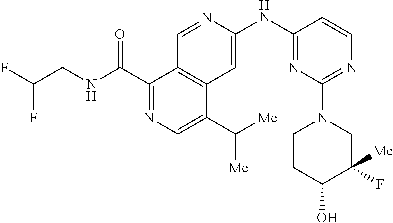 [SMILES: CC(C)c1cnc(C(=O)NCC(F)F)c2cnc(Nc3ccnc(N4CC[C@@H](O)[C@@](C)(F)C4)n3)cc12] |
| | R³NH₂: 2,2-difluoroethan-1-amine |
| | HPLC-8 (Gradient (% organic): 35-48%) |
| | 20 mg, 35.2%, as light-yellow solid |
| | LCMS m/z = 504 [M + H]⁺ ¹HNMR (300 MHz, DMSO-d₆) δ: 10.40 (s, 1H), 10.19 |
| | (d, 1H), 9.24 (t, 1H), 8.74 (s, 1H), 8.54 (s, 1H), 8.05 (d, 1H), 6.46 (d, 1H), |
| | 6.41-5.91 (m, 1H), 5.05 (d, 1H), 4.80-4.54 (m, 2H), 3.77 (tt, 2H), 3.57 (dq, |
| | 2H), 3.36-3.07 (m, 2H), 3.23-3.05 (m, 2H), 1.81-1.63 (m, 2H), 1.49-1.26 |
| | (m, 9H). |
| 68 | N-cyclopropyl-6-((2-((3S,4R)-3-fluoro-4-hydroxy-3-methylpiperidin-1- |
| | yl)pyrimidin-4-yl)amino)-4-isopropyl-2,7-naphthyridine-1-carboxamide |
| | 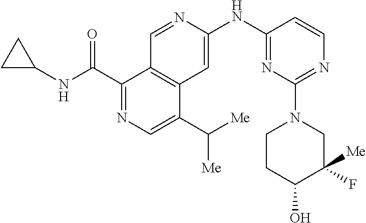 [SMILES: CC(C)c1cnc(C(=O)NC2CC2)c2cnc(Nc3ccnc(N4CC[C@@H](O)[C@@](C)(F)C4)n3)cc12] |
| | R³NH₂: cyclopropanamine |
| | HPLC-8 (Gradient (% organic): 33-47%) |
| | 12 mg, 22.2% yield, light yellow solid. |
| | LCMS m/z = 480 [M + H]⁺ ¹HNMR (300 MHz, DMSO-d₆) δ: 10.38 (s, 1H), 10.08 |
| | (s, 1H), 8.87 (d, 1H), 8.72 (s, 1H), 8.48 (s, 1H), 8.05 (d, 1H), 6.46 (d, 1H), |
| | 5.05 (d, 1H), 4.70 (dd, 2H), 3.54 (dd, 2H), 3.71-3.29 (m, 4H), 3.27-3.10 |
| | (m, 1H), 2.95 (dd, 1H), 1.83-1.66 (m, 2H), 1.46-1.27 (m, 9H), 0.84-0.60 |
| | (m, 4H) |
| 69 | 6-((2-((3S,4R)-3-fluoro-4-hydroxy-3-methylpiperidin-1-yl)pyrimidin-4- |
| B | yl)amino)-N,4-diisopropyl-2,7-naphthyridine-1-carboxamide |
| | 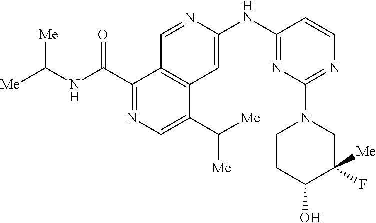 [SMILES: CC(C)NC(=O)c1ncc(C(C)C)c2cc(Nc3ccnc(N4CC[C@@H](O)[C@@](C)(F)C4)n3)ncc12] |
| | R³NH₂: propan-2-amine |
| | HPLC-12 (Gradient (% organic): 30-57%) |
| | 28 mg, 58.2% yield, light yellow solid |
| | LCMS m/z = 482 [M + H]⁺ ¹HNMR (400 MHz, DMSO-d₆) δ: 10.39 (s, 1H), 10.09 |
| | (s, 1H), 8.82-8.59 (m, 2H), 8.51 (s, 1H), 8.05 (d, 1H), 6.46 (d, 1H), 5.05 (d, |
| | 1H), 4.70 (dd, 2H), 4.30-4.03 (m, 1H), 3.57 (dd, 2H), 3.21-3.03 (m, 2H), |
| | 1.87-1.64 (m, 2H), 1.46-1.27 (m, 9H), 1.21 (d, 6H) |
| 70 | 6-((2-((3S,4R)-3-fluoro-4-hydroxy-3-methylpiperidin-1-yl)pyrimidin-4- |
| A | yl)amino)-N-(2-hydroxy-2-methylpropyl)-4-isopropyl-2,7-naphthyridine- |
| | 1-carboxamide |
| | 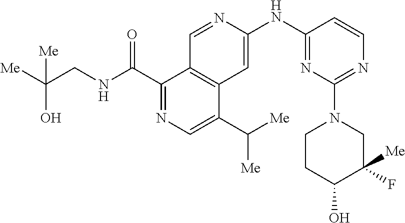 [SMILES: CC(C)c1cnc(C(=O)NCC(C)(C)O)c2cnc(Nc3ccnc(N4CC[C@@H](O)[C@@](C)(F)C4)n3)cc12] |
| | R³NH₂: 1-amino-2-methylpropan-2-ol |
| | HPLC-11 (Gradient (% organic): 20-40%) |
| | 24 mg, 41.2% yield, light yellow solid |
| | LCMS m/z = 512 [M + H]⁺; ¹HNMR (300 MHz, DMSO-d₆) δ: 10.42 (s, 1H), |
| | 10.31 (s, 1H), 8.75 (s, 1H), 8.68 (t, 1H), 8.58 (s, 1H), 8.07 (d, 1H), 6.48 (d, |
| | 1H), 5.08 (d, 1H), 4.83-4.56 (m, 3H), 3.70-3.46 (m, 2H), 3.25-3.09 (m, |
| | 4H), 1.90-1.64 (m, 2H), 1.50-1.28 (m, 9H), 1.17 (s, 6H). |
| 71 | 6-((2-((3S,4R)-3-fluoro-4-hydroxy-3-methylpiperidin-1-yl)pyrimidin-4- |
| C | yl)amino)-N-((R)-1-fluoropropan-2-yl)-4-isopropyl-2,7-naphthyridine-1- |
| | carboxamide |
| | 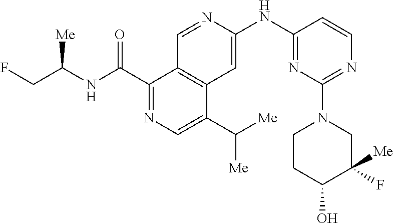 [SMILES: CC(C)c1cnc(C(=O)N[C@H](C)CF)c2cnc(Nc3ccnc(N4CC[C@@H](O)[C@@](C)(F)C4)n3)cc12] |
| | R³NH₂: (2R)-1-fluoropropan-2-amine |
| | HPLC-12 (Gradient (% organic): 30-57%) |
| | 18 mg, 36.1% yield, light yellow solid |
| | LCMS m/z = 500 [M + H]⁺ ¹HNMR (300 MHz, DMSO-d₆) δ: 10.40 (s, 1H), 10.12 |
| | (s, 1H), 8.89 (d, 1H), 8.74 (s, 1H), 8.53 (s, 1H), 8.05 (d, 1H), 6.46 (d, 1H), |
| | 5.05 (d, 1H), 4.80-4.51 (m, 3H), 4.47-4.28 (m, 2H), 3.74-3.45 (m, 2H), |
| | 3.24-3.05 (m, 2H), 1.85-1.68 (m, 2H), 1.56-1.29 (m, 9H), 1.21 (d, 3H) |
| 72 | 6-((2-((3S,4R)-3-fluoro-4-hydroxy-3-methylpiperidin-1-yl)pyrimidin-4- |
| C | yl)amino)-N-((S)-1-fluoropropan-2-yl)-4-isopropyl-2,7-naphthyridine-1- |
| | carboxamide |
| | 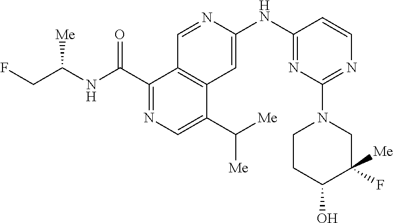 [SMILES: CC(C)c1cnc(C(=O)N[C@@H](C)CF)c2cnc(Nc3ccnc(N4CC[C@@H](O)[C@@](C)(F)C4)n3)cc12] |
| | R³NH₂: (2S)-1-fluoropropan-2-amine |
| | HPLC-12 (Gradient (% organic): 30-57%) |
| | 18 mg, 36.1% yield, light yellow solid |
| | LCMS m/z = 500 [M + H]⁺ ¹HNMR (300 MHz, DMSO-d₆) δ ppm: 10.40 (s, 1H), |
| | 10.13 (s, 1H), 8.89 (d, 1H), 8.74 (s, 1H), 8.53 (s, 1H), 8.05 (d, 1H), 6.47 (s, |
| | 1H), 5.05 (d, 1H), 4.85-4.49 (m, 3H), 4.45-4.29 (m, 2H), 3.58 (dd, 2H), |
| | 3.27-3.03 (m, 2H), 1.85-1.63 (m, 2H), 1.50-1.27 (m, 9H), 1.21 (d, 3H) |
| 73 | 6-((2-((3S,4R)-3-fluoro-4-hydroxy-3-methylpiperidin-1-yl)pyrimidin-4- |
| C | yl)amino)-4-isopropyl-N-((1s,3s)-3-methoxycyclobutyl)-2,7- |
| | naphthyridine-1-carboxamide |
| | 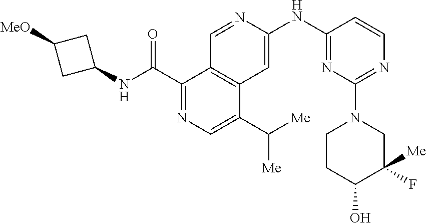 [SMILES: CO[C@H]1C[C@@H](NC(=O)c2ncc(C(C)C)c3cc(Nc4ccnc(N5CC[C@@H](O)[C@@](C)(F)C5)n4)ncc23)C1] |
| | R³NH₂: (1s,3s)-3-methoxycyclobutan-1-amine |
| | HPLC-12 (Gradient (% organic): 30-57%) |
| | 20 mg, 38.2% yield, as light yellow solid. |
| | LCMS m/z = 524 [M + H]⁺; 1H-NMR (300 MHz, DMSO-d₆) δ: 10.39 (s, 1H), |
| | 10.03 (s, 1H), 9.08 (d, 1H), 8.73 (s, 1H), 8.51 (s, 1H), 8.05 (d, 1H), 6.46 (d, |
| | 1H), 5.05 (d, 1H), 4.87-4.51 (m, 2H), 4.29-3.92 (m, 1H), 3.74-3.40 (m, |
| | 3H), 3.24-2.90 (m, 5H), 2.61 (s, 2H), 2.19-1.94 (m, 2H), 1.85-1.66 (m, |
| | 2H), 1.50-1.26 (m, 9H) |
| 74 | 6-((2-((3S,4R)-3-fluoro-4-hydroxy-3-methylpiperidin-1-yl)pyrimidin-4- |
| C | yl)amino)-4-isopropyl-N-((1r,3r)-3-methoxycyclobutyl)-2,7-naphthyridine- |
| | 1-carboxamide |
| | 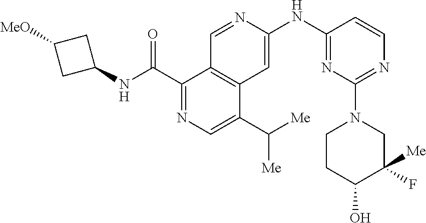 [SMILES: CO[C@H]1C[C@H](NC(=O)c2ncc(C(C)C)c3cc(Nc4ccnc(N5CC[C@@H](O)[C@@](C)(F)C5)n4)ncc23)C1] |
| | R³NH₂: (1r,3r)-3-methoxycyclobutan-1-amine |
| | HPLC-12 (Gradient (% organic): 30-57%) |
| | 20 mg, 38.2% yield, as light yellow solid. |
| | LCMS m/z = 524 [M + H]⁺; 1H-NMR (300 MHz, DMSO-d₆) δ: 10.39 (s, 1H), |
| | 10.06 (d, 1H), 9.17 (d, 1H), 8.72 (s, 1H), 8.51 (s, 1H), 8.04 (d, 1H), 6.45 |
| | (d, 1H), 5.05 (d, 1H), 4.79-4.60 (m, 2H), 4.52 (p, 1H), 4.00 (dd, 1H), 3.66- |
| | 3.45 (m, 2H), 3.15 (s, 3H), 2.42-2.28 (m, 3H), 2.27 (dd, 1H), 1.77-1.71 (m, |
| | 2H), 1.44-1.30 (m, 9H) |
| 75 | N-(3,3-difluorocyclobutyl)-6-((2-((3S,4R)-3-fluoro-4-hydroxy-3- |
| B | methylpiperidin-1-yl)pyrimidin-4-yl)amino)-4-isopropyl-2,7- |
| | naphthyridine-1-carboxamide |
| | 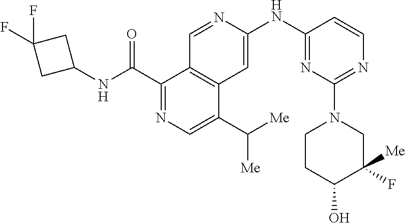 [SMILES: CC(C)c1cnc(C(=O)NC2CC(F)(F)C2)c2cnc(Nc3ccnc(N4CC[C@@H](O)[C@@](C)(F)C4)n3)cc12] |
| | R³NH₂: 3,3-difluorocyclobutan-1-amine |
| | HPLC-12 (Gradient (% organic): 30-57%) |
| | 24 mg, 45.4% yield, light yellow solid |
| | LCMS m/z = 530 [M + H]⁺ ¹HNMR (300 MHz, DMSO-d₆) δ: 10.41 (s, 1H), 10.13 |
| | (s, 1H), 9.43 (d, 1H), 8.74 (s, 1H), 8.53 (s, 1H), 8.05 (d, 1H), 6.46 (d, 1H), |
| | 5.05 (d, 1H), 4.82-4.50 (m, 2H), 4.45-4.13 (m, 1H), 3.71-3.49 (m, 2H), |
| | 3.27-2.81 (m, 6H), 1.85-1.65 (m, 2H), 1.48-1.21 (m, 9H). |
| 76 | N-(1,1-dioxidothietan-3-yl)-6-((2-((3S,4R)-3-fluoro-4-hydroxy-3- |
| C | methylpiperidin-1-yl)pyrimidin-4-yl)amino)-4-isopropyl-2,7- |
| | naphthyridine-1-carboxamide |
| | 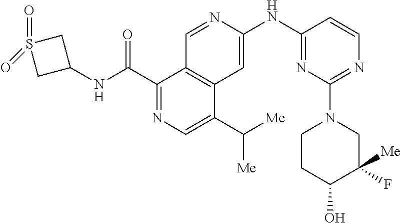 [SMILES: CC(C)c1cnc(C(=O)NC2CS(=O)(=O)C2)c2cnc(Nc3ccnc(N4CC[C@@H](O)[C@@](C)(F)C4)n3)cc12] |
| | R³NH₂: 3-aminothietane 1,1-dioxide |
| | HPLC-12 (Gradient (% organic): 30-57%) |
| | 28 mg, 51.6% yield as a light yellow solid |
| | LCMS m/z = 544 [M + H]⁺; 1H-NMR (300 MHz, DMSO-d₆) δ: 10.43 (s, 1H), |
| | 10.14 (s, 1H), 9.68 (s, 1H), 8.74 (s, 1H), 8.54 (s, 1H), 8.05 (d, 1H), 6.46 (d, |
| | 1H), 5.05 (d, 1H), 4.79-4.54 (m, 5H), 4.50-4.39 (m, 1H), 3.66-3.47 (m, |
| | 2H), 3.36-3.07 (m, 2H), 1.85-1.57 (m, 2H), 1.49-1.13 (m, 9H) |

A no reaction work up was conducted
B the reaction solids were filtered off and the filtrate concentrated in vacuo. The crude was purified by HPLC
C the reaction solids were filtered off, the filtrate concentrated in vacuo and the crude purified by silica gel column chromatography eluting with DCM/MeOH. The crude was purified by HPLC

### Example 64A

### 6-((2-((3S,4R)-3-fluoro-4-hydroxy-3-methylpiperidin-1-yl)pyrimidin-4-yl)amino)-4-isopropyl-N-methyl-2,7-naphthyridine-1-carboxamide

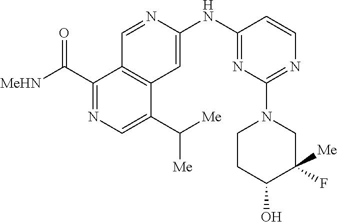 [SMILES: CNC(=O)c1ncc(C(C)C)c2cc(Nc3ccnc(N4CC[C@@H](O)[C@@](C)(F)C4)n3)ncc12]

A mixture of 6-chloro-4-isopropyl-N-methyl-2,7-naphthyridine-1-carboxamide (Preparation 99, 1.6 g, 6.08 mmol), (3S,4R)-1-(4-aminopyrimidin-2-yl)-3-fluoro-3-methylpiperidin-4-ol (Preparation 8, 1.37 g, 6.08 mmol), Cs₂CO₃ (3.96 g, 12.16 mmol) and Brettphos Pd G3 (551 mg, 0.608 mmol) in dioxane (20 mL) was heated to 120° C. for 2 h under N₂ atmosphere. The cooled reaction mixture was filtered, the filtrate concentrated under vacuum and the crude purified by HPLC-8 (Gradient (% organic): 27-40%) to give the title compound as an off-white solid (560 mg, 20.3%).

### Examples 77 to 81

The compounds in the following table were prepared from (3S,4R)-1-(4-aminopyrimidin-2-yl)-3-fluoro-3-methylpiperidin-4-ol (Preparation 8) and the appropriate chloro compound, following a similar procedure to that described in Example 64A (alternative synthesis).

| Example No | Name/Structure/R-Hal/HPLC/Data |
|---|---|
| 77 | N-(2-(dimethylamino)ethyl)-6-((2-((3S,4R)-3-fluoro-4-hydroxy-3- |
| | methylpiperidin-1-yl)pyrimidin-4-yl)amino)-4-isopropyl-2,7- |
| | naphthyridine-1-carboxamide |
| | 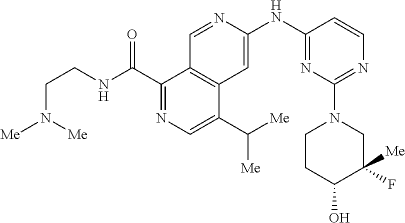 [SMILES: CC(C)c1cnc(C(=O)NCCN(C)C)c2cnc(Nc3ccnc(N4CC[C@@H](O)[C@@](C)(F)C4)n3)cc12] |
| | R-Hal: 6-chloro-N-(2-(dimethylamino)ethyl)-4-isopropyl-2,7-naphthyridine-1- |
| | carboxamide (Preparation 100) |
| | HPLC-8 (Gradient (% organic): 30-40%) |
| | 35 mg, 27.6% as a light-yellow solid. |
| | LCMS m/z = 511 [M + H]⁺ ¹HNMR (300 MHz, DMSO-d₆) δ: 10.37 (s, 1H), |
| | 10.27 (s, 1H), 8.86-8.68 (m, 2H), 8.53 (s, 1H), 8.05 (d, 1H), 6.46 (d, 1H), |
| | 5.03 (d, 1H), 4.82-4.55 (m, 2H), 3.65-3.49 (m, 2H), 3.44 (q, 2H), 3.15 (dd, |
| | 2H), 2.48-2.44 (m, 2H), 2.20 (s, 7H), 1.81-1.64 (m, 2H), 1.47-1.22 (m, |
| | 9H) |
| 78 | 6-((2-((3S,4R)-3-fluoro-4-hydroxy-3-methylpiperidin-1-yl)pyrimidin-4- |
| A | yl)amino)-4-isopropyl-N-(tetrahydro-2H-pyran-4-yl)-2,7-naphthyridine-1- |
| | carboxamide |
| | 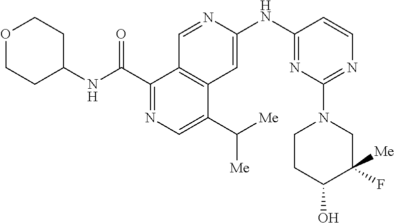 [SMILES: CC(C)c1cnc(C(=O)NC2CCOCC2)c2cnc(Nc3ccnc(N4CC[C@@H](O)[C@@](C)(F)C4)n3)cc12] |
| | R-Hal: 6-Chloro-4-isopropyl-N-(tetrahydro-2H-pyran-4-yl)-2,7-naphthyridine-1- |
| | carboxamide (Preparation 101) |
| | HPLC-2 (Gradient (% organic): 32-42%) |
| | 61 mg, 46%, as a white solid. |
| | LCMS m/z = 523 [M + H]⁺; ¹HNMR (400 MHz, DMSO-d₆) δ: 10.40 (s, 1H), |
| | 10.08 (s, 1H), 8.84 (d, 1H), 8.74 (s, 1H), 8.53 (s, 1H), 8.06 (d, 1H), 6.48 |
| | (d, 1H), 5.06 (d, 1H), 4.90-4.49 (m, 2H), 4.27-4.04 (m, 1H), 3.97-3.82 (m, |
| | 2H), 3.58 (dd, 2H), 3.47-3.36 (m, 2H), 3.23-3.10 (m, 2H), 1.93-1.59 (m, |
| | 6H), 1.48-1.26 (m, 9H) |
| 79 | 6-((2-((3S,4R)-3-fluoro-4-hydroxy-3-methylpiperidin-1-yl)pyrimidin-4- |
| A | yl)amino)-N-((1r,3r)-3-hydroxycyclobutyl)-4-isopropyl-2,7- |
| | naphthyridine-1-carboxamide |
| | 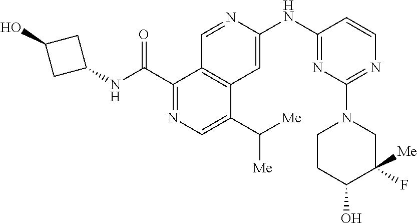 [SMILES: CC(C)c1cnc(C(=O)N[C@H]2C[C@H](O)C2)c2cnc(Nc3ccnc(N4CC[C@@H](O)[C@@](C)(F)C4)n3)cc12] |
| | R-Hal: 6-chloro-N-((1r,3r)-3-hydroxycyclobutyl)-4-isopropyl-2,7-naphthyridine- |
| | 1-carboxamide (Preparation 104) |
| | HPLC-2 (Gradient (% organic): 32-42%) |
| | 42 mg, 27.8 mg, as white solid |
| | LCMS m/z = 509 [M + H]⁺; 1H-NMR (300 MHz, DMSO-d₆) δ: 10.41 (s, 1H), |
| | 10.04 (s, 1H), 9.09 (d, 1H), 8.74 (s, 1H), 8.53 (s, 1H), 8.06 (d, 1H), 6.47 |
| | (d, 1H), 5.06 (d, 2H), 4.84-4.61 (m, 2H), 4.54 (q, 1H), 4.34 (d, 1H), 3.57 |
| | (dt, 2H), 3.25-3.14 (m, 2H), 2.36 (dt, 2H), 2.20 (dd, 2H), 1.84-1.68 (m, |
| | 2H), 1.49-1.32 (m, 9H). |
| 80 | 6-((2-((3S,4R)-3-fluoro-4-hydroxy-3-methylpiperidin-1-yl)pyrimidin-4- |
| B | yl)amino)-N-(1-(2-fluoroethyl)azetidin-3-yl)-4-isopropyl-2,7- |
| | naphthyridine-1-carboxamide |
| | 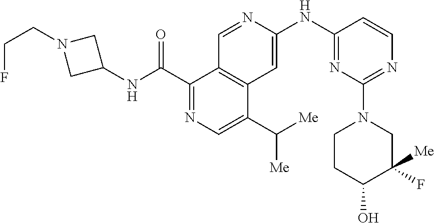 [SMILES: CC(C)c1cnc(C(=O)NC2CN(CCF)C2)c2cnc(Nc3ccnc(N4CC[C@@H](O)[C@@](C)(F)C4)n3)cc12] |
| | R-Hal: 6-chloro-N-(1-(2-fluoroethyl)azetidin-3-yl)-4-isopropyl-2,7- |
| | naphthyridine-1-carboxamide (Preparation 102) |
| | HPLC-2 (Gradient (% organic): 30-50%) |
| | 14 mg, 18.2% as a yellow solid. |
| | LCMS m/z = 541 [M + H]⁺; 1H-NMR (300 MHz, DMSO-d₆) δ: 10.43 (s, 1H), |
| | 10.10 (s, 1H), 9.29 (d, 1H), 8.76 (s, 1H), 8.55 (s, 1H), 8.07 (d, 1H), 6.48 |
| | (d, 1H), 4.82-4.55 (m, 3H), 4.49 (t, 1H), 4.37 (t, 1H), 3.75-3.48 (m, 4H), |
| | 3.22-3.07 (m, 4H), 2.73 (dt, 2H), 1.84-1.67 (m, 2H), 1.51-1.25 (m, 9H) |
| 81 | N-((1r,3r)-3-cyanocyclobutyl)-6-((2-((3S,4R)-3-fluoro-4-hydroxy-3- |
| B | methylpiperidin-1-yl)pyrimidin-4-yl)amino)-4-isopropyl-2,7- |
| | naphthyridine-1-carboxamide |
| | 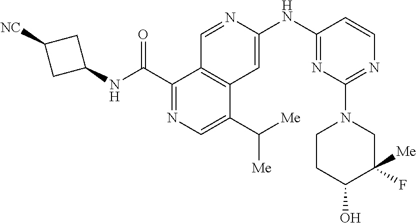 [SMILES: CC(C)c1cnc(C(=O)N[C@H]2C[C@@H](C#N)C2)c2cnc(Nc3ccnc(N4CC[C@@H](O)[C@@](C)(F)C4)n3)cc12] |
| | R-Hal: 6-chloro-N-((1r,3r)-3-cyanocyclobutyl)-4-isopropyl-2,7-naphthyridine-1- |
| | carboxamide (Preparation 103) |
| | HPLC-12(Gradient (% organic): 30-57%) |
| | 56 mg, 35.5% as a light yellow solid. |
| | LCMS m/z = 519 [M + H]⁺; 1H-NMR (400 MHz, DMSO-d₆) δ: 10.43 (s, 1H), |
| | 10.14 (s, 1H), 9.40 (d, 1H), 8.75 (s, 1H), 8.54 (s, 1H), 8.07 (d, 1H), 6.48 |
| | (d, 1H), 5.07 (d, 1H), 4.87-4.51 (m, 3H), 3.59 (tt, 2H), 3.19 (td, 2H), 2.63 |
| | (dd, 4H), 1.89-1.67 (m, 2H), 1.48-1.31 (m, 9H) |

A the crude product was purified by prep TLC (DCM/MeOH)
B the reaction was partitioned between EtOAc and water, the layers separated, the aqueous phase extracted with EtOAc and the combined organics washed with brine, dried and concentrated in vacuo. The crude was purified by silica gel column chromatography (DCM/MeOH) and the product further purified by HPLC.

### Example 82

### 6-((2-((4S,5R)-5-fluoro-4-hydroxy-3, 3-dimethylpiperidin-1l-yl)pyrimidin-4-yl)amino)-4-isopropyl-N-methyl-2,7-naphthyridine-1-carboxamide or 6-((2-((4R,5S)-5-fluoro-4-hydroxy-3,3-dimethylpiperidin-1-yl)pyrimidin-4-yl)amino)-4-isopropyl-N-methyl-2,7-naphthyridine-1-carboxamide

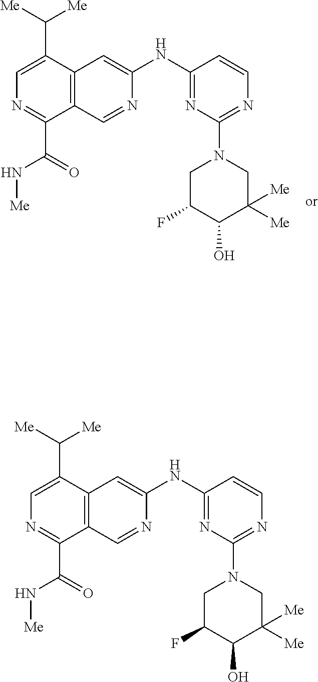 [SMILES: CNC(=O)c1ncc(C(C)C)c2cc(Nc3ccnc(N4C[C@@H](F)[C@@H](O)C(C)(C)C4)n3)ncc12 ; CNC(=O)c1ncc(C(C)C)c2cc(Nc3ccnc(N4C[C@H](F)[C@H](O)C(C)(C)C4)n3)ncc12]

The title compound was obtained as a light yellow solid, 23.4 mg, 29.3% yield, from 6-chloro-4-isopropyl-N-methyl-2,7-naphthyridine-1-carboxamide (Preparation 99) and the compound of Peak 2 of Preparation 21 and 22, following a similar procedure to that described in Example 64A (alternative synthesis).

HPLC-2 (Gradient (% organic): 25-50%); LCMS m/z=468 [M+H]⁺; ¹HNMR (300 MHz, DMSO-d₆) δ: 10.42 (s, 1H), 10.21 (s, 1H), 8.90 (d, 1H), 8.74 (s, 1H), 8.52 (s, 1H), 8.07 (d, 1H), 6.49 (d, 1H), 5.19 (d, 1H), 4.80 (d, 1H), 4.39-4.18 (m, 1H), 4.12-3.93 (m, 1H), 3.82 (d, 1H), 3.67-3.42 (m, 3H), 2.89 (d, 3H), 1.42 (t, 6H), 1.06-0.89 (m, 6H).

### Example 83

### 6-((2-((3S,4R)-3-fluoro-4-hydroxy-3-methylpiperidin-1-yl)pyrimidin-4-yl)amino)-N-((1s,3s)-3-hydroxy-3-methylcyclobutyl)-4-isopropyl-2,7-naphthyridine-1-carboxamide

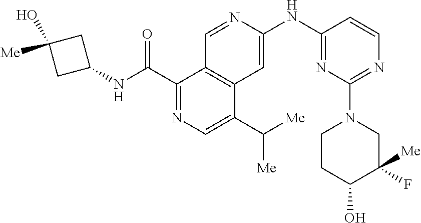 [SMILES: CC(C)c1cnc(C(=O)N[C@H]2C[C@@](C)(O)C2)c2cnc(Nc3ccnc(N4CC[C@@H](O)[C@@](C)(F)C4)n3)cc12]

6-Chloro-N-((1s,3s)-3-hydroxy-3-methylcyclobutyl)-4-isopropyl-2,7-naphthyridine-1-carboxamide (Preparation 106, 110 mg, 0.33 mmol) in dioxane (3 mL), was added to (3S,4R)-1-(4-aminopyrimidin-2-yl)-3-fluoro-3-methylpiperidin-4-ol (Preparation 8, 74.4 mg, 0.33 mmol), RuPhos Pd G3 (27.5 mg, 0.33 mmol) and Cs₂CO₃ (214 mg, 0.66 mmol), the vial sealed and the reaction stirred at 100° C. for 16 h. The cooled solution was concentrated in vacuo and the residue was purified by Prep-TLC with DCM/MeOH (20:1). The crude product was purified by HPLC-2 (Gradient (% organic): 25-35%) to afford the title compound, 38.2 mg, 71.7% as white solid. LCMS m/z=534 [M+H]⁺; 1H-NMR (400 MHz, DMSO-d₆) δ: 10.42 (s, 1H), 10.08 (s, 1H), 8.98 (d, 1H), 8.75 (s, 1H), 8.53 (s, 1H), 8.07 (d, 1H), 6.48 (d, 1H), 5.07 (d, 1H), 5.01 (s, 1H), 4.83-4.61 (m, 2H), 4.08 (p, 1H), 3.67-3.47 (m, 2H), 3.28-3.10 (m, 2H), 2.37 (td, 2H), 2.17 (dd, 2H), 1.81-1.73 (m, 2H), 1.48-1.33 (m, 9H), 1.29 (s, 3H)

### Example 84

### N-((1r,3r)-3-ethoxycyclobutyl)-6-((2-((3S,4R)-3-fluoro-4-hydroxy-3-methylpiperidin-1-yl)pyrimidin-4-yl)amino)-4-isopropyl-2,7-naphthyridine-1-carboxamide

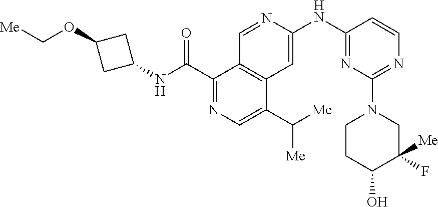 [SMILES: CCO[C@H]1C[C@H](NC(=O)c2ncc(C(C)C)c3cc(Nc4ccnc(N5CC[C@@H](O)[C@@](C)(F)C5)n4)ncc23)C1]

The title compound was obtained as a white solid, 94.7 mg, 55.8% yield, from 6-chloro-N-((1r,3r)-3-ethoxycyclobutyl)-4-isopropyl-2,7-naphthyridine-1-carboxamide (Preparation 105) and (3S,4R)-1-(4-aminopyrimidin-2-yl)-3-fluoro-3-methylpiperidin-4-ol (Preparation 8) following the procedure described in Example 83. LCMS m/z=538 [M+H]⁺; 1H-NMR (400 MHz, DMSO-d₆) δ 10.42 (s, 1H), 10.07 (d, 1H), 9.19 (d, 1H), 8.75 (s, 1H), 8.54 (s, 1H), 8.07 (d, 1H), 6.48 (d, 1H), 5.07 (d, 1H), 4.84-4.60 (m, 2H), 4.54 (q, 1H), 4.13 (td, 1H), 3.58 (tq, 2H), 3.29-3.05 (m, 2H), 2.42-2.22 (m, 4H), 1.83-1.69 (m, 2H), 1.48-1.30 (m, 9H), 1.14 (t, 3H).

### Example 85

### 6-((2-((3S,4R)-3-fluoro-4-hydroxy-3-methylpiperidin-1-yl)pyrimidin-4-yl)amino)-N-((3S,4R)-4-fluoropyrrolidin-3-yl)-4-isopropyl-2,7-naphthyridine-1-carboxamide trifluoroacetate and 6-((2-((3S,4R)-3-fluoro-4-hydroxy-3-methylpiperidin-1-yl)pyrimidin-4-yl)amino)-N-((3R,4S)-4-fluoropyrrolidin-3-yl)-4-isopropyl-2,7-naphthyridine-1-carboxamide trifluoroacetate

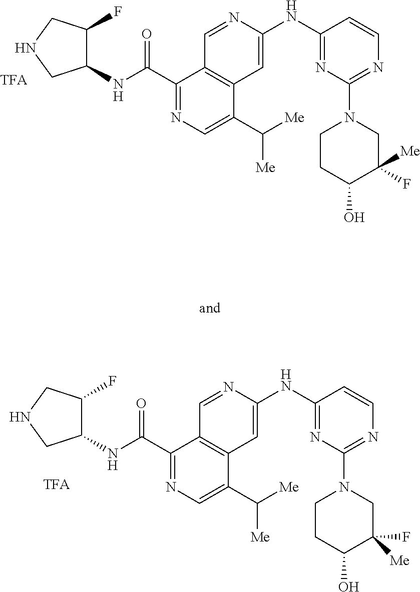 [SMILES: CC(C)c1cnc(C(=O)N[C@H]2CNC[C@H]2F)c2cnc(Nc3ccnc(N4CC[C@@H](O)[C@@](C)(F)C4)n3)cc12 ; CC(C)c1cnc(C(=O)N[C@@H]2CNC[C@@H]2F)c2cnc(Nc3ccnc(N4CC[C@@H](O)[C@@](C)(F)C4)n3)cc12]

A solution of the compound of Preparation 109 (50 mg, 0.08 mmol) and TFA (2 mL) in DCM (2 mL) was stirred for 12 h at rt. The resulting mixture was evaporated under reduced pressure to afford the title compound, 25 mg, 59.6% as yellow oil. LCMS m/z=527 [M+H]⁺

### Example 86

### 6-((2-((3S,4R)-3-fluoro-4-hydroxy-3-methylpiperidin-1-yl)pyrimidin-4-yl)amino)-N-((3R,4R)-4-fluoropyrrolidin-3-yl)-4-isopropyl-2,7-naphthyridine-1-carboxamide trifluoroacetate and 6-((2-((3S,4R)-3-fluoro-4-hydroxy-3-methylpiperidin-1-yl)pyrimidin-4-yl)amino)-N-((3S,4S)-4-fluoropyrrolidin-3-yl)-4-isopropyl-2,7-naphthyridine-1-carboxamide trifluoroacetate

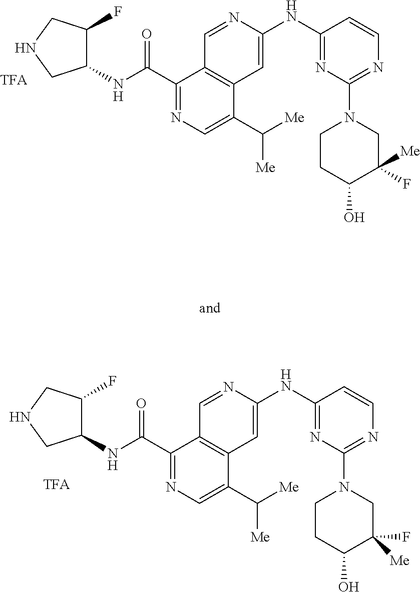 [SMILES: CC(C)c1cnc(C(=O)N[C@@H]2CNC[C@H]2F)c2cnc(Nc3ccnc(N4CC[C@@H](O)[C@@](C)(F)C4)n3)cc12 ; CC(C)c1cnc(C(=O)N[C@H]2CNC[C@@H]2F)c2cnc(Nc3ccnc(N4CC[C@@H](O)[C@@](C)(F)C4)n3)cc12]

The title compound was obtained as a yellow oil, from the compound of Preparation 110, following the procedure described in Example 85. LCMS m/z=527 [M+H]⁺

### Examples 87 and 88

### N-((3S,4R)-4-fluoro-1-methylpyrrolidin-3-yl)-6-((2-((3S,4R)-3-fluoro-4-hydroxy-3-methylpiperidin-1-yl)pyrimidin-4-yl)amino)-4-isopropyl-2,7-naphthyridine-1-carboxamide and N-((3R,4S)-4-fluoro-1-methylpyrrolidin-3-yl)-6-((2-((3S,4R)-3-fluoro-4-hydroxy-3-methylpiperidin-1-yl)pyrimidin-4-yl)amino)-4-isopropyl-2,7-naphthyridine-1-carboxamide

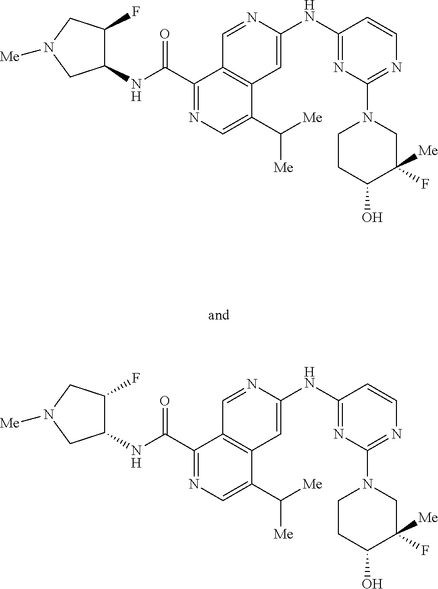 [SMILES: CC(C)c1cnc(C(=O)N[C@H]2CN(C)C[C@H]2F)c2cnc(Nc3ccnc(N4CC[C@@H](O)[C@@](C)(F)C4)n3)cc12 ; CC(C)c1cnc(C(=O)N[C@@H]2CN(C)C[C@@H]2F)c2cnc(Nc3ccnc(N4CC[C@@H](O)[C@@](C)(F)C4)n3)cc12]

A mixture of the compound of Example 85 (100 mg, 0.19 mmol), AcOH (10 mg, 0.167 mmol), HCHO (6 mg, 0.136 mmol) and NaBH₃CN (23 mg, 0.366 mmol) in MeOH (1 mL) was stirred for 1 h at rt. The reaction was quenched by the addition of water (1 mL), the resulting solids were filtered off and the filtrate concentrated in vacuo. The residue was purified by silica gel column eluting with DCM/MeOH (10:1). The resulting product was further purified by Chiral SFC-2 to afford the title compounds.

Peak 1; 10 mg, 9.7% yield. LCMS m/z=541 [M+H]⁺; ¹H-NMR (400 MHz, DMSOd₆) δ: 10.41 (s, 1H), 10.22 (s, 1H), 8.81 (d, 1H), 8.75 (s, 1H), 8.57 (s, 1H), 8.07 (d, 1H), 6.48 (d, 1H), 5.25 (d, 1H), 5.05 (d, 1H), 4.79-4.36 (m, 3H), 3.69-3.48 (m, 2H), 3.09 (d, 3H), 3.23-2.58 (m, 6H), 2.32 (s, 3H), 1.87-1.69 (m, 2H), 1.49-1.24 (m, 9H).

Peak 2; 10 mg, 9.7% yield. LCMS m/z=541 [M+H]⁺; ¹H-NMR (400 MHz, DMSO-d₆) δ ppm: 10.41 (s, 1H), 10.22 (s, 1H), 8.81 (d, 1H), 8.75 (s, 1H), 8.57 (s, 1H), 8.07 (d, 1H), 6.48 (d, 1H), 5.25 (d, 1H), 5.05 (d, 1H), 4.88-4.40 (m, 2H), 3.58 (dt, 2H), 3.22-2.58 (m, 6H), 2.32 (s, 3H), 1.83-1.67 (m, 2H), 1.48-1.30 (m, 9H).

### Examples 89 and 90

### N-((3R,4R)-4-fluoro-1-methylpyrrolidin-3-yl)-6-((2-((3S,4R)-3-fluoro-4-hydroxy-3-methylpiperidin-1-yl)pyrimidin-4-yl)amino)-4-isopropyl-2,7-naphthyridine-1-carboxamide and N-((3S,4S)-4-fluoro-1-methylpyrrolidin-3-yl)-6-((2-((3S,4R)-3-fluoro-4-hydroxy-3-methylpiperidin-1-yl)pyrimidin-4-yl)amino)-4-isopropyl-2,7-naphthyridine-1-carboxamide

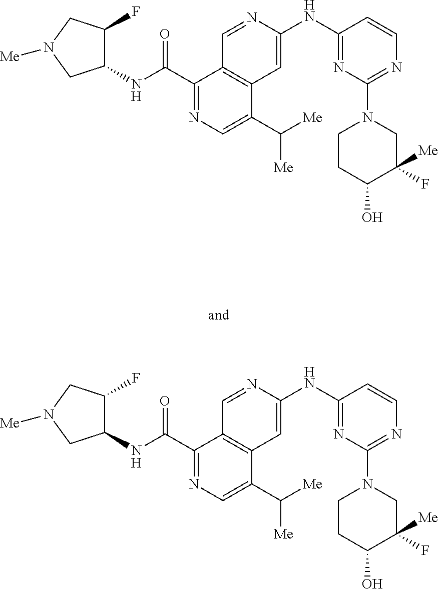 [SMILES: CC(C)c1cnc(C(=O)N[C@@H]2CN(C)C[C@H]2F)c2cnc(Nc3ccnc(N4CC[C@@H](O)[C@@](C)(F)C4)n3)cc12 ; CC(C)c1cnc(C(=O)N[C@H]2CN(C)C[C@@H]2F)c2cnc(Nc3ccnc(N4CC[C@@H](O)[C@@](C)(F)C4)n3)cc12]

The title compounds were obtained from the compound of Example 86 following the procedure described in Example 87 and 88 above. The product was further purified by chiral SFC-2 to afford the title compounds.

Peak 1; 13 mg. LCMS m/z=541 [M+H]⁺; ¹H-NMR (400 MHz, DMSO-d₆) δ: 10.42 (s, 1H), 10.18 (s, 1H), 9.16 (d, 1H), 8.75 (s, 1H), 8.54 (s, 1H), 8.07 (d, 1H), 6.48 (d, 1H), 5.29-5.08 (m, 1H), 5.05 (d, 1H), 4.84-4.30 (m, 3H), 3.67-3.44 (m, 2H), 3.26-3.04 (m, 3H), 2.97-2.62 (m, 3H), 2.29 (s, 3H), 1.81-1.70 (m, 2H), 1.50-1.23 (m, 9H).

Peak 2; LCMS m/z=541 [M+H]⁺; 1H-NMR (400 MHz, DMSO-d₆) δ ppm 10.42 (s, 1H), 10.18 (s, 1H), 9.16 (d, 1H), 8.75 (s, 1H), 8.54 (s, 1H), 8.07 (d, 1H), 6.48 (d, 1H), 5.17 (d, 1H), 5.05 (d, 1H), 4.82-4.39 (m, 3H), 3.56 (dd, 2H), 3.26-3.06 (m, 3H), 2.97-2.61 (m, 3H), 2.29 (s, 3H), 1.82-1.68 (m, 2H), 1.48-1.32 (m, 9H).

### Example 91

### (3S,4R)-3-fluoro-1-(4-((5-isopropyl-8-(methylamino)isoquinolin-3-yl)amino)pyrimidin-2-yl)-3-methylpiperidin-4-ol

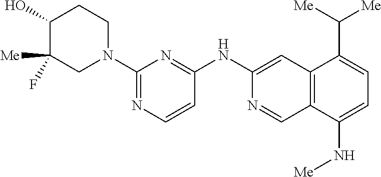 [SMILES: CNc1ccc(C(C)C)c2cc(Nc3ccnc(N4CC[C@@H](O)[C@@](C)(F)C4)n3)ncc12]

TFA (2 mL) was added to a solution of the compound of Preparation 58 (50 mg, 0.0954 mmol) in DCM (6 mL) at rt. The resulting mixture was stirred at rt for 1 h and then evaporated to dryness in vacuo. The residue was purified by HPLC-14 to afford the title compound as an off-white solid (12 mg). LCMS m/z=425 [M+H]⁺; ¹H-NMR (300 MHz, DMSO-d₆) δ: 9.83 (s, 1H), 9.27 (s, 1H), 8.57 (s, 1H), 7.98 (d, 1H), 7.38 (d, 1H), 6.66 (d, 1H), 6.43 (d, 1H), 6.34 (d, 1H), 5.04 (d, 1H), 4.73 (q, 2H), 3.51 (s, 2H), 3.13 (d, 2H), 2.84 (d, 3H), 1.74 (s, 2H), 1.41-1.24 (m, 9H).

### Example 92

### (3S,4R)-3-fluoro-1-(4-((5-isopropyl-8-(methylamino)-2,7-naphthyridin-3-yl)amino)pyrimidin-2-yl)piperidin-4-ol

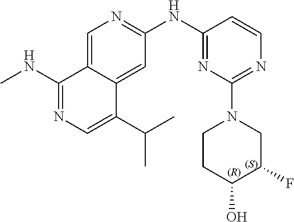

The title compound was obtained by the following procedure.

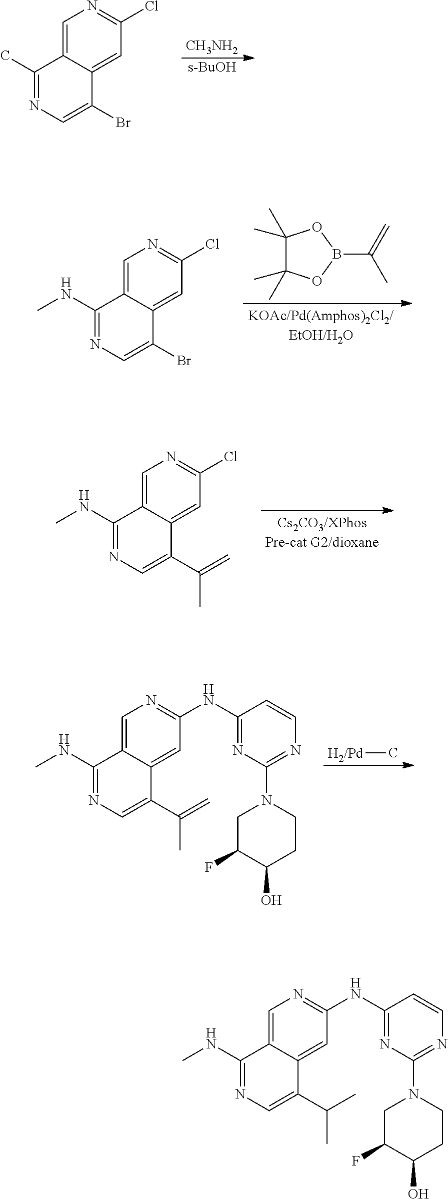

Step 1: 4-bromo-6-chloro-N-methyl-2,7-naphthyridin-1-amine. To a mixture of 4-bromo-1,6-dichloro-2,7-naphthyridine (0.20 g, 719.60 umol, 1.00 eq) and methanamine (72.88 mg, 1.08 mmol, 1.50 eq, HCl) in s-BuOH (5.00 mL) was added Et₃N (218.45 mg, 2.16 mmol, 300.48 uL, 3.00 eq) at 25° C., the reaction mixture was stirred at 110° C. for 16 hrs under N2. LCMS showed starting material was consumed and the desired ms of the product was detected. The reaction mixture was concentrated and residue was purified by acidic prep-HPLC (TFA) to yield the title compound (0.15 g, 388.05 umol, yield: 54%, TFA) was obtained as a yellow solid. LC-MS: (ES, m/z)=271 [M+1]

Step 2: 6-chloro-N-methyl-4-(prop-1-en-2-yl)-2,7-naphthyridin-1-amine. To a mixture of 4-bromo-6-chloro-N-methyl-2,7-naphthyridin-1-amine (0.07 g, 181.09 umol, 1.00 eq, TFA) and 2-isopropenyl-4,4,5,5-tetramethyl-1,3,2-dioxaborolane (30.43 mg, 181.09 umol, 1.00 eq) in EtOH (10.00 mL) and H₂O (1.50 mL) was added KOAc (35.54 mg, 362.18 umol, 2.00 eq) and Pd(Amphos)₂Cl₂ (12.82 mg, 18.11 umol, 12.82 uL, 0.10 eq) at 25° C., the reaction mixture was stirred at 80° C. for 16 hrs under N₂. LCMS showed starting material was consumed and the desired ms of the product was detected. The reaction mixture was concentrated. The residue was purified by acidic prep-HPLC (TFA) to yield the title compound (0.06 g, 172.55 umol, yield: 47%, TFA) as a yellow solid. LC-MS: (ES, m/z)=234 [M+1]

Step 3: (3S,4R)-3-fluoro-1-(4-((8-(methylamino)-5-(prop-1-en-2-yl)-2,7-naphthyridin-3-yl)amino)pyrimidin-2-yl)piperidin-4-ol. To a mixture of 6-chloro-N-methyl-4-(prop-1-en-2-yl)-2,7-naphthyridin-1-amine (0.06 g, 172.55 umol, 1.00 eq, TFA) and (3S,4R)-1-(4-aminopyrimidin-2-yl)-3-fluoropiperidin-4-ol (36.62 mg, 172.55 umol, 1.00 eq) in dioxane (5.00 mL) was added Cs₂CO₃ (112.44 mg, 345.11 umol, 2.00 eq) and XPhos-Pd-G2 (13.58 mg, 17.26 umol, 0.10 eq) at 25° C., the reaction mixture was stirred at 120° C. for 16 hrs under N₂. TLC (PE:EtOAc=0:1) showed reaction was completed, the reaction mixture was concentrated and purified by prep-TLC (PE:EtOAc=0:1, Rf=0.4) to give the title compound (0.05 g, 122.11 umol, yield: 70.77%) as a brown oil. LC-MS: (ES, m/z)=410 [M+1]

Step 4: (3S,4R)-3-fluoro-1-(4-((5-isopropyl-8-(methylamino)-2,7-naphthyridin-3-yl)amino)pyrimidin-2-yl)piperidin-4-ol. To a mixture of (3S,4R)-3-fluoro-1-(4-((8-(methylamino)-5-(prop-1-en-2-yl)-2,7-naphthyridin-3-yl)amino)pyrimidin-2-yl)piperidin-4-ol (0.05 g, 122.11 umol, 1.00 eq) in MeOH (5.00 mL) was added 10% Pd/C (0.05 g, 48.84 umol, 50% purity) at 25° C., the reaction mixture was stirred at 25° C. for 16 hrs under H₂ (15 psi). Once reaction determined complete by LCMS the reaction mixture was concentrated and purified by basic prep-HPLC (column: Waters Xbridge Prep OBD C18 150*30 10 u; mobile phase: [water (0.04% NH₃H₂O)-ACN]; B %: 25%-55%, 10 min) to yield the title compound (4.80 mg, 11.49 umol, yield: 9%) as a brown solid. LC-MS: (ES, m/z)=412 [M+1];

¹H-NMR (400 MHz, CD₃OD) δ ppm 9.13 (s, 1H), 8.52 (s, 1H), 7.98 (d, 1H, J=6.0 Hz), 7.81 (s, 1H), 6.38 (d, 1H, J=5.6 Hz), 4.78-4.77 (m, 1H), 4.65-4.60 (m, 2H), 4.40-4.36 (m, 1H), 3.99-3.94 (m, 1H), 3.70-3.60 (m, 1H), 3.48-3.45 (m, 1H), 3.39-3.34 (m, 1H), 3.03 (s, 3H), 1.93-1.82 (m, 2H), 1.36 (dd, 1H, J=6.4, 5.2 Hz).

LC-MS (mobile phase: from 95% [water+10 mM NH₄HCO₃] and 5% CH₃CN, under this condition for 0.4 min, then changed to 10% [water+10 mM NH₄HCO₃] and 90% CH₃CN in 2.6 min, then changed to 100% CH₃CN in 0.85 min, finally changed to 95% [water+10 mM NH₄HCO₃] and 5% CH₃CN in 0.01 min, then under this condition for 0.64 min. The flow is 0.8 mL·min⁻¹ all along.) Purity is 98.463%, Rt=2.560, MS Calcd.: 411.5, MS Found: 412.2 ([M+1]⁺).

### Example 93

### (3S,4R)-3-fluoro-1-(4-((5-isopropyl-8-(methylamino)-2,7-naphthyridin-3-yl)amino)pyrimidin-2-yl)-3-methylpiperidin-4-ol

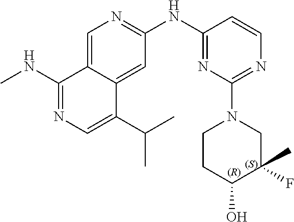

The title compound was obtained by the following procedure.

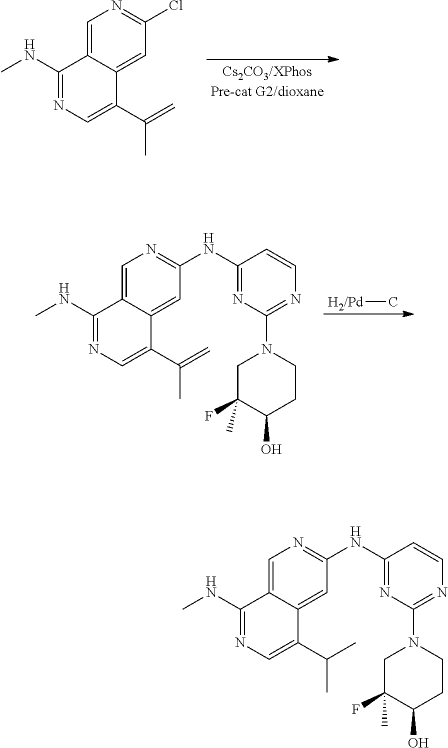

Step 1: (3S,4R)-3-fluoro-3-methyl-1-(4-((8-(methylamino)-5-(prop-1-en-2-yl)-2,7-naphthyridin-3-yl)amino)pyrimidin-2-yl)piperidin-4-ol: To a mixture of 6-chloro-4-isopropenyl-N-methyl-2,7-naphthyridin-1-amine (45 mg, 192.56 umol, 1 eq) and (3S,4R)-1-(4-aminopyrimidin-2-yl)-3-fluoro-3-methylpiperidin-4-ol (43.57 mg, 192.56 umol, 1 eq) in dioxane (5 mL) was added Cs₂CO₃ (125.48 mg, 385.12 umol, 2 eq) and XPHOS-PD-G2 (15.15 mg, 19.26 umol, 0.1 eq) in one portion under N₂. The mixture was stirred at 120° C. for 16 hours. LCMS showed the reaction was complete. The mixture was filtered and the filtrate was concentrated. The crude product was purified by prep-TLC (Petroleum ether/Ethyl acetate=0/1) to give the title compound (70 mg, crude) as a yellow oil.

Step 2: (3S,4R)-3-fluoro-1-(4-((5-isopropyl-8-(methylamino)-2,7-naphthyridin-3-yl)amino)pyrimidin-2-yl)-3-methylpiperidin-4-ol: To a solution of (3S,4R)-3-fluoro-3-methyl-1-(4-((8-(methylamino)-5-(prop-1-en-2-yl)-2,7-naphthyridin-3-yl)amino)pyrimidin-2-yl)piperidin-4-ol (70 mg, 165.29 umol, 1 eq) in MeOH (10 mL) was added Pd/C (30 mg, 10% purity). The suspension was degassed under vacuum and purged with H₂ several times. The mixture was stirred under H₂ (15 psi) at 25° C. for 16 hours. LCMS showed the reaction was complete. The mixture was filtered and the filtrate was concentrated. The crude product was purified by prep-HPLC (column: Waters Xbridge 150*25 5 u; mobile phase: [water (0.04% NH₃ H₂O+10 mM NH₄HCO₃)-ACN]; B %: 10%-40%, 10 min) to give the title compound (8.7 mg, 20.45 umol, 12.37% yield, 100% purity) as a gray solid.

¹H NMR (400 MHz, CD₃OD) δ ppm 9.19 (s, 1H), 8.51 (s, 1H), 8.01 (d, 1H, J=6.0 Hz), 7.79 (s, 1H), 6.44 (d, 1H, J=6.0 Hz), 4.76-4.68 (m, 2H), 3.73-3.68 (m, 1H), 3.42-3.37 (m, 1H), 3.28-3.21 (m, 2H), 3.07 (s, 3H), 1.95-1.86 (m, 2H), 1.47 (d, 3H, J=20.8 Hz), 1.39 (d, 6H, J=6.0 Hz).

LC-MS (mobile phase: from 85% [water+10 mM NH₄HCO₃] and 15% CH₃CN, under this condition for 0.4 min, then changed to 100% CH₃CN in 2.6 min, finally changed to 85% [water+10 mM NH₄HCO₃] and 15% CH₃CN in 0.66 min, then under this condition for 0.84 min. The flow is 0.8 mL·min⁻¹ all along.) Purity is 100.000%, Rt=2.347, MS Calcd.: 425.5, MS Found: 426.2 ([M+1]⁺).

### Example 94

### (3S,4R)-1-(4-((8-(dimethylamino)-5-isopropyl-2,7-naphthyridin-3-yl)amino)pyrimidin-2-yl)-3-fluoropiperidin-4-ol

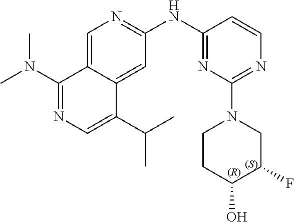

The title compound was obtained by the following procedure.

To a mixture of 6-chloro-N,N-dimethyl-4-(propan-2-yl)-2,7-naphthyridin-1-amine (95.1 mg, 380 μmol), (3S,4R)-1-(4-aminopyrimidin-2-yl)-3-fluoropiperidin-4-ol (80.6 mg, 380 μmol), Brettphos Pd G3 (34.3 mg, 38.0 μmol), Cs₂CO₃ (371 mg, 1.14 mmol) dissolved in 1,4-Dioxane (20 mL) was heated at 100° C. for 3 h under nitrogen. The reaction was quenched with water and extracted with ethyl acetate. The organic layers were combined and concentrated under vacuum. The residue was further purified by Prep-HPLC as following conditions: Column: XBridge Prep C18 OBD Column, 5 um, 19*150 mm; Mobile Phase A: Water (10 mmol/L NH₄HCO₃+0.1% NH₃·H₂O), Mobile Phase B: ACN; Flow rate: 20 mL/min; Gradient: 37% B to 40% B in 7 min; 254,220 nm. This resulted in the title product (55.6 mg) as a white solid.

¹H NMR (300 MHz, DMSO-d6) δ 10.12 (s, 1H), 9.19 (s, 1H), 8.54 (s, 1H), 8.08-7.98 (m, 2H), 6.47 (d, J=5.6 Hz, 1H), 5.13 (d, J=5.1 Hz, 1H), 4.82-4.52 (m, 2H), 4.35 (d, J=13.0 Hz, 1H), 4.00-3.80 (m, 1H), 3.70-3.50 (m, 1H), 3.50-3.30 (m, 2H), 3.10 (s, 6H), 1.81-1.61 (m, 3H), 1.32 (dd, J=6.8, 3.4 Hz, 6H).

### Example 95

### (3S,4R)-3-fluoro-1-(4-((1-isopropyl-4-(methylamino)pyrido[3,4-d]pyridazin-7-yl)aminopyrimidin-2-yl)piperidin-4-ol

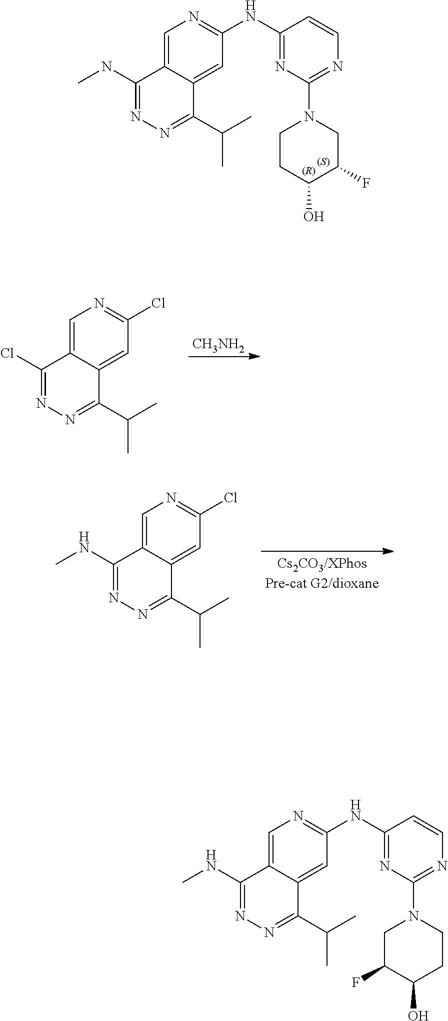

Step 1: 7-chloro-1-isopropyl-N-methylpyrido[3,4-d]pyridazin-4-amine. A mixture of 4,7-dichloro-1-isopropylpyrido[3,4-d]pyridazine (34.00 mg, 140.44 umol, 1.00 eq), methanamine hydrochloride (9.48 mg, 140.44 umol, 1.00 eq) and Et₃N (28.42 mg, 280.87 umol, 39.09 uL, 2.00 eq) in sec-butyl alcohol (1.00 mL) was stirred at 90° C. for 16 hrs. Once reaction complete the mixture was concentrated under reduced pressure and purified by acidic prep-HPLC (TFA) to give the title compound (20.00 mg, 57.03 umol, yield: 40.61%, TFA) as a light brown oil. LC-MS: (ES, m/z)=237 [M+1].

Step 2: (3S,4R)-3-fluoro-1-(4-((1-isopropyl-4-(methylamino)pyrido[3,4-d]pyridazin-7-yl)amino)pyrimidin-2-yl)piperidin-4-ol. A mixture of 7-chloro-1-isopropyl-N-methylpyrido[3,4-d]pyridazin-4-amine (20.00 mg, 57.03 umol, 1.00 eq, TFA), (3S,4R)-1-(4-aminopyrimidin-2-yl)-3-fluoro-piperidin-4-ol (14.52 mg, 68.43 umol, 1.20 eq), Cs₂CO₃ (37.16 mg, 114.05 umol, 2.00 eq) and XPhos-Pd-G2 (4.49 mg, 5.70 umol, 0.10 eq) in dioxane (2.00 mL) was stirred at 120° C. for 16 hrs under N₂. The next day the reaction was filtered and concentrated to give the crude product. The crude product was purified by acidic prep-HPLC (column: Nano-micro Kromasil C18 100*30 mm 5 um; mobile phase: [water (0.225% FA)-ACN]; B %: 1%-30%, 15 min) to yield the title compound (12.00 mg, 25.34 umol, yield: 44%) obtained as a yellow gum. LC-MS: (ES, m/z)=413 [M+1]; ¹H-NMR (400 MHz, CD3OD) δ ppm 9.31 (s, 1H), 8.68 (s, 1H), 8.06 (d, 1H, J=5.6 Hz), 6.44 (d, 1H, J=5.6 Hz), 4.80-4.67 (m, 1H), 4.61-4.57 (m, 1H), 4.35 (d, 1H, J=13.2 Hz), 4.08-3.94 (m, 1H), 3.71-3.58 (m, 2H), 3.47 (t, 1H, J=9.6 Hz), 3.16 (s, 3H), 1.94-1.82 (m, 2H), 1.42 (t, 6H, J=6.4 Hz).

### Table of Compounds prepared by the synthetic methods disclosed above

TABLE 1

| Example | Structure |
|---|---|
| 1 | 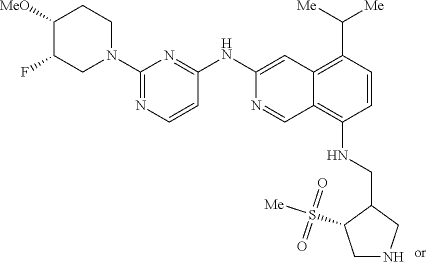 [SMILES: CO[C@@H]1CCN(c2nccc(Nc3cc4c(C(C)C)ccc(NCC5CNC[C@@H]5S(C)(=O)=O)c4cn3)n2)C[C@@H]1F] |
| | 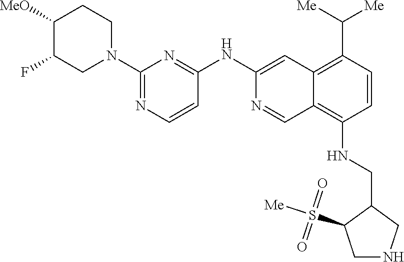 [SMILES: CO[C@@H]1CCN(c2nccc(Nc3cc4c(C(C)C)ccc(NCC5CNC[C@H]5S(C)(=O)=O)c4cn3)n2)C[C@@H]1F] |
| 2 | 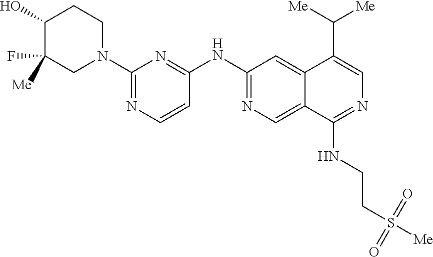 [SMILES: CC(C)c1cnc(NCCS(C)(=O)=O)c2cnc(Nc3ccnc(N4CC[C@@H](O)[C@@](C)(F)C4)n3)cc12] |
| 3 | 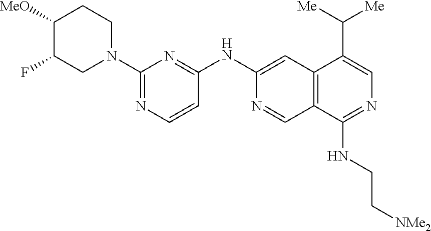 [SMILES: CO[C@@H]1CCN(c2nccc(Nc3cc4c(C(C)C)cnc(NCCN(C)C)c4cn3)n2)C[C@@H]1F] |
| 4 | 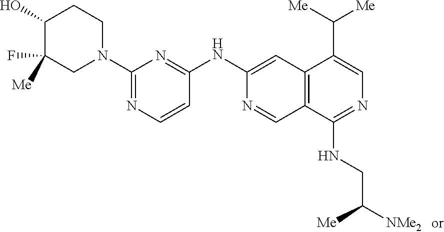 [SMILES: CC(C)c1cnc(NC[C@H](C)N(C)C)c2cnc(Nc3ccnc(N4CC[C@@H](O)[C@@](C)(F)C4)n3)cc12] |
| | 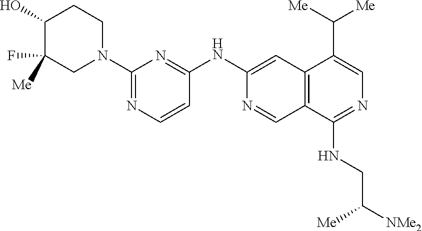 [SMILES: CC(C)c1cnc(NC[C@@H](C)N(C)C)c2cnc(Nc3ccnc(N4CC[C@@H](O)[C@@](C)(F)C4)n3)cc12] |
| 5 | 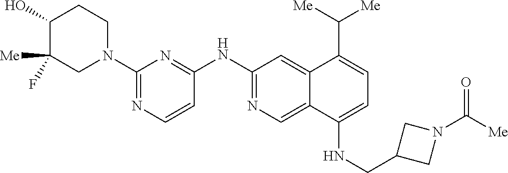 [SMILES: CC(=O)N1CC(CNc2ccc(C(C)C)c3cc(Nc4ccnc(N5CC[C@@H](O)[C@@](C)(F)C5)n4)ncc23)C1] |
| 6 | 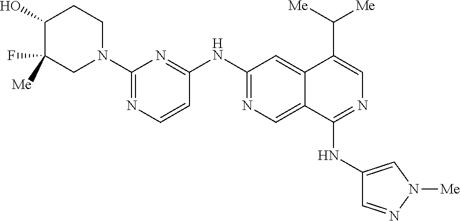 [SMILES: CC(C)c1cnc(Nc2cnn(C)c2)c2cnc(Nc3ccnc(N4CC[C@@H](O)[C@@](C)(F)C4)n3)cc12] |
| 7 | 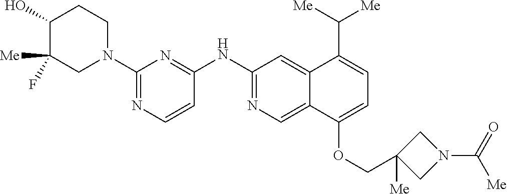 [SMILES: CC(=O)N1CC(C)(COc2ccc(C(C)C)c3cc(Nc4ccnc(N5CC[C@@H](O)[C@@](C)(F)C5)n4)ncc23)C1] |
| 8 | 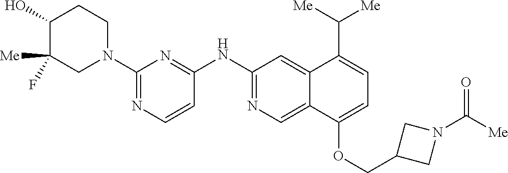 [SMILES: CC(=O)N1CC(COc2ccc(C(C)C)c3cc(Nc4ccnc(N5CC[C@@H](O)[C@@](C)(F)C5)n4)ncc23)C1] |
| 9 | 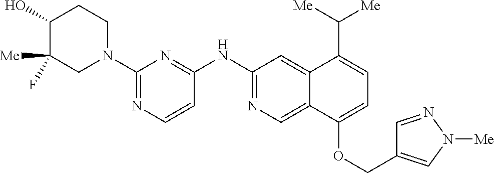 [SMILES: CC(C)c1ccc(OCc2cnn(C)c2)c2cnc(Nc3ccnc(N4CC[C@@H](O)[C@@](C)(F)C4)n3)cc12] |
| 10 | 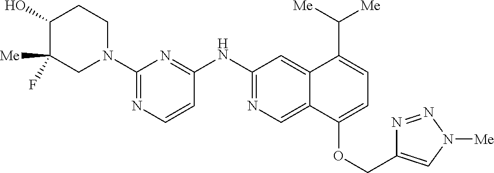 [SMILES: CC(C)c1ccc(OCc2cn(C)nn2)c2cnc(Nc3ccnc(N4CC[C@@H](O)[C@@](C)(F)C4)n3)cc12] |
| 11 | 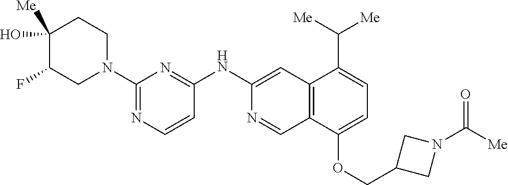 [SMILES: CC(=O)N1CC(COc2ccc(C(C)C)c3cc(Nc4ccnc(N5CC[C@@](C)(O)[C@@H](F)C5)n4)ncc23)C1] |
| 12 | 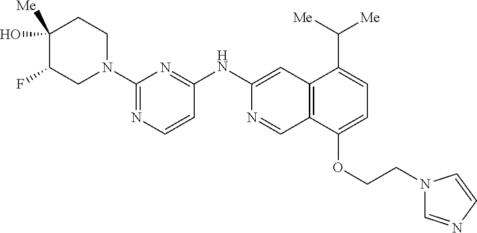 [SMILES: CC(C)c1ccc(OCCn2ccnc2)c2cnc(Nc3ccnc(N4CC[C@@](C)(O)[C@@H](F)C4)n3)cc12] |
| 13 | 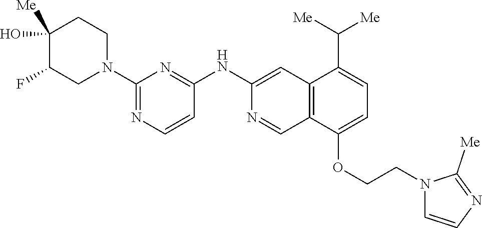 [SMILES: Cc1nccn1CCOc1ccc(C(C)C)c2cc(Nc3ccnc(N4CC[C@@](C)(O)[C@@H](F)C4)n3)ncc12] |
| 14 | 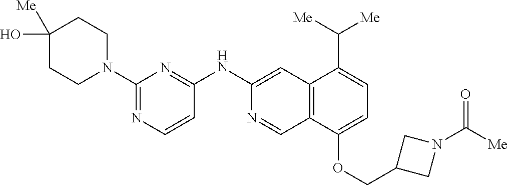 [SMILES: CC(=O)N1CC(COc2ccc(C(C)C)c3cc(Nc4ccnc(N5CCC(C)(O)CC5)n4)ncc23)C1] |
| 15 | 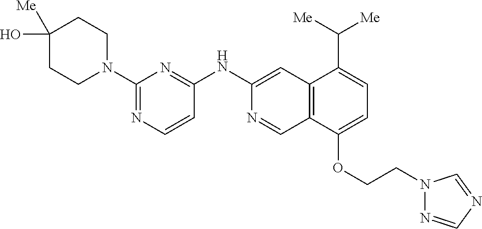 [SMILES: CC(C)c1ccc(OCCn2cncn2)c2cnc(Nc3ccnc(N4CCC(C)(O)CC4)n3)cc12] |
| 16 | 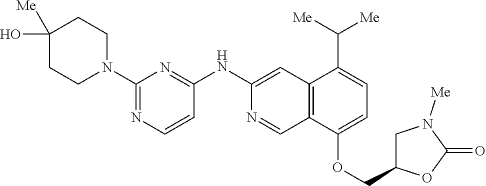 [SMILES: CC(C)c1ccc(OC[C@H]2CN(C)C(=O)O2)c2cnc(Nc3ccnc(N4CCC(C)(O)CC4)n3)cc12] |
| 17 | 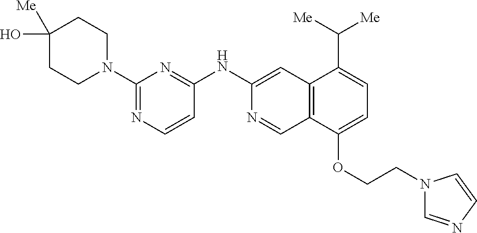 [SMILES: CC(C)c1ccc(OCCn2ccnc2)c2cnc(Nc3ccnc(N4CCC(C)(O)CC4)n3)cc12] |
| 18 | 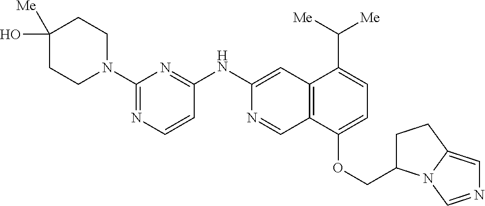 [SMILES: CC(C)c1ccc(OCC2CCc3cncn32)c2cnc(Nc3ccnc(N4CCC(C)(O)CC4)n3)cc12] |
| 19 | 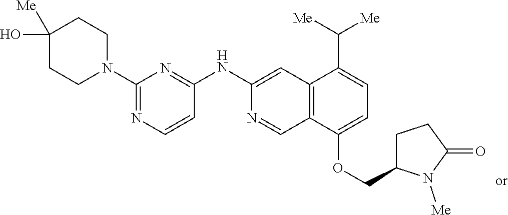 [SMILES: CC(C)c1ccc(OC[C@H]2CCC(=O)N2C)c2cnc(Nc3ccnc(N4CCC(C)(O)CC4)n3)cc12] |
| | 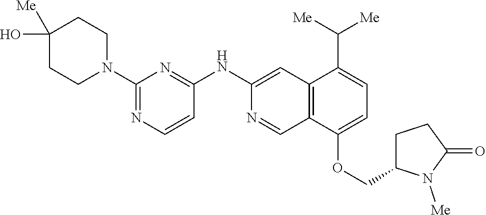 [SMILES: CC(C)c1ccc(OC[C@@H]2CCC(=O)N2C)c2cnc(Nc3ccnc(N4CCC(C)(O)CC4)n3)cc12] |
| 20 | 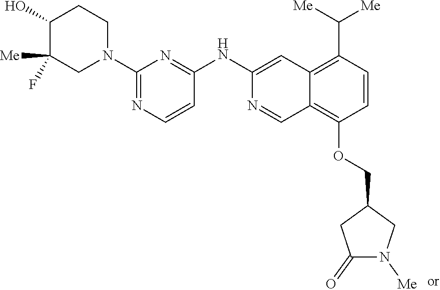 [SMILES: CC(C)c1ccc(OC[C@@H]2CC(=O)N(C)C2)c2cnc(Nc3ccnc(N4CC[C@@H](O)[C@@](C)(F)C4)n3)cc12] |
| | 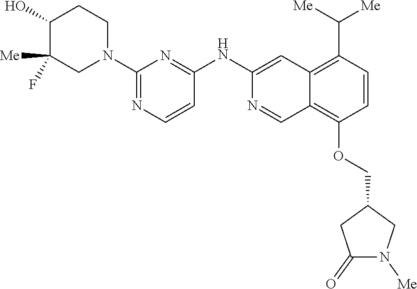 [SMILES: CC(C)c1ccc(OC[C@H]2CC(=O)N(C)C2)c2cnc(Nc3ccnc(N4CC[C@@H](O)[C@@](C)(F)C4)n3)cc12] |
| 21 | 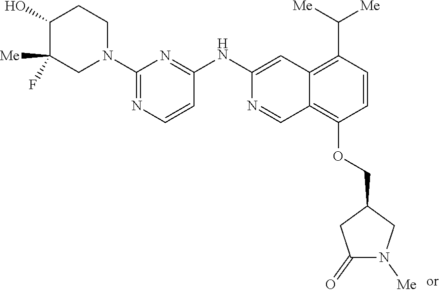 [SMILES: CC(C)c1ccc(OC[C@@H]2CC(=O)N(C)C2)c2cnc(Nc3ccnc(N4CC[C@@H](O)[C@@](C)(F)C4)n3)cc12] |
| | 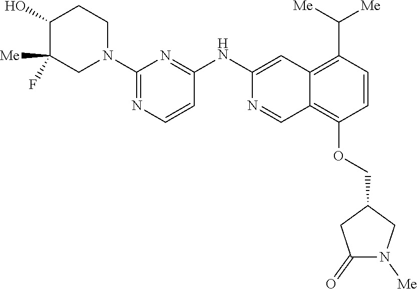 [SMILES: CC(C)c1ccc(OC[C@H]2CC(=O)N(C)C2)c2cnc(Nc3ccnc(N4CC[C@@H](O)[C@@](C)(F)C4)n3)cc12] |
| 22 | 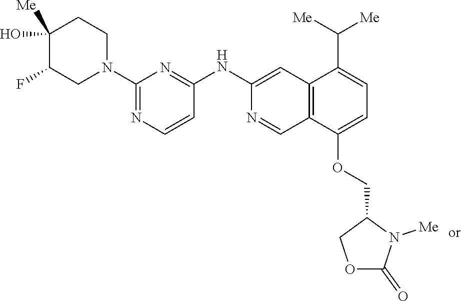 [SMILES: CC(C)c1ccc(OC[C@H]2COC(=O)N2C)c2cnc(Nc3ccnc(N4CC[C@@](C)(O)[C@@H](F)C4)n3)cc12] |
| | 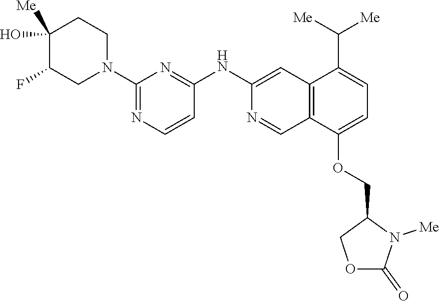 [SMILES: CC(C)c1ccc(OC[C@@H]2COC(=O)N2C)c2cnc(Nc3ccnc(N4CC[C@@](C)(O)[C@@H](F)C4)n3)cc12] |
| 23 | 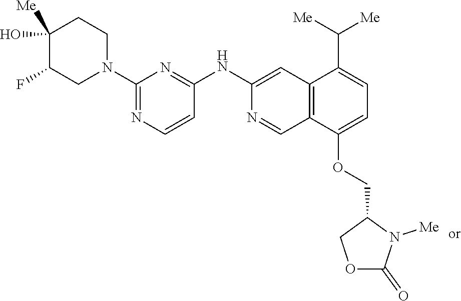 [SMILES: CC(C)c1ccc(OC[C@H]2COC(=O)N2C)c2cnc(Nc3ccnc(N4CC[C@@](C)(O)[C@@H](F)C4)n3)cc12] |
| | 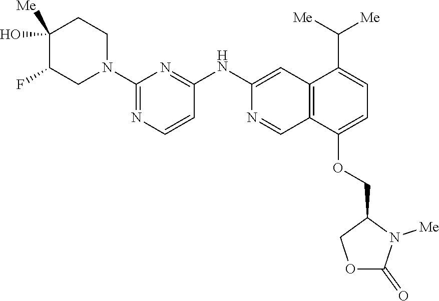 [SMILES: CC(C)c1ccc(OC[C@@H]2COC(=O)N2C)c2cnc(Nc3ccnc(N4CC[C@@](C)(O)[C@@H](F)C4)n3)cc12] |
| 24 | 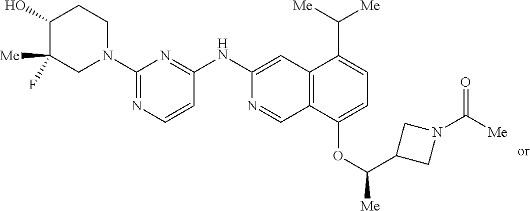 [SMILES: CC(=O)N1CC([C@@H](C)Oc2ccc(C(C)C)c3cc(Nc4ccnc(N5CC[C@@H](O)[C@@](C)(F)C5)n4)ncc23)C1] |
| | 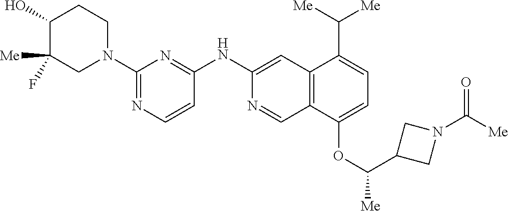 [SMILES: CC(=O)N1CC([C@H](C)Oc2ccc(C(C)C)c3cc(Nc4ccnc(N5CC[C@@H](O)[C@@](C)(F)C5)n4)ncc23)C1] |
| 25 | 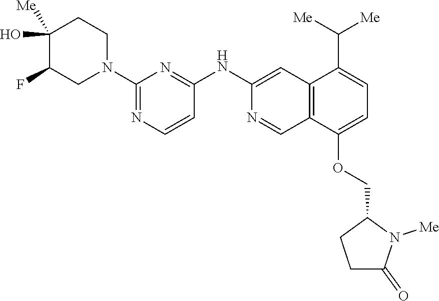 [SMILES: CC(C)c1ccc(OC[C@H]2CCC(=O)N2C)c2cnc(Nc3ccnc(N4CC[C@](C)(O)[C@H](F)C4)n3)cc12] |
| 26 | 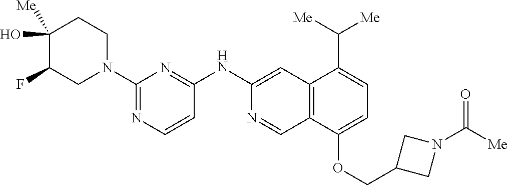 [SMILES: CC(=O)N1CC(COc2ccc(C(C)C)c3cc(Nc4ccnc(N5CC[C@](C)(O)[C@H](F)C5)n4)ncc23)C1] |
| 27 | 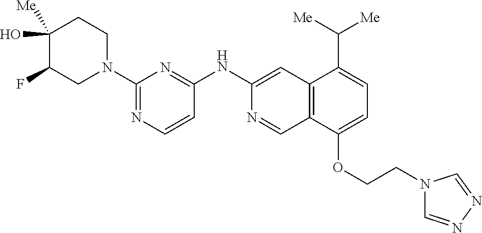 [SMILES: CC(C)c1ccc(OCCn2cnnc2)c2cnc(Nc3ccnc(N4CC[C@](C)(O)[C@H](F)C4)n3)cc12] |
| 28 | 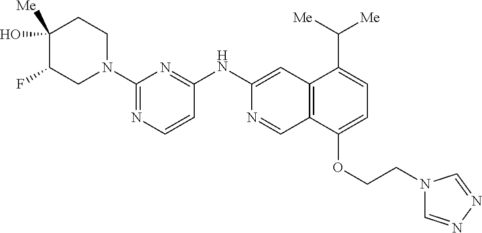 [SMILES: CC(C)c1ccc(OCCn2cnnc2)c2cnc(Nc3ccnc(N4CC[C@@](C)(O)[C@@H](F)C4)n3)cc12] |
| 29 | 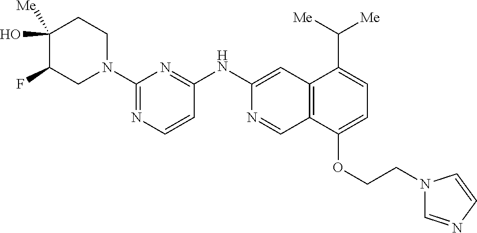 [SMILES: CC(C)c1ccc(OCCn2ccnc2)c2cnc(Nc3ccnc(N4CC[C@](C)(O)[C@H](F)C4)n3)cc12] |
| 30 | 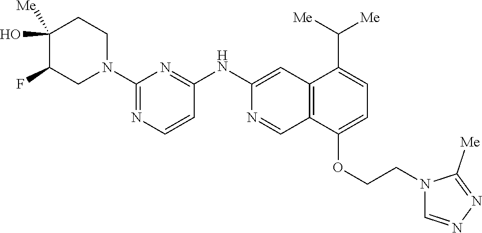 [SMILES: Cc1nncn1CCOc1ccc(C(C)C)c2cc(Nc3ccnc(N4CC[C@](C)(O)[C@H](F)C4)n3)ncc12] |
| 31 | 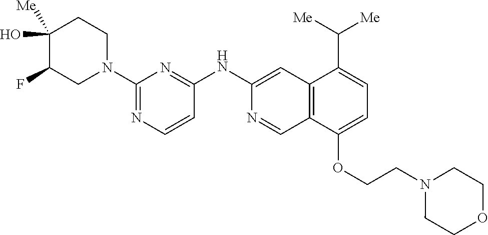 [SMILES: CC(C)c1ccc(OCCN2CCOCC2)c2cnc(Nc3ccnc(N4CC[C@](C)(O)[C@H](F)C4)n3)cc12] |
| 32 | 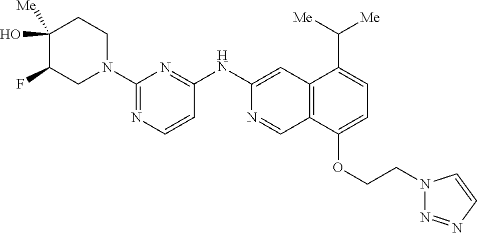 [SMILES: CC(C)c1ccc(OCCn2ccnn2)c2cnc(Nc3ccnc(N4CC[C@](C)(O)[C@H](F)C4)n3)cc12] |
| 33 | 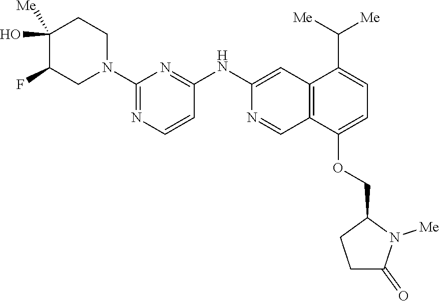 [SMILES: CC(C)c1ccc(OC[C@@H]2CCC(=O)N2C)c2cnc(Nc3ccnc(N4CC[C@](C)(O)[C@H](F)C4)n3)cc12] |
| 34 | 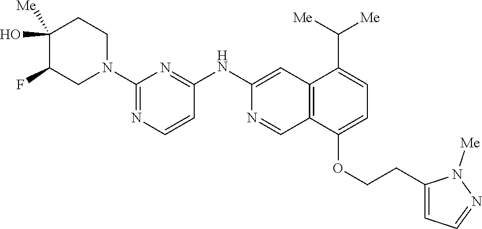 [SMILES: CC(C)c1ccc(OCCc2ccnn2C)c2cnc(Nc3ccnc(N4CC[C@](C)(O)[C@H](F)C4)n3)cc12] |
| 35 | 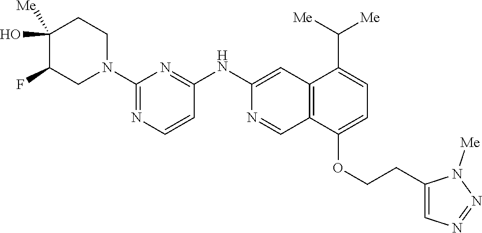 [SMILES: CC(C)c1ccc(OCCc2cnnn2C)c2cnc(Nc3ccnc(N4CC[C@](C)(O)[C@H](F)C4)n3)cc12] |
| 36 | 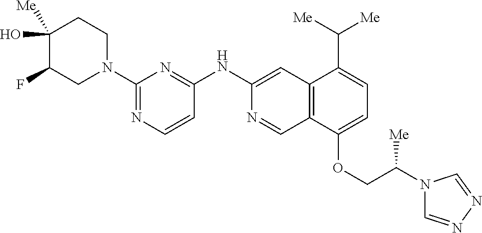 [SMILES: CC(C)c1ccc(OC[C@H](C)n2cnnc2)c2cnc(Nc3ccnc(N4CC[C@](C)(O)[C@H](F)C4)n3)cc12] |
| 37 | 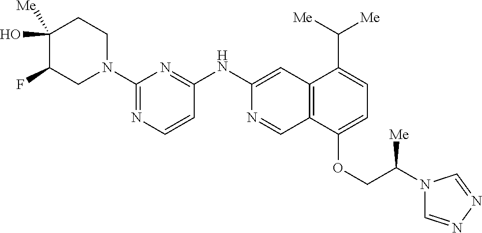 [SMILES: CC(C)c1ccc(OC[C@@H](C)n2cnnc2)c2cnc(Nc3ccnc(N4CC[C@](C)(O)[C@H](F)C4)n3)cc12] |
| 38 | 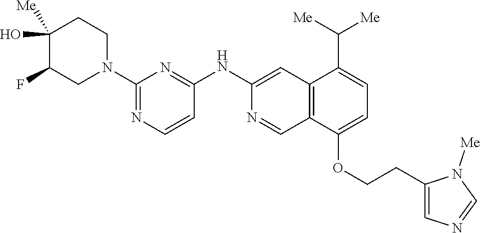 [SMILES: CC(C)c1ccc(OCCc2cncn2C)c2cnc(Nc3ccnc(N4CC[C@](C)(O)[C@H](F)C4)n3)cc12] |
| 39 | 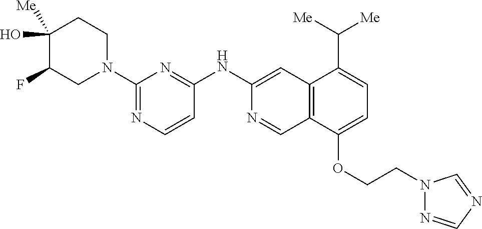 [SMILES: CC(C)c1ccc(OCCn2cncn2)c2cnc(Nc3ccnc(N4CC[C@](C)(O)[C@H](F)C4)n3)cc12] |
| 40 | 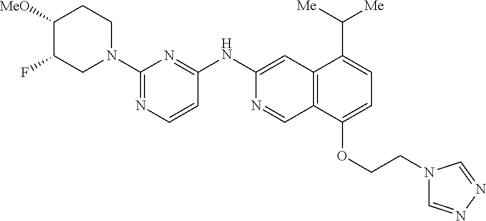 [SMILES: CO[C@@H]1CCN(c2nccc(Nc3cc4c(C(C)C)ccc(OCCn5cnnc5)c4cn3)n2)C[C@@H]1F] |
| 41 | 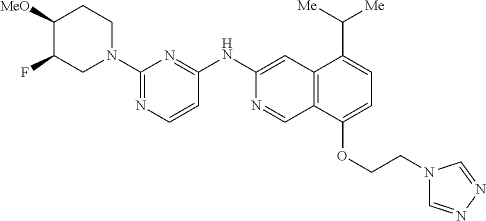 [SMILES: CO[C@H]1CCN(c2nccc(Nc3cc4c(C(C)C)ccc(OCCn5cnnc5)c4cn3)n2)C[C@H]1F] |
| 42 | 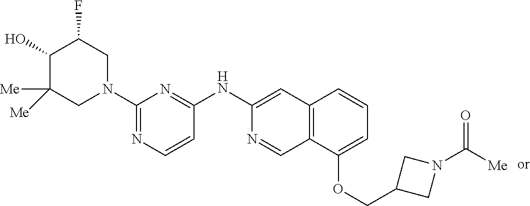 [SMILES: CC(=O)N1CC(COc2cccc3cc(Nc4ccnc(N5C[C@@H](F)[C@@H](O)C(C)(C)C5)n4)ncc23)C1] |
| | 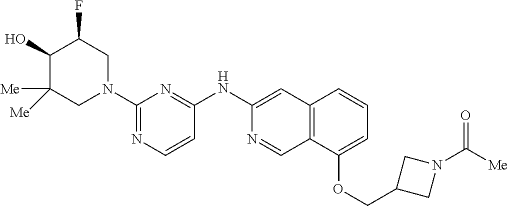 [SMILES: CC(=O)N1CC(COc2cccc3cc(Nc4ccnc(N5C[C@H](F)[C@H](O)C(C)(C)C5)n4)ncc23)C1] |
| 43 | 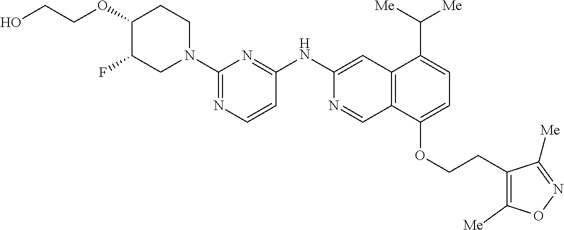 [SMILES: Cc1noc(C)c1CCOc1ccc(C(C)C)c2cc(Nc3ccnc(N4CC[C@@H](OCCO)[C@@H](F)C4)n3)ncc12] |
| 44 | 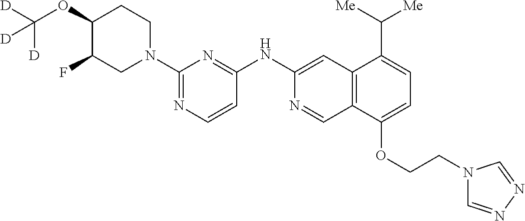 [SMILES: [2H]C([2H])([2H])O[C@H]1CCN(c2nccc(Nc3cc4c(C(C)C)ccc(OCCn5cnnc5)c4cn3)n2)C[C@H]1F] |
| 45 | 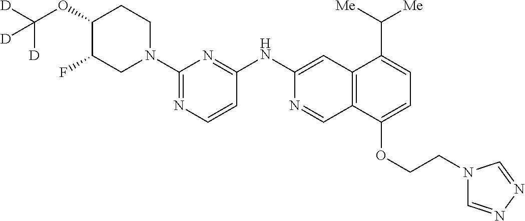 [SMILES: [2H]C([2H])([2H])O[C@@H]1CCN(c2nccc(Nc3cc4c(C(C)C)ccc(OCCn5cnnc5)c4cn3)n2)C[C@@H]1F] |
| 46 | 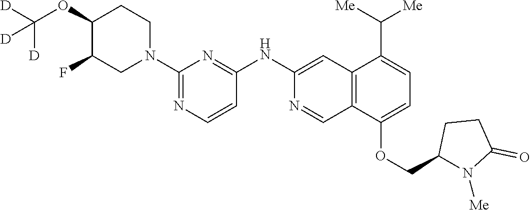 [SMILES: [2H]C([2H])([2H])O[C@H]1CCN(c2nccc(Nc3cc4c(C(C)C)ccc(OC[C@H]5CCC(=O)N5C)c4cn3)n2)C[C@H]1F] |
| 47 | 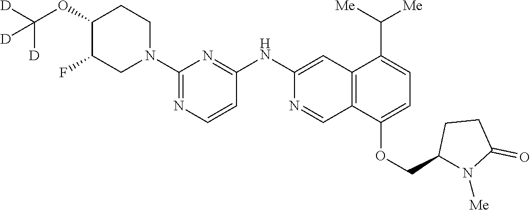 [SMILES: [2H]C([2H])([2H])O[C@@H]1CCN(c2nccc(Nc3cc4c(C(C)C)ccc(OC[C@H]5CCC(=O)N5C)c4cn3)n2)C[C@@H]1F] |
| 48 | 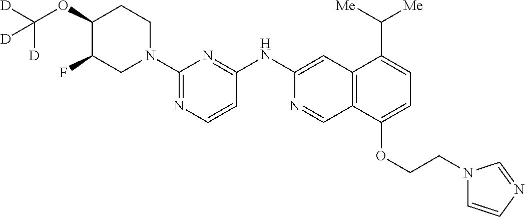 [SMILES: [2H]C([2H])([2H])O[C@H]1CCN(c2nccc(Nc3cc4c(C(C)C)ccc(OCCn5ccnc5)c4cn3)n2)C[C@H]1F] |
| 49 | 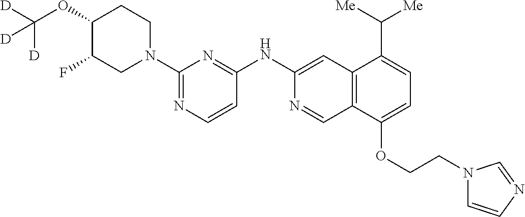 [SMILES: [2H]C([2H])([2H])O[C@@H]1CCN(c2nccc(Nc3cc4c(C(C)C)ccc(OCCn5ccnc5)c4cn3)n2)C[C@@H]1F] |
| 50 | 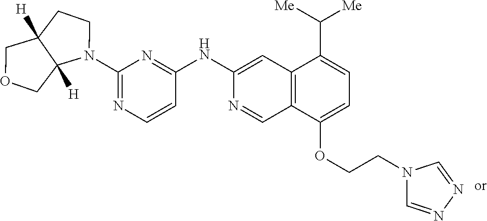 [SMILES: CC(C)c1ccc(OCCn2cnnc2)c2cnc(Nc3ccnc(N4CC[C@H]5COC[C@H]54)n3)cc12] |
| | 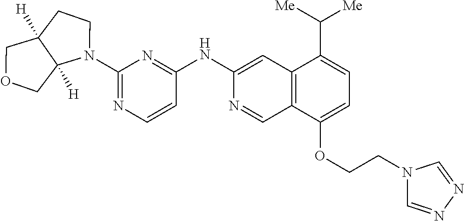 [SMILES: CC(C)c1ccc(OCCn2cnnc2)c2cnc(Nc3ccnc(N4CC[C@@H]5COC[C@@H]54)n3)cc12] |
| 51 | 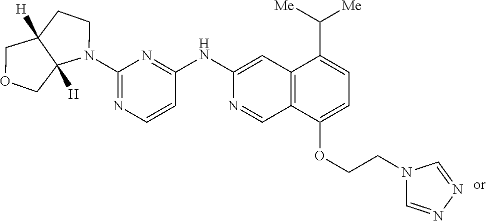 [SMILES: CC(C)c1ccc(OCCn2cnnc2)c2cnc(Nc3ccnc(N4CC[C@H]5COC[C@H]54)n3)cc12] |
| | 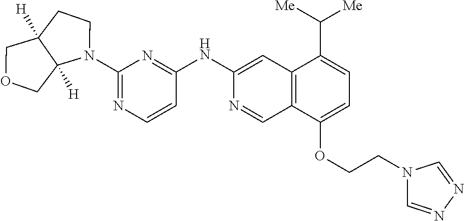 [SMILES: CC(C)c1ccc(OCCn2cnnc2)c2cnc(Nc3ccnc(N4CC[C@@H]5COC[C@@H]54)n3)cc12] |
| 52 | 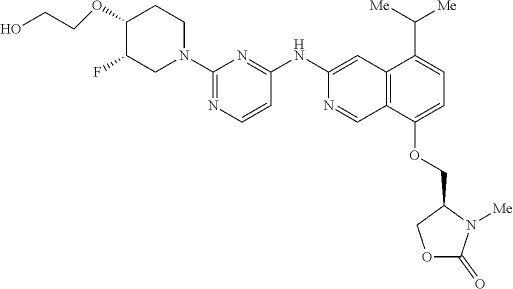 [SMILES: CC(C)c1ccc(OC[C@@H]2COC(=O)N2C)c2cnc(Nc3ccnc(N4CC[C@@H](OCCO)[C@@H](F)C4)n3)cc12] |
| 53 | 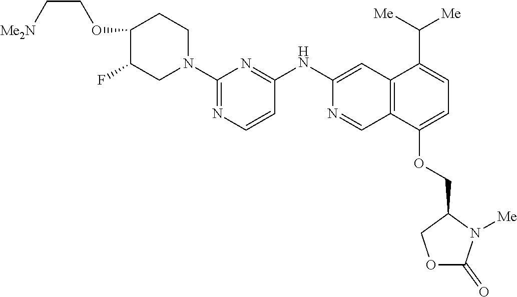 [SMILES: CC(C)c1ccc(OC[C@@H]2COC(=O)N2C)c2cnc(Nc3ccnc(N4CC[C@@H](OCCN(C)C)[C@@H](F)C4)n3)cc12] |
| 54 | 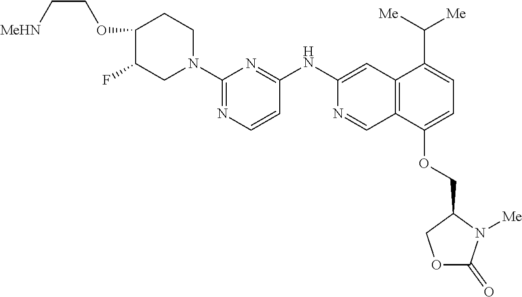 [SMILES: CNCCO[C@@H]1CCN(c2nccc(Nc3cc4c(C(C)C)ccc(OC[C@@H]5COC(=O)N5C)c4cn3)n2)C[C@@H]1F] |
| 55 | 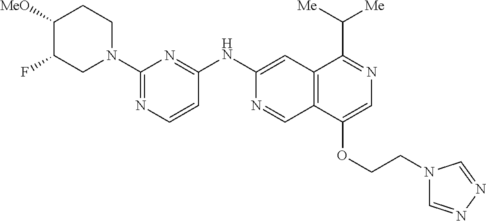 [SMILES: CO[C@@H]1CCN(c2nccc(Nc3cc4c(C(C)C)ncc(OCCn5cnnc5)c4cn3)n2)C[C@@H]1F] |
| 56 | 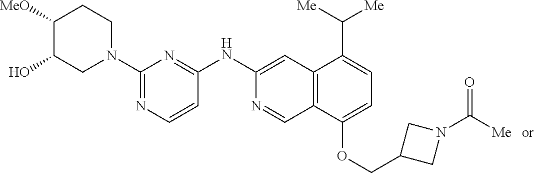 [SMILES: CO[C@@H]1CCN(c2nccc(Nc3cc4c(C(C)C)ccc(OCC5CN(C(C)=O)C5)c4cn3)n2)C[C@@H]1O] |
| | 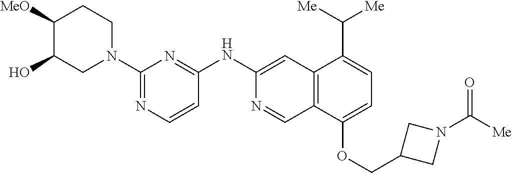 [SMILES: CO[C@H]1CCN(c2nccc(Nc3cc4c(C(C)C)ccc(OCC5CN(C(C)=O)C5)c4cn3)n2)C[C@H]1O] |
| 57 | 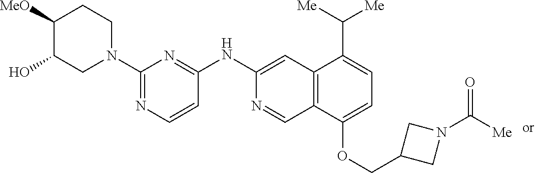 [SMILES: CO[C@H]1CCN(c2nccc(Nc3cc4c(C(C)C)ccc(OCC5CN(C(C)=O)C5)c4cn3)n2)C[C@@H]1O] |
| | 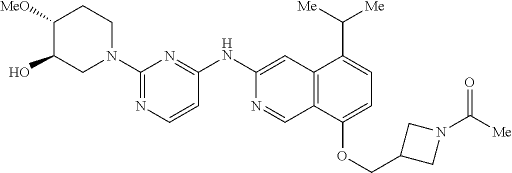 [SMILES: CO[C@@H]1CCN(c2nccc(Nc3cc4c(C(C)C)ccc(OCC5CN(C(C)=O)C5)c4cn3)n2)C[C@H]1O] |
| 58 | 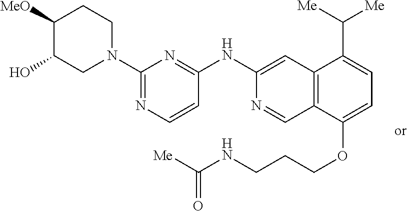 [SMILES: CO[C@H]1CCN(c2nccc(Nc3cc4c(C(C)C)ccc(OCCCNC(C)=O)c4cn3)n2)C[C@@H]1O] |
| | 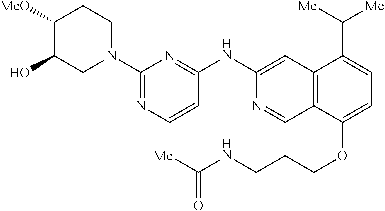 [SMILES: CO[C@@H]1CCN(c2nccc(Nc3cc4c(C(C)C)ccc(OCCCNC(C)=O)c4cn3)n2)C[C@H]1O] |
| 59 | 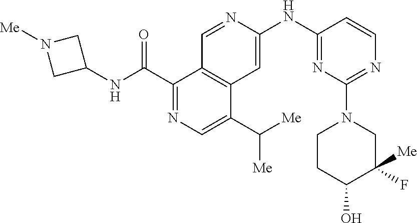 [SMILES: CC(C)c1cnc(C(=O)NC2CN(C)C2)c2cnc(Nc3ccnc(N4CC[C@@H](O)[C@@](C)(F)C4)n3)cc12] |
| 60 | 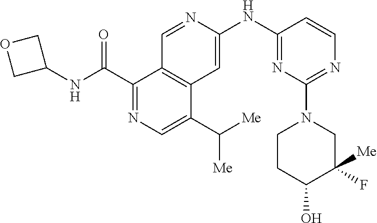 [SMILES: CC(C)c1cnc(C(=O)NC2COC2)c2cnc(Nc3ccnc(N4CC[C@@H](O)[C@@](C)(F)C4)n3)cc12] |
| 61 | 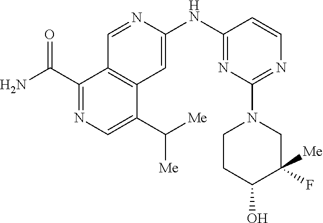 [SMILES: CC(C)c1cnc(C(N)=O)c2cnc(Nc3ccnc(N4CC[C@@H](O)[C@@](C)(F)C4)n3)cc12] |
| 62 | 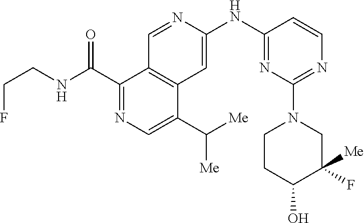 [SMILES: CC(C)c1cnc(C(=O)NCCF)c2cnc(Nc3ccnc(N4CC[C@@H](O)[C@@](C)(F)C4)n3)cc12] |
| 63 | 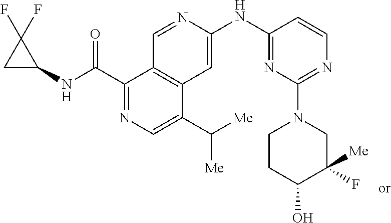 [SMILES: CC(C)c1cnc(C(=O)N[C@H]2CC2(F)F)c2cnc(Nc3ccnc(N4CC[C@@H](O)[C@@](C)(F)C4)n3)cc12] |
| | 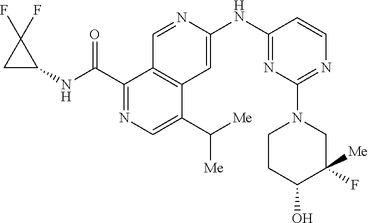 [SMILES: CC(C)c1cnc(C(=O)N[C@@H]2CC2(F)F)c2cnc(Nc3ccnc(N4CC[C@@H](O)[C@@](C)(F)C4)n3)cc12] |
| 64 | 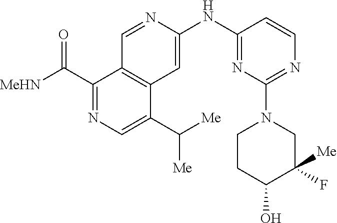 [SMILES: CNC(=O)c1ncc(C(C)C)c2cc(Nc3ccnc(N4CC[C@@H](O)[C@@](C)(F)C4)n3)ncc12] |
| 65 | 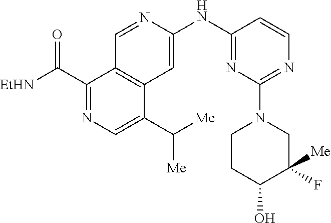 |
| 66 | 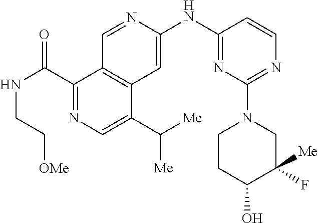 [SMILES: COCCNC(=O)c1ncc(C(C)C)c2cc(Nc3ccnc(N4CC[C@@H](O)[C@@](C)(F)C4)n3)ncc12] |
| 67 | 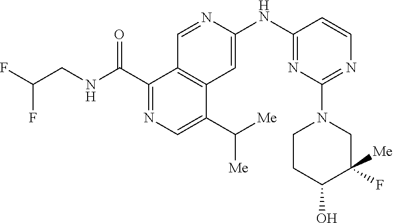 [SMILES: CC(C)c1cnc(C(=O)NCC(F)F)c2cnc(Nc3ccnc(N4CC[C@@H](O)[C@@](C)(F)C4)n3)cc12] |
| 68 | 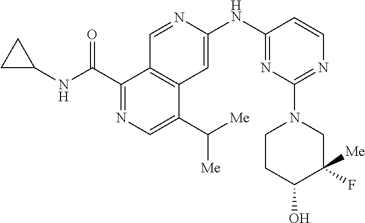 [SMILES: CC(C)c1cnc(C(=O)NC2CC2)c2cnc(Nc3ccnc(N4CC[C@@H](O)[C@@](C)(F)C4)n3)cc12] |
| 69 | 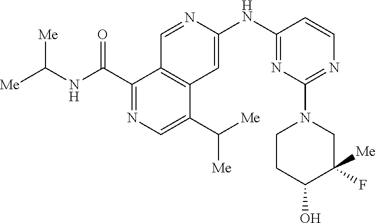 [SMILES: CC(C)NC(=O)c1ncc(C(C)C)c2cc(Nc3ccnc(N4CC[C@@H](O)[C@@](C)(F)C4)n3)ncc12] |
| 70 | 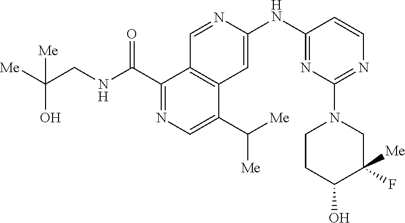 [SMILES: CC(C)c1cnc(C(=O)NCC(C)(C)O)c2cnc(Nc3ccnc(N4CC[C@@H](O)[C@@](C)(F)C4)n3)cc12] |
| 71 | 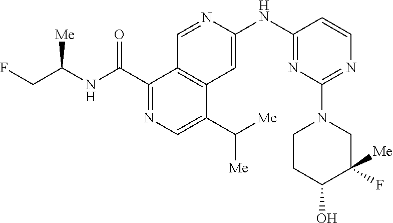 [SMILES: CC(C)c1cnc(C(=O)N[C@H](C)CF)c2cnc(Nc3ccnc(N4CC[C@@H](O)[C@@](C)(F)C4)n3)cc12] |
| 72 | 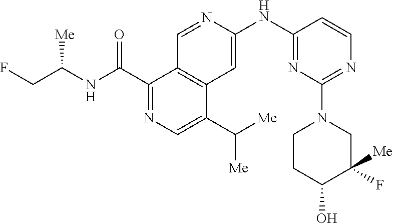 [SMILES: CC(C)c1cnc(C(=O)N[C@@H](C)CF)c2cnc(Nc3ccnc(N4CC[C@@H](O)[C@@](C)(F)C4)n3)cc12] |
| 73 | 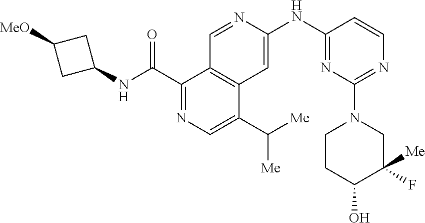 [SMILES: CO[C@H]1C[C@@H](NC(=O)c2ncc(C(C)C)c3cc(Nc4ccnc(N5CC[C@@H](O)[C@@](C)(F)C5)n4)ncc23)C1] |
| 74 | 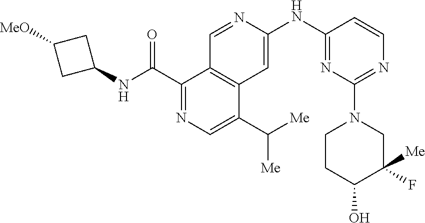 [SMILES: CO[C@H]1C[C@H](NC(=O)c2ncc(C(C)C)c3cc(Nc4ccnc(N5CC[C@@H](O)[C@@](C)(F)C5)n4)ncc23)C1] |
| 75 | 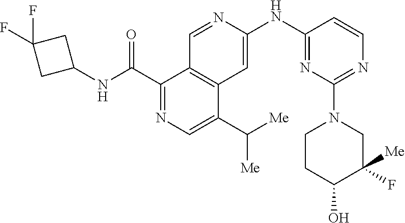 [SMILES: CC(C)c1cnc(C(=O)NC2CC(F)(F)C2)c2cnc(Nc3ccnc(N4CC[C@@H](O)[C@@](C)(F)C4)n3)cc12] |
| 76 | 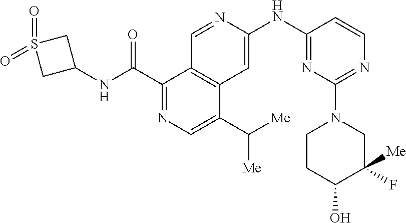 [SMILES: CC(C)c1cnc(C(=O)NC2CS(=O)(=O)C2)c2cnc(Nc3ccnc(N4CC[C@@H](O)[C@@](C)(F)C4)n3)cc12] |
| 77 | 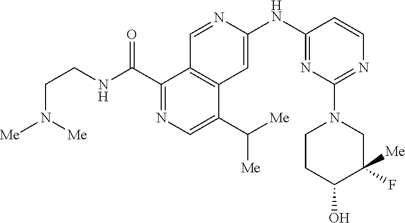 [SMILES: CC(C)c1cnc(C(=O)NCCN(C)C)c2cnc(Nc3ccnc(N4CC[C@@H](O)[C@@](C)(F)C4)n3)cc12] |
| 78 | 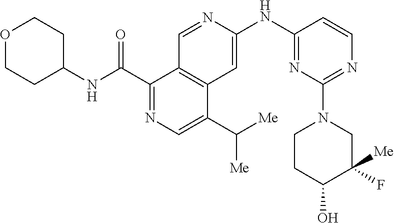 [SMILES: CC(C)c1cnc(C(=O)NC2CCOCC2)c2cnc(Nc3ccnc(N4CC[C@@H](O)[C@@](C)(F)C4)n3)cc12] |
| 79 | 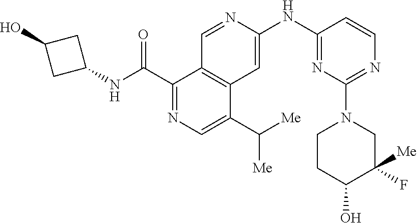 [SMILES: CC(C)c1cnc(C(=O)N[C@H]2C[C@H](O)C2)c2cnc(Nc3ccnc(N4CC[C@@H](O)[C@@](C)(F)C4)n3)cc12] |
| 80 | 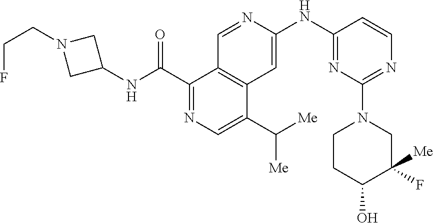 [SMILES: CC(C)c1cnc(C(=O)NC2CN(CCF)C2)c2cnc(Nc3ccnc(N4CC[C@@H](O)[C@@](C)(F)C4)n3)cc12] |
| 81 | 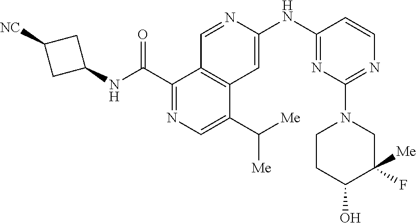 [SMILES: CC(C)c1cnc(C(=O)N[C@H]2C[C@@H](C#N)C2)c2cnc(Nc3ccnc(N4CC[C@@H](O)[C@@](C)(F)C4)n3)cc12] |
| 82 | 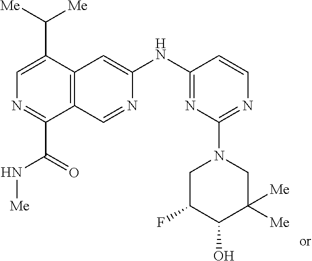 [SMILES: CNC(=O)c1ncc(C(C)C)c2cc(Nc3ccnc(N4C[C@@H](F)[C@@H](O)C(C)(C)C4)n3)ncc12] |
| | 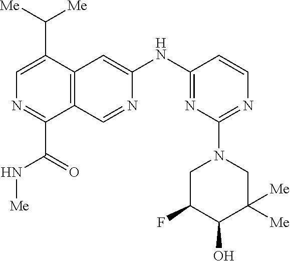 [SMILES: CNC(=O)c1ncc(C(C)C)c2cc(Nc3ccnc(N4C[C@H](F)[C@H](O)C(C)(C)C4)n3)ncc12] |
| 83 | 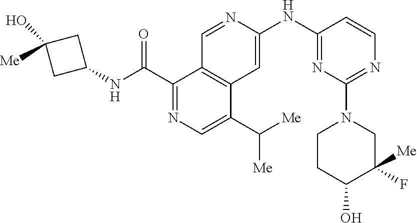 [SMILES: CC(C)c1cnc(C(=O)N[C@H]2C[C@@](C)(O)C2)c2cnc(Nc3ccnc(N4CC[C@@H](O)[C@@](C)(F)C4)n3)cc12] |
| 84 | 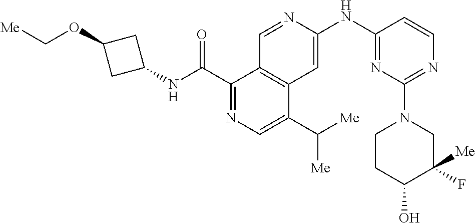 [SMILES: CCO[C@H]1C[C@H](NC(=O)c2ncc(C(C)C)c3cc(Nc4ccnc(N5CC[C@@H](O)[C@@](C)(F)C5)n4)ncc23)C1] |
| 85 | 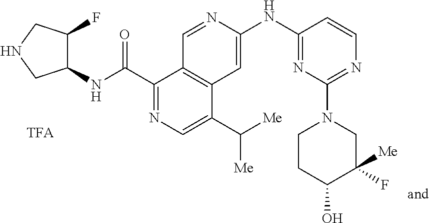 [SMILES: CC(C)c1cnc(C(=O)N[C@H]2CNC[C@H]2F)c2cnc(Nc3ccnc(N4CC[C@@H](O)[C@@](C)(F)C4)n3)cc12] |
| | 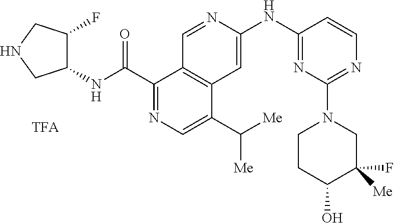 [SMILES: CC(C)c1cnc(C(=O)N[C@@H]2CNC[C@@H]2F)c2cnc(Nc3ccnc(N4CC[C@@H](O)[C@@](C)(F)C4)n3)cc12] |
| 86 | 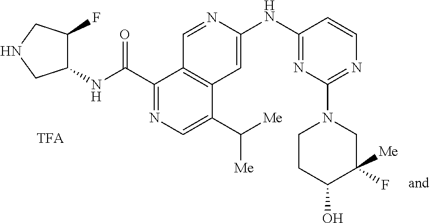 [SMILES: CC(C)c1cnc(C(=O)N[C@@H]2CNC[C@H]2F)c2cnc(Nc3ccnc(N4CC[C@@H](O)[C@@](C)(F)C4)n3)cc12] |
| | 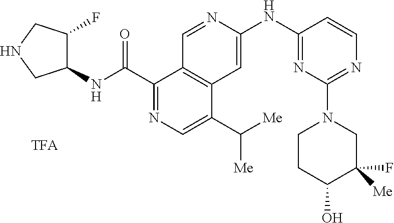 [SMILES: CC(C)c1cnc(C(=O)N[C@H]2CNC[C@@H]2F)c2cnc(Nc3ccnc(N4CC[C@@H](O)[C@@](C)(F)C4)n3)cc12] |
| 87 | 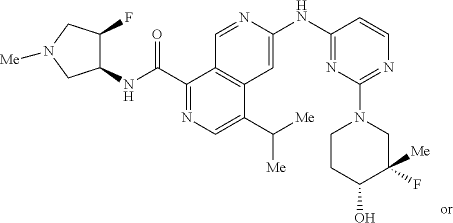 [SMILES: CC(C)c1cnc(C(=O)N[C@H]2CN(C)C[C@H]2F)c2cnc(Nc3ccnc(N4CC[C@@H](O)[C@@](C)(F)C4)n3)cc12] |
| | 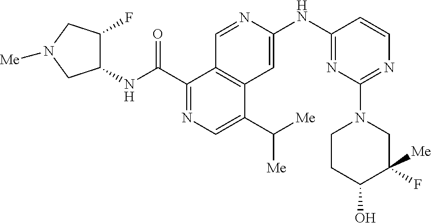 [SMILES: CC(C)c1cnc(C(=O)N[C@@H]2CN(C)C[C@@H]2F)c2cnc(Nc3ccnc(N4CC[C@@H](O)[C@@](C)(F)C4)n3)cc12] |
| 88 | 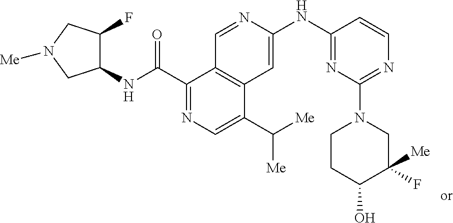 [SMILES: CC(C)c1cnc(C(=O)N[C@H]2CN(C)C[C@H]2F)c2cnc(Nc3ccnc(N4CC[C@@H](O)[C@@](C)(F)C4)n3)cc12] |
| | 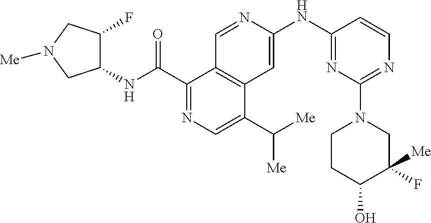 [SMILES: CC(C)c1cnc(C(=O)N[C@@H]2CN(C)C[C@@H]2F)c2cnc(Nc3ccnc(N4CC[C@@H](O)[C@@](C)(F)C4)n3)cc12] |
| 89 | 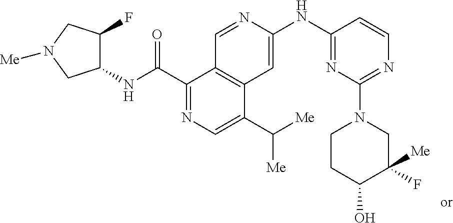 [SMILES: CC(C)c1cnc(C(=O)N[C@@H]2CN(C)C[C@H]2F)c2cnc(Nc3ccnc(N4CC[C@@H](O)[C@@](C)(F)C4)n3)cc12] |
| | 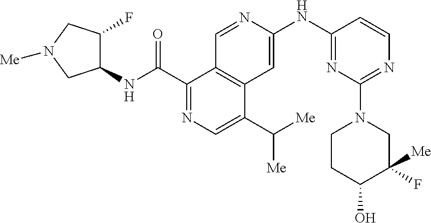 [SMILES: CC(C)c1cnc(C(=O)N[C@H]2CN(C)C[C@@H]2F)c2cnc(Nc3ccnc(N4CC[C@@H](O)[C@@](C)(F)C4)n3)cc12] |
| 90 | 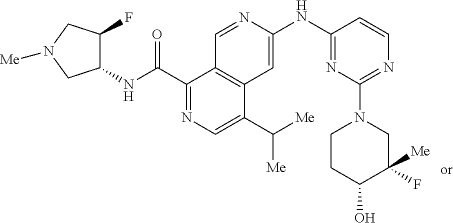 [SMILES: CC(C)c1cnc(C(=O)N[C@@H]2CN(C)C[C@H]2F)c2cnc(Nc3ccnc(N4CC[C@@H](O)[C@@](C)(F)C4)n3)cc12] |
| | 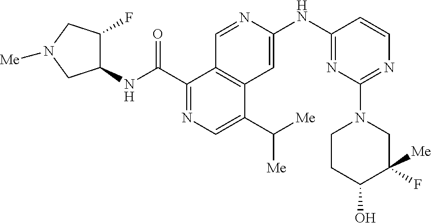 [SMILES: CC(C)c1cnc(C(=O)N[C@H]2CN(C)C[C@@H]2F)c2cnc(Nc3ccnc(N4CC[C@@H](O)[C@@](C)(F)C4)n3)cc12] |
| 91 | 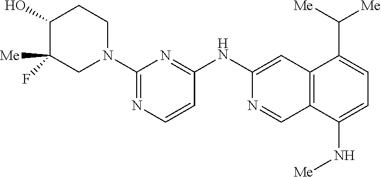 [SMILES: CNc1ccc(C(C)C)c2cc(Nc3ccnc(N4CC[C@@H](O)[C@@](C)(F)C4)n3)ncc12] |
| 92 | 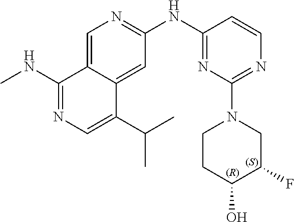 |
| 93 | 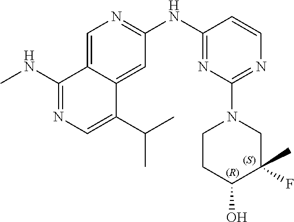 |
| 94 | 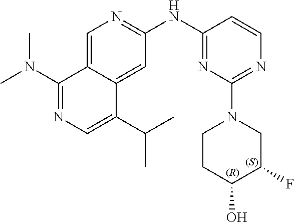 |
| 95 | 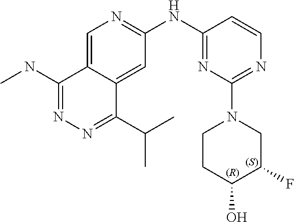 |

### Biological Example 1. Biochemical EGFR Inhibition Assays

Inhibitory effects of the compounds of the disclosure were measured in biochemical assays that measure the phosphorylation activity of EGFR enzyme phosphorylates 2.5 micromolar 5-FAM-EEPLYWSFPAKKK-CONH₂ peptide substrate (FL-Peptide 22, PerkinElmer, 760366) in the presence of adenosine-5′-triphosphate (ATP) and varying concentrations of the test compound in 100 mM 2-[4-(2-hydroxyethyl)piperazin-1-yl] ethanesulfonic acid (HEPES), pH 7.5, 10 mM MgCl₂, 0.015% Brij-35, 1 mM dithiothreitol (DTT), 1.0% dimehylsulfoxide (DMSO). Assays were performed at 1.0 mM ATP or at ATP Km of the EGFR enzymes. Reactions proceeded until between 10% to 20% total peptides were phosphorylated at room temperature (25° C.) and were terminated with 35 mM 2,2′,2″,2′″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following enzyme forms of EGFR were examples that were used in these assays:
EGFR WT (SignalChem, E10-112G)
EGFR (L858R T790M C797S) (SignalChem, E10-122VG)
EGFR (d746-750) T790M C797S (SignalChem, E10-122UG)
EGFR L858R (SignalChem, E10-122BG)
EGFR (d746-750) (SignalChem, E10-122JG)

### Biological Example 2. NCI-H1975 pEGFR AlphaLISA Assays

Inhibitory effects of the compounds of the disclosure were evaluated in cellular assays that measure level of intracellular phosphorylation of EGFR in NCI-H1975 cell line that harbors the EGFR L858R T790M mutations (ATCC, CRL-5908) using AlphaLISA sureFire ultra p-EGFR (Tyr1068) assay kit (PerkinElmer, ALSU-PEGFR-A50K). The NCI-H1975 cells were seeded at 12.5K/well in 22 μL into 384 well opti plate (PerkinElmer, 6007299) and adhering overnight at 37C/5% CO₂. On the next day, the test compounds and DMSO control were added into H1975 cell plate followed by incubation at 37C/5% CO₂ for 4-5 hours. The cells were then spin down in the 384-well plate and lysed with 10 μL of 1× AlphaLISA lysis buffer followed by shaking at 600 rpm for 10 minutes at room temperature. After that, 5 μL of an acceptor bead mix was added to each well followed by incubation at room temperature for 1.5-2 h in dark. Then 5 μL of a donor bead mix was added to each well followed by overnight incubation at room temperature in dark. On the next day, the plate was read at a compatible plate reader to obtain pEGFR signal. Percent of pEGFR inhibition was plotted against log concentration of compounds to generate IC₅₀ values.

Biological assay data of the test compounds are provided in Table 2 below. For inhibitory activity against EGFR LRTMCS mutant, the following designations are used: ≤15 nM=A; >15-20 nM=B; >20-30 nM=C; >30-100 nM=D and >100=E. For inhibition of phosphorylation of mutant EGFR in cells: ≤10 nM=A; >10-20 nM=B; >20-30 nM=C; >30-50 nM=D; and >50 nM=E. All compounds of Formula (I) had inhibitory activity against EGFR LRTMCS mutant of <10 micromolar except the compounds in Table 3.

TABLE 2
Tabularized Data:

| | | Ext pEGFR H1975 |
|---|---|---|
| | LRTMCS (PH) | GMean IC50 (nM) |
| | IC50 (nM) | Protocol: @Pharmaron |
| Example | @ 1 mM ATP (Num) | AlphaLISA (Num) |
| 1 | A | D |
| 2 | A | B |
| 3 | B | E |
| 4 | D | E |
| 5 | A | C |
| 6 | C | E |
| 7 | B | D |
| 8 | A | A |
| 9 | A | E |
| 10 | A | D |
| 11 | C | E |
| 12 | A | E |
| 13 | A | D |
| 14 | C | E |
| 15 | A | D |
| 16 | C | E |
| 17 | A | E |
| 18 | A | C |
| 19 | A | B |
| 20 | A | B |
| 21 | A | A |
| 22 | B | E |
| 23 | B | D |
| 24 | A | A |
| 25 | B | E |
| 26 | B | E |
| 27 | A | E |
| 28 | A | C |
| 29 | A | C |
| 30 | A | E |
| 31 | A | E |
| 32 | A | E |
| 33 | B | D |
| 34 | B | E |
| 35 | B | D |
| 36 | B | E |
| 37 | B | D |
| 38 | B | E |
| 39 | B | E |
| 40 | A | E |
| 41 | A | C |
| 42 | C | E |
| 43 | B | E |
| 44 | B | E |
| 45 | B | E |
| 46 | B | E |
| 47 | B | E |
| 48 | B | E |
| 49 | B | E |
| 50 | C | E |
| 51 | C | E |
| 52 | C | E |
| 53 | B | E |
| 54 | B | E |
| 55 | B | E |
| 56 | A | D |
| 57 | B | E |
| 58 | A | C |
| 59 | A | D |
| 60 | B | D |
| 61 | C | E |
| 62 | A | E |
| 63 | B | E |
| 64 | B | E |
| 65 | B | E |
| 66 | C | E |
| 67 | A | E |
| 68 | B | E |
| 69 | C | E |
| 70 | D | E |
| 71 | C | E |
| 72 | B | E |
| 73 | A | C |
| 74 | A | C |
| 75 | A | D |
| 76 | A | D |
| 77 | A | D |
| 78 | B | D |
| 79 | A | D |
| 80 | A | C |
| 81 | A | C |
| 82 | B | E |
| 83 | A | D |
| 84 | A | C |
| 85 | A | B |
| 86 | C | E |
| 87 | A | B |
| 88 | A | B |
| 89 | B | E |
| 90 | A | B |
| 91 | A | C |
| 92 | D | E |
| 93 | A | B |
| 94 | D | E |
| 95 | E | E |

Additional compounds not disclosed herein were also tested in the assays described in Biological Examples 1 and 2 and all but three had inhibitory activity less than 10 micromolar for both assays. The three following compounds had inhibitory activity greater than 10 micromolar in Biological Assay 1.

TABLE 3

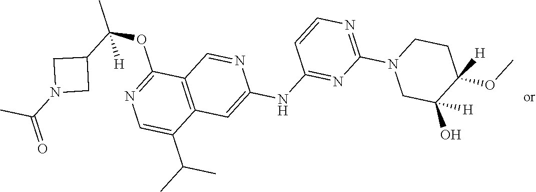 [SMILES: CO[C@H]1CCN(c2nccc(Nc3cc4c(C(C)C)cnc(O[C@H](C)C5CN(C(C)=O)C5)c4cn3)n2)C[C@@H]1O]

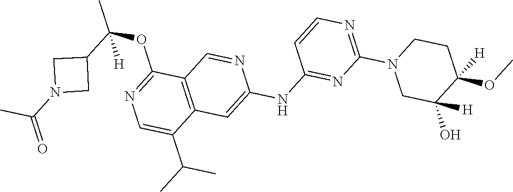 [SMILES: CO[C@@H]1CCN(c2nccc(Nc3cc4c(C(C)C)cnc(O[C@H](C)C5CN(C(C)=O)C5)c4cn3)n2)C[C@H]1O]

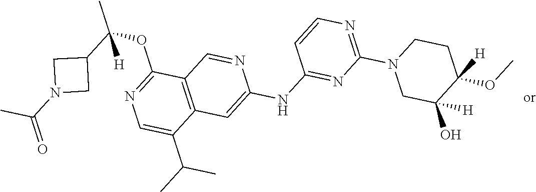 [SMILES: CO[C@H]1CCN(c2nccc(Nc3cc4c(C(C)C)cnc(O[C@@H](C)C5CN(C(C)=O)C5)c4cn3)n2)C[C@@H]1O]

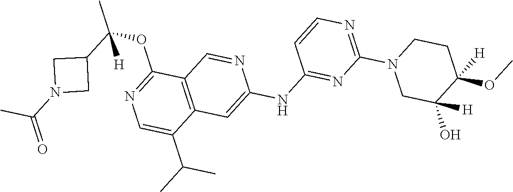 [SMILES: CO[C@@H]1CCN(c2nccc(Nc3cc4c(C(C)C)cnc(O[C@@H](C)C5CN(C(C)=O)C5)c4cn3)n2)C[C@H]1O]

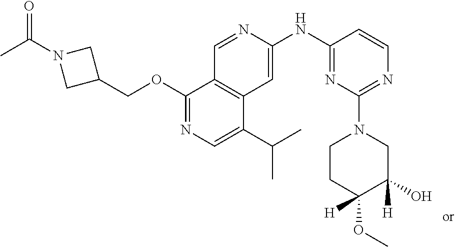 [SMILES: CO[C@@H]1CCN(c2nccc(Nc3cc4c(C(C)C)cnc(OCC5CN(C(C)=O)C5)c4cn3)n2)C[C@@H]1O]

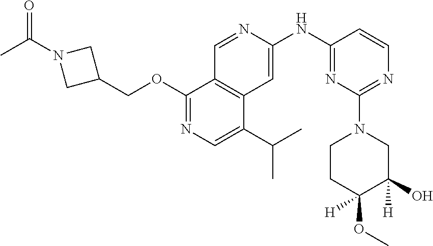 [SMILES: CO[C@H]1CCN(c2nccc(Nc3cc4c(C(C)C)cnc(OCC5CN(C(C)=O)C5)c4cn3)n2)C[C@H]1O]

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A compound of Formula (I) 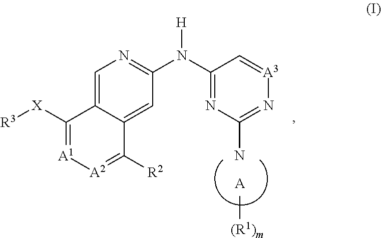
or a pharmaceutically acceptable salt thereof, wherein:
each A¹, A², and A³ is independently N or CR; wherein each R is independently H, halogen, or CH₃;
Ring A is 4-12 membered heterocyclyl;
each R¹ is independently halogen, CN, OH, NR^{a}R^{b}, C₁-C₄ alkyl, C₁-C₄ alkoxy, C₃-C₆ cycloalkyl, or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is H, halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H, or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4 to 12-membered heterocycyl, and 5 to 10 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl); or
R³ is C₃-C₆cycloalkyl optionally substituted with 1 to 3 groups selected from C₁-C₄alkyl, halogen, OR^{a}, CN, NR^{a}R^{b}, NHC(O)CH₃, and S(O)₂CH₃, or
R³ is 4 to 10 membered heterocyclyl or 5 to 6-membered heteroaryl, each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

2. The compound of claim 1 or a pharmaceutically acceptable salt thereof, wherein A³ is CR.

3. The compound of any one of claims 1-2 or a pharmaceutically acceptable salt thereof, wherein Ring A is a 5-6 membered heterocyclyl.

4. The compound of claim 3 or a pharmaceutically acceptable salt thereof, wherein R² is C₁-C₄ alkyl.

5. The compound of claim 1, wherein the compound is a compound of formula (II) 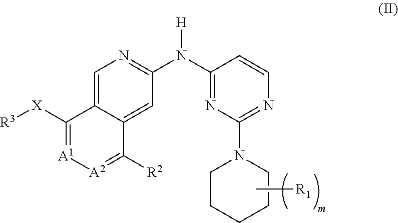
or a pharmaceutically acceptable salt thereof.

6. The compound of claim 5 or a pharmaceutically acceptable salt thereof, wherein:
each A¹, and A² is independently N or CR; wherein each R is independently H, halogen, or CH₃;
each R¹ is independently halogen, CN, OH, NR^{a}R^{b}, C₁-C₄ alkyl, C₁-C₄ alkoxy, C₃-C₆ cycloalkyl or —O—C₃-C₆ cycloalkyl, wherein the alkyl, alkoxy or cycloalkyl represented by R¹ is optionally substituted with 1 to 3 groups selected from deuterium, halogen, OH, NR^{a}R^{b}, C₁-C₂ alkyl, and C₁-C₂ alkoxy; and/or
m is 0, 1, 2, 3, 4, 5, or 6;
R² is halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H, or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-6-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6 membered heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

7. The compound of claim 6 or a pharmaceutically acceptable salt thereof, wherein X is O and R³ is C₁-C₄alkyl optionally substituted with a 4 to 6-member heterocyclyl, 5-member heteroaryl, or 5-member heterocyclyl fused to a 5-member heteroaryl, wherein each heteroaryl or heterocyclyl is each optionally substituted with 1-3 groups selected from halogen, C₁-C₄alkyl, ═O, and —C(O)CH₃.

8. The compound of claim 6 or a pharmaceutically acceptable salt thereof, wherein X is NH and R³ is C₁-C₄alkyl optionally substituted with S(O)₂CH₃ or a 4 to 6-member heterocyclyl optionally substituted S(O)₂CH₃.

9. The compound of claim 6 or a pharmaceutically acceptable salt thereof, wherein X is —C(O)—NH—, and R³ is H, or C₁-C₄alkyl, wherein the alkyl is optionally substituted with 1 to 3 groups selected from halogen, OR^{a}, NR^{a}R^{b}.

10. The compound of claim 6 or a pharmaceutically acceptable salt thereof, wherein X is —C(O)—NH—, and R³ is C₃-C₆cycloalkyl optionally substituted with 1 to 3 groups selected from C₁-C₄alkyl, halogen, OR^{a}, and CN.

11. The compound of claim 6 or a pharmaceutically acceptable salt thereof, wherein X is —C(O)—NH—, and R³ is 4 to 6 membered heterocyclyl optionally substituted with 1 to 3 groups selected from halogen, and C₁-C₄ alkyl, wherein the alkyl is optionally substituted with 1 to 3 halo.

12. The compound of claim 1 or a pharmaceutically acceptable salt thereof, wherein the compound is a compound of formula (IIa) 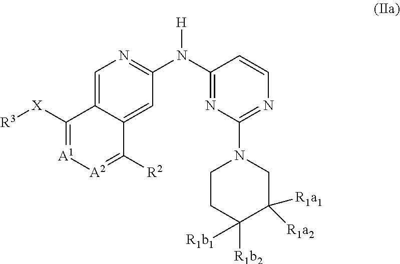
or a pharmaceutically acceptable salt thereof;
A¹ is N or CR and A² is CR; or A¹ is CR and A² is N, wherein each R is independently H, halogen, or CH₃;
each R₁ₐ₁, R₁ₐ₂, R_{1b1}, and R_{1b2} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R₁ₐ₁, R₁ₐ₂, R_{1b1}, and R_{1b2} is optionally substituted with 1 to 3 groups selected from deuterium, halogen, OH, NR^{a}R^{b}, C₁-C₂ alkyl, and C₁-C₂ alkoxy, for example, R₁ₐ₁ is H, F or OH and R₁ₐ₂ is H, F or CH₃; R_{1b2} is OH, OCH₃₀r OCD₃, and R_{1b1} is H or CH₃;
R² is halogen, C₁-C₄ alkyl, C₁-C₄ alkoxy, or C₃-C₆ cycloalkyl, wherein the alkyl, alkoxy or cycloalkyl represented by R² is optionally substituted with 1 to 3 groups selected from halogen;
X is O, NH, or —C(O)—NH—, wherein the R³ is attached to the NH— of —C(O)NH—;
R³ is H or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-5-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, and S(O)₂CH₃;
R³ is 4 to 6 member heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

13. The compound of claim 1, wherein the compound is a compound of formula (IIb) 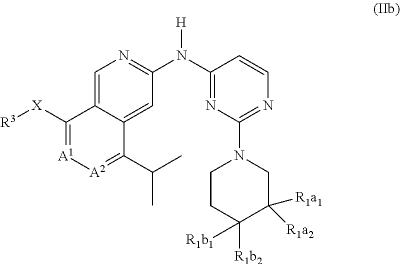
or a pharmaceutically acceptable salt thereof, wherein:
A¹ is N or CR and A² is CR or A¹ is CR and A² is N, wherein each R is independently H, halogen, or CH₃;
each R₁ₐ₁, R₁ₐ₂, R_{1b1}, and R_{1b2} is independently H, halogen, CN, OH, NR^{a}R^{b}, C₁-C₄ alkyl, C₁-C₄ alkoxy, C₃-C₆ cycloalkyl or —O—C₃-C₆ cycloalkyl; wherein the alkyl, alkoxy or cycloalkyl represented by R₁ₐ₁, R_{1a 2}, R_{1b1}, and R_{1b2} is optionally substituted with 1 to 3 groups selected from deuterium, halogen, OH, NR^{a}R^{b}, C₁-C₂ alkyl, and C₁-C₂ alkoxy;
X is O, NH, or —C(O)—NH—, wherein the R³ is attached to the NH— of —C(O)NH—, for example, R₁ₐ₁ is H, F or OH and R₁ₐ₂ is H, F or CH₃; R_{1b2} is OH, OCH₃ or OCD₃, and R_{1b1} is H or CH₃;
R³ is H or C₁-C₄alkyl, wherein the alkyl is optionally substituted with 1 to 3 groups selected from halogen, OR^{a}, CN, NR^{a}R^{b}, NHC(O)CH₃, S(O)₂CH₃, 4-6-membered heterocycyl, and 5 membered heteroaryl, wherein the heterocycyl and the heteroaryl are optionally substituted with 1 to 3 groups selected from halogen, C₁-C₄alkyl, —C(O)CH₃, ═O and —SO₂—(C₁-C₄alkyl), or the heterocyclyl is fused to a 5-member heteroaryl; or
R³ is C₃-C₆cycloalkyl optionally substituted with 1 to 3 groups selected from C₁-C₄alkyl, halogen, OR^{a}, CN, NR^{a}R^{b}, NHC(O)CH₃, S(O)₂CH₃;
R³ is 4 to 6-member heterocyclyl or 5 to 6 membered heteroaryl each optionally substituted with 1 to 3 groups selected from halogen and C₁-C₄ alkyl, wherein the alkyl is optionally substituted with 1 to 3 halo; and
each R^{a} and R^{b} is independently H or C₁-C₄ alkyl.

14. A pharmaceutical composition comprising a pharmaceutically acceptable carrier and a compound of any one of claims 1-13, or a pharmaceutically acceptable salt thereof.

15. A method of treating a cancer, comprising administering a subject in need thereof an effective amount of a compound of any of claims 1-14, or a pharmaceutically acceptable salt thereof, or a pharmaceutical composition of claim 14.

16. The method of claim 15, wherein the cancer is non-small cell lung cancer.

17. The method of claim 14 or 15, wherein the cancer in the subject in need thereof has metastasized.

18. The method of any one of claims 15-17, wherein the cancer is characterized by: i) epidermal growth factor receptor EGFR L858R mutation and/or exon 19 deletion; and ii) T790M mutation.

19. The method of claim 18, wherein the cancer is further characterized by epidermal growth factor receptor (EGFR) C797S mutation.

20. The method of any one of claims 15-19, further comprises administering the subject in need thereof an effective amount of afatinib, osimertinib, erlotinib, or gefitinib.

21. A method of inhibiting epidermal growth factor receptor (EGFR), comprising administering to a subject in need thereof an effective amount of a compound of any of claims 1-13, or a pharmaceutically acceptable salt thereof, or a pharmaceutical composition of claim 14.
